(19) United States
(12) Patent Application Publication
(10) Pub. No.: US 2018/0237441 A1
(43) Pub. Date: Aug. 23, 2018

(54) Chemical Compounds

(71) Applicant: GlaxoSmithKline Intellectual Property (No.2) Limited, Brentford, Middlesex (GB)

(72) Inventors: Jeffrey Michael AXTEN, Stevenage (GB); Alain Claude-Marie DAUGAN, London (GB); Nicolas Eric FAUCHER, Stevenage (GB); Raghava Reddy KETHIRI, London (GB); Rajendra KRISTAM, Stevenage (GB); Chandregowda VENKATESHAPPA, Stevenage (GB)

(21) Appl. No.: 15/759,888

(22) PCT Filed: Sep. 15, 2016
(86) PCT No.: PCT/IB2016/055504

§ 371 Date: Mar. 14, 2018

Related U.S. Application Data
(60) Provisional application No. 62/218,740, filed on Sep. 15, 2015.
(60) Provisional application No. 62/318,321, filed on Apr. 5, 2016.

(51) Int. Cl. C07D 487/04; A61K 31/519; A61K 45/06; C07D 495/04

(52) U.S. Cl. CPC C07D 487/04; C07D 495/04; A61K 45/06; A61K 31/519

(57) ABSTRACT

The invention is directed to substituted pyrrolidinone and imidazolidinone derivatives. Specifically, the invention is directed to compounds according to Formula I:

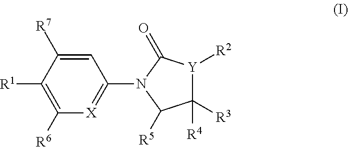

wherein R¹, R², R³, R⁴, R⁵, R⁶, R⁷, X and Y are as defined herein.

The compounds of the invention are inhibitors of PERK and can be useful in the treatment of cancer, pre-cancerous syndromes and diseases/injuries associated with activated unfolded protein response pathways, such as Alzheimer's disease, spinal cord injury, traumatic brain injury, ischemic stroke, stroke, Parkinson's disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation. Accordingly, the invention is further directed to pharmaceutical compositions comprising a compound of the invention. The invention is still further directed to methods of inhibiting PERK activity and treatment of disorders associated therewith using a compound of the invention or a pharmaceutical composition comprising a compound of the invention.

### FIELD OF THE INVENTION

The present invention relates to substituted pyrrolidinone and imidazolidinone derivatives that are inhibitors of the activity of the protein kinase R (PKR)-like ER kinase, PERK. The present invention also relates to pharmaceutical compositions comprising such compounds and methods of using such compounds in the treatment of cancer, pre-cancerous syndromes and diseases/injuries associated with activated unfolded protein response pathways, such as Alzheimer's disease, neuropathic pain, spinal cord injury, traumatic brain injury, ischemic stroke, stroke, Parkinson's disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation.

### BACKGROUND OF THE INVENTION

The unfolded protein response (UPR) is a signal transduction pathway that allows cells to survive stress caused by the presence of misfolded or unfolded proteins or protein aggregates (Walter and Ron, 2011), (Hetz, 2012). Environmental stresses that perturb protein folding and maturation in the endoplasmic reticulum (ER) also can lead to activation of the UPR (Feldman et al., 2005), (Koumenis and Wouters, 2006). UPR activating stress stimuli include hypoxia, disruption of protein glycosylation (glucose deprivation), depletion of luminal ER calcium, or changes in ER redox status, among others (Ma and Hendershot, 2004), (Feldman et al., 2005). These perturbations result in disruption of ER redox homeostasis and the accumulation of unfolded or mis-folded proteins in the ER. Cellular responses include transcriptional reprogramming to increase the level of chaperone proteins to enhance protein re-folding, degradation of the mis-folded proteins, and translational arrest to decrease the burden of client proteins entering the ER (Ron, D. 2002), (Harding et al., 2002). These pathways also regulate cell survival by modulating apoptosis (Ma and Hendershot, 2004), (Feldman et al., 2005), and autophagy (Rouschop et al. 2010), and can trigger cell death under conditions of prolonged ER stress (Woehlbier and Hetz, 2011).

Three ER membrane proteins have been identified as primary effectors of the UPR: protein kinase R (PKR)-like ER kinase [PERK, also known as eukaryotic initiation factor 2A kinase 3 (EIF2AK3), pancreatic ER kinase, or pancreatic eIF2α kinase (PEK)], inositol-requiring gene 1 α/β (IRE1), and activating transcription factor 6 (ATF6) (Ma and Hendershot, 2004), (Hetz, 2012). Under normal conditions these proteins are held in the inactive state through binding of the ER chaperone GRP78 (BiP) to their luminal sensor domain. Accumulation of unfolded proteins in the ER leads to release of GRP78 from these sensors resulting in activation of these UPR effectors (Ma et al., 2002), (Hetz, 2012).

PERK is a type I ER membrane protein containing a stress-sensing domain facing the ER lumen, a transmembrane segment, and a cytosolic kinase domain (Shi et al., 1998), (Harding et al., 1999), (Sood et al., 2000). Release of GRP78 from the stress-sensing domain of PERK results in oligomerization and autophosphorylation at multiple serine, threonine and tyrosine residues (Ma et al., 2001), (Su et al., 2008). Phenotypes of PERK knockout mice include diabetes, due to loss of pancreatic islet cells, skeletal abnormalities, and growth retardation (Harding et al., 2001), (Zhang et al., 2006), (lida et al., 2007). These features are similar to those seen in patients with Wolcott-Rallison syndrome, who carry germline mutations in the PERK gene (Julier and Nicolino, 2010). The major substrate for PERK is the eukaryotic initiation factor 2a (eIF2a), which PERK phosphorylates at serine-51 (Marciniak et al., 2006) in response to ER stress or treatment with pharmacological inducers of ER stress such as thapsigargin and tunicamycin. This site is also phosphorylated by other EIF2AK family members [(general control non-derepressed 2 (GCN2), PKR, and heme-regulated kinase (HRI)] in response to different stimuli.

Phosphorylation of eIF2α converts it to an inhibitor of the guanine nucleotide exchange factor (GEF) eIF2B which is required for efficient turnover of GDP for GTP in the eIF2 protein synthesis complex. As a result, the inhibition of eIF2B by P-eIF2α causes a general decrease in translation initiation and thus a reduction in global protein synthesis (Harding et al. 2002). Paradoxically, translation of specific mRNAs is enhanced when the UPR is activated and eIF2α is phosphorylated. For example, the transcription factor ATF4 has 5′-upstream open reading frames (uORFs) that normally represses ATF4 synthesis during normal global protein synthesis. However, when PERK is activated under stress and P-eIF2α inhibits eIF2B, the lower levels of ternary translation complex allows for selective enhanced translation of ATF4 (Jackson et al. 2010). Therefore, when ER stress ensues, PERK activation causes an increase in ATF4 translation, which transcriptionally upregulates downstream target genes such as CHOP (transcription factor C/EBP homologous protein). This transcriptional reprogramming modulates cell survival pathways and can lead to the induction of pro-apoptotic genes.

The activation of PERK and the UPR is associated with human neurodegenerative conditions such as Alzheimer's disease, Parkinson's disease, Huntington's disease, amyotrophic lateral sclerosis (ALS), progressive supranuclear palsy (PSP), dementias, and prion diseases including Creutzfeldt-Jakob Disease (CJD), (Doyle et al. 2011), (Paschen 2004), (Salminen et al. 2009), (Stutzbach et al. 2013). The common hallmark of all these diseases is the presence of malformed/misfolded or aggregated protein deposits (e.g tau tangles, Lewy bodies, α-synuclein, Aβ plaques, mutant prion proteins) believed to contribute to the underlying disease pathophysiology, neuron loss, and cognitive decline (Prusiner, 2012), (Doyle et al. 2011). The fate of a cell (e.g a neuron) enduring unfolded or misfolded protein stress is under control of PERK. A cell enduring ER stress may restore proteostasis and return to normal, or if the stress is insurmountable, sustained PERK activation may lead to cell death through ATF4/CHOP driven autophagy coupled with the inability to synthesize vital proteins because of the persistent translational repression. Activated PERK and associated biological markers of PERK activation are detected in post-mortem brain tissue of Alzheimer's disease patients and in human prion disease (Ho et al. 2012), (Hoozemans et al, 2009) (Unterberger et al. 2006). Furthermore, P-eIF2α (the product of PERK activation) correlates with levels of BACE1 in post-mortem brain tissue of Alzheimer's disease patients (O'Connor et al. 2008). Recently, the small molecule PERK inhibitor GSK2606414 was shown to provide a neuroprotective effect and prevent clinical signs of disease in prion infected mice (Moreno et al. 2013), consistent with previous results derived from genetic manipulation of the UPR/PERK/eIF2α pathway (Moreno et al. 2012). Involvement of the pathway in ALS (Kanekura et. al., 2009 and Nassif et. al. 2010), spinal cord injury (Ohri et al. 2011) and traumatic brain injury (Tajiri et al. 2004) is also reported. Taken together these data suggest that the UPR and PERK represent a promising node of drug intervention as a means to halt or reverse the clinical progression and associated cognitive impairments of a wide range of neurodegenerative diseases.

Tumor cells experience episodes of hypoxia and nutrient deprivation during their growth due to inadequate blood supply and aberrant blood vessel function (Brown and Wilson, 2004), (Blais and Bell, 2006). Thus, they are likely to be dependent on active UPR signaling to facilitate their growth. Consistent with this, mouse fibroblasts derived from PERK−/−, XBP1−/−, and ATF4−/− mice, and fibroblasts expressing mutant eIF2α show reduced clonogenic growth and increased apoptosis under hypoxic conditions in vitro and grow at substantially reduced rates when implanted as tumors in nude mice (Koumenis et al., 2002), (Romero-Ramirez et al., 2004), (Bi et al., 2005). Human tumor cell lines carrying a dominant negative PERK that lacks kinase activity also showed increased apoptosis in vitro under hypoxia and impaired tumor growth in vivo (Bi et al., 2005). In these studies, activation of the UPR was observed in regions within the tumor that coincided with hypoxic areas. These areas exhibited higher rates of apoptosis compared to tumors with intact UPR signaling. Further evidence supporting the role of PERK in promoting tumor growth is the observation that the number, size, and vascularity of insulinomas arising in transgenic mice expressing the SV40-T antigen in the insulin-secreting beta cells, was profoundly reduced in PERK^{-/-} mice compared to wild-type control (Gupta et al., 2009). Activation of the UPR has also been observed in clinical specimens. Human tumors, including those derived from cervical carcinomas, glioblastomas (Bi et al., 2005), lung cancers (Jorgensen et al., 2008) and breast cancers (Ameri et al., 2004), (Davies et al., 2008) show elevated levels of proteins involved in UPR, compared to normal tissues. Therefore, inhibiting the unfolded protein response with compounds that block the activity of PERK and other components of the UPR is expected to have utility as anticancer agents. Recently, this hypothesis was supported by two small molecule inhibitors of PERK that were shown to inhibit the growth of human tumor xenografts in mice (Axten et al. 2012 and Atkins et al. 2013).

Loss of endoplasmic reticulum homeostasis and accumulation of misfolded proteins can contribute to a number of disease states (Wek and Cavener 2007), (Zhang and Kaufman 2006). Inhibitors of PERK may be therapeutically useful for the treatment of a variety of human diseases such as Alzheimer's disease and frontotemporal dementias, Parkinson's disease, Huntington's disease, amyotrophic lateral sclerosis (ALS), progressive supranuclear palsy (PSP), and other tauopathies such chronic traumatic encephalopathy (CTE) (Nijholt, D. A., et al. 2012), (Lucke-Wold, B. P., et al. 2016), spinal cord injury, traumatic brain injury, stroke, Creutzfeldt-Jakob Disease (CJD) and related prion diseases, such as fatal familial insomnia (FFI), Gerstmann-Sträussler-Scheinker Syndrome, and vanishing white matter (VWM) disease. Inhibitors of PERK may also be useful for effective treatment of cancers, particularly those derived from secretory cell types, such as pancreatic and neuroendocrine cancers, multiple myeloma, or for use in combination as a chemosensitizer to enhance tumor cell killing. A PERK inhibitor may also be useful for myocardial infarction, cardiovascular disease, atherosclerosis (McAlpine et al., 2010, Civelek et al. 2009, Liu and Dudley 2016), arrhythmias, and kidney disease (Dickhout et al., 2011, Cybulsky, A. V., et al. 2005). A PERK inhibitor may also be useful in stem cell or organ transplantation to prevent damage to the organ and in the transportation of organs for transplantation (Inagi et al., 2014), (Cunard, 2015), (Dickhout et al., 2011), (van Galen, P., et al. (2014). A PERK inhibitor is expected to have diverse utility in the treatment of numerous diseases in which the underlying pathology and symptoms are associated with dysregulaton of the unfolded protein response.

### REFERENCES

Ameri, K., Lewis, C. E., Raida, M., Sowter, H., Hai, T., and Harris, A. L. (2004). Anoxic induction of ATF-4 through HIF-1-independent pathways of protein stabilization in human cancer cells, Blood 103, 1876-82.
Atkins, C.; Liu, Q.; Minthorn, E.; Zhang, S.; Figueroa, D. J.; Moss, K.; Stanley, T. B.; Sanders, B.; Goetz, A.; Gaul, N.; Choudhry, A. E.; Alsaid, H.; Jucker, B. M.; Axten, J. M.; Kumar, R. (2013) Characterization of a novel PERK kinase inhibitor with anti-tumor and anti-angiogenic activity. Cancer Res. 74, 1993-2002.
Axten, J. M.; Medina, J. R.; Feng, Y.; Shu, A.; Romeril, S. P.; Grant, S. W.; Li, W. H. H.; Heerding, D. A.; Minthorn, E.; Mencken, T.; Atkins, C.; Liu, Q.; Rabindran, S.; Kumar, R.; Hong, X.; Goetz, A.; Stanley, T.; Taylor, J. D.; Sigethy, S. D.; Tomberlin, G. H.; Hassell, A. M.; Kahler, K. M.; Shewchuk, L. M.; Gampe, R. T. (2012) Discovery of 7-methyl-5-(1-{[3-(trifluoromethyl)phenyl]acetyl}-2,3-dihydro-1H-indol-5-yl)-7H-pyrrolo[2,3-d]pyrimidin-4-amine (GSK2606414), a potent and selective first-in-class inhibitor of protein kinase R (PKR)-like endoplasmic reticulum kinase (PERK). J. Med. Chem. 55, 7193-7207.
Bi, M., Naczki, C., Koritzinsky, M., Fels, D., Blais, J., Hu, N., Harding, H., Novoa, I., Varia, M., Raleigh, J., et al. (2005). ER stress-regulated translation increases tolerance to extreme hypoxia and promotes tumor growth, EMBO J 24, 3470-81.
Blais, J., and Bell, J. C. (2006). Novel therapeutic target: the PERKs of inhibiting the integrated stress response, Cell Cycle 5, 2874-7.
Civelek, M., et al. (2009). Chronic endoplasmic reticulum stress activates unfolded protein response in arterial endothelium in regions of susceptibility to atherosclerosis. Circ Res 105(5). 453-461.
Cunard, R (2015). Endoplasmic Reticulum Stress in the Diabetic Kidney, the Good, the Bad and the Ugly, J. Clin. Med. 4, 715-740.
Cybulsky, A. V., et al. (2005). Role of the endoplasmic reticulum unfolded protein response in glomerular epithelial cell injury. J Biol Chem 280(26): 24396-24403.
Davies, M. P., Barraclough, D. L., Stewart, C., Joyce, K. A., Eccles, R. M., Barraclough, R., Rudland, P. S., and Sibson, D. R. (2008). Expression and splicing of the unfolded protein response gene XBP-1 are significantly associated with clinical outcome of endocrine-treated breast cancer, Int J Cancer 123, 85-8.
Dickhout, J. G.; Carlisle, R. E.; Austin, R. C.; (2011). Interrelationship Between Cardiac Hypertrophy, Heart Failure, and Chronic Kidney, Cire Res. 108, 629-642.
Doyle, K. M.; Kennedy, D.; Gorman, A. M.; Gupta, S.; Healy, S. J. M.; Samali, A. (2011) Unfolded proteins and endoplasmic reticulum stress in neurodegenerative disorders J. Cell. Mol. Med. 15, 2025-2039.
Feldman, D. E., Chauhan, V., and Koong, A. C. (2005). The unfolded protein response: a novel component of the hypoxic stress response in tumors, Mol Cancer Res 3, 597-605.
Gupta, S., McGrath, B., and Cavener, D. R. (2009). PERK regulates the proliferation and development of insulin-secreting beta-cell tumors in the endocrine pancreas of mice, PLoS One 4, e8008.
Hamanaka, R. B., Bennett, B. S., Cullinan, S. B., and Diehl, J. A. (2005). PERK and GCN2 contribute to eIF2alpha phosphorylation and cell cycle arrest after activation of the unfolded protein response pathway, Mol Biol Cell 16, 5493-501.
Harding, H. P.; Zhang, Y.; Ron, D. (1999) Protein translation and folding are coupled by an endoplasmic-reticulumresident kinase Nature 397, 271-274.
Harding, H. P., Zeng, H., Zhang, Y., Jungries, R., Chung, P., Plesken, H., Sabatini, D. D., and Ron, D. (2001). Diabetes mellitus and exocrine pancreatic dysfunction in perk−/− mice reveals a role for translational control in secretory cell survival, Mol Cell 7, 1153-63.
Harding, H. P.; Calfon, M.; Urano, F.; Novoa, I.; Ron, D. (2002) TRANSCRIPTIONAL AND TRANSLATIONAL CONTROL IN THE MAMMALIAN UNFOLDEDPROTEIN RESPONSE.
Annu. Rev. Cell Dev. Biol. 18, 575-599 Hetz, C. (2012). The unfolded protein response: controlling cell fate decisions under ER stress and beyond. Nat. Rev. Mol. Cell Bio. 13, 89-102.
Ho, Y.; Yang, X.; Lau, J. C; Hung, C. H.; Wuwongse, S; Zhang, Q.; Wang, J.; Baum, L.; So, K.; Chang, R. C. (2012) Endoplasmic Reticulum Stress Induces Tau Pathology and Forms a Vicious Cycle: Implication in Alzheimer's Disease Pathogenesis J. Alzheimer's Disease 28, 839-854.
Hoozemans. J. J. M.; van Haastert, E. S.; Nijholt, D. A. T.; Rozemuller, A. J. M.; Eikelenboom, P.; Scheper, W. (2009) The Unfolded Protein Response Is Activated in Pretangle Neurons in Alzheimer's Disease Hippocampus Am. J. Path. 174, 1241-1251.
Iida, K., Li, Y., McGrath, B. C., Frank, A., and Cavener, D. R. (2007). PERK eIF2 alpha kinase is required to regulate the viability of the exocrine pancreas in mice, BMC Cell Biol 8, 38.
Inagi, R., Ishimoto, Y, and Nangaku, M. (2014). Proteostasis in endoplasmic reticulum—new mechanisms in kidney disease, Nat. Rev. Nephrol. 10, 369-378.
Jackson, R. J.; Hellen, C. U. T.; Pestova, T. V. (2010) The mechanism of eukaryotic translation initiation and principles of its regulation Nat. Rev. Mol. Cell Biol. 10, 113-127.
Jorgensen, E., Stinson, A., Shan, L., Yang, J., Gietl, D., and Albino, A. P. (2008). Cigarette smoke induces endoplasmic reticulum stress and the unfolded protein response in normal and malignant human lung cells, BMC Cancer 8, 229. Julier, C.; Nicolino, M. (2010) Wolcott-Rallison syndrome Orphanet J. Rare Diseases 5 (29) 1-13. http://www.ojrd.com/content/5/1/29
Liu, M. and S. C. Dudley, Jr. (2016) Role for the Unfolded Protein Response in Heart Disease and Cardiac Arrhythmias Int J Mol Sci 17(1) pii: E52. doi: 10.3390/ijms17010052
Lucke-Wold, B. P., et al. (2016). Endoplasmic reticulum stress implicated in chronic traumatic encephalopathy. J Neurosurg 124(3): 687-702.
Kanekura, K.; Suzuki, H.; Aiso, S.; Matsuoka, M. ER Stress and Unfolded Protein Response in Amyotrophic Lateral Sclerosis Molecular Neurobiology (2009), 39(2), 81-89.
Koumenis, C., Naczki, C., Koritzinsky, M., Rastani, S., Diehl, A., Sonenberg, N., Koromilas, A., and Wouters, B. G. (2002). Regulation of protein synthesis by hypoxia via activation of the endoplasmic reticulum kinase PERK and phosphorylation of the translation initiation factor eIF2alpha, Mol Cell Biol 22, 7405-16.
Koumenis, C., and Wouters, B. G. (2006). “Translating” tumor hypoxia: unfolded protein response (UPR)-dependent and UPR-independent pathways, Mol Cancer Res 4, 423-36.
Ma, K., Vattem, K. M., and Wek, R. C. (2002). Dimerization and release of molecular chaperone inhibition facilitate activation of eukaryotic initiation factor-2 kinase in response to endoplasmic reticulum stress, J Biol Chem 277, 18728-35.
Ma, Y., and Hendershot, L. M. (2004). The role of the unfolded protein response in tumour development: friend or foe?, Nat Rev Cancer 4, 966-77.
Ma, Y., Lu, Y., Zeng, H., Ron, D., Mo, W., and Neubert, T. A. (2001). Characterization of phosphopeptides from protein digests using matrix-assisted laser desorption/ionization time-of-flight mass spectrometry and nanoelectrospray quadrupole time-of-flight mass spectrometry, Rapid Commun Mass Spectrom 15, 1693-700.
Marciniak, S. J., Garcia-Bonilla, L., Hu, J., Harding, H. P., and Ron, D. (2006). Activation-dependent substrate recruitment by the eukaryotic translation initiation factor 2 kinase PERK, J Cell Biol 172, 201-9.
McAlpine, C. S.; Bowes, A. J.; and Werstuck, G. H. (2010) Diabetes, hyperglycemia and accelerated atherosclerosis: evidence supporting a role for endoplasmic reticulum (ER) stress signaling. Cardiovascular & Hematological Disorders: Drug Targets 10(2), 151-157.
Moreno, J. A; Radford, H.; Peretti, D.; Steinert, J. R.; Verity, N.; Martin, M. G.; Halliday, M.; Morgan, J.; Dinsdale, D.; Ortori, C. A.; Barrett, D. A.; Tsaytler, P.; Bertolotti, A.; Willis, A. E.; Bushell, M.; Mallucci, G. R. (2012) Sustained translational repression by eIF2a-P mediates prion neurodegeneration. Nature 485, 507-512.
Moreno, J. A.; Halliday, M.; Molloy, C.; Radford, H.; Verity, N.; Axten, J. M.; Ortori, C. A.; Willis, A. E.; Fischer, P. M.; Barrett, D. A.; Mallucci, G. R. (2013) Oral Treatment Targeting the Unfolded Protein Response Prevents Neurodegeneration and Clinical Disease in Prion-Infected Mice. Science Trans. Med, 5, 206ra138. doi: 10.1126/scitranslmed.3006767
Nassif, M.; Matus, S.; Castillo, K.; and Hetz, C. (2010) Amyotrophic Lateral Sclerosis Pathogenesis: A Journey Through the Secretory Pathway Antioxidants & Redox Signaling 13(12), 1955-1989.
Nijholt, D. A., et al. (2012). The unfolded protein response is associated with early tau pathology in the hippocampus of tauopathies. J Pathol 226(5): 693-702.
O'Connor, T.; Sadleir, K. R.; Maus, E.; Velliquette, R. A.; Zhao, J.; Cole, S. L.; Eimer, W. A.; Hitt, B.; Bembinster, L. A.; Lammich, S. Lichtenthaler, S. F., Hebert, S. S., De Strooper, B., Haass, C., Bennett, D. A., Vassar, R. (2008) Phosphorylation of the translation initiation factor eIF2α increases BACE1 levels and promotes amyloidogenesis. Neuron, 60(6), 988-1009.
Ohri, S. S.; Maddie, M. A.; Zhao, Y.; Qiu, M. S.; Hetman, M.; Whittemore, S. R. (2011) Attenuating the Endoplasmic Reticulum Stress Response Improves Functional Recovery After Spinal Cord Injury. Glia 59, 1489-1502.
Paschen, W. (2004) Endoplasmic reticulum dysfunction in brain pathology: Critical role of protein synthesis Current Neurovascular Research, 1(2), 173-181.
Prusiner, S. B. (2012) A Unifying Role for Prions in Neurodegenerative Diseases Science 336, 1511-1513.
Romero-Ramirez, L., Cao, H., Nelson, D., Hammond, E., Lee, A. H., Yoshida, H., Mori, K., Glimcher, L. H., Denko, N. C., Giaccia, A. J., et al. (2004). XBP1 is essential for survival under hypoxic conditions and is required for tumor growth, Cancer Res 64, 5943-7.
Ron, D. (2002) Translational control in the endoplasmic reticulum stress response. J. Clin. Invest. 110, 1383-1388.
Rouschop, K. M., van den Beucken, T., Dubois, L., Niessen, H., Bussink, J., Savelkouls, K., Keulers, T., Mujcic, H., Landuyt, W., Voncken, J. W., et al. (2010) The unfolded protein response protects human tumor cells during hypoxia through regulation of the autophagy genes MAP1LC3B and ATG5, J Clin Invest 120, 127-41.
Salminen, A.; Kauppinen, A.; Suuronen, T.; Kaarniranta, K.; Ojala, J. ER stress in Alzheimer's disease: a novel neuronal trigger for inflammation and Alzheimer's pathology. Journal of Neuroinflammation (2009), 6:41.
Shi, Y., Vattem, K. M., Sood, R., An, J., Liang, J., Stramm, L., and Wek, R. C. (1998). Identification and characterization of pancreatic eukaryotic initiation factor 2 alpha-subunit kinase, PEK, involved in translational control, Mol Cell Biol 18, 7499-509.
Sood, R., Porter, A. C., Ma, K., Quilliam, L. A., and Wek, R. C. (2000). Pancreatic eukaryotic initiation factor-2alpha kinase (PEK) homologues in humans, Drosophila melanogaster and Caenorhabditis elegans that mediate translational control in response to endoplasmic reticulum stress, Biochem J 346 Pt 2, 281-93.
Su, Q., Wang, S., Gao, H. Q., Kazemi, S., Harding, H. P., Ron, D., and Koromilas, A. E. (2008). Modulation of the eukaryotic initiation factor 2 alpha-subunit kinase PERK by tyrosine phosphorylation, J Biol Chem 283, 469-75.
Stutzbach, L. D., Xie, S. X., Naj, A. C., Albin, R., Gilman, S., Lee, V. M. Y, Trojanowski, J. Q., Devlin, B., Schellenberg, G. D. (2013) The unfolded protein response is activated in disease-affected brain regions in progressive supranuclear palsy and Alzheimer's disease, Acta Neuropath. Comm. 1, 31, http://www.actaneurocomms.org/content1/1/31 Tabas, I.; Seimon, T.; Timmins, J.; Li, G.; Lim, W. Macrophage apoptosis in advanced atherosclerosis Annals of the New York Academy of Sciences (2009), 1173(S1), E40-E45.
Tajiri, S.; Oyadomari, S.; Yano, S.; Morioka, M.; Gotoh, T.; Hamada, J. I; Ushio, Y.; Mori, M. (2004) Ischemia-induced neuronal cell death is mediated by the endoplasmic reticulum stress pathway involving CHOP. Cell Death and Diff. 11, 403-415.
Unterberger, U.; Hoftberger, R.; Gelpi, E.; Flicker, H.; Budka, H.; Voigtlander, T. (2006) Endoplasmic Reticulum Stress Features Are Prominent in Alzheimer Disease but Not in Prion Diseases In Vivo J. Neuropathol. Exp. Neurol. 65, 348-357.
van Galen, P., et al. (2014). The unfolded protein response governs integrity of the haematopoietic stem-cell pool during stress. Nature 510(7504): 268-272.
Wek, R. C. and D. R. Cavener (2007). Translational control and the unfolded protein response. Antioxid Redox Signal 9(12): 2357-2371.
Walter, P.; Ron, D. (2011) The Unfolded Protein Response: From Stress Pathway to Homeostatic Regulation. Science 334, 1081-1086.
Woehlbier, U.; Hetz, C. Modulating stress responses by the UPRosome: A matter of life and death. Trends Biochem. Sciences 36, 329-337 Zhang, W., Feng, D., Li, Y., lida, K., McGrath, B., and Cavener, D. R. (2006). PERK EIF2AK3 control of pancreatic beta cell differentiation and proliferation is required for postnatal glucose homeostasis, Cell Metab 4, 491-7.
Zhang, K. and R. J. Kaufman (2006). The unfolded protein response: a stress signaling pathway critical for health and disease. Neurology 66(2 Suppl 1): S102-109

It is an object of the instant invention to provide novel compounds that are inhibitors of PERK.

It is also an object of the present invention to provide pharmaceutical compositions that comprise a pharmaceutical carrier and compounds of Formula (I).

It is an object of the instant invention to provide novel compounds that are inhibitors of PERK.

It is also an object of the present invention to provide pharmaceutical compositions that comprise a pharmaceutical carrier and compounds of Formula (I).

It is also an object of the present invention to provide a method for treating neurodegenerative diseases, cancer, and other diseases/injuries associated with activated unfolded protein response pathways such as: Alzheimer's disease, neuropathic pain, spinal cord injury, traumatic brain injury, ischemic stroke, stroke, Parkinson disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation that comprises administering novel inhibitors of PERK activity.

### SUMMARY OF THE INVENTION

The invention is directed to substituted pyrrolidinone and imidazolidinone derivatives. Specifically, the invention is directed to compounds according to Formula I:

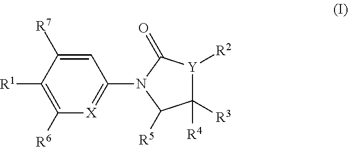

wherein R¹, R², R³, R⁴, R⁵, R⁶, R⁷, X and Y are as defined below; or a salt thereof including a pharmaceutically acceptable salt thereof.

The present invention also relates to the discovery that the compounds of Formula (I) are active as inhibitors of PERK.

This invention also relates to a method of treating cancer, which comprises administering to a subject in need thereof an effective amount of a PERK inhibiting compound of Formula (I).

This invention also relates to a method of treating Alzheimer's disease, which comprises administering to a subject in need thereof an effective amount of a PERK inhibiting compound of Formula (I).

This invention also relates to a method of treating Parkinson's disease, which comprises administering to a subject in need thereof an effective amount of a PERK inhibiting compound of Formula (I).

This invention also relates to a method of treating amyotrophic lateral sclerosis, which comprises administering to a subject in need thereof an effective amount of a PERK inhibiting compound of Formula (I).

This invention also relates to a method of treating Huntington's disease, which comprises administering to a subject in need thereof an effective amount of a PERK inhibiting compound of Formula (I).

This invention also relates to a method of treating Creutzfeldt-Jakob Disease, which comprises administering to a subject in need thereof an effective amount of a PERK inhibiting compound of Formula (I).

This invention also relates to a method of treating progressive supranuclear palsy (PSP), which comprises administering to a subject in need thereof an effective amount of a PERK inhibiting compound of Formula (I).

This invention also relates to a method of treating dementia, which comprises administering to a subject in need thereof an effective amount of a PERK inhibiting compound of Formula (I).

This invention also relates to a method of treating spinal cord injury, which comprises administering to a subject in need thereof an effective amount of a PERK inhibiting compound of Formula (I).

This invention also relates to a method of treating traumatic brain injury, which comprises administering to a subject in need thereof an effective amount of a PERK inhibiting compound of Formula (I).

This invention also relates to a method of treating ischemic stroke, which comprises administering to a subject in need thereof an effective amount of a PERK inhibiting compound of Formula (I).

This invention also relates to a method of treating diabetes, which comprises administering to a subject in need thereof an effective amount of a PERK inhibiting compound of Formula (I).

This invention also relates to a method of treating a disease state selected from: myocardial infarction, cardiovascular disease, atherosclerosis, ocular diseases, and arrhythmias, which comprises administering to a subject in need thereof an effective amount of a PERK inhibiting compound of Formula (I).

This invention also relates to a method of using the compounds of Formula (I) in organ transplantation and in the transportation of organs for transplantation.

In a further aspect of the invention there is provided novel processes and novel intermediates useful in preparing the presently invented PERK inhibiting compounds.

Included in the present invention are pharmaceutical compositions that comprise a pharmaceutical carrier and compounds useful in the methods of the invention.

Also included in the present invention are methods of co-administering the presently invented PERK inhibiting compounds with further active ingredients.

The invention also relates to a compound of Formula (I) or a pharmaceutically acceptable salt thereof for use in therapy.

The invention also relates to a compound of Formula (I) or a pharmaceutically acceptable salt thereof for use in the treatment of Alzheimer's disease.

The invention also relates to a compound of Formula (I) or a pharmaceutically acceptable salt thereof for use in the treatment of Parkinson's disease.

The invention also relates to a compound of Formula (I) or a pharmaceutically acceptable salt thereof for use in the treatment of amyotrophic lateral sclerosis.

The invention also relates to a compound of Formula (I) or a pharmaceutically acceptable salt thereof for use in the treatment of Huntington's disease.

The invention also relates to a compound of Formula (I) or a pharmaceutically acceptable salt thereof for use in the treatment of Creutzfeldt-Jakob Disease.

The invention also relates to a compound of Formula (I) or a pharmaceutically acceptable salt thereof for use in the treatment of progressive supranuclear palsy (PSP).

The invention also relates to a compound of Formula (I) or a pharmaceutically acceptable salt thereof for use in the treatment of dementia.

The invention also relates to a compound of Formula (I) or a pharmaceutically acceptable salt thereof for use in the treatment of spinal cord injury.

The invention also relates to the use of a compound of Formula (I) or a pharmaceutically acceptable salt thereof in the preparation of a medicament for the treatment of traumatic brain injury.

The invention also relates to the use of a compound of Formula (I) or a pharmaceutically acceptable salt thereof in the preparation of a medicament for the treatment of diabetes.

The invention also relates to the use of a compound of Formula (I) or a pharmaceutically acceptable salt thereof in the preparation of a medicament for the treatment of a disease state selected from: myocardial infarction, cardiovascular disease, atherosclerosis, ocular diseases, and arrhythmias.

The invention also relates to the use of a compound of Formula (I) or a pharmaceutically acceptable salt thereof in the preparation of a medicament for the treatment of chronic traumatic encephalopathy (CTE).

The invention also relates to the use of a compound of Formula (I) or a pharmaceutically acceptable salt thereof in the preparation of a medicament for use in organ transplantation and in the transportation of organs for transplantation.

Included in the present invention are pharmaceutical compositions that comprise a pharmaceutical carrier and a compound of Formula (I) or a pharmaceutically acceptable salt thereof.

The invention also relates to a pharmaceutical composition as defined above for use in therapy.

### DETAILED DESCRIPTION OF THE INVENTION

This invention relates to novel compounds of Formula (I) and to the use of compounds of Formula (I) in the methods of the invention:

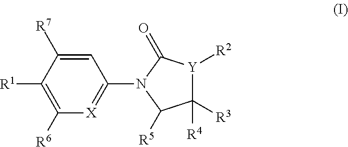

wherein:
R¹ is selected from:
   bicycloheteroaryl,
   substituted bicycloheteroaryl,
   heteroaryl, and
   substituted heteroaryl,
   where said substituted bicycloheteroaryl and said substituted heteroaryl are
   substituted with from one to five substituents independently selected from:
      fluoro,
      chloro,
      bromo,
      iodo,
      C₁₋₆alkyl,
      C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
      —OH,
      hydroxyC₁₋₆alkyl,
      —COOH,
      tetrazole,
      cycloalkyl,
      oxo,
      —OC₁₋₆alkyl,
      —CF₃,
      —CF₂H,
      —CFH₂,
      —C₁₋₆alkylOC₁₋₄alkyl,
      CONH₂,
      —CON(H)C₁₋₃alkyl,
      —CH₂CH₂N(H)C(O)OCH₂aryl,
      diC₁₋₄alkylaminoC₁₋₄alkyl,
      aminoC₁₋₆alkyl,
      —CN,
      heterocycloalkyl,
      heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
      —NO₂,
      —NH₂,
      —N(H)C₁₋₃alkyl, and
      —N(C₁₋₃alkyl)₂;
R² is selected from:
   hydrogen,
   —NH₂,
   —N(H)C₁₋₃alkyl,
   —N(C₁₋₃alkyl)₂,
   —OH,
   cycloalkyl,
   benzyl,
   aryl,
   heterocycloalkyl,
   heteroaryl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R³ is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   heteroaryl,
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   bicycloheteroaryl,
   bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —OCF₃, and —CN, and
   cycloalkyl;
   R⁴ and R⁵ are each independently selected from hydrogen and C₁₋₆alkyl,
      or R⁴ and R⁵ taken together with the carbon atoms to which they are attached represent a 3 or 4 member cycloalkyl; and
   R⁶ is selected from: hydrogen, C₁₋₄alkyl, —CF₃, —C(H)F₂, —CH₂F, fluoro, chloro, bromo and iodo;
   R⁷ is selected from: hydrogen, C₁₋₄alkyl, —CF₃, —C(H)F₂, —CH₂F, fluoro, chloro, bromo and iodo;
   Y is CR⁹⁰ or N,
      where R⁹⁰ is selected from: hydrogen, C₁₋₄alkyl, cycloalkyl, —OH, —NH₂, —CN, and —CF₃; and
   X is CR¹⁰⁰ or N,
      where R¹⁰⁰ is selected from: hydrogen, —CH₃, —CF₃, fluoro, chloro, bromo and iodo;
         and salts thereof.
This invention also relates to pharmaceutically acceptable salts of the compounds of Formula (I).
Suitably, in the compounds of Formula (I), Y is CH.
Suitably, in the compounds of Formula (I), Y is N.
Suitably, in the compounds of Formula (I), X is CR¹⁰⁰, where R¹⁰⁰ is selected from: hydrogen, —CH₃, fluoro, chloro, bromo and iodo.
Suitably, in the compounds of Formula (I), X is N.
Suitably, in the compounds of Formula (I), R¹ is a substituted thieno[2,3-d]pyrimidinyl.
Suitably, in the compounds of Formula (I), R¹ is selected from: substituted pyrrolo[2,3d]pyrimidin-5-yl and substituted pyrazolo[3,4d]pyrimidin-5-yl.
Suitably, in the compounds of Formula (I), R¹ is selected from: 4-amino-7-methyl-7H-pyrrolo[2,3-d]pyrimidin-5-yl and 4-amino-1-methyl-1H-pyrazolo[3,4-d]pyrimidin-3-yl.
Suitably, in the compounds of Formula (I), R⁷ is hydrogen.
Included in the compounds of Formula (I) are compounds of Formula (II):

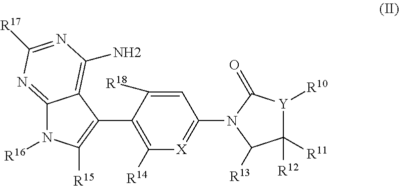

wherein:
R¹⁰ is selected from:
   hydrogen,
   —NH₂,
   —N(H)C₁₋₃alkyl,
   —N(C₁₋₃alkyl)₂,
   —OH,
   cycloalkyl,
   phenyl,
   heterocycloalkyl,
   heteroaryl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R¹¹ is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   cycloalkyl,
   heteroaryl, and
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —OC(H)F₂, —C(H)F₂, —OCH₂F, —CH₂F, —OCF₃, —NH₂ and —CN;
R¹² and R¹³ are each independently selected from hydrogen and C₁₋₆alkyl,
   or R¹² and R¹³ taken together with the carbon atoms to which they are attached represent a 3 or 4 member cycloalkyl;
R¹⁴ is selected from: hydrogen, C₁₋₄alkyl, —CF₃, —C(H)F₂, —CH₂F, fluoro and chloro;
R¹⁵ is selected from hydrogen and C₁₋₆alkyl;
R¹⁶ is selected from:
   hydrogen,
   cycloalkyl,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R¹⁷ is selected from: hydrogen and —CH₃;
R¹⁸ is selected from: hydrogen, C₁₋₄alkyl, —CF₃, —C(H)F₂, —CH₂F, fluoro and chloro;
Y is CR⁹¹ or N,
   where R⁹¹ is selected from: hydrogen, C₁₋₄alkyl, cycloalkyl, —OH, —NH₂, —CN, and —CF₃; and
X is CR¹⁰¹ or N,
   where R¹⁰¹ is selected from: hydrogen, fluoro and chloro;
      and salts thereof.

This invention also relates to pharmaceutically acceptable salts of the compounds of Formula (II).

Suitably, in the compounds of Formula (II), Y is CH.

Suitably, in the compounds of Formula (II), Y is N.

Suitably, in the compounds of Formula (II), X is CR¹⁰¹, where R¹⁰¹ is selected from: hydrogen, fluoro and chloro.

Suitably, in the compounds of Formula (II), X is N.

Suitably, in the compounds of Formula (II), R¹⁸ is hydrogen.

Included in the compounds of Formula (I) are compounds of Formula (IIa):

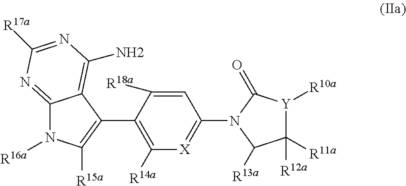

wherein:
R^{10a} is selected from:
   hydrogen,
   —NH₂,
   —N(H)C₁₋₃alkyl,
   —N(C₁₋₃alkyl)₂,
   —OH,
   cycloalkyl,
   phenyl,
   benzyl,
   heterocycloalkyl,
   heteroaryl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R^{11a} is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
   cycloalkyl,
   heteroaryl, and
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —OC(H)F₂, —C(H)F₂, —OCH₂F, —CH₂F, —OCF₃, —NH₂ and —CN;
R^{12a} and R^{13a} are each independently selected from hydrogen and C₁₋₆alkyl,
   or R^{12a} and R^{13a} taken together with the carbon atoms to which they are attached represent a 3 or 4 member cycloalkyl;
R^{14a} is selected from: hydrogen, C₁₋₄alkyl, —CF₃, —C(H)F₂, —CH₂F, fluoro and chloro;
R^{15a} is selected from hydrogen and C₁₋₆alkyl;
R^{16a} is selected from:
   hydrogen,
   cycloalkyl,
   heterocycloalkyl,
   heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, —NO₂, —NH₂ and —CN,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R^{17a} is selected from: hydrogen and —CH₃;
R^{18a} is selected from: hydrogen, C₁₋₄alkyl, —CF₃, —C(H)F₂, —CH₂F, fluoro and chloro;
Y is CR^{91a} or N,
   where R^{91a} is selected from: hydrogen, C₁₋₄alkyl, cycloalkyl, —OH, —NH₂, —CN, and —CF₃; and
X is CR^{101a} or N,
   where R^{101a} is selected from: hydrogen, fluoro and chloro;
      and salts thereof.

This invention also relates to pharmaceutically acceptable salts of the compounds of Formula (IIa).

Suitably, in the compounds of Formula (IIa), Y is CH.

Suitably, in the compounds of Formula (IIa), Y is N.

Suitably, in the compounds of Formula (IIa), X is CR^{101a}, where R^{101a} is selected from: hydrogen, fluoro and chloro.

Suitably, in the compounds of Formula (IIa), X is N.

Suitably, in the compounds of Formula (II), R^{18a} is hydrogen.

Included in the compounds of Formula (I) are compounds of Formula (III):

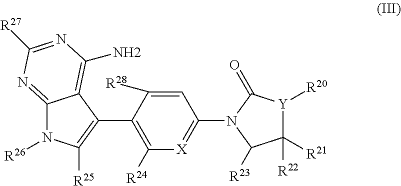

wherein:
R²⁰ is selected from:
   hydrogen,
   cycloalkyl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R²¹ is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄akyl, —NO₂, —NH₂ and —CN,
   cycloalkyl,
   heteroaryl, and
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄akyl, —NO₂, —NH₂ and —CN;
R²² and R²³ are each independently selected from hydrogen and C₁₋₆alkyl,
   or R²² and R²³ taken together with the carbon atoms to which they are attached represent a 3 or 4 member cycloalkyl;
R²⁴ is selected from: hydrogen, methyl, —CF₃, fluoro and chloro;
R²⁵ is selected from hydrogen and C₁₋₆alkyl;
R²⁶ is selected from:
   hydrogen,
   cycloalkyl,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R²⁷ is selected from: hydrogen and —CH₃;
R²⁸ is selected from: hydrogen, methyl, —CF₃, fluoro and chloro;
Y is CH or N; and
X is CR¹⁰² or N,
   where R¹⁰² is selected from: hydrogen, fluoro and chloro;
      and salts thereof.

This invention also relates to pharmaceutically acceptable salts of the compounds of Formula (III).

Suitably, in the compounds of Formula (III), Y is CH.

Suitably, in the compounds of Formula (III), Y is N.

Suitably, in the compounds of Formula (III), X is CR¹⁰², where R¹⁰² is selected from: hydrogen, fluoro and chloro.

Suitably, in the compounds of Formula (III), X is N.

Suitably, in the compounds of Formula (III), R²⁸ is hydrogen.

Included in the compounds of Formula (I) are compounds of Formula (IIIa):

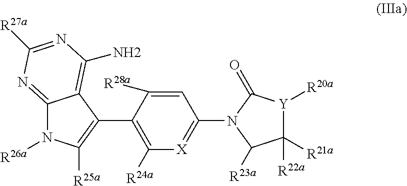

wherein:
R^{20a} is selected from:
   hydrogen,
   cycloalkyl,
   benzyl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R^{21a} is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN,
   cycloalkyl,
   heteroaryl, and
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN;
R^{22a} and R^{23a} are each independently selected from hydrogen and C₁₋₆alkyl,
   or R^{22a} and R^{23a} taken together with the carbon atoms to which they are attached represent a 3 or 4 member cycloalkyl;
R^{24a} is selected from: hydrogen, methyl, —CF₃, fluoro and chloro;
R^{25a} is selected from hydrogen and C₁₋₆alkyl;
R^{26a} is selected from:
   hydrogen,
   cycloalkyl,
   heterocycloalkyl,
   heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, —NO₂, —NH₂ and —CN,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R^{27a} is selected from: hydrogen and —CH₃;
R^{28a} is selected from: hydrogen, methyl, —CF₃, fluoro and chloro;
Y is CH or N; and
X is CR^{102a} or N,
   where R^{102a} is selected from: hydrogen, fluoro and chloro;
      and salts thereof.

This invention also relates to pharmaceutically acceptable salts of the compounds of Formula (IIIa).

Suitably, in the compounds of Formula (IIIa), Y is CH.

Suitably, in the compounds of Formula (IIIa), Y is N.

Suitably, in the compounds of Formula (IIIa), X is CR^{102a}, where R^{102a} is selected from: hydrogen, fluoro and chloro.

Suitably, in the compounds of Formula (IIIa), X is N.

Suitably, in the compounds of Formula (IIIa), R^{28a} is hydrogen.

Included in the compounds of Formula (I) are compounds of Formula (IV):

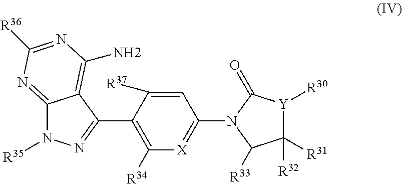

wherein:
R³⁰ is selected from:
   hydrogen,
   cycloalkyl,
   phenyl,
   heterocycloalkyl,
   heteroaryl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R³¹ is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN,
   cycloalkyl,
   heteroaryl, and
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN;
R³² and R³³ are each independently selected from hydrogen and C₁₋₆alkyl,
   or R³² and R³³ taken together with the carbon atoms to which they are attached represent a 3 or 4 member cycloalkyl;
R³⁴ is selected from: hydrogen, methyl, —CF₃, fluoro and chloro;
R³⁵ is selected from:
   hydrogen,
   cycloalkyl,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R³⁶ is selected from: hydrogen and —CH₃;
R³⁷ is selected from: hydrogen, methyl, —CF₃, fluoro and chloro;
Y is CH or N; and
X is CR¹⁰³ or N,
   where R¹⁰³ is selected from: hydrogen, fluoro and chloro;
      and salts thereof.

This invention also relates to pharmaceutically acceptable salts of the compounds of Formula (IV).

Suitably, in the compounds of Formula (IV), Y is CH.

Suitably, in the compounds of Formula (IV), Y is N.

Suitably, in the compounds of Formula (IV), X is CR¹⁰³, where R¹⁰³ is selected from: hydrogen, fluoro and chloro.

Suitably, in the compounds of Formula (IV), X is N.

Suitably, in the compounds of Formula (IV), R³⁷ is hydrogen.

Included in the compounds of Formula (I) are compounds of Formula (IVa):

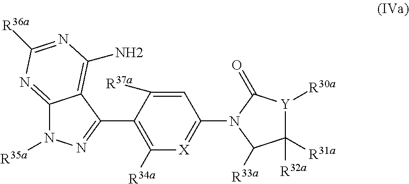

wherein:
R^{30a} is selected from:
   hydrogen,
   cycloalkyl,
   phenyl,
   benzyl,
   heterocycloalkyl,
   heteroaryl,
   C₁₋₆alkyl, and
   C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R^{31a} is selected from:
   aryl,
   aryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN,
   cycloalkyl,
   heteroaryl, and
   heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN;
R^{32a} and R^{33a} are each independently selected from hydrogen and C₁₋₆alkyl,
   or R^{32a} and R^{33a} taken together with the carbon atoms to which they are attached represent a 3 or 4 member cycloalkyl;
R^{34a} is selected from: hydrogen, methyl, —CF₃, fluoro and chloro;
R^{35a} is selected from:
   hydrogen,
   cycloalkyl,
   heterocycloalkyl,
   heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, —NO₂, —NH₂ and —CN,
   C₁₋₆alkyl, and
   C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R^{36a} is selected from: hydrogen and —CH₃;
R^{37a} is selected from: hydrogen, methyl, —CF₃, fluoro and chloro;
Y is CH or N; and
X is CR^{103a} or N,
   where R^{103a} is selected from: hydrogen, fluoro and chloro;
      and salts thereof.

This invention also relates to pharmaceutically acceptable salts of the compounds of Formula (IV).

Suitably, in the compounds of Formula (IV), Y is CH.

Suitably, in the compounds of Formula (IV), Y is N.

Suitably, in the compounds of Formula (IV), X is CR^{103a} where R^{103a} is selected from: hydrogen, fluoro and chloro.

Suitably, in the compounds of Formula (IV), X is N.

Suitably, in the compounds of Formula (IV), R^{37a} is hydrogen.

Included in the compounds of Formula (I) are:
1-(4-(4-amino-7-methyl-7H-pyrrolo[2,3-d]pyrimidin-5-yl)-3-fluorophenyl)-4-(2,5-difluorophenyl)-3-ethylimidazolidin-2-one;
1-(4-(4-Amino-7-methyl-7H-pyrrolo[2,3-d]pyrimidin-5-yl)-3-fluorophenyl)-4-(3,5-difluorophenyl)pyrrolidin-2-one;
1-(4-(4-Amino-7-methyl-7H-pyrrolo[2,3-d]pyrimidin-5-yl)-3-methylphenyl)-4-(3,5-difluorophenyl)pyrrolidin-2-one;
1-(4-(4-Amino-7-methyl-7H-pyrrolo[2,3-d]pyrimidin-5-yl)-3-fluorophenyl)-4-cyclohexylpyrrolidin-2-one;
1-(4-(4-amino-7-methyl-7H-pyrrolo[2,3-d]pyrimidin-5-yl)-3-fluorophenyl)-4-(2,5-difluorophenyl)-3-isobutylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one;
1-(4-(4-amino-7-(2,2-difluoroethyl)-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-3-benzyl-4-(2,5-difluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)imidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-ethylimidazolidin-2-one;
1-(4-(4-amino-7-methyl-7H-pyrrolo[2,3-d]pyrimidin-5-yl)-3-fluorophenyl)-4-cyclopentylpyrrolidin-2-one;
1-(4-(4-amino-7-methyl-7H-pyrrolo[2,3-d]pyrimidin-5-yl) phenyl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(3,5-dimethylphenyl)pyrrolidin-2-one,
1-(4-(4-amino-7-methyl-7H-pyrrolo[2,3-d]pyrimidin-5-yl)-3-fluorophenyl)-4-(2,5-difluorophenyl)pyrrolidin-2-one;
1-(5-(4-amino-7-methyl-7H-pyrrolo[2,3-d]pyrimidin-5-yl)pyridin-2-yl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(6-methylpyridin-2-yl)pyrrolidin-2-one;
1-(4-(4-amino-7-methyl-7H-pyrrolo[2,3-d]pyrimidin-5-yl)-3-fluorophenyl)-4-(2,4-difluorophenyl)pyrrolidin-2-one;
1-(4-(4-amino-7-methyl-7H-pyrrolo[2,3-d]pyrimidin-5-yl)-3-fluorophenyl)-4-(3-chloro-2-fluorophenyl) pyrrolidin-2-one;
1-(4-(4-amino-7-methyl-7H-pyrrolo[2,3-d]pyrimidin-5-yl)-3-fluorophenyl)-3-ethyl-4-(m-tolyl)imidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-7-methyl-7H-pyrrolo[2,3-d]pyrimidin-5-yl)-3-fluorophenyl)-3-methyl-4-(m-tolyl)imidazolidin-2-one;
1-(5-(4-amino-7-methyl-7H-pyrrolo[2,3-d]pyrimidin-5-yl)pyridin-2-yl)-4-(2,5-difluorophenyl)-3-methylimidazolidin-2-one;
1-(5-(4-amino-7-methyl-7H-pyrrolo[2,3-d]pyrimidin-5-yl)pyridin-2-yl)-4-(2,5-difluorophenyl)-3-ethylimidazolidin-2-one;
1-(4-(4-aminothieno[2,3-d]pyrimidin-5-yl)-3-fluorophenyl)-4-(2,5-difluorophenyl)-3-ethylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-3-methyl-4-(3-(trifluoromethyl)phenyl)imidazolidin-2-one;
1-(4-(4-amino-7-methyl-7H-pyrrolo[2,3-d]pyrimidin-5-yl)-3-fluorophenyl)-3-ethyl-4-(3-(trifluoromethyl)phenyl)imidazolidin-2-one;
1-(4-(4-amino-7-methyl-7H-pyrrolo[2,3-d]pyrimidin-5-yl)-3-fluorophenyl)-3-methyl-4-(6-methylpyridin-2-yl)imidazolidin-2-one;
1-(4-(4-amino-7-methyl-7H-pyrrolo[2,3-d]pyrimidin-5-yl)-3-fluorophenyl)-3-ethyl-4-(6-methylpyridin-2-yl)imidazolidin-2-one;
1-(4-(4-amino-7-methyl-7H-pyrrolo[2,3-d]pyrimidin-5-yl)-3-fluorophenyl)-4-(5-cyclopropyl-2-fluorophenyl)-3-methylimidazolidin-2-one;
1-(4-(4-amino-7-methyl-7H-pyrrolo[2,3-d]pyrimidin-5-yl)-3-fluorophenyl)-4-(5-cyclopropyl-2-fluorophenyl)-3-ethylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-isopropyl-7H-pyrrolo[2,3-d]pyrimidin-5-yl)-3-fluorophenyl)-4-(2,4-difluorophenyl)-3-ethylimidazolidin-2-one;
1-(4-(4-amino-7-isopropyl-7H-pyrrolo[2,3-d]pyrimidin-5-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3-(difluoromethoxy) phenyl)-3-ethylimidazolidin-2-one;
1-(4-(4-amino-7-methyl-7H-pyrrolo[2,3-d]pyrimidin-5-yl)-3-fluorophenyl)-4-(3-(difluoromethoxy) phenyl)-3-methylimidazolidin-2-one;
1-(4-(4-amino-7-methyl-7H-pyrrolo[2,3-d]pyrimidin-5-yl)-3-fluorophenyl)-3-cyclopropyl-4-(2,5-difluorophenyl)imidazolidin-2-one;
1-(4-(4-amino-7-methyl-7H-pyrrolo[2,3-d]pyrimidin-5-yl)-3-fluorophenyl)-4-(3,4-difluorophenyl)-3-ethylimidazolidin-2-one;
1-(4-(4-amino-7-methyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-ethylimidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one;
1-(4-(4-amino-7-methyl-7H-pyrrolo[2,3-d]pyrimidin-5-yl)-3-fluorophenyl)-3-ethyl-4-(4-fluorophenyl)imidazolidin-2-one;
1-(4-(4-amino-7-methyl-7H-pyrrolo[2,3-d]pyrimidin-5-yl)-3-fluorophenyl)-4-(4-fluorophenyl)-3-methyl imidazolidin-2-one;
1-(4-(4-amino-7-methyl-7H-pyrrolo[2,3-d]pyrimidin-5-yl)-3-fluorophenyl)-3-ethyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,4,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,4,6-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)-3-ethyl-4-methylimidazolidin-2-one;
1-(4-(4-amino-7-methyl-7H-pyrrolo[2,3-d]pyrimidin-5-yl)-3-fluorophenyl)-4-(2,5-difluorophenyl)-3,4-dimethylimidazolidin-2-one;
4-(4-(4-amino-7-methyl-7H-pyrrolo[2,3-d]pyrimidin-5-yl)-3-fluorophenyl)-1-(2,5-difluorophenyl)-2-ethyl-2,4-diazabicyclo[3.1.0]hexan-3-one;
4-(4-(4-amino-7-methyl-7H-pyrrolo[2,3-d]pyrimidin-5-yl)-3-fluorophenyl)-1-(2,5-difluorophenyl)-2-methyl-2,4-diazabicyclo[3.1.0]hexan-3-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one;
1-(4-(4-amino-6,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-1-methyl-1H-pyrazolo[3,4-d]pyrimidin-3-yl)-3-fluorophenyl)-4-(2,5-difluorophenyl)-3-ethylimidazolidin-2-one; and
1-(4-(4-amino-1-methyl-1H-pyrazolo[3,4-d]pyrimidin-3-yl)-3-fluorophenyl)-4-(2,5-difluorophenyl)-3-methylimidazolidin-2-one;
   and salts thereof including pharmaceutically acceptable salts thereof.

Included in the compounds of Formula (I) are:
1-(4-(4-amino-7-methyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-ethylimidazolidin-2-one, (enantiomer 1);
1-(4-(4-amino-7-methyl-7H-pyrrolo[2,3-d]pyrimidin-5-yl)-3-fluorophenyl)-4-(2,5-difluorophenyl)-3-ethylimidazolidin-2-one, (enantiomer 2);
1-(4-(4-Amino-7-methyl-7H-pyrrolo[2,3-d]pyrimidin-5-yl)-3-fluorophenyl)-4-(3,5-difluorophenyl)pyrrolidin-2-one;
1-(4-(4-Amino-7-methyl-7H-pyrrolo[2,3-d]pyrimidin-5-yl)-3-methylphenyl)-4-(3,5-difluorophenyl)pyrrolidin-2-one;
1-(4-(4-Amino-7-methyl-7H-pyrrolo[2,3-d]pyrimidin-5-yl)-3-fluorophenyl)-4-cyclohexylpyrrolidin-2-one;
1-(4-(4-amino-7-methyl-7H-pyrrolo[2,3-d]pyrimidin-5-yl)-3-fluorophenyl)-3-methyl-4-(pyridin-2-yl) imidazolidin-2-one;
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one, enantiomer 1;
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one, enantiomer 2;
1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one, enantiomer 1;
1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one, enantiomer 2;
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one, enantiomer 1;
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one, enantiomer 2;
1-(4-(4-amino-7-methyl-7H-pyrrolo[2,3-d]pyrimidin-5-yl)-3-fluorophenyl)-4-(2,5-difluorophenyl)-3-isobutylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one;
1-(4-(4-amino-7-(2,2-difluoroethyl)-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-3-benzyl-4-(2,5-difluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)imidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-ethylimidazolidin-2-one;
1-(4-(4-amino-7-methyl-7H-pyrrolo[2,3-d]pyrimidin-5-yl)-3-fluorophenyl)-4-cyclopentylpyrrolidin-2-one;
1-(4-(4-amino-7-methyl-7H-pyrrolo[2,3-d]pyrimidin-5-yl) phenyl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(3,5-dimethylphenyl)pyrrolidin-2-one;
1-(4-(4-amino-7-methyl-7H-pyrrolo[2,3-d]pyrimidin-5-yl)-3-fluorophenyl)-4-(2,5-difluorophenyl)pyrrolidin-2-one;
1-(5-(4-amino-7-methyl-7H-pyrrolo[2,3-d]pyrimidin-5-yl)pyridin-2-yl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(6-methylpyridin-2-yl)pyrrolidin-2-one;
1-(4-(4-amino-7-methyl-7H-pyrrolo[2,3-d]pyrimidin-5-yl)-3-fluorophenyl)-4-(2,4-difluorophenyl)pyrrolidin-2-one;
1-(4-(4-amino-7-methyl-7H-pyrrolo[2,3-d]pyrimidin-5-yl)-3-fluorophenyl)-4-(3-chloro-2-fluorophenyl) pyrrolidin-2-one;
1-(4-(4-amino-7-methyl-7H-pyrrolo[2,3-d]pyrimidin-5-yl)-3-fluorophenyl)-3-ethyl-4-(m-tolyl)imidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one (enantiomer 2);
1-(4-(4-amino-7-methyl-7H-pyrrolo[2,3-d]pyrimidin-5-yl)-3-fluorophenyl)-3-methyl-4-(m-tolyl)imidazolidin-2-one;
1-(5-(4-amino-7-methyl-7H-pyrrolo[2,3-d]pyrimidin-5-yl)pyridin-2-yl)-4-(2,5-difluorophenyl)-3-methylimidazolidin-2-one;
1-(5-(4-amino-7-methyl-7H-pyrrolo[2,3-d]pyrimidin-5-yl)pyridin-2-yl)-4-(2,5-difluorophenyl)-3-ethylimidazolidin-2-one;
1-(4-(4-aminothieno[2,3-d]pyrimidin-5-yl)-3-fluorophenyl)-4-(2,5-difluorophenyl)-3-ethylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one (enantiomer 1);
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one (enantiomer 2);
1-(4-(4-amino-7-methyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-7-methyl-7H-pyrrolo[2,3-d]pyrimidin-5-yl)-3-fluorophenyl)-4-(2,4-difluorophenyl)-3-methylimidazolidin-2-one (enantiomer 2);
1-(4-(4-amino-7-methyl-7H-pyrrolo[2,3-d]pyrimidin-5-yl)-3-fluorophenyl)-4-(5-cyclopropyl-2-fluorophenyl)-3-ethylimidazolidin-2-one;
1-(4-(4-amino-7-methyl-7H-pyrrolo[2,3-d]pyrimidin-5-yl)-3-fluorophenyl)-4-(5-cyclopropyl-2-fluorophenyl)-3-methylimidazolidin-2-one;
1-(4-(4-amino-7-methyl-7H-pyrrolo[2,3-d]pyrimidin-5-yl)-3-fluorophenyl)-3-ethyl-4-(6-methylpyridin-2-yl)imidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one (enantiomer 1);
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one (enantiomer 2);
1-(4-(4-amino-7-methyl-7H-pyrrolo[2,3-d]pyrimidin-5-yl)-3-fluorophenyl)-4-(5-chloro-2-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one (enantiomer 1);
1-(4-(4-amino-7-methyl-7H-pyrrolo[2,3-d]pyrimidin-5-yl)-3-fluorophenyl)-3-ethyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(4-fluorophenyl)imidazolidin-2-one;
1-(4-(4-amino-7-methyl-7H-pyrrolo[2,3-d]pyrimidin-5-yl)-3-fluorophenyl)-4-cyclohexyl-3-methylimidazolidin-2-one;
1-(4-(4-amino-7-methyl-7H-pyrrolo[2,3-d]pyrimidin-5-yl)-3-fluorophenyl)-4-(4-fluorophenyl)-3-methylimidazolidin-2-one (enantiomer 1);
1-(4-(4-amino-7-methyl-7H-pyrrolo[2,3-d]pyrimidin-5-yl)-3-fluorophenyl)-3-methyl-4-(pyridin-3-yl)imidazolidin-2-one;
1-(4-(4-amino-2,7-dimethyl-7H-pyrrolo[2,3-d]pyrimidin-5-yl)-3-fluorophenyl)-4-cyclohexyl-3-methylimidazolidin-2-one (enantiomer 1);
1-(4-(4-amino-7-methyl-7H-pyrrolo[2,3-d]pyrimidin-5-yl)-3-fluorophenyl)-4-cyclohexyl-3-methylimidazolidin-2-one (enantiomer 1);
1-(4-(4-amino-7-methyl-7H-pyrrolo[2,3-d]pyrimidin-5-yl)-3-fluorophenyl)-4-cyclohexyl-3-methylimidazolidin-2-one (enantiomer 2);
1-(4-(4-amino-2,7-dimethyl-7H-pyrrolo[2,3-d]pyrimidin-5-yl)-3-fluorophenyl)-4-(4-methoxyphenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-4-(2,4-difluorophenyl)-3-ethylimidazolidin-2-one (enantiomer 2);
1-(4-(4-amino-7-methyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-3-methyl-4-(2,4,6-trifluorophenyl)imidazolidin-2-one (enantiomer 1);
1-(4-(4-amino-2,7-dimethyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-4-(3-(difluoromethoxy) phenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-ethylimidazolidin-2-one;
1-(4-(4-amino-7-methyl-7H-pyrrolo[2,3-d]pyrimidin-5-yl)-3-fluorophenyl)-3-(2,2-difluoroethyl)-4-(2,4-difluorophenyl)imidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(2,4-difluorophenyl)-3-(2,2,2-trifluoroethyl)imidazolidin-2-one;
1-(4-(4-amino-2,7-dimethyl-7H-pyrrolo[2,3-d]pyrimidin-5-yl)-3-fluorophenyl)-4-(3,4-difluorophenyl)-3-methylimidazolidin-2-one (enantiomer 1);
1-(4-(4-amino-7-methyl-7H-pyrrolo[2,3-d]pyrimidin-5-yl)-3-fluorophenyl)-4-(2,6-difluorophenyl)-3-methylimidazolidin-2-one;
1-(4-(4-amino-7-(2,2,2-trifluoroethyl)-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-(2,2,2-trifluoroethyl)imidazolidin-2-one (enantiomer 1);
1-(4-(4-amino-7-methyl-7H-pyrrolo[2,3-d]pyrimidin-5-yl)-3-fluorophenyl)-4-(3-fluoro-5-(trifluoromethyl)phenyl)-3-methylimidazolidin-2-one;
1-(4-(4-amino-7-methyl-7H-pyrrolo[2,3-d]pyrimidin-5-yl)-3-fluorophenyl)-3-ethyl-4-(3-fluoro-5-(trifluoromethyl) phenyl)imidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one (enantiomer 1);
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one (enantiomer 2);
1-(4-(4-amino-7-methyl-7H-pyrrolo[2,3-d]pyrimidin-5-yl)-3-fluorophenyl)-3-ethyl-4-(3-fluoro-5-(trifluoromethyl)phenyl)imidazolidin-2-one (enantiomer 1);
1-(4-(4-amino-7-methyl-7H-pyrrolo[2,3-d]pyrimidin-5-yl)-3-fluorophenyl)-3-ethyl-4-(3-fluoro-5-(trifluoromethyl) phenyl)imidazolidin-2-one (enantiomer 1);
1-(4-(4-amino-7-cyclopropyl-2-methyl-7H-pyrrolo[2,3-d]pyrimidin-5-yl)-3-fluorophenyl)-4-(4-fluorophenyl)-3-methylimidazolidin-2-one (enantiomer 1);
1-(4-(4-amino-7-cyclopropyl-2-methyl-7H-pyrrolo[2,3-d]pyrimidin-5-yl)-3-fluorophenyl)-4-(4-fluorophenyl)-3-methylimidazolidin-2-one;
1-(4-(4-amino-7-isopropyl-7H-pyrrolo[2,3-d]pyrimidin-5-yl)-3-fluorophenyl)-4-(2,4-difluorophenyl)-3-methylimidazolidin-2-one;
1-(4-(4-amino-6,7-dimethyl-7H-pyrrolo[2,3-d]pyrimidin-5-yl)-3-fluorophenyl)-4-(2,4-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,4-difluorophenyl)-3-methylimidazolidin-2-one;
1-(4-(4-amino-7-isopropyl-2-methyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-7-methyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,4-difluorophenyl)-3-ethylimidazolidin-2-one;
1-(4-(4-amino-1-methyl-1H-pyrazolo[3,4-d]pyrimidin-3-yl)-3-fluorophenyl)-4-(2,4-difluorophenyl)-3-ethylimidazolidin-2-one;
1-(4-(4-amino-1-methyl-1H-pyrazolo[3,4-d]pyrimidin-3-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one (enantiomer 1);
1-(4-(4-amino-7-methyl-7H-pyrrolo[2,3-d]pyrimidin-5-yl)-3-fluorophenyl)-4-(3,5-difluorophenyl)-3-methylimidazolidin-2-one (enantiomer 2);
1-(4-(4-amino-7-methyl-7H-pyrrolo[2,3-d]pyrimidin-5-yl)-3-fluorophenyl)-4-(3-methoxyphenyl)-3-methylimidazolidin-2-one;
1-(4-(4-amino-7-methyl-7H-pyrrolo[2,3-d]pyrimidin-5-yl)-3-fluorophenyl)-4-(3-methoxyphenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-4-(3,5-difluorophenyl)-3-methylimidazolidin-2-one (enantiomer 1);
1-(4-(4-amino-2,7-dimethyl-7H-pyrrolo[2,3-d]pyrimidin-5-yl)-3,5-difluorophenyl)-4-(2,4-difluorophenyl)-3-methylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,4-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; and
1-(4-(4-amino-7-ethyl-2-methyl-7H-pyrrolo[2,3-d]pyrimidin-5-yl)-3-fluorophenyl)-4-(2,4-difluorophenyl)-3-methylimidazolidin-2-one;
   and pharmaceutically acceptable salts thereof.

Included in the compounds of Formula (I) are:
1-(4-(4-amino-7-methyl-7H-pyrrolo[2,3-d]pyrimidin-5-yl)-3-fluorophenyl)-3-methyl-4-(3-(trifluoromethyl)phenyl)imidazolidin-2-one;
1-(4-(4-amino-7-methyl-7H-pyrrolo[2,3-d]pyrimidin-5-yl)-3-fluorophenyl)-3-ethyl-4-(3-(trifluoromethyl)phenyl)imidazolidin-2-one;
1-(4-(4-amino-7-methyl-7H-pyrrolo[2,3-d]pyrimidin-5-yl)-3-fluorophenyl)-3-methyl-4-(6-methylpyridin-2-yl)imidazolidin-2-one;
1-(4-(4-amino-7-isopropyl-7H-pyrrolo[2,3-d]pyrimidin-5-yl)-3-fluorophenyl)-4-(2,4-difluorophenyl)-3-ethylimidazolidin-2-one;
1-(4-(4-amino-7-methyl-7H-pyrrolo[2,3-d]pyrimidin-5-yl)-3-fluorophenyl)-4-(3-(difluoromethoxy) phenyl)-3-ethylimidazolidin-2-one;
1-(4-(4-amino-7-methyl-7H-pyrrolo[2,3-d]pyrimidin-5-yl)-3-fluorophenyl)-3-cyclopropyl-4-(2,5-difluorophenyl)imidazolidin-2-one;
1-(4-(4-amino-7-methyl-7H-pyrrolo[2,3-d]pyrimidin-5-yl)-3-fluorophenyl)-3-ethyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,4,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,4,6-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)-3-ethyl-4-methylimidazolidin-2-one;
1-(4-(4-amino-7-methyl-7H-pyrrolo[2,3-d]pyrimidin-5-yl)-3-fluorophenyl)-4-(2,5-difluorophenyl)-3,4-dimethylimidazolidin-2-one;
4-(4-(4-amino-7-methyl-7H-pyrrolo[2,3-d]pyrimidin-5-yl)-3-fluorophenyl)-1-(2,5-difluorophenyl)-2-ethyl-2,4-diazabicyclo[3.1.0]hexan-3-one;
4-(4-(4-amino-7-methyl-7H-pyrrolo[2,3-d]pyrimidin-5-yl)-3-fluorophenyl)-1-(2,5-difluorophenyl)-2-methyl-2,4-diazabicyclo[3.1.0]hexan-3-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one;
1-(4-(4-amino-6,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-1-methyl-1H-pyrazolo[3,4-d]pyrimidin-3-yl)-3-fluorophenyl)-4-(2,5-difluorophenyl)-3-ethylimidazolidin-2-one; and
1-(4-(4-amino-1-methyl-1H-pyrazolo[3,4-d]pyrimidin-3-yl)-3-fluorophenyl)-4-(2,5-difluorophenyl)-3-methylimidazolidin-2-one;
   and salts thereof including pharmaceutically acceptable salts thereof.

Included in the compounds of Formula (I) are:
1-(4-(4-amino-7-methyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-ethylimidazolidin-2-one, (enantiomer 1);
1-(4-(4-amino-7-methyl-7H-pyrrolo[2,3-d]pyrimidin-5-yl)-3-fluorophenyl)-4-(2,5-difluorophenyl)-3-ethylimidazolidin-2-one, (enantiomer 2);
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one, (enantiomer 1);
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one, (enantiomer 1);
1-(4-(4-amino-7-methyl-7H-pyrrolo[2,3-d]pyrimidin-5-yl)-3-fluorophenyl)-4-cyclohexyl-3-methylimidazolidin-2-one;
1-(4-(4-amino-7-methyl-7H-pyrrolo[2,3-d]pyrimidin-5-yl)-3-fluorophenyl)-4-(4-fluorophenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one (enantiomer 1); and
1-(4-(4-amino-7-methyl-7H-pyrrolo[2,3-d]pyrimidin-5-yl)-3-fluorophenyl)-4-(3,5-difluorophenyl)-3-methylimidazolidin-2-one (enantiomer 1);
   and pharmaceutically acceptable salts thereof.

Included in the compounds of Formula (I) are:
1-(4-(4-amino-7-methyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-7-methyl-7H-pyrrolo[2,3-d]pyrimidin-5-yl)-3-fluorophenyl)-4-(3,5-difluorophenyl)-3-methylimidazolidin-2-one (enantiomer 1);
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one (enantiomer 2);
1-(4-(4-amino-7-methyl-7H-pyrrolo[2,3-d]pyrimidin-5-yl)-3-fluorophenyl)-4-(3,4-difluorophenyl)-3-methylimidazolidin-2-one (enantiomer 1); and
1-(4-(4-amino-7-methyl-7H-pyrrolo[2,3-d]pyrimidin-5-yl)-3-fluorophenyl)-4-(4-fluorophenyl)-3-methylimidazolidin-2-one (enantiomer 1);
   and salts thereof including pharmaceutically acceptable salts thereof.

Included in the compounds of Formula (I) are:
1-(4-(4-amino-7-methyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-ethylimidazolidin-2-one (enantiomer 1);
1-(4-(4-amino-7-methyl-7H-pyrrolo[2,3-d]pyrimidin-5-yl)-3-fluorophenyl)-4-(2,5-difluorophenyl)-3-ethylimidazolidin-2-one (enantiomer 2);
1-(4-(4-Amino-7-methyl-7H-pyrrolo[2,3-d]pyrimidin-5-yl)-3-fluorophenyl)-4-(3,5-difluorophenyl)pyrrolidin-2-one;
1-(4-(4-Amino-7-methyl-7H-pyrrolo[2,3-d]pyrimidin-5-yl)-3-methylphenyl)-4-(3,5-difluorophenyl)pyrrolidin-2-one;
1-(4-(4-Amino-7-methyl-7H-pyrrolo[2,3-d]pyrimidin-5-yl)-3-fluorophenyl)-4-cyclohexylpyrrolidin-2-one;
1-(4-(4-amino-7-methyl-7H-pyrrolo[2,3-d]pyrimidin-5-yl)-3-fluorophenyl)-3-methyl-4-(pyridin-2-yl) imidazolidin-2-one;
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one (enantiomer 2);
1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one (enantiomer 1);
1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one (enantiomer 2);
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one (enantiomer 1);
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one (enantiomer 2);
1-(4-(4-amino-7-methyl-7H-pyrrolo[2,3-d]pyrimidin-5-yl)-3-fluorophenyl)-3-methyl-4-(pyridin-4-yl)imidazolidin-2-one;
1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one (enantiomer 1); and
1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one (enantiomer 2);
   and salts thereof including pharmaceutically acceptable salts thereof.

Included in the compounds of Formula (I) are:
1-(4-(4-amino-7-methyl-7H-pyrrolo[2,3-d]pyrimidin-5-yl)-3-fluorophenyl)-4-(2,5-difluorophenyl)-3-isobutylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one;
1-(4-(4-amino-7-(2,2-difluoroethyl)-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-3-benzyl-4-(2,5-difluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)imidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-ethylimidazolidin-2-one;
1-(4-(4-amino-7-methyl-7H-pyrrolo[2,3-d]pyrimidin-5-yl)-3-fluorophenyl)-4-cyclopentylpyrrolidin-2-one;
1-(4-(4-amino-7-methyl-7H-pyrrolo[2,3-d]pyrimidin-5-yl)phenyl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(3,5-dimethylphenyl)pyrrolidin-2-one;
1-(4-(4-amino-7-methyl-7H-pyrrolo[2,3-d]pyrimidin-5-yl)-3-fluorophenyl)-4-(2,5-difluorophenyl)pyrrolidin-2-one;
1-(5-(4-amino-7-methyl-7H-pyrrolo[2,3-d]pyrimidin-5-yl)pyridin-2-yl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(6-methylpyridin-2-yl)pyrrolidin-2-one;
1-(4-(4-amino-7-methyl-7H-pyrrolo[2,3-d]pyrimidin-5-yl)-3-fluorophenyl)-4-(2,4-difluorophenyl)pyrrolidin-2-one;
1-(4-(4-amino-7-methyl-7H-pyrrolo[2,3-d]pyrimidin-5-yl)-3-fluorophenyl)-4-(3-chloro-2-fluorophenyl)pyrrolidin-2-one;
1-(4-(4-amino-7-methyl-7H-pyrrolo[2,3-d]pyrimidin-5-yl)-3-fluorophenyl)-3-ethyl-4-(m-tolyl)imidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-methyl-4-(m-tolyl)imidazolidin-2-one;
1-(5-(4-amino-7-methyl-7H-pyrrolo[2,3-d]pyrimidin-5-yl)pyridin-2-yl)-4-(2,5-difluorophenyl)-3-methylimidazolidin-2-one;
1-(5-(4-amino-7-methyl-7H-pyrrolo[2,3-d]pyrimidin-5-yl)pyridin-2-yl)-4-(2,5-difluorophenyl)-3-ethylimidazolidin-2-one;
1-(4-(4-aminothieno[2,3-d]pyrimidin-5-yl)-3-fluorophenyl)-4-(2,5-difluorophenyl)-3-ethylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one; (enantiomer 1)
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one; (enantiomer 2)
1-(4-(4-amino-7-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(5-cyclopropyl-2-fluorophenyl)-3-ethylimidazolidin-2-one;
1-(4-(4-amino-7-methyl-7H-pyrrolo[2,3-d]pyrimidin-5-yl)-3-fluorophenyl)-4-(5-cyclopropyl-2-fluorophenyl)-3-methylimidazolidin-2-one;
1-(4-(4-amino-7-methyl-7H-pyrrolo[2,3-d]pyrimidin-5-yl)-3-fluorophenyl)-3-ethyl-4-(6-methylpyridin-2-yl)imidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(5-chloro-2-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one; (enantiomer1)
1-(4-(4-amino-7-methyl-7H-pyrrolo[2,3-d]pyrimidin-5-yl)-3-fluorophenyl)-3-ethyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(4-fluorophenyl)imidazolidin-2-one;
1-(4-(4-amino-7-methyl-7H-pyrrolo[2,3-d]pyrimidin-5-yl)-3-fluorophenyl)-4-cyclohexyl-3-methylimidazolidin-2-one;
1-(4-(4-amino-7-methyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-3-methyl-4-(pyridin-3-yl)imidazolidin-2-one;
1-(4-(4-amino-2,7-dimethyl-7H-pyrrolo[2,3-d]pyrimidin-5-yl)-3-fluorophenyl)-4-cyclohexyl-3-methylimidazolidin-2-one (enantiomer 1)
1-(4-(4-amino-7-methyl-7H-pyrrolo[2,3-d]pyrimidin-5-yl)-3-fluorophenyl)-4-cyclohexyl-3-methylimidazolidin-2-one; (enantiomer 1)
1-(4-(4-amino-7-methyl-7H-pyrrolo[2,3-d]pyrimidin-5-yl)-3-fluorophenyl)-4-cyclohexyl-3 methylimidazolidin-2-one; (enantiomer 2)
1-(4-(4-amino-2,7-dimethyl-7H-pyrrolo[2,3-d]pyrimidin-5-yl)-3-fluorophenyl)-4-(4-methoxyphenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(2,4-difluorophenyl)-3-ethylimidazolidin-2-one; (enantiomer 2)
1-(4-(4-amino-7-methyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-3-methyl-4-(2,4,6-trifluorophenyl)imidazolidin-2-one; (enantiomer 1)
1-(4-(4-amino-2,7-dimethyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-4-(3-(difluoromethoxy)phenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-ethylimidazolidin-2-one;
1-(4-(4-amino-7-methyl-7H-pyrrolo[2,3-d]pyrimidin-5-yl)-3-fluorophenyl)-3-(2,2-difluoroethyl)-4-(2,4-difluorophenyl)imidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(2,4-difluorophenyl)-3-(2,2,2-trifluoroethyl)imidazolidin-2-one;
1-(4-(4-amino-2,7-dimethyl-7H-pyrrolo[2,3-d]pyrimidin-5-yl)-3-fluorophenyl)-4-(3,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,6-difluorophenyl)-3-methylimidazolidin-2-one;
1-(4-(4-amino-7-(2,2,2-trifluoroethyl)-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-(2,2,2-trifluoroethyl)imidazolidin-2-one; (enantiomer 1)
1-(4-(4-amino-7-methyl-7H-pyrrolo[2,3-d]pyrimidin-5-yl)-3-fluorophenyl)-4-(3-fluoro-5-(trifluoromethyl)phenyl)-3-methylimidazolidin-2-one;
1-(4-(4-amino-7-methyl-7H-pyrrolo[2,3-d]pyrimidin-5-yl)-3-fluorophenyl)-3-ethyl-4-(3-fluoro-5-(trifluoromethyl) phenyl)imidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one; (enantiomer 1)
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-ethyl-4-(3-fluoro-5-(trifluoromethyl)phenyl)imidazolidin-2-one; (enantiomer 1)
1-(4-(4-amino-7-methyl-7H-pyrrolo[2,3-d]pyrimidin-5-yl)-3-fluorophenyl)-3-ethyl-4-(3-fluoro-5-(trifluoromethyl)phenyl)imidazolidin-2-one; (enantiomer 1)
1-(4-(4-amino-7-cyclopropyl-2-methyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-7-cyclopropyl-2-methyl-7H-pyrrolo[2,3-d]pyrimidin-5-yl)-3-fluorophenyl)-4-(4-fluorophenyl)-3-methylimidazolidin-2-one;
1-(4-(4-amino-7-isopropyl-7H-pyrrolo[2,3-d]pyrimidin-5-yl)-3-fluorophenyl)-4-(2,4-difluorophenyl)-3-methylimidazolidin-2-one;
1-(4-(4-amino-6,7-dimethyl-7H-pyrrolo[2,3-d]pyrimidin-5-yl)-3-fluorophenyl)-4-(2,4-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,4-difluorophenyl)-3-methylimidazolidin-2-one;
1-(4-(4-amino-7-isopropyl-2-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,4-difluorophenyl)-3-ethylimidazolidin-2-one;
1-(4-(4-amino-1-methyl-1H-pyrazolo[3,4-d]pyrimidin-3-yl)-3-fluorophenyl)-4-(2,4-difluorophenyl)-3-ethylimidazolidin-2-one;
1-(4-(4-amino-1-methyl-1H-pyrazolo[3,4-d]pyrimidin-3-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(3-methoxyphenyl)-3-methylimidazolidin-2-one;
1-(4-(4-amino-7-methyl-7H-pyrrolo[2,3-d]pyrimidin-5-yl)-3-fluorophenyl)-4-(3-methoxyphenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(3,5-difluorophenyl)-3-methylimidazolidin-2-one; (enantiomer 1)
1-(4-(4-amino-2,7-dimethyl-7H-pyrrolo[2,3-d]pyrimidin-5-yl)-3,5-difluorophenyl)-4-(2,4-difluorophenyl)-3-methylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,4-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one;
1-(4-(4-amino-7-ethyl-2-methyl-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(5-fluoropyridin-3-yl)-3-methylimidazolidin-2-one; (enantiomer 1)
1-(4-(4-amino-7-methyl-7H-pyrrolo[2,3-d]pyrimidin-5-yl)-3-fluorophenyl)-4-(5-fluoro-6-methylpyridin-2-yl)-3-methylimidazolidin-2-one;
1-(4-(4-amino-7-methyl-7H-pyrrolo[2,3-d]pyrimidin-5-yl)-3-fluorophenyl)-4-(5-fluoro-6-methylpyridin-2-yl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,4-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,4-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,4-difluorophenyl)-3-methylimidazolidin-2-one; (enantiomer 2)
1-(4-(4-amino-7-methyl-7H-pyrrolo[2,3-d]pyrimidin-5-yl)-3-methylphenyl)-4-(3,5-difluorophenyl)-3-methylimidazolidin-2-one;
1-(4-(4-amino-7-cyclopropyl-7H-pyrrolo[2,3-d]pyrimidin-5-yl)-3-methylphenyl)-4-(3,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-chlorophenyl)-4-(3,5-difluorophenyl)-3-methylimidazolidin-2-one;
1-(4-(4-amino-7-cyclopropyl-7H-pyrrolo[2,3-d]pyrimidin-5-yl)-3-chlorophenyl)-4-(3,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one; (enantiomer 1)
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one; (enantiomer 2)
1-(4-(4-amino-7-cyclopropyl-7H-pyrrolo[2,3-d]pyrimidin-5-yl)-3-fluorophenyl)-4-(3-fluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3-fluorophenyl)-3-methylimidazolidin-2-one; (enantiomer 2)
1-(4-(4-amino-7-methyl-7H-pyrrolo[2,3-d]pyrimidin-5-yl)-3-chlorophenyl)-4-(3,5-difluorophenyl)-3-methylimidazolidin-2-one; (enantiomer 1)
1-(4-(4-amino-7-methyl-7H-pyrrolo[2,3-d]pyrimidin-5-yl)-3-chlorophenyl)-4-(3,5-difluorophenyl)-3-methylimidazolidin-2-one; (enantiomer 2) 1-(4-(4-amino-7-cyclopropyl-7H-pyrrolo[2,3-d]pyrimidin-5-yl)-3-chlorophenyl)-4-(3,5-difluorophenyl)-3-methylimidazolidin-2-one; (enantiomer 1)
1-(4-(4-amino-7-cyclopropyl-7H-pyrrolo[2,3-d]pyrimidin-5-yl)-3-chlorophenyl)-4-(3,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(3-chloro-4-fluorophenyl)-3-methylimidazolidin-2-one;
1-(4-(4-amino-7-cyclopropyl-7H-pyrrolo[2,3-d]pyrimidin-5-yl)-3-fluorophenyl)-4-(3-chloro-4-fluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-4-fluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3-chloro-4-fluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(6-chloropyridin-3-yl)-3-methylimidazolidin-2-one;
1-(4-(4-amino-2,7-dimethyl-7H-pyrrolo[2,3-d]pyrimidin-5-yl)-3-fluorophenyl)-4-(6-chloropyridin-3-yl)-3-methylimidazolidin-2-one; (enantiomer 2)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-cycloheptyl-3-methylimidazolidin-2-one;
1-(4-(4-amino-2,7-dimethyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one; (enantiomer 1)
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-3-methyl-4-(2,3,5-trifluorophenyl)imidazolidin-2-one; (enantiomer 1) 1-(4-(4-amino-7-cyclopropyl-7H-pyrrolo[2,3-d]pyrimidin-5-yl)-3-fluorophenyl)-3-methyl-4-(2,3,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(2,3-dihydrobenzofuran-5-yl)-3-methylimidazolidin-2-one;
1-(4-(4-amino-7-cyclopropyl-7H-pyrrolo[2,3-d]pyrimidin-5-yl)-3-fluorophenyl)-4-(3-chlorophenyl)-3-methylimidazolidin-2-one;
1-(4-(4-amino-7-cyclopropyl-7H-pyrrolo[2,3-d]pyrimidin-5-yl)-3-fluorophenyl)-3-methyl-4-phenylimidazolidin-2-one;
1-(4-(4-amino-7-methyl-7H-pyrrolo[2,3-d]pyrimidin-5-yl)-3-fluorophenyl)-4-(2,5-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-cyclopropyl-7H-pyrrolo[2,3-d]pyrimidin-5-yl)-3-fluorophenyl)-4-(3-chlorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3-chlorophenyl)-3-methylimidazolidin-2-one; (enantiomer 2)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one;
1-(4-(4-amino-7-cyclopropyl-7H-pyrrolo[2,3-d]pyrimidin-5-yl)-3-fluorophenyl)-3-methyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one; (enantiomer 1)
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-3-methyl-4-(2,4,5-trifluorophenyl)imidazolidin-2-one; (enantiomer 1)
1-(4-(4-amino-7-cyclopropyl-7H-pyrrolo[2,3-d]pyrimidin-5-yl)-3-fluorophenyl)-3-methyl-4-(2,4,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-4-(4-fluorophenyl)-3-methylimidazolidin-2-one;
1-(4-(4-amino-7-cyclopropyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(4-fluorophenyl)-3-methylimidazolidin-2-one; (enantiomer 2)
1-(4-(4-amino-7-cyclopropyl-7H-pyrrolo[2,3-d]pyrimidin-5-yl)-3-fluorophenyl)-3-methyl-4-phenylimidazolidin-2-one; (enantiomer 1)
1-(4-(4-amino-7-cyclopropyl-7H-pyrrolo[2,3-d]pyrimidin-5-yl)-3-fluorophenyl)-3-methyl-4-phenylimidazolidin-2-one; (enantiomer 2)
1-(4-(4-amino-7-cyclopropyl-7H-pyrrolo[2,3-d]pyrimidin-5-yl)-3-fluorophenyl)-4-(4-chlorophenyl)-3-methylimidazolidin-2-one;
1-(4-(4-amino-7-cyclopropyl-7H-pyrrolo[2,3-d]pyrimidin-5-yl)-3-fluorophenyl)-4-(4-chlorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,3-di hydrobenzofuran-5-yl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,5-difluorophenyl)-3-ethylimidazolidin-2-one;
1-(4-(4-amino-7-methyl-7H-pyrrolo[2,3-d]pyrimidin-5-yl)-3-fluorophenyl)-4-(3,5-difluorophenyl)-3-ethylimidazolidin-2-one; (enantiomer 1)
1-(4-(4-amino-7-methyl-7H-pyrrolo[2,3-d]pyrimidin-5-yl)-3-fluorophenyl)-4-(3,5-difluorophenyl)-3-ethylimidazolidin-2-one; (enantiomer 2)
1-(4-(4-amino-7-cyclopropyl-7H-pyrrolo[2,3-d]pyrimidin-5-yl)-3-fluorophenyl)-4-(3,5-difluorophenyl)-3-ethylimidazolidin-2-one; (enantiomer 1) and
1-(4-(4-amino-7-cyclopropyl-7H-pyrrolo[2,3-d]pyrimidin-5-yl)-3-fluorophenyl)-4-(3,5-difluorophenyl)-3-ethylimidazolidin-2-one; (enantiomer 2) and salts thereof including pharmaceutically acceptable salts thereof.
Included in the compounds of Formula (I) are:
1-(4-(4-amino-7-methyl-7H-pyrrolo[2,3-d]pyrimidin-5-yl)-3-fluorophenyl)-3-methyl-4-(3-(trifluoromethyl)phenyl)imidazolidin-2-one;
1-(4-(4-amino-7-methyl-7H-pyrrolo[2,3-d]pyrimidin-5-yl)-3-fluorophenyl)-3-ethyl-4-(3-(trifluoromethyl)phenyl)imidazolidin-2-one;
1-(4-(4-amino-7-methyl-7H-pyrrolo[2,3-d]pyrimidin-5-yl)-3-fluorophenyl)-3-methyl-4-(6-methylpyridin-2-yl)imidazolidin-2-one;
1-(4-(4-amino-7-isopropyl-7H-pyrrolo[2,3-d]pyrimidin-5-yl)-3-fluorophenyl)-4-(2,4-difluorophenyl)-3-ethylimidazolidin-2-one;
1-(4-(4-amino-7-methyl-7H-pyrrolo[2,3-d]pyrimidin-5-yl)-3-fluorophenyl)-4-(3-(difluoromethoxy) phenyl)-3-ethylimidazolidin-2-one;
1-(4-(4-amino-7-methyl-7H-pyrrolo[2,3-d]pyrimidin-5-yl)-3-fluorophenyl)-3-cyclopropyl-4-(2,5-difluorophenyl)imidazolidin-2-one;
1-(4-(4-amino-7-methyl-7H-pyrrolo[2,3-d]pyrimidin-5-yl)-3-fluorophenyl)-3-ethyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,4,6-trifluorophenyl)imidazolidin-2-one;
1-(4-(4-amino-7-methyl-7H-pyrrolo[2,3-d]pyrimidin-5-yl)-3-fluorophenyl)-3-ethyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)-3-ethyl-4-methylimidazolidin-2-one;
1-(4-(4-amino-7-methyl-7H-pyrrolo[2,3-d]pyrimidin-5-yl)-3-fluorophenyl)-4-(2,5-difluorophenyl)-3,4-dimethylimidazolidin-2-one;
4-(4-(4-amino-7-methyl-7H-pyrrolo[2,3-d]pyrimidin-5-yl)-3-fluorophenyl)-1-(2,5-difluorophenyl)-2-ethyl-2,4-diazabicyclo[3.1.0]hexan-3-one;
4-(4-(4-amino-7-methyl-7H-pyrrolo[2,3-d]pyrimidin-5-yl)-3-fluorophenyl)-1-(2,5-difluorophenyl)-2-methyl-2,4-diazabicyclo[3.1.0]hexan-3-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-1-methyl-1H-pyrazolo[3,4-d]pyrimidin-3-yl)-3-fluorophenyl)-4-(2,5-difluorophenyl)-3-ethylimidazolidin-2-one;
1-(4-(4-amino-1-methyl-1H-pyrazolo[3,4-d]pyrimidin-3-yl)-3-fluorophenyl)-4-(2,5-difluorophenyl)-3-methylimidazolidin-2-one;
   and salts thereof including pharmaceutically acceptable salts thereof.

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I).

The salts, including pharmaceutically acceptable salts, of the compounds of the invention are readily prepared by those of skill in the art.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

As used herein, when enantiomers are isolated in entiomerically enriched form with unknown absolute chemistry, they are assigned as enantiomer 1 or enantiomer 2 based on their respective retention times. For the given set of purification conditions by chiral HPLC, representative examples are Examples 2, 3, 10, 11, 12 and 13, the first enantiomer to elute is assigned as “enantiomer 1” and the slower eluting enantiomer is assigned as “enantiomer 2”.

The compounds according to Formula (I) may also contain double bonds or other centers of geometric asymmetry. Where the stereochemistry of a center of geometric asymmetry present in Formula (I), or in any chemical structure illustrated herein, is not specified, the structure is intended to encompass the trans (E) geometric isomer, the cis (Z) geometric isomer, and all mixtures thereof. Likewise, all tautomeric forms are also included in Formula (I) whether such tautomers exist in equilibrium or predominately in one form.

The compounds of Formula (I) or salts, including pharmaceutically acceptable salts, thereof may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates wherein water is the solvent that is incorporated into the crystalline lattice are typically referred to as “hydrates.” Hydrates include stoichiometric hydrates as well as compositions containing vaiable amounts of water.

The skilled artisan will further appreciate that certain compounds of Formula (I) or salts, including pharmaceutically acceptable salts thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification.

The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

### Definitions

“Alkyl” refers to a hydrocarbon chain having the specified number of “member atoms”. For example, C₁-C₆ alkyl refers to an alkyl group having from 1 to 6 member atoms. Alkyl groups may be saturated, unsaturated, straight or branched. Representative branched alkyl groups have one, two, or three branches. Alkyl includes methyl, ethyl, ethylene, propyl (n-propyl and isopropyl), butene, butyl (n-butyl, isobutyl, and t-butyl), pentyl and hexyl.
“Alkoxy” refers to an —O-alkyl group wherein “alkyl” is as defined herein. For example, C₁-C₄alkoxy refers to an alkoxy group having from 1 to 4 member atoms. Representative branched alkoxy groups have one, two, or three branches. Examples of such groups include methoxy, ethoxy, propoxy, and butoxy.
“Aryl” refers to an aromatic hydrocarbon ring. Aryl groups are monocyclic, bicyclic, and tricyclic ring systems having a total of five to fourteen ring member atoms, wherein at least one ring system is aromatic and wherein each ring in the system contains 3 to 7 member atoms, such as phenyl, naphthalene, tetrahydronaphthalene and biphenyl. Suitably aryl is phenyl.
“Bicycloheteroaryl” refers to two fused aromatic rings containing from 1 to 6 heteroatoms as member atoms. Bicycloheteroaryl groups containing more than one heteroatom may contain different heteroatoms. Bicycloheteroaryl rings have from 6 to 11 member atoms. Bicycloheteroaryl includes: 1H-pyrrolo[3,2-c]pyridinyl, 1H-pyrazolo[4,3-c]pyridinyl, 1H-pyrazolo[3,4-d]pyrimidinyl, 1H-pyrrolo[2,3-d]pyrimidinyl, 7H-pyrrolo[2,3-d]pyrimidinyl, thieno[3,2-c]pyridinyl, thieno[2,3-d]pyrimidinyl, furo[2,3-c]pyridinyl, furo[2,3-d]pyrimidinyl, indolyl, isoindolyl, indolizinyl, indazolyl, purinyl, quinolinyl, isoquinolinyl, quinoxalinyl, quinazolinyl, pteridinyl, cinnolinyl, azabenzimidazolyl, tetrahydrobenzimidazolyl, benzimidazolyl, benopyranyl, benzoxazolyl, benzofuranyl, isobenzofuranyl, benzothiazolyl, benzothienyl, imidazo[4.5-c]pyridinyl, imidazo[4.5-b]pyridinyl, furopyridinyl and napthyridinyl.
Suitably “Bicycloheteroaryl” includes: 1H-pyrazolo[3,4-d]pyrimidinyl, 1H-pyrrolo[2,3-d]pyrimidinyl, 7H-pyrrolo[2,3-d]pyrimidinyl, thieno[3,2-c]pyridinyl, thieno[2,3-d]pyrimidinyl, furo[2,3-c]pyridinyl, indolyl, isoindolyl, indolizinyl, indazolyl, purinyl, quinolinyl, isoquinolinyl, quinoxalinyl, quinazolinyl, pteridinyl, cinnolinyl, azabenzimidazolyl, tetrahydrobenzimidazolyl, benzimidazolyl, benopyranyl, benzoxazolyl, benzofuranyl, isobenzofuranyl, benzothiazolyl, benzothienyl, imidazo[4.5-c]pyridinyl, imidazo[4.5-b]pyridinyl, furopyrimidinyl and napthyridinyl. Suitably 1H-pyrazolo[3,4-d]pyrimidinyl, 1H-pyrrolo[2,3-d]pyrimidinyl, thieno[3,2-c]pyridinyl, thieno[2,3-d]pyrimidinyl, indazolyl, quinolinyl, quinazolinyl or benzothiazolyl. Suitably 1H-pyrazolo[3,4-d]pyrimidinyl, thieno[2,3-d]pyrimidinyl or 1H-pyrrolo[2,3-d]pyrimidinyl. Suitably 1H-pyrrolo[2,3-d]pyrimidinyl.
“Cycloalkyl”, unless otherwise defined, refers to a saturated or unsaturated non aromatic hydrocarbon ring having from three to seven carbon atoms. Cycloalkyl groups are monocyclic ring systems. For example, C₃-C₇ cycloalkyl refers to a cycloalkyl group having from 3 to 7 member atoms. Examples of cycloalkyl as used herein include: cyclopropyl, cyclobutyl, cyclopentyl, cyclohexyl, cyclobutenyl, cyclopentenyl, cyclohexenyl and cycloheptyl.
“Halo” refers to the halogen radicals fluoro, chloro, bromo, and iodo.
“Heteroaryl” refers to a monocyclic aromatic 4 to 8 member ring containing from 1 to 7 carbon atoms and containing from 1 to 4 heteroatoms, provided that when the number of carbon atoms is 3, the aromatic ring contains at least two heteroatoms. Heteroaryl groups containing more than one heteroatom may contain different heteroatoms. Heteroaryl includes: pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, tetrazinyl. Suitably, “heteroaryl” includes: pyrazolyl, pyrrolyl, isoxazolyl, pyridinyl, pyrimidinyl, pyridazinyl, and imidazolyl.
“Heterocycloalkyl” refers to a saturated or unsaturated non-aromatic ring containing 4 to 12 member atoms, of which 1 to 11 are carbon atoms and from 1 to 6 are heteroatoms. Heterocycloalkyl groups containing more than one heteroatom may contain different heteroatoms. Heterocycloalkyl groups are monocyclic ring systems or a monocyclic ring fused with an aryl ring or to a heteroaryl ring having from 3 to 6 member atoms. Heterocycloalkyl includes: pyrrolidinyl, tetrahydrofuranyl, dihydrofuranyl, pyranyl, tetrahydropyranyl, dihydropyranyl, tetrahydrothienyl, pyrazolidinyl, oxazolidinyl, oxetanyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, 1,3oxazolidin-2-onyl, hexahydro-1H-azepinyl, 4,5,6,7,tetrahydro-1H-benzimidazolyl, piperidinyl, 1,2,3,6-tetrahydro-pyridinyl and azetidinyl.
“Heteroatom” refers to a nitrogen, sulphur or oxygen atom.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

Ac (acetyl);
Ac₂O (acetic anhydride);
ACN (acetonitrile);
AIBN (azobis(isobutyronitrile));
BINAP (2,2′-bis(diphenylphosphino)-1,1′-binaphthyl);
BMS (borane—dimethyl sulphide complex);
Bn (benzyl);
Boc (tert-Butoxycarbonyl);
Boc₂O (di-tert-butyl dicarbonate);
BOP (Benzotriazole-1-yl-oxy-tris-(dimethylamino)-phosphonium hexafluorophosphate);
CAN (cerric ammonium nitrate);
Cbz (benzyloxycarbonyl);
CSI (chlorosulfonyl isocyanate);
CSF (cesium fluoride);
DABCO (1,4-Diazabicyclo[2.2.2]octane);
DAST (Diethylamino)sulfur trifluoride);
DBU (1,8-Diazabicyclo[5.4.0]undec-7-ene);

### DCC (Dicyclohexyl Carbodiimide);

DCE (1,2-dichloroethane);
DCM (dichloromethane);
DDQ (2,3-Dichloro-5,6-dicyano-1,4-benzoquinone);
ATP (adenosine triphosphate);
Bis-pinacolatodiboron (4,4,4′,4′,5,5,5′,5′-Octamethyl-2,2′-bi-1,3,2-dioxaborolane);
BSA (bovine serum albumin);
C18 (refers to 18-carbon alkyl groups on silicon in HPLC stationary phase)
CH₃CN (acetonitrile) Cy (cyclohexyl);
DCM (dichloromethane);
DIPEA (Hunig's base, N-ethyl-N-(1-methylethyl)-2-propanamine);
Dioxane (1,4-dioxane);
DMAP (4-dimethylaminopyridine);
DME (1,2-dimethoxyethane);
DMEDA (N,N′-dimethylethylenediamine);

### DMF (N,N-dimethylformamide);

DMSO (dimethylsulfoxide);
DPPA (diphenyl phosphoryl azide);
EDC (N-(3-dimethylaminopropyl)-N′ethylcarbodiimide);
EDTA (ethylenediaminetetraacetic acid);
EtOAc (ethyl acetate);
EtOH (ethanol);
Et₂O (diethyl ether);
HEPES (4-(2-hydroxyethyl)-1-piperazine ethane sulfonic acid);
HATU (O-(7-Azabenzotriazol-1-yl)-N,N,N′,N′-tetramethyluronium hexafluorophosphate);
HOAt (1-hydroxy-7-azabenzotriazole);
HOBt (1-hydroxybenzotriazole);
HOAc (acetic acid);
HPLC (high pressure liquid chromatography);
HMDS (hexamethyldisilazide);

### Hunig's Base (N,N-Diisopropylethylamine);

IPA (isopropyl alcohol);
Indoline (2,3-dihydro-1H-indole);
KHMDS (potassium hexamethyldisilazide);
LAH (lithium aluminum hydride);
LDA (lithium diisopropylamide);
LHMDS (lithium hexamethyldisilazide)
MeOH (methanol);
MTBE (methyl tert-butyl ether);
mCPBA (m-chloroperbezoic acid);
NaHMDS (sodium hexamethyldisilazide);

### NBS (N-bromosuccinimide);

PE (petroleum ether);
Pd₂(dba)₃ (Tris(dibenzylideneacetone)dipalladium(0);
Pd(dppf)Cl₂. DCM Complex ([1,1′-Bis(diphenylphosphino)ferrocene]dichloropalladium(II).dichloromethane complex);
PyBOP (benzotriazol-1-yl-oxytripyrrolidinophosphonium hexafluorophosphate);
PyBrOP (bromotripyrrolidinophosphonium hexafluorophosphate);
RPHPLC (reverse phase high pressure liquid chromatography);
RT (room temperature);
Sat. (saturated)
SFC (supercritical fluid chromatography);
SGC (silica gel chromatography);
SM (starting material);
TCL (thin layer chromatography);
TEA (triethylamine);
TEMPO (2,2,6,6-Tetramethylpiperidine 1-oxyl, free radical);
TFA (trifluoroacetic acid); and
THF (tetrahydrofuran).
All references to ether are to diethyl ether and brine refers to a saturated aqueous solution of NaCl.

### Compound Preparation

The compounds according to Formula (I) are prepared using conventional organic synthetic methods. A suitable synthetic route is depicted below in the following general reaction schemes. All of the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

As used in the Schemes, “x” and “r” groups represent corresponding positional groups on any of Formulas I to IV. As used in the Schemes, compounds of formula “r” and “T” represent all corresponding R¹ substituents of Formula (I). The compounds of Formulas I to IV can be prepared generally as described in the Schemes using appropriate substitutions for starting materials.

Compounds of the invention were prepared generally according to Scheme 1. 3-amino-3-(aryl/heteroaryl/alkyl/cycloalkyl) propanoic acids B were prepared by reacting corresponding aldehyde, malonic acid and ammonium acetate. The amino acid B was protected with Boc to obtain intermediate C. The amino acid C was cyclized to imidazolidinone D using DPPA. N-substituted imidazolidinone F were obtained by arylation of D using aryl or heteroaryl halide E. Deprotection of the Boc group was performed using acid, and subsequent N-alkylation afforded the tri-substituted imidazolidinone compounds H. After conversion to the boronate ester I, palladium catalyzed Suzuki-Miyaura reaction with the bicycloheteroaryl bromide J produced the compound K, which represents the structure of the compounds of the invention. Alternatively, the boronate ester was formed In situ and reacted with the bromide J under similar Suzuki-Miyaura reaction condition to obtain compounds K of the invention. In several examples enantiomers of the racemate compound K were separated by chiral HPLC chromatography. In some examples when r3=H, boronate ester formation of F followed by Suzuki-Miyaura coupling reaction was performed with J and removal of the protecting group to obtain compounds of the present invention.

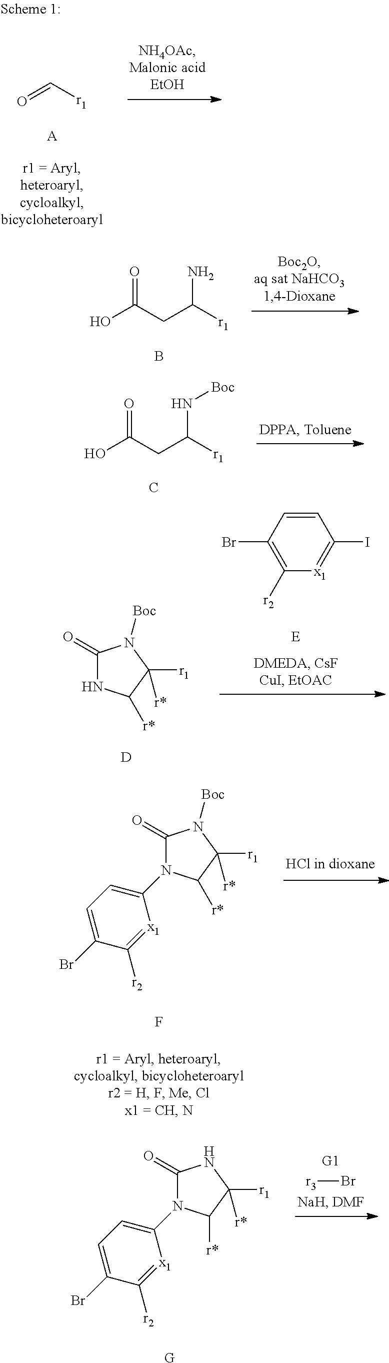

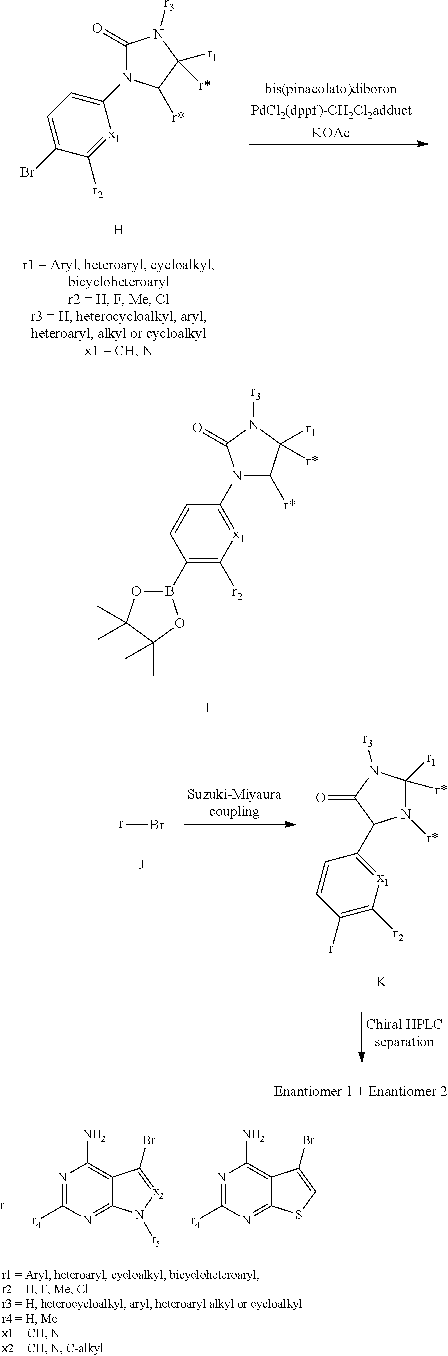

The intermediates G synthesized in Scheme 1 were alternatively synthesized using scheme 1a. Aldehyde A was reacted with t-butylsulfoxamine A1 imine to afford derivative A2. Nucleophilic addition of ethyl acetate to A2 yielded sulfinamidopropionate derivative A3. Ester hydrolysis of A3 gave the acid A4, which was cyclized to imidazolidinone A5 using DPPA. N-arylsubstituted imidazolidinone A6 were obtained by arylation of A5 using aryl or heteroaryl halide E. Deprotection of the t-butyl sulfoxide was performed using acid to give racemic intermediate G. Chirally pure compounds can be prepared by using chiral pure starting material t-butylsulfoxamine A1.

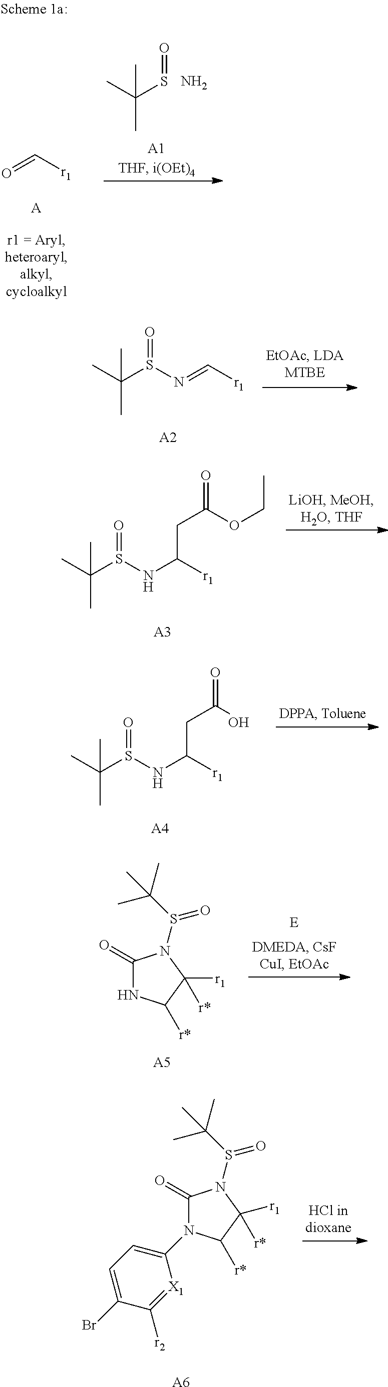

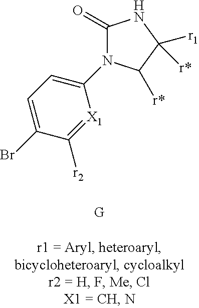

4-substituted pyrrolidinone compounds U were prepared according to Scheme 2. Substituted methyl acrylate M was prepared from corresponding aldehyde L by Wittig reaction. Nucleophilic addition of nitromethane at beta position of acrylate formed the methyl nitrobutanoate intermediate N. Nitro group reduction was performed using Pd/C under hydrogen atmosphere to obtain either methyl aminobutanoate O or aminobutanoicacid P. 4-substituted pyrrolidinone intermediate Q was prepared from the intermediate O by refluxing in an organic solvent such as methanol or by acid-amine coupling of intermediate P using coupling reagent such as T3P. N-arylation of intermediate Q with heteroaryl halide E resulted with 1,4-substituted pyrrolidinone intermediate R. After conversion to the boronate ester S, palladium catalyzed Suzuki-Miyaura reaction with the bicycloheteroaryl bromide T produced the compound U, which represents the structure of the compounds of the invention, in some examples boronate ester formation and Suzuki-Miyaura reaction were performed in situ to give compounds of the invention.

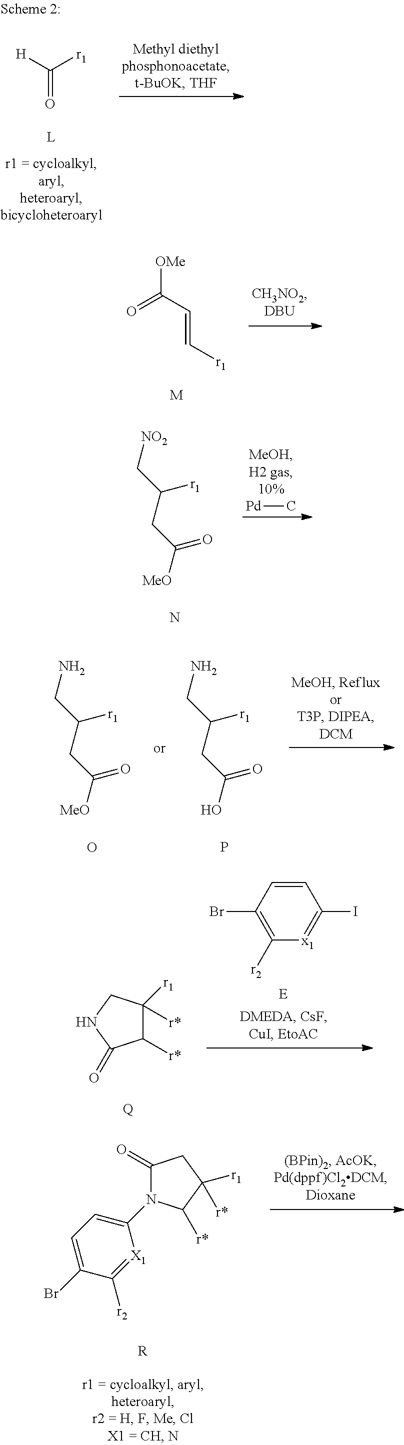

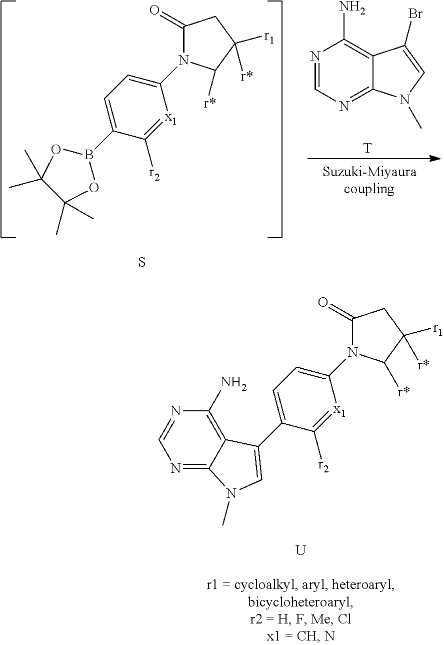

### METHODS OF USE

The compounds according to Formula (I) and pharmaceutically acceptable salts thereof are inhibitors of PERK. These compounds are potentially useful in the treatment of conditions wherein the underlying pathology is attributable to (but not limited to) activation of the UPR pathway, for example, neurodegenerative disorders, cancer, cardiovascular and metabolic diseases. Accordingly, in another aspect the invention is directed to methods of treating such conditions.

Suitably, the present invention relates to a method for treating or lessening the severity of breast cancer, including inflammatory breast cancer, ductal carcinoma, and lobular carcinoma.

Suitably the present invention relates to a method for treating or lessening the severity of colon cancer.

Suitably the present invention relates to a method for treating or lessening the severity of pancreatic cancer, including insulinomas, adenocarcinoma, ductal adenocarcinoma, adenosquamous carcinoma, acinar cell carcinoma, and glucagonoma.

Suitably the present invention relates to a method for treating or lessening the severity of skin cancer, including melanoma, including metastatic melanoma.

Suitably the present invention relates to a method for treating or lessening the severity of lung cancer including small cell lung cancer, non-small cell lung cancer, squamous cell carcinoma, adenocarcinoma, and large cell carcinoma.

Suitably the present invention relates to a method for treating or lessening the severity of cancers selected from the group consisting of brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

Suitably the present invention relates to a method for treating or lessening the severity of neurodegenerative diseases/injury, such as Alzheimer's disease, spinal cord injury, traumatic brain injury, ischemic stroke, stroke, Parkinson disease, metabolic syndrome, metabolic disorders, Huntington's disease, Creutzfeldt-Jakob Disease, fatal familial insomnia, Gerstmann-Sträussler-Scheinker syndrome, and related prion diseases, progressive supranuclear palsy, amyotrophic lateral sclerosis, and other diseases associated with UPR activation including: neuropathic pain, diabetes, myocardial infarction, cardiovascular disease, inflammation, fibrosis, chronic and acute diseases of the liver, fatty liver disease, liver steatosis, liver fibrosis chronic and acute diseases of the lung, lung fibrosis, chronic and acute diseases of the kidney, kidney fibrosis, chronic traumatic encephalopathy (CTE), neurodegeneration, dementia, frontotemporal dementias, tauopathies, Pick's disease, Neimann-Pick's disease, amyloidosis cognitive impairment, atherosclerosis, ocular diseases, and arrhythmias.

Suitably the present invention relates to a method preventing organ damage during and after organ transplantation and in the transportation of organs for transplantation. The method of preventing organ damage during and after organ transplantation will comprise the in vivo administration of a compound of Formula (I). The method of preventing organ damage during the transportation of organs for transplantation will comprise adding a compound of Formula (I) to the solution housing the organ during transportation.

The compounds of this invention inhibit angiogenesis which is implicated in the treatment of ocular diseases. Nature Reviews Drug Discovery 4, 711-712 (September 2005). Suitably the present invention relates to a method for treating or lessening the severity of ocular diseases/angiogenesis. In embodiments of methods according to the invention, the disorder of ocular diseases, including vascular leakage can be: edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

In some embodiments, the neovascular age-related macular degeneration is wet age-related macular degeneration. In other embodiments, the neovascular age-related macular degeneration is dry age-related macular degeneration and the patient is characterized as being at increased risk of developing wet age-related macular degeneration.

The methods of treatment of the invention comprise administering an effective amount of a compound according to Formula (I) or a pharmaceutically acceptable salt, thereof to a patient in need thereof.

The invention also provides a compound according to Formula (I) or a pharmaceutically-acceptable salt thereof for use in medical therapy, and particularly in therapy for: cancer, pre-cancerous syndromes, Alzheimer's disease, neuropathic pain, spinal cord injury, traumatic brain injury, ischemic stroke, stroke, diabetes, Parkinson disease, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, arrhythmias, in organ transplantation and in the transportation of organs for transplantation. Thus, in further aspect, the invention is directed to the use of a compound according to Formula (I) or a pharmaceutically acceptable salt thereof in the preparation of a medicament for the treatment of a disorder characterized by activation of the UPR, such as cancer.

By the term “treating” and derivatives thereof as used herein, in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

Prophylactic therapy is appropriate when a subject has, for example, a strong family history of cancer or is otherwise considered at high risk for developing cancer, or when a subject has been exposed to a carcinogen.

As used herein, the term “effective amount” and derivatives thereof means that amount of a drug or pharmaceutical agent that will elicit the biological or medical response of a tissue, system, animal or human that is being sought, for instance, by a researcher or clinician.

Furthermore, the term “therapeutically effective amount” and derivatives thereof means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” or “subject” refers to a human or other animal. Suitably the patient or subject is a human.

The compounds of Formula (I) or pharmaceutically acceptable salts thereof may be administered by any suitable route of administration, including systemic administration. Systemic administration includes oral administration, and parenteral administration, Parenteral administration refers to routes of administration other than enteral, transdermal, or by inhalation, and is typically by injection or infusion. Parenteral administration includes intravenous, intramuscular, and subcutaneous injection or infusion.

The compounds of Formula (I) or pharmaceutically acceptable salts thereof may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect.

Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Additionally, the compounds of Formula (I) or pharmaceutically-acceptable salts thereof may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome or overcome a side effect or other difficulty encountered with the compound. Where a —COOH or —OH group is present, pharmaceutically acceptable esters can be employed, for example methyl, ethyl, and the like for —COOH, and acetate maleate and the like for —OH, and those esters known in the art for modifying solubility or hydrolysis characteristics.

The compounds of Formula (I) and pharmaceutically acceptable salts thereof may be co-administered with at least one other active agent known to be useful in the treatment of cancer or pre-cancerous syndromes.

By the term “co-administration” as used herein is meant either simultaneous administration or any manner of separate sequential administration of a PERK inhibiting compound, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; and inhibitors of cancer metabolism.

Examples of a further active ingredient or ingredients (anti-neoplastic agent) for use in combination or co-administered with the presently invented PERK inhibiting compounds are chemotherapeutic agents.

Suitably, the pharmaceutically active compounds of the invention are used in combination with a VEGFR inhibitor, suitably 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt thereof, which is disclosed and claimed in in International Application No. PCT/US01/49367, having an International filing date of Dec. 19, 2001, International Publication Number WO02/059110 and an International Publication date of Aug. 1, 2002, the entire disclosure of which is hereby incorporated by reference, and which is the compound of Example 69. 5-[[4-[(2,3-dimethyl-2H-indazol-6-yl)methylamino]-2-pyrimidinyl]amino]-2-methylbenzenesulfonamide can be prepared as described in International Application No. PCT/US01/49367.

Suitably, 5-[[4-[(2,3-dimethyl-2H-indazol-6-yl)methylamino]-2-pyrimidinyl]amino]-2-methylbenzenesulfonamide is in the form of a monohydrochloride salt. This salt form can be prepared by one of skill in the art from the description in International Application No. PCT/US01/49367, having an International filing date of Dec. 19, 2001.

5-[[4-[(2,3-dimethyl-2H-indazol-6-yl)methylamino]-2-pyrimidinyl]amino]-2-methylbenzenesulfonamide is sold commercially as the monohydrochloride salt and is known by the generic name pazopanib and the trade name Votrient®.

Pazopanib is implicated in the treatment of cancer and ocular diseases/angiogenesis. Suitably the present invention relates to the treatment of cancer and ocular diseases/angiogenesis, suitably age-related macular degeneration, which method comprises the administration of a compound of Formula (I) alone or in combination with pazopanib.

In one embodiment, the cancer treatment method of the claimed invention includes the co-administration a compound of Formula (I) and/or a pharmaceutically acceptable salt thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, cell cycle signaling inhibitors; proteasome inhibitors; and inhibitors of cancer metabolism.

Suitably, the compounds of Formula (I) and pharmaceutically acceptable salts thereof may be co-administered with at least one other active agent known to be useful in the treatment of neurodegenerative diseases/injury.

Suitably, the compounds of Formula (I) and pharmaceutically acceptable salts thereof may be co-administered with at least one other active agent known to be useful in the treatment of diabetes.

Suitably, the compounds of Formula (I) and pharmaceutically acceptable salts thereof may be co-administered with at least one other active agent known to be useful in the treatment of cardiovascular disease.

Suitably, the compounds of Formula (I) and pharmaceutically acceptable salts thereof may be co-administered with at least one other active agent known to be useful in the treatment of ocular diseases.

Suitably, the compounds of Formula (I) and pharmaceutically acceptable salts thereof may be co-administered with at least one other active agent known to be useful for preventing organ damage during and after organ transplantation and in the transportation of organs for transplantation.

### Compositions

The pharmaceutically active compounds within the scope of this invention are useful as PERK inhibitors in mammals, particularly humans, in need thereof.

The present invention therefore provides a method of treating cancer, neurodegeneration and other conditions requiring PERK inhibition, which comprises administering an effective amount of a compound of Formula (I) or a pharmaceutically acceptable salt thereof. The compounds of Formula (I) also provide for a method of treating the above indicated disease states because of their demonstrated ability to act as PERK inhibitors. The drug may be administered to a patient in need thereof by any conventional route of administration, including, but not limited to, intravenous, intramuscular, oral, topical, subcutaneous, intradermal, intraocular and parenteral. Suitably, a PERK inhibitor may be delivered directly to the brain by intrathecal or intraventricular route, or implanted at an appropriate anatomical location within a device or pump that continuously releases the PERK inhibitor drug.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical compositions are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-500 mg/kg of active compound, preferably 0.001-100 mg/kg. When treating a human patient in need of a PERK inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Suitably oral dosage units for human administration preferably contain from 0.5 to 1,000 mg of active compound. Oral administration, which uses lower dosages, is preferred. Parenteral administration, at high dosages, however, also can be used when safe and convenient for the patient.

Optimal dosages to be administered may be readily determined by those skilled in the art, and will vary with the particular PERK inhibitor in use, the strength of the preparation, the mode of administration, and the advancement of the disease condition. Additional factors depending on the particular patient being treated will result in a need to adjust dosages, including patient age, weight, diet, and time of administration.

When administered to prevent organ damage in the transportation of organs for transplantation, a compound of Formula (I) is added to the solution housing the organ during transportation, suitably in a buffered solution.

The method of this invention of inducing PERK inhibitory activity in mammals, including humans, comprises administering to a subject in need of such activity an effective PERK inhibiting amount of a pharmaceutically active compound of the present invention.

The invention also provides for the use of a compound of Formula (I) or a pharmaceutically acceptable salt thereof in the manufacture of a medicament for use as a PERK inhibitor.

The invention also provides for the use of a compound of Formula (I) or a pharmaceutically acceptable salt thereof in the manufacture of a medicament for use in therapy.

The invention also provides for the use of a compound of Formula (I) or a pharmaceutically acceptable salt thereof in the manufacture of a medicament for use in treating cancer, pre-cancerous syndromes, Alzheimer's disease, neuropathic pain, spinal cord injury, traumatic brain injury, ischemic stroke, stroke, Parkinson disease, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Neimann-Pick's disease, amyloidosis, cognitive impairment, atherosclerosis, ocular diseases, and arrhythmias.

The invention also provides for the use of a compound of Formula (I) or a pharmaceutically acceptable salt thereof in the manufacture of a medicament for use in preventing organ damage during the transportation of organs for transplantation.

The invention also provides for a pharmaceutical composition for use as a PERK inhibitor which comprises a compound of Formula (I) or a pharmaceutically acceptable salt thereof and a pharmaceutically acceptable carrier.

The invention also provides for a pharmaceutical composition for use in the treatment of cancer which comprises a compound of Formula (I) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a PERK inhibitor.

The invention also provides a pharmaceutical composition comprising from 0.5 to 1,000 mg of a compound of Formula (I) or pharmaceutically acceptable salt thereof and from 0.5 to 1,000 mg of a pharmaceutically acceptable excipient.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### Examples 1 to 3

### 1-(4-(4-amino-7-methyl-7H-pyrrolo[2,3-d]pyrimidin-5-yl)-3-fluorophenyl)-4-(2,5-difluorophenyl)-3-ethylimidazolidin-2-one and enantiomers

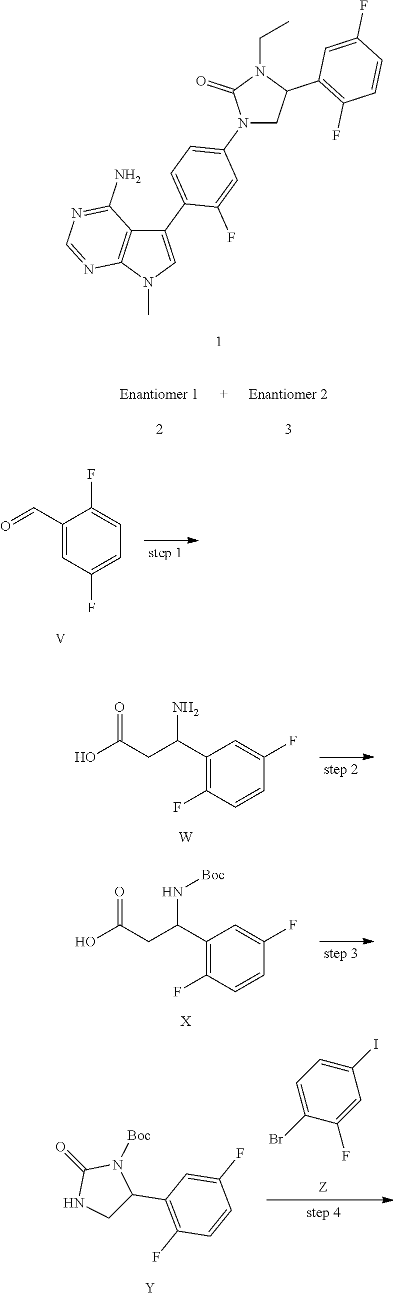

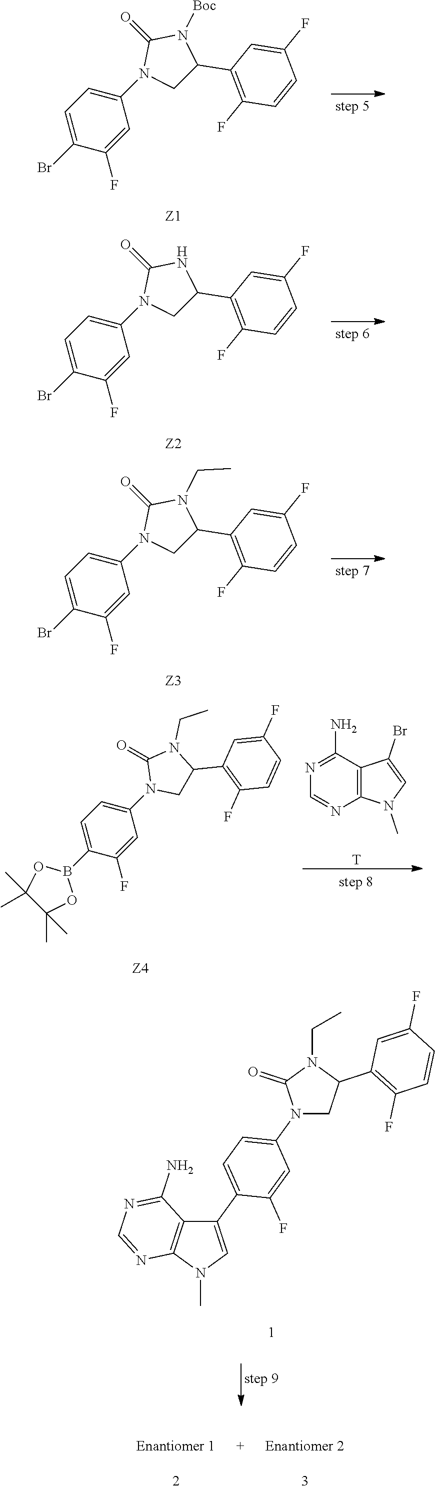

### Step 1

To a stirred solution of 2,5-difluorobenzaldehyde (20.0 g, 140.74 mmol, 1 equiv), malonic acid (17.56 g, 170.0 mmol, 1.2 equiv) and ammonium acetate (21.7 g, 282.0 mmol, 2 equiv) in EtOH (250 mL) was heated at 80° C. for 16 h. After completion of starting material the reaction mixture was cooled to room temperature, the solid obtained was filtered and washed with n-pentane and dried to give the 3-amino-3-(2,5-difluorophenyl)propanoic acid as off white solid (16.0 g, 56.0%). LC-MS (ES) m/z=202.0 [M+H]⁺. ¹H NMR (400 MHz, DMSO-d6) δ ppm 2.40-2.42 (m, 2H), 4.40 (t, J=6.0 Hz, 1H), 5.6-6.8 (br. s, 2H), 7.07-7.12 (m, 1H), 7.13-7.21 (m, 1H), 7.34-7.39 (m, 1H).

### Step 2

To a stirred solution of 3-amino-3-(2,5-difluorophenyl)propanoic acid (16.0 g, 80.0 mmol, 1 equiv), in dioxane (150 mL) and sat. NaHCO₃ solution (150 mL) was added Boc₂O (27.4 mL, 120.0 mmol, 1.5 equiv) at room temperature. The reaction mixture was stirred at RT for O/N. After consumption of the starting material, the reaction mixture was washed with hexane (2×100 mL) and then aqueous layer was acidified with citric acid solution and extracted with EtOAc, and concentrated to give the 3-((tert-butoxycarbonyl)amino)-3-(2,5-difluorophenyl)propanoic acid as white solid (25.0 g, crude). LC-MS (ES) m/z=302.1 [M+H]⁺—100. ¹H NMR (400 MHz, DMSO-d6) δ ppm 1.32 (s, 9H), 2.58-2.65 (m, 2H), 5.13 (s, 1H), 7.09-7.18 (m, 3H), 7.45-7.47 (m, 1H), 12.29 (s, 1H).

### Step 3

Run 1; To a stirred solution of 3-((tert-butoxycarbonyl)amino)-3-(2,5-difluorophenyl)propanoic acid (10.0 g, 33.2 mmol, 1 equiv) in toluene (100 mL) was added DPPA (8.6 mL, 40.0 mmol, 1.2 equiv), & TEA (11.58 mL, 83.0 mmol, 2.5 equiv) at room temperature. The reaction mixture was stirred at RT for 30 min and then heated 75° C. and stirred overnight. After consumption of the starting material, the reaction mixture was cooled, diluted with EtOAc and washed with water. The organic layer was separated & dried over Na₂SO₄, the organic solvent was concentrated to give the tert-butyl 5-(2,5-difluorophenyl)-2-oxoimidazolidine-1-carboxylate as off white solid (4.47 g, 45.0%). LC-MS (ES) m/z=298.2 [M+H]⁺—56. ¹H NMR (400 MHz, DMSO-d6) δ ppm 1.29 (s, 9H), 3.02-3.05 (m, 1H), 3.74 (t, J=9.2 Hz, 1H), 5.31-5.34 (m, 1H), 7.01-7.05 (m, 1H), 7.17-7.23 (m, 1H), 7.25-7.31 (m, 1H), 7.46 (s, 1H).

Run 2: To a stirred solution of 3-((tert-butoxycarbonyl)amino)-3-(2,5-difluorophenyl)propanoic acid (15.0 g, 49.80 mmol, 1 equiv), in toluene (150 mL) was added DPPA (13.0 mL, 59.8 mmol, 1.2 equiv) & TEA (17.4 mL, 124.5 mmol, 2.5 equiv) at room temperature. The reaction mixture at RT for 30 min and then heated to 75° C. and stirred overnight. After completion of starting material, the reaction mixture was cooled, diluted with EtOAc and washed with water. The organic layer was separated & dried over Na₂SO₄ and concentrated to give the tert-butyl 5-(2,5-difluorophenyl)-2-oxoimidazolidine-1-carboxylate as off white solid (11.4 g, 76.0%). LC-MS (ES) m/z=298.2 [M+H]⁺—56. ¹H NMR (400 MHz, DMSO-d6) δ ppm 1.21 (s, 9H), 3.02-3.05 (m, 1H), 3.74 (t, J=9.6 Hz, 1H), 5.31-5.34 (m, 1H), 7.01-7.05 (m, 1H), 7.17-7.22 (m, 1H), 7.25-7.31 (m, 1H), 7.47 (s, 1H).

### Step 4

Run 1; To a stirred solution of tert-butyl 5-(2,5-difluorophenyl)-2-oxoimidazolidine-1-carboxylate (4.47 g, 15.0 mmol, 1.0 equiv), 1-bromo-2-fluoro-4-iodobenzene (5.41 g, 18.0 mmol, 1.2 equiv), and CsF (5.67 g, 37.5 mmol, 2.5 equiv) in EtOAc (80 mL) was added DMEDA (0.16 mL, 1.5 mmol, 0.1 equiv) followed by the addition of CuI (0.143 g, 0.75 mmol, 0.05 equiv). The reaction mixture was stirred at room temperature for 30 h. After consumption of the starting material the reaction mixture was filtered through Celite. The filtrate was washed with water. The organic phase was dried over Na₂SO₄, filtered and evaporated to obtain crude product. The crude product was purified by flash column chromatography (100-200 Silicagel, 40 g column) using 15% EtOAc in hexane as mobile phase to afford the desired product tert-butyl 3-(4-bromo-3-fluorophenyl)-5-(2,5-difluorophenyl)-2-oxoimidazolidine-1-carboxylate (6.2 g, 87.79%) as a pale yellow solid. ¹H NMR (400 MHz, DMSO-d6) δ ppm 1.25 (s, 9H), 3.67-3.05 (m, 1H), 4.29 (t, J=9.6 Hz, 1H), 5.42-5.46 (m, 1H), 7.22-7.33 (m, 3H), 7.36-7.38 (m, 1H), 7.63-7.71 (m, 2H).

Run 2: To a stirred solution of tert-butyl 5-(2,5-difluorophenyl)-2-oxoimidazolidine-1-carboxylate (11.4 g, 38.22 mmol, 1.0 equiv), 1-bromo-2-fluoro-4-iodobenzene (13.8 g, 45.86 mmol, 1.2 equiv), and CsF (14.45 g, 95.55 mmol, 2.5 equiv) in EtOAc (200 mL) was added DMEDA (0.42 mL, 3.82 mmol, 0.1 equiv) followed by the addition of CuI (0.364 g, 1.91 mmol, 0.05 equiv). The reaction mixture was stirred at room temperature for 30 h. After consumption of the starting material the reaction mixture was filtered through Celite. The filtrate was washed with water. The organic phase was dried over Na₂SO₄, filtered and evaporated to afford the crude product. The crude product was purified by flash column chromatography (100-200 Silicagel, 80 g column) using 15% EtOAc in hexane as mobile phase to afford the desired product tert-butyl 3-(4-bromo-3-fluorophenyl)-5-(2,5-difluorophenyl)-2-oxoimidazolidine-1-carboxylate (12.85 g, 71.34%) as a pale yellow solid. ¹H NMR (400 MHz, DMSO-d6) δ ppm 1.25 (s, 9H), 3.67-3.70 (m, 1H), 4.29 (t, J=9.6 Hz, 1H), 5.42-5.46 (m, 1H), 7.22-7.33 (m, 3H), 7.36-7.38 (m, 1H), 7.63-7.71 (m, 2H).

### Step 5

Run 1; To a stirred solution of tert-butyl 3-(4-bromo-3-fluorophenyl)-5-(2,5-difluorophenyl)-2-oxoimidazolidine-1-carboxylate (6.2 g, 13.15 mmol, 1.0 equiv), in 1,4-dioxane (70 mL) was added 20% HCl in dioxane (70 mL) at room temperature and the reaction mixture was stirred at room temperature for 8 h. After consumption of the starting material the reaction mixture was concentrated and basified with aq. NaHCO₃ solution. The reaction mixture was extracted with DCM, and the organic layer was dried over Na₂SO₄, and concentrated to give the 1-(4-bromo-3-fluorophenyl)-4-(2,5-difluorophenyl)imidazolidin-2-one as off white solid (4.7 g, 96.3%). LC-MS (ES) m/z=371.9, 373.0 [M+H]⁺. ¹H NMR (400 MHz, DMSO-d6) δ ppm 3.65-3.69 (m, 1H), 4.30 (t, J=9.2 Hz, 1H), 5.07 (t, J=7.2 Hz, 1H), 7.18-7.32 (m, 4H), 7.56 (t, J=8.4 Hz, 1H), 7.67-7.70 (m, 1H), 7.83 (s, 1H).

Run 2: To a stirred solution of tert-butyl 3-(4-bromo-3-fluorophenyl)-5-(2,5-difluorophenyl)-2-oxoimidazolidine-1-carboxylate (12.85 g, 27.27 mmol, 1.0 equiv), in 1,4-dioxane (150 mL) was added 20% HCl in dioxane (130 mL) at room temperature and the reaction mixture was stirred at room temperature for 8 h. After consumption of the starting material the reaction mixture was concentrated and basified with aq. NaHCO₃ solution. The reaction mixture was extracted with DCM. The organic layer was dried over Na₂SO₄ and concentrated to give the 1-(4-bromo-3-fluorophenyl)-4-(2,5-difluorophenyl)imidazolidin-2-one as off white solid (9.5 g, 93.87%). LC-MS (ES) m/z=371.0, 373.0 [M+H]⁺. ¹H NMR (400 MHz, DMSO-d6) δ ppm 3.65-3.69 (m, 1H), 4.31 (t, J=10.0 Hz, 1H), 5.08 (t, J=7.4 Hz, 1H), 7.19-7.31 (m, 4H), 7.56 (t, J=8.4 Hz, 1H), 7.67-7.70 (m, 1H), 7.83 (s, 1H).

### Step 6

Run 1; To a stirred suspension of 1-(4-bromo-3-fluorophenyl)-4-(2,5-difluorophenyl)imidazolidin-2-one (4.2 g, 11.32 mmol, 1 equiv) in DMF (70 mL) was added 60% NaH (0.54 g, 13.6 mmol, 1.2 equiv) in portion wise at 0° C. under N₂ atmosphere. The reaction mixture was stirred for 20 min, a solution of ethyl Iodide (1.1 mL, 13.6 mmol, 1.2 equiv) in DMF was added and the reaction mixture was stirred for 2 h at room temperature. After consumption of the starting material the reaction mixture was quenched with ice water. The solid was filtered and triturated with n-pentane and dried to give the 1-(4-bromo-3-fluorophenyl)-4-(2,5-difluorophenyl)-3-ethylimidazolidin-2-one as pale brown solid (3.35 g, 74.1%). LCMS (ES) m/z=399.0, 401.0 [M+H]⁺. ¹H NMR (400 MHz, DMSO-d6) δ ppm 0.95 (t, J=7.2 Hz, 3H), 2.73-2.78 (m, 1H), 3.37-3.42 (m, 1H), 3.67-3.70 (m, 1H), 4.23 (t, J=9.6 Hz, 1H), 5.07-5.11 (m, 1H), 7.24-7.33 (m, 4H), 7.58 (t, J=8.8 Hz, 1H), 7.69-7.72 (m, 1H).

Run 2: To a stirred suspension of 1-(4-bromo-3-fluorophenyl)-4-(2,5-difluorophenyl)imidazolidin-2-one (9.5 g, 25.6 mmol, 1 equiv) in DMF (100 mL) was added 60% NaH (1.23 g, 30.72 mmol, 1.2 equiv) in portion wise at 0° C. under N₂ atmosphere. The reaction mixture was stirred for 20 min, a solution of ethyl Iodide (2.5 mL, 30.70 mmol, 1.2 equiv) in DMF was added and the reaction mixture stirred for 2 h at room temperature. After consumption of the starting material, the reaction mixture was quenched with ice water, the solid was filtered and triturated with n-pentane and dried to give the 1-(4-bromo-3-fluorophenyl)-4-(2,5-difluorophenyl)-3-ethylimidazolidin-2-one as pale brown solid (9.75 g, 95.4%). LCMS (ES) m/z=399.0, 401.0 [M+H]⁺. ¹H NMR (400 MHz, DMSO-d6) δ ppm 0.95 (t, J=7.2 Hz, 3H), 2.70-2.80 (m, 1H), 3.36-3.42 (m, 1 H), 3.66-3.70 (m, 1H), 4.23 (t, J=9.6 Hz, 1H), 5.07-5.11 (m, 1H), 7.18-7.34 (m, 4H), 7.57 (t, J=8.4 Hz, 1H), 7.67-7.72 (m, 1H).

### Step 7

Run 1; To a stirred solution of 1-(4-bromo-3-fluorophenyl)-4-(2,5-difluorophenyl)-3-ethylimidazolidin-2-one (3.35 g, 8.4 mmol) in 1,4-dioxane (80 mL) was added bis(pinacolato)diboron (2.132 g, 8.4 mmol, 1 equiv), and potassium acetate (2.47 g, 25.2 mmol, 3 equiv). The reaction mixture was degassed with argon for 10 min. PdCl₂(dppf)-CH₂Cl₂ adduct (0.685 g, 0.84 mmol, 0.1 equiv) was added and degassed with argon for further 10 min. The reaction mixture was stirred for 16 hours at 100° C. in a sealed vessel. The reaction mixture was filtered over celite and the filtrate was concentrated to obtain the crude product. The crude product was purified over silica gel flash column chromatography. The compound eluted out in 15% EtOAc:Hexanes. The pure fractions were evaporated to obtain 4-(2,5-difluorophenyl)-3-ethyl-1-(3-fluoro-4-(4,4,5,5-tetramethyl-1,3,2-dioxaborolan-2-yl)phenyl)imidazolidin-2-one (4.03 g, crude) as a pale yellow oil. LCMS (ES) m/z=447.1 (57.4% by LCMS), 365.1 (39.96% by LCMS) [M+H]⁺.

Run 2: To a stirred solution of 1-(4-bromo-3-fluorophenyl)-4-(2,5-difluorophenyl)-3-ethylimidazolidin-2-one (9.75 g, 24.43 mmol) in 1,4-dioxane (150 mL) was added bis(pinacolato)diboron (6.20 g, 24.43 mmol, 1 equiv), and potassium acetate (7.2 g, 73.0 mmol, 3 equiv). The reaction mixture was degassed with argon for 10 min. PdCl₂(dppf)-CH₂Cl₂ adduct (1.994 g, 2.44 mmol, 0.1 equiv) was added and degassed with N₂ for further 10 min. The reaction mixture was stirred for 16 hours at 100° C. in a sealed vessel. The reaction mixture was filtered over celite and the filtrate was concentrated to get crude product. The crude product was purified using silica gel flash column chromatography. The compound eluted out in 15-% EtOAc:Hexanes. The pure fractions were evaporated to obtain 4-(2,5-difluorophenyl)-3-ethyl-1-(3-fluoro-4-(4,4,5,5-tetramethyl-1,3,2-dioxaborolan-2-yl)phenyl)imidazolidin-2-one (10.5 g, crude) as a pale yellow oil. LCMS (ES) m/z=447.1 (44.5% by LCMS), 365.1 (25.0% by LCMS) [M+H]⁺.

### Step 8

Run 1; To a stirred solution of 4-(2,5-difluorophenyl)-3-ethyl-1-(3-fluoro-4-(4,4,5,5-tetramethyl-1,3,2-dioxaborolan-2-yl)phenyl)imidazolidin-2-one (4.03 g, 9.02 mmol, 1 equiv), 5-bromo-7-methyl-7H-pyrrolo[2,3-d]pyrimidin-4-amine (2.05 g, 9.02 mmol, 1 equiv) and potassium phosphate (3.83 g, 18.05 mmol, 2 equiv) in 1,4-dioxane:water (70 mL: 25 mL), Pd₂(dba)₃ (0.413 g, 0.45 mmol, 0.05 equiv) was added and the reaction mixture was degassed with argon for 5 min. Tri-tert-butylphosphonium tetrafluoroborate (0.262 g, 0.903 mmol, 0.1 equiv) was added and the reaction mixture was further degassed for 5 min. The vial was sealed and the reaction mixture was heated to 100° C. for 16 h. The reaction mixture was cooled & filtered through celite and the filtrate was concentrated to obtain crude compound. Crude compound was purified by flash column chromatography using silicagel column, and the compound was eluted at 2% MeOH:DCM, the pure fractions were evaporated to obtain, 1-(4-(4-amino-7-methyl-7H-pyrrolo[2,3-d]pyrimidin-5-yl)-3-fluorophenyl)-4-(2,5-difluorophenyl)-3-ethylimidazolidin-2-one (1.5 g, 35.63%) as off white solid. LCMS (ES) m/z=467.1 [M+H]⁺. ¹H NMR (400 MHz, DMSO-d6) δ ppm 0.97 (t, J=6.8 Hz, 3H), 2.75-2.80 (m, 1H), 3.35-3.46 (m, 1H), 3.64-3.76 (m, 4H), 4.29 (t, J=9.6 Hz, 1H), 5.11 (t, J=6.4 Hz, 1H), 5.92 (br. s., 2H), 7.24 (s, 1H), 7.27-7.42 (m, 5H), 7.69 (d, J=12.8 Hz, 1H), 8.12 (s, 1H); HPLC: 98.53% purity @270 nm.

Run 2: To a stirred solution of 4-(2,5-difluorophenyl)-3-ethyl-1-(3-fluoro-4-(4,4,5,5-tetramethyl-1,3,2-dioxaborolan-2-yl)phenyl)imidazolidin-2-one (10.5 g, 23.52 mmol, 1 equiv), 5-bromo-7-methyl-7H-pyrrolo[2,3-d]pyrimidin-4-amine (5.34 g, 23.52 mmol, 1 equiv) and potassium phosphate (9.98 g, 47.0 mmol, 2 equiv) in 1,4-dioxane:water (150 mL: 50 mL). Pd₂(dba)₃ (1.07 g, 1.2 mmol, 0.05 equiv) was added and the reaction mixture was degassed with argon for 5 min. Tri-tert-butylphosphonium tetrafluoroborate (0.682 g, 2.352 mmol, 0.1 equiv) was added and the reaction mixture was further degassed for 10 min. The vial was sealed and the reaction mixture was heated to 100° C. for 16 h. The reaction mixture was filtered through celite and the filtrate was concentrated to obtain crude compound. Crude product was purified by flash column chromatography using silicagel column. Compound was eluted at 2% MeOH:DCM, the fractions were evaporated to obtain, 1-(4-(4-amino-7-methyl-7H-pyrrolo[2,3-d]pyrimidin-5-yl)-3-fluorophenyl)-4-(2,5-difluorophenyl)-3-ethylimidazolidin-2-one (2.8 g pure, 25.52% yield and 0.6 g with 96% purity by LCMS) as off white solid. LCMS (ES) m/z=467.1 [M+H]⁺. ¹H NMR (400 MHz, DMSO-d6) δ ppm 0.97 (t, J=7.2 Hz, 3H), 2.75-2.80 (m, 1H), 3.40-3.46 (m, 1H), 3.71-3.76 (m, 4H), 4.29 (t, J=9.6 Hz, 1H), 5.11 (t, J=6.8 Hz, 1H), 5.92 (br. s., 2H), 7.24 (s, 1H), 7.27-7.42 (m, 5H), 7.69 (d, J=13.2 Hz, 1H), 8.12 (s, 1H): HPLC: 99.49% purity @254 nm.

### Run 3 (In Situ Preparation of Boronate Ester Z9 Followed by Suzuki-Miyaura Coupling):

To a stirred solution of 1-(4-bromo-3-fluorophenyl)-4-(2,5-difluorophenyl)-3-ethylimidazolidin-2-one (0.36 g, 0.902 mmol, 1.0 equiv) in 1,4-dioxane (20 mL) was added bis(pinacolato)diboron (0.229 g, 0.902 mmol, 1.0 equiv), potassium acetate (0.265 g, 2.706 mmol, 3.0 equiv), and the mixture was degassed with nitrogen for 10 min. PdCl₂(dppf)-CH₂Cl₂ adduct (0.036 g, 0.045 mmol, 0.05 equiv) was added and the mixture was again degassed with nitrogen for 10 min. The reaction mixture was stirred for 3 h at 100° C. in a sealed vessel. The reaction mixture was cooled to room temperature, 5-bromo-7-methyl-7H-pyrrolo[2,3-d]pyrimidin-4-amine (0.204 g, 0.902 mmol, 1.0 equiv) and saturated aqueous NaHCO₃ (6 mL) was added to the reaction mixture and nitrogen gas was bubbled through the mixture for 10 min. PdCl₂(dppf)-CH₂Cl₂ adduct (0.036 g, 0.045 mmol, 0.05 equiv) was added to the reaction mixture, the vessel was sealed and the reaction mixture was stirred overnight at 100° C. The reaction mixture was cooled to room temperature and filtered through celite, washed with 5% methanol in DCM. The filtrate was dried over Na₂SO₄ and concentrated. The crude material was purified by flash column chromatography using 24 g silica gel column using 2% MeOH in DCM as an eluent to obtain 1-(4-(4-amino-7-methyl-7H-pyrrolo[2,3-d]pyrimidin-5-yl)-3-fluorophenyl)-4-(2,5-difluorophenyl)-3-ethylimidazolidin-2-one as off white solid (0.085 g, 20%). LCMS (ES) m/z=467.2 [M+H]⁺. ¹H NMR (400 MHz, DMSO-d6) δ ppm 0.97 (t, J=6.8 Hz, 3H), 2.73-2.82 (m, 1H), 3.38-3.54 (m, 1H), 3.71 (s, 3H), 3.73-3.76 (m, 1H), 4.29 (t, J=10 Hz, 1H), 5.09-5.13 (m, 1H), 5.92 (br. s, 2H), 7.24 (s, 1H), 7.25-7.36 (m, 4H), 7.40-7.42 (m, 1H), 7.69 (m, J=1.6, 13.2 Hz, 1H), 8.12 (s, 1H). 99.66% of purity by HPLC @274 nM.

### Enantiomer Separation:

0.5 g of racemic compound 1-(4-(4-amino-7-methyl-7H-pyrrolo[2,3-d]pyrimidin-5-yl)-3-fluorophenyl)-4-(2,5-difluorophenyl)-3-ethylimidazolidin-2-one was separated to enantiomers 1 and 2 by chiral HPLC. Preparative HPLC conditions: Column: CHIRALPAK IA (250 mm×20 mm×5 μm); Mobile phase: 0.1% DEA in 100% MEOH; Flow rate: 9 mL/min. Pure fractions at retention time 26.84 min were concentrated to obtain enantiomer 1 as white solid (0.155 g, 31% yield). LCMS (ES) m/z=467.1 [M+H]⁺. ¹H NMR (400 MHz, DMSO-d6) δ ppm 0.97 (t, J=7.2 Hz, 3H), 2.75-2.80 (m, 1H), 3.40-3.46 (m, 1H), 3.71-3.76 (m, 4H), 4.29 (t, J=9.6 Hz, 1H), 5.11 (t, J=6.4 Hz, 1H), 5.92 (br. s., 2H), 7.24 (s, 1H), 7.26-7.42 (m, 5H), 7.69 (d, J=13.2 Hz, 1H), 8.11 (s, 1H): HPLC Analytical conditions: Column: CHIRALPAK IA (250 mm×4.6 mm×5 μm); Mobile phase: 0.1% DEA in 100% MEOH; Flow rate: 0.5 mL/min: 99.99% purity, retention time 29.148 min.

Pure fractions at retention time 30.94 min were concentrated to obtain enantiomer 2 as off white solid (0.147 g, 29.4% yield). LCMS (ES) m/z=467.1 [M+H]⁺. ¹H NMR (400 MHz, DMSO-d6) δ ppm 0.97 (t, J=7.2 Hz, 3H), 2.75-2.80 (m, 1H), 3.40-3.46 (m, 1H), 3.71-3.76 (m, 4H), 4.29 (t, J=9.2 Hz, 1H), 5.10-5.13 (m, 1H), 5.92 (br. s., 2H), 7.24 (s, 1 H), 7.27-7.36 (m, 4H), 7.40-7.42 (m, 1H), 7.69 (d, J=12.8 Hz, 1H), 8.12 (s, 1H):): HPLC Analytical conditions: Column: CHIRALPAK IA (250 mm×4.6 mm×5 μm); Mobile phase: 0.1% DEA in 100% MEOH; Flow rate: 0.5 mL/min 97.33% purity, retention time 34.674 min (2.67% enantiomer 1, retention time 29.698 min)

### Example 4

### 1-(4-(4-Amino-7-methyl-7H-pyrrolo[2,3-d]pyrimidin-5-yl)-3-fluorophenyl)-4-(3,5-difluorophenyl)pyrrolidin-2-one

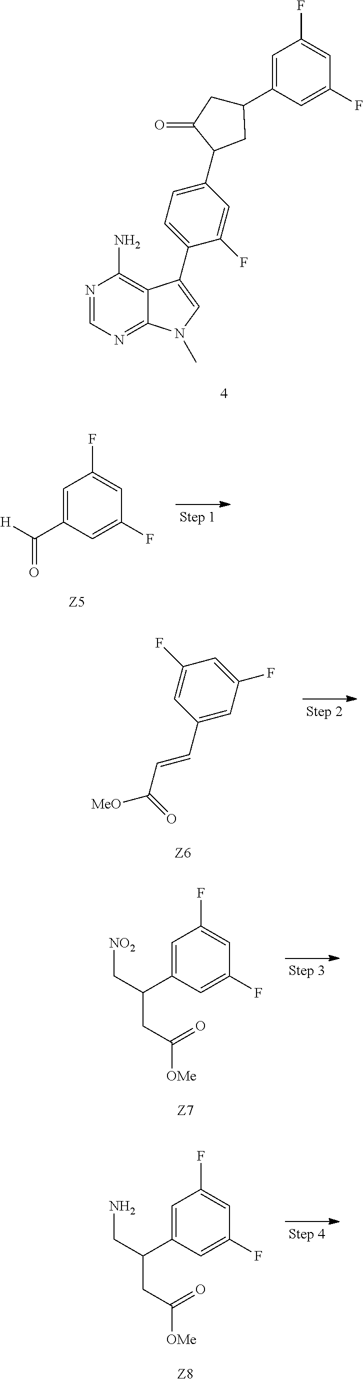 [SMILES: Cn1cc(-c2ccc(C3CC(c4cc(F)cc(F)c4)CC3=O)cc2F)c2c(N)ncnc21 ; O=Cc1cc(F)cc(F)c1 ; COC(=O)/C=C/c1cc(F)cc(F)c1 ; COC(=O)CC(C[N+](=O)[O-])c1cc(F)cc(F)c1 ; COC(=O)CC(CN)c1cc(F)cc(F)c1]

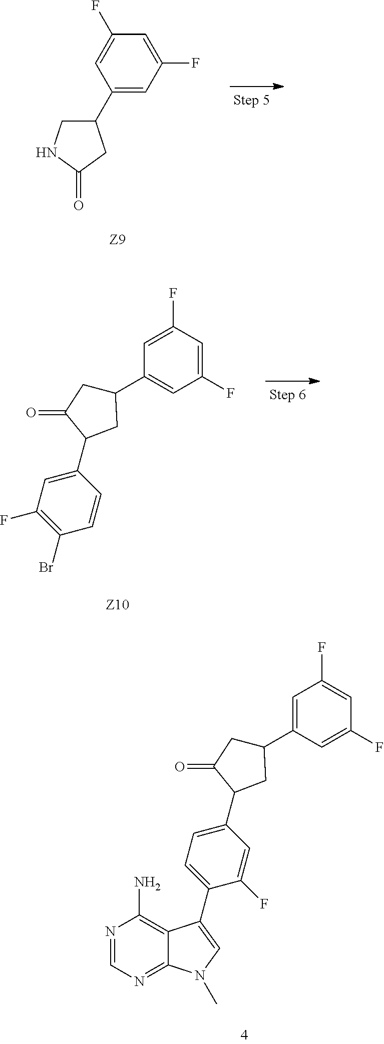 [SMILES: O=C1CC(c2cc(F)cc(F)c2)CN1 ; O=C1CC(c2cc(F)cc(F)c2)CC1c1ccc(Br)c(F)c1 ; Cn1cc(-c2ccc(C3CC(c4cc(F)cc(F)c4)CC3=O)cc2F)c2c(N)ncnc21]

### Step 1

A stirred solution of potassium tertiary butoxide (2.37 g, 21.12 mmol, 1.2 equiv), in THF (50 mL) was cooled to 0° C., and then methyl 2-(diethoxyphosphoryl)acetate (3.88 mL, 21.12 mmol, 1.2 equiv) was slowly added under argon atmosphere. The reaction mixture was stirred for 30 min at 0° C. 3,5-difluorobenzaldehyde (2.5 g, 17.6 mmol, 1.0 equiv) was added to the reaction mixture drop wise and then ice bath was removed. The reaction mixture was stirred at room temperature for 3 h. After consumption of the starting material, the reaction mixture was quenched with water and extracted with EtOAc (2×50 mL). The organic layer was washed with brine, dried over sodium sulphate, filtered and concentrated to give the (E)-methyl 3-(3,5-difluorophenyl)acrylate as white solid (2.15 g, 61.78% yield). ¹H NMR (400 MHz, DMSO-d6) δ ppm 3.72 (s, 2H), 6.78 (d, J=16.4 Hz, 1H), 7.22-7.35 (m, 1H), 7.53 (d, J=6.8 Hz, 2H), 7.63 (t, J=16.4 Hz, 1H).

### Step 2

Nitromethane (10 mL) was stirred in a round bottom flask, cooled to −10° C. and DBU (1.63 mL, 10.85 mmol, 1 equiv) was added rapidly, followed by (E)-methyl 3-(3,5-difluorophenyl)acrylate (2.15 g, 10.85 mmol, 1 equiv). The reaction mixture was stirred at −10° C. for 2 h. The reaction mixture was slowly warmed to room temperature and stirred for 5 h at room temperature. After consumption of the starting material, the reaction mixture was quenched with water and acidified with 1N HCl and extracted with EtOAc. The organic layer was washed with brine and dried over sodium sulphate, filtered & concentrated to give crude product. The crude product was purified by flash column chromatography using a silica gel column, and the compound was eluted at 10% EtOAc in Hexane. Fractions containing product were concentrated to give methyl 3-(3,5-difluorophenyl)-4-nitrobutanoate as semi solid (2.15 g, 76.5% yield). ¹H NMR (400 MHz, DMSO-d6) δ ppm 2.66-2.86 (m, 2H), 3.53 (s, 3H), 3.81-2.87 (m, 1H), 4.86-5.04 (m, 2H), 7.08-7.15 (m, 3H).

### Step 3

To a stirred solution of methyl 3-(3,5-difluorophenyl)-4-nitrobutanoate (2.15 g, 8.3 mmol, 1.0 equiv) in MeOH (40 mL) was added 10% Pd/C (0.215 g) and the mixture was degassed with nitrogen followed by hydrogen. The reaction mixture was stirred under hydrogen atmosphere (H₂ bladder) for 16 h at room temperature. After consumption of the starting material, the mixture was filtered through a celite bed and concentrated to give methyl 4-amino-3-(3,5-difluorophenyl)butanoate as off white solid (2.5 g, crude). LCMS (ES) m/z=230.3 [M+H]⁺.

### Step 4

A stirred solution of methyl 4-amino-3-(3,5-difluorophenyl)butanoate (2.5 g, 11.0 mmol, 1.0 equiv) in MeOH (40 mL) was heated to 60° C. & stirred for 16 h. After completion of starting material, the reaction mixture was concentrated to give 4-(3,5-difluorophenyl)pyrrolidin-2-one as semi solid (1.5 g, crude). LCMS (ES) m/z=198.1 [M+H]⁺.

### Step 5

To a stirred solution of 4-(3,5-difluorophenyl)pyrrolidin-2-one (1.5 g, 7.61 mmol, 1.0 equiv) in EtOAc (30 mL) were added 1-bromo-2-fluoro-4-iodobenzene (2.3 g, 7.61 mmol, 1.0 equiv), DMEDA (0.09 mL, 0.761 mmol, 0.1 equiv), CsF (2.87 g, 19.025 mmol, 2.5 equiv) followed by CuI (0.072 g, 0.381 mmol, 0.05 equiv) and the reaction mixture was stirred at room temperature for 16 h. After completion of the starting material the reaction mixture was filtered through Celite. The filtrate was washed with water, and extracted with EtOAc. The organic phase was dried over Na₂SO₄, filtered and evaporated to obtain crude product. The crude product was purified by flash column chromatography using a silica gel column, and the compound was eluted at 17.0% EtOAc in Hexane. Fractions containing pure compound were concentrated to give 1-(4-bromo-3-fluorophenyl)-4-(3,5-difluorophenyl)pyrrolidin-2-one (0.610 g, 21.7% yield) as pale yellow solid. ¹H NMR (400 MHz, DMSO-d6) δ ppm 2.76-2.93 (m, 2H), 3.75-3.89 (m, 2H), 4.19 (t, J=8.8 Hz, 1H), 6.98-7.06 (m, 2H), 7.10-7.13 (m, 1H), 7.47-7.50 (m, 1H), 7.69 (t, J=8.4 Hz, 1H), 7.79-7.83 (m, 1H).

### Step 6

To a stirred solution of 1-(4-bromo-3-fluorophenyl)-4-(3,5-difluorophenyl)pyrrolidin-2-one (0.610 g, 1.65 mmol, 1.0 equiv) in 1,4-dioxane (18.0 mL) was added bis(pinacolato)diboron (0.419 g, 1.65 mmol, 1.0 equiv), and potassium acetate (0.485 g, 4.95 mmol, 3.0 equiv). The mixture was degassed with Argon for 10 min, PdCl₂(dppf).CH₂Cl₂ complex (0.067 g, 0.0825 mmol, 0.05 equiv) was added and again degassed with argon for 10 min. The reaction mixture was stirred for 5 h at 100° C. in a sealed vessel. The reaction was cooled to room temperature, 5-bromo-7-methyl-7H-pyrrolo[2,3-d]pyrimidin-4-amine (0.374 g, 1.65 mmol, 1.0 equiv) and saturated aqueous NaHCO₃ (6 mL) were added, and degassed with argon for 10 min. PdCl₂(dppf).CH₂Cl₂ complex (0.067 g, 0.0825 mmol, 0.05 equiv) was added, the vessel was sealed, and the reaction mixture was stirred overnight at 100° C. The crude mixture was filtered through Celite and the filtrate was dried over Na₂SO₄ and concentrated to give crude product. The crude product was purified by flash column chromatography using a silica gel column. The compound was eluted at 3-4% MeOH in DCM. Compound containing pure fractions were concentrated to give 1-(4-(4-amino-7-methyl-7H-pyrrolo[2,3-d]pyrimidin-5-yl)-3-fluorophenyl)-4-(3,5-difluorophenyl)pyrrolidin-2-one (0.1 g, 13.8% yield) as off white solid. LCMS (ES) m/z=438.2 [M+H]⁺. ¹H NMR (400 MHz, DMSO-d6) δ ppm 2.66-2.84 (m, 1H), 2.90-2.97 (m, 1H), 3.74 (s, 3H), 3.78-3.84 (m, 1H), 3.92 (t, J=9.2 Hz, 1H), 4.26 (t, J=8.8 Hz, 1H), 5.96 (br, s. 2H), 7.11-7.20 (m, 3H), 7.30 (s, 1H), 7.41 (t, J=8.8 Hz, 1H), 7.57 (d, J=7.2 Hz, 1H), 7.79 (d, J=12.8 Hz, 1H), 8.14 (s, 1H).

### Example 5

### 1-(4-(4-Amino-7-methyl-7H-pyrrolo[2,3-d]pyrimidin-5-yl)-3-methyl phenyl)-4-(3,5-difluorophenyl)pyrrolidin-2-one

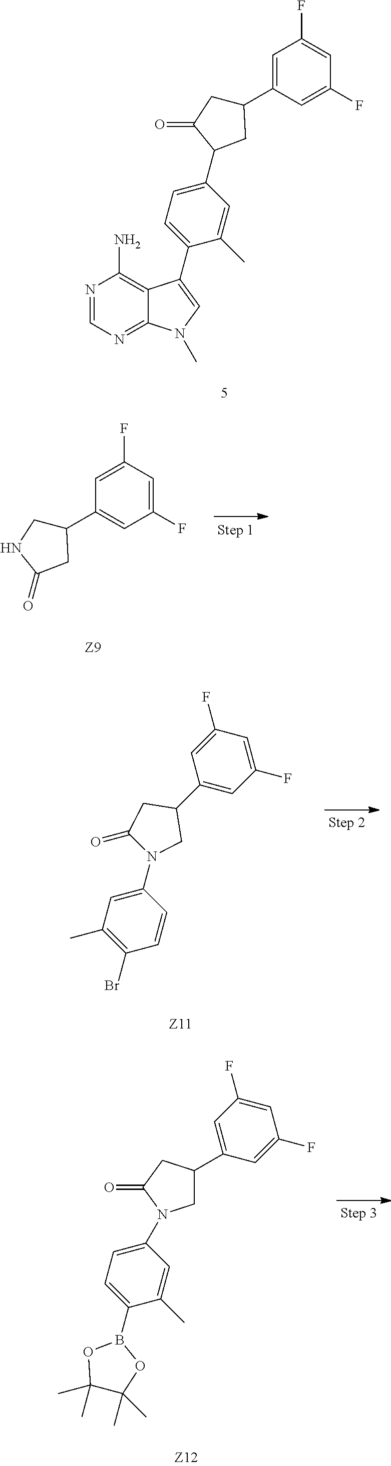 [SMILES: Cc1cc(C2CC(c3cc(F)cc(F)c3)CC2=O)ccc1-c1cn(C)c2ncnc(N)c12 ; O=C1CC(c2cc(F)cc(F)c2)CN1 ; Cc1cc(N2CC(c3cc(F)cc(F)c3)CC2=O)ccc1Br ; Cc1cc(N2CC(c3cc(F)cc(F)c3)CC2=O)ccc1B1OC(C)(C)C(C)(C)O1]

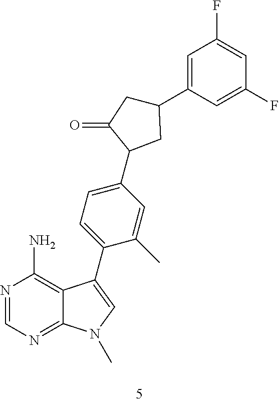 [SMILES: Cc1cc(C2CC(c3cc(F)cc(F)c3)CC2=O)ccc1-c1cn(C)c2ncnc(N)c12]

### Step 1

To a stirred solution of 4-(3,5-difluorophenyl)pyrrolidin-2-one (1.0 g, 5.1 mmol, 1.0 equiv) in EtOAc (30 mL) was added 1-bromo-4-iodo-2-methylbenzene (1.51 g, 5.1 mmol, 1.0 equiv), DMEDA (0.05 mL, 0.51 mmol, 0.1 equiv), CsF (1.93 g, 12.75 mmol, 2.5 equiv) & CuI (0.049 g, 0.255 mmol, 0.05 equiv) and the reaction mixture was stirred at room temperature for 16 h. After consumption of the starting material the reaction mixture was filtered through Celite. The filtrate was washed with water, and extracted with EtOAc. The organic phase was dried over Na₂SO₄, filtered and evaporated to give crude product. The crude product was purified by flash column chromatography using a silica gel column, and the compound was eluted at 20-22% EtOAc in Hexane. Fractions containing pure product were combined and concentrated to give 1-(4-bromo-3-methylphenyl)-4-(3,5-difluorophenyl)pyrrolidin-2-one (0.7 g, 37.7% yield) as a pale yellow solid. ¹H NMR (400 MHz, DMSO-d6) δ ppm 2.40 (s, 3H), 2.69-2.75 (m, 1H), 2.98-3.04 (m, 1H), 3.64-3.72 (m, 1H), 3.80-3.84 (m, 1H), 4.14-4.19 (m, 1H), 6.71-6.79 (m, 1H), 6.78-6.83 (m, 2H), 7.29-7.31 (m, 1H), 7.52 (t, J=9.2 Hz, 2H).

### Step 2

To a stirred solution of 1-(4-bromo-3-methylphenyl)-4-(3,5-difluorophenyl)pyrrolidin-2-one (0.7 g, 1.912 mmol, 1 equiv) in 1,4-dioxane (18 mL) was added bis(pinacolato)diboron (0.488 g, 1.912 mmol, 1 equiv), and potassium acetate (0.562 g, 5.74 mmol, 3 equiv). The reaction mixture was degassed with N₂ for 10 min, and then PdCl₂(dppf)-CH₂Cl₂ adduct (0.156 g, 0.1912 mmol, 0.1 equiv) was added and the mixture was degassed with N₂ for additional 10 min. The reaction mixture was stirred for 4 h at 100° C. in a sealed vessel. The reaction mixture was cooled to room temperature and filtered over Celite. The filtrate was concentrated and the crude product was purified using silica gel flash column chromatography. The compound eluted out in 17-20% EtOAc:Hexanes. The fractions containing pure compound were evaporated to give 4-(3,5-difluorophenyl)-1-(3-methyl-4-(4,4,5,5-tetramethyl-1,3,2-dioxaborolan-2-yl)phenyl)pyrrolidin-2-one (0.7 g, 88.6%) as off white solid. LCMS (ES) m/z=414.2 [M+H]⁺. ¹H NMR (400 MHz, DMSO-d6) δ ppm 1.26 (s, 12H), 2.44 (s, 3H), 2.69-2.88 (m, 2H), 3.70-3.79 (m, 1H), 3.84 (t, J=9.2 Hz, 1H), 4.11-4.18 (m, 1H), 7.08-7.13 (m, 1H), 7.16 (d, J=6.8 Hz, 2H), 7.46 (s, 1H), 7.49-7.51 (m, 1H), 7.59 (d, J=8.0 Hz, 1H).

### Step 3

To a stirred solution of 4-(3,5-difluorophenyl)-1-(3-methyl-4-(4,4,5,5-tetramethyl-1,3,2-dioxaborolan-2-yl)phenyl)pyrrolidin-2-one (0.7 g, 1.7 mmol, 1 equiv), 5-bromo-7-methyl-7H-pyrrolo[2,3-d]pyrimidin-4-amine (0.385 g, 1.7 mmol, 1 equiv) and potassium phosphate (0.722 g, 3.4 mmol, 2 equiv) in 1,4-dioxane:water (20 mL: 6 mL), Pd₂(dba)₃ (0.078 g, 0.085 mmol, 0.05 equiv) was added and the reaction mixture was degassed with N₂ for 5 min, tri-tert-butylphosphonium tetrafluoroborate (0.05 g, 0.17 mmol, 0.1 equiv) was added and the reaction mixture was further degassed for 5 min. The vial was sealed and the reaction mixture was heated to 100° C. and stirred for overnight. The reaction mixture was cooled to room temperature & filtered through Celite. The filtrate was concentrated to obtain crude product. The crude product was purified by flash column chromatography using a silica gel column, and compound was eluted at 3-4% MeOH:DCM. The pure fractions were evaporated to give 1-(4-(4-amino-7-methyl-7H-pyrrolo[2,3-d]pyrimidin-5-yl)-3-methylphenyl)-4-(3,5-difluorophenyl)pyrrolidin-2-one (0.350 g, 47.67%) as off white solid. LCMS (ES) m/z=434.4 [M+H]⁺. ¹H NMR (400 MHz, DMSO-d6) δ ppm 2.18 (s, 3H), 2.72-2.79 (m, 1H), 2.85-2.91 (m, 1H), 3.72 (s, 3H), 3.75-3.81 (m, 1H), 3.90 (t, J=8.8 Hz, 1H), 4.21 (t, J=8.8 Hz, 1H), 5.65 (br. s, 2H), 7.11 (t, J=9.2 Hz, 2H), 7.18 (d, J=7.6 Hz, 2H), 7.22 (d, J=8.8 Hz, 1H), 7.61 (s, 2H), 8.11 (s, 1H). HPLC: 99.89% purity @254 nm.

### Example 6

### 1-(4-(4-Amino-7-methyl-7H-pyrrolo[2,3-d]pyrimidin-5-yl)-3-fluorophenyl)-4-cyclohexyl pyrrolidin-2-one

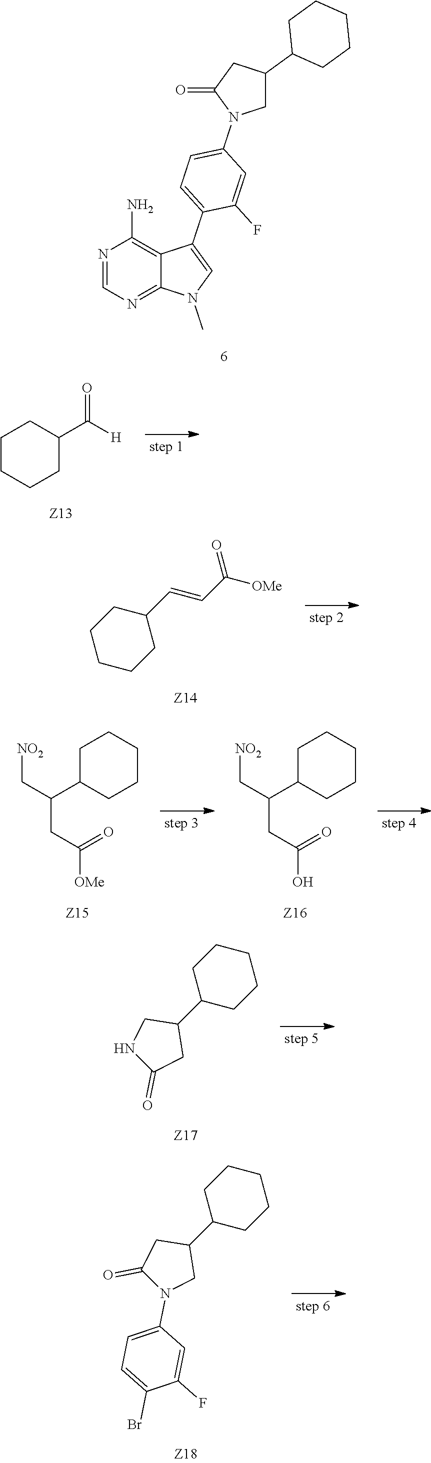

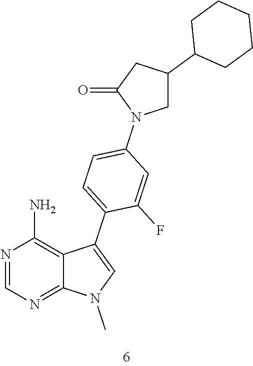 [SMILES: Cn1cc(-c2ccc(N3CC(C4CCCCC4)CC3=O)cc2F)c2c(N)ncnc21]

### Step 1

Methyl 2-(diethoxyphosphoryl)acetate (6.14 mL, 33.4 mmol, 1.5 equiv) in THF (56 mL) was cooled to 0° C. & stirred for 15 min. Potassium tertiary butoxide (3.45 g, 30.8 mmol, 1.4 equiv) was added and the resulting solution was stirred for an additional 10 min at 0° C. The reaction mixture was then warmed quickly to room temperature and stirred for 2 h. The reaction mixture was added drop wise to cyclohexanecarbaldehyde (2.5 g, 22.3 mmol, 1.0 equiv) in THF (10.2 mL) at −78° C. The reaction mixture was allowed to stir for an additional 15 min at −78° C. & then warmed rapidly to 4° C. for 2 h and allowed to stir at room temperature for 18 h. The reaction mixture was quenched with saturated aqueous NH₄Cl and water. The reaction mixture was extracted with EtOAc (3×20 mL). The combined organic phase was washed with brine, dried over Na₂SO₄, evaporated and purified by silica gel flash chromatography. The product was eluted over a solvent gradient of 0 to 3% EtOAc in Hexane. Fractions containing product were concentrated to afford the desired product (E)-methyl 3-cyclohexylacrylate as colourless oil (4.8 g, crude). LC-MS (ES) m/z 169.2 [M+H]⁺. ¹H NMR (400 MHz, CDCl₃) δ 1.65-1.67 (m, 3H), 1.73-1.75 (m, 5H), 2.08-2.15 (m, 1H), 2.96 (d, J=21.6 Hz, 1H), 3.65-3.76 (m, 5H), 4.10-4.19 (m, 1H), 5.75 (d, J=16 Hz, 1H), 6.88-6.93 (m, 1H).

### Step 2

Nitro methane (20 mL) was cooled with stirring to −10° C. and DBU (1.35 mL, 8.9 mmol, 1.0 equiv) was added. A solution of (E)-methyl 3-cyclohexylacrylate (1.5 g, 8.9 mmol, 1 equiv) in nitro methane (2 mL) was then added and stirring was continued for 2 h at −10° C. and then allowed to stir at room temperature for 3 h. Water was added to the reaction mixture and quenched with 1N HCl. The reaction mixture was extracted with EtOAc (3×10 mL), the combined organic layers were washed with brine, dried over Na₂SO₄ and evaporated to afford the desired product methyl 3-cyclohexyl-4-nitrobutanoate as colorless oil (1.29 g, 67%). LC-MS (ES) m/z 230.2 [M+H]⁺. ¹H NMR (400 MHz, CDCl₃) δ 0.99-1.12 (m, 2H), 1.13-1.25 (m, 1H), 1.28-1.36 (m, 2H), 1.40-1.47 (m, 1H), 1.68-1.77 (m, 5H), 2.33-2.39 (m, 1H), 2.49 (t, J=10.8 Hz, 1H), 2.53-2.61 (m, 1H), 3.68 (s, 3H), 4.47 (d, J=6.4 Hz, 2H).

### Step 3

To a degassed solution of methyl 3-cyclohexyl-4-nitrobutanoate (1.28 g, 5.97 mmol) in MeOH (15 mL) under nitrogen was added 10% Pd/C (contains 50% moisture) (1 g) carefully. The reaction mixture was evacuated with hydrogen using a hydrogen bladder and stirred under hydrogen atmosphere overnight at ambient temperature. The reaction mixture was filtered and washed thoroughly with MeOH (3×5 mL). The filtrate was evaporated to afford a mixture of 4-amino-3-cyclohexylbutanoic acid and 4-cyclohexylpyrrolidin-2-one (0.96 g, crude) and it was forwarded to next step as such. LC-MS (ES) m/z 186.2 [M+H]⁺.

### Step 4

To a stirred solution of 4-amino-3-cyclohexylbutanoic acid (0.95 g, 5.13 mmol, 1 equiv) in DCM (20 mL) at rooms temperature was slowly added DIPEA (2.7 mL, 15.4 mmol, 3 equiv) followed by T₃P (4.53 mL, 7.69 mmol, 1.5 equiv) (>50% w/w in EtOAc). The resulting solution was stirred at ambient temperature for 5 h. The reaction mixture was quenched with water, diluted with DCM and the two layers were separated. The combined organic phases were washed with 1N HCl followed by brine, dried over Na₂SO₄, evaporated to afford the crude product 4-cyclohexylpyrrolidin-2-one as off-white solid (0.99 g, crude). LC-MS (ES) m/z 168.2 [M+H]⁺.

### Step 5

To a stirred solution of 4-cyclohexylpyrrolidin-2-one (0.5 g, 3 mmol, 1 equiv) and 1-bromo-2-fluoro-4-iodobenzene (1.33 g, 4.5 mmol, 1.5 equiv) in EtOAc (25 mL) was added cesium fluoride (1.14 g, 7.5 mmol, 2.5 equiv), N,N′-dimethylethylenediamine (0.03 mL, 0.3 mmol, 0.1 equiv) and CuI (0.03 g, 0.15 mmol, 0.05 equiv) at ambient temperature and the reaction mixture was stirred overnight at the same temperature. The reaction mixture was filtered through celite. The celite pad was washed thoroughly with EtOAc (2×10 mL). The filtrate was washed with brine, dried over Na₂SO₄ and concentrated. The crude product was purified by silica gel flash column chromatography using 12 g silica gel column. The product was eluted over a solvent gradient of 9-10% EtOAc:Hexane. Fractions containing product were concentrated to give desired product 1-(4-bromo-3-fluorophenyl)-4-cyclohexylpyrrolidin-2-one as white solid (0.33 g, 32%). LC-MS (ES) m/z=340.1, 342.1 [M+H]⁺. ¹H NMR (400 MHz, CDCl₃) δ 0.95-1.05 (m, 2H), 1.31-1.42 (m, 4H), 1.68-1.74 (m, 5H), 2.15-2.26 (m, 1H), 2.31-2.38 (m, 1H), 2.62-2.69 (m, 1H), 3.50 (t, J=8.8 Hz, 1H), 3.82 (t, J=9.2 Hz, 1H), 7.25-7.27 (m, 1H), 7.48 (t, J=8.4 Hz, 1H), 7.60-7.63 (m, 1H).

### Step 6

To a mixture of 1-(4-bromo-3-fluorophenyl)-4-cyclohexylpyrrolidin-2-one (0.32 g, 0.94 mmol, 1.0 equiv), bis(pinacolato)diboron (0.31 g, 1.2 mmol, 1.3 equiv), and potassium acetate (0.28 g, 2.82 mmol, 3.0 equiv) was added 1,4-dioxane (12 mL), and the mixture was degassed with Ar for 5 min. Pd(dppf)Cl₂.DCM complex (0.04 g, 0.05 mmol, 0.05 equiv) was added and again degassed with argon for 5 min. The reaction mixture was heated in a sealed vessel for 6 h at 100° C. After consumption of the starting material, the reaction mixture was cooled to ambient temperature. 5-bromo-7-methyl-7H-pyrrolo[2,3-d]pyrimidin-4-amine (0.24 g, 1.03 mmol, 1.1 equiv) and saturated aqueous NaHCO₃ (8 mL) were added and argon gas was thoroughly bubbled through the mixture for 5 min. Pd(dppf)Cl₂.DCM complex (0.08 g, 0.1 mmol, 0.1 equiv) was added, the vessel was sealed, and the reaction mixture was heated to 100° C. & stirred for overnight. After consumption of the starting material, the reaction mixture was cooled to ambient temperature and partitioned between EtOAc (25 mL) and water (10 mL), the two layers were separated and the combined organics were washed with brine (20 mL), dried over Na₂SO₄, filtered and evaporated. The crude product was purified by silica gel flash chromatography using a solvent gradient of 2-3% MeOH in DCM. The fractions containing product were concentrated in vacuo to give desired product. It was re-purified over C-18 column using flash column chromatography. The compound was eluted over 40% ACN in water with 0.01% formic acid. The pure fractions were evaporated, neutralized with aqueous saturated NaHCO₃ and extracted into 10% MeOH in DCM (3×15 mL). The combined organic phase was dried over Na₂SO₄, concentrated to afford the desired product 1-(4-(4-amino-7-methyl-7H-pyrrolo[2,3-d]pyrimidin-5-yl)-3-fluorophenyl)-4-cyclohexylpyrrolidin-2-one as white solid (0.05 g, 14%). LC-MS (ES) m/z=408.2 [M+H]⁺. ¹H NMR (400 MHz, DMSO-d6) δ 1.62-1.76 (m, 6H), 2.17 (t, J=8.4 Hz, 1H), 2.30-2.36 (m, 1H), 2.58-2.60 (m, 2H), 3.57 (d, J=8.8 Hz, 1H), 3.72 (s, 3H), 3.90 (t, J=8.8 Hz, 1H), 5.94 (br. s., 2H), 7.27 (s, 1H), 7.36 (t, J=8.4 Hz, 1H), 7.54 (d, J=7.2 Hz, 1H), 7.76 (d, J=12.8 Hz, 1H), 8.12 (s, 1H).

### Example 7

### 1-(4-(4-amino-7-methyl-7H-pyrrolo[2,3-d]pyrimidin-5-yl)-3-fluorophenyl)-3-methyl-4-(pyridin-2-yl)imidazolidin-2-one

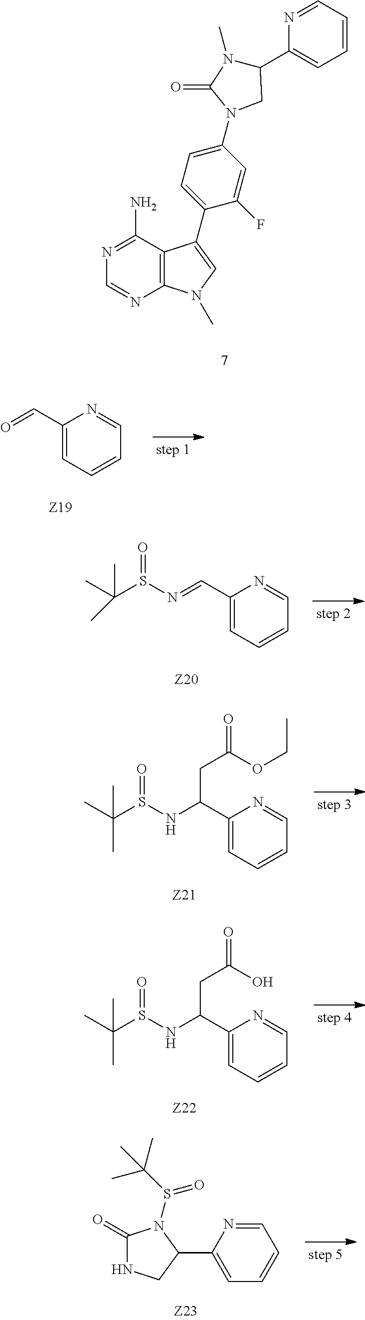 [SMILES: CN1C(=O)N(c2ccc(-c3cn(C)c4ncnc(N)c34)c(F)c2)CC1c1ccccn1 ; O=Cc1ccccn1 ; CC(C)(C)S(=O)/N=C/c1ccccn1 ; CCOC(=O)CC(NS(=O)C(C)(C)C)c1ccccn1 ; CC(C)(C)S(=O)NC(CC(=O)O)c1ccccn1 ; CC(C)(C)S(=O)N1C(=O)NCC1c1ccccn1]

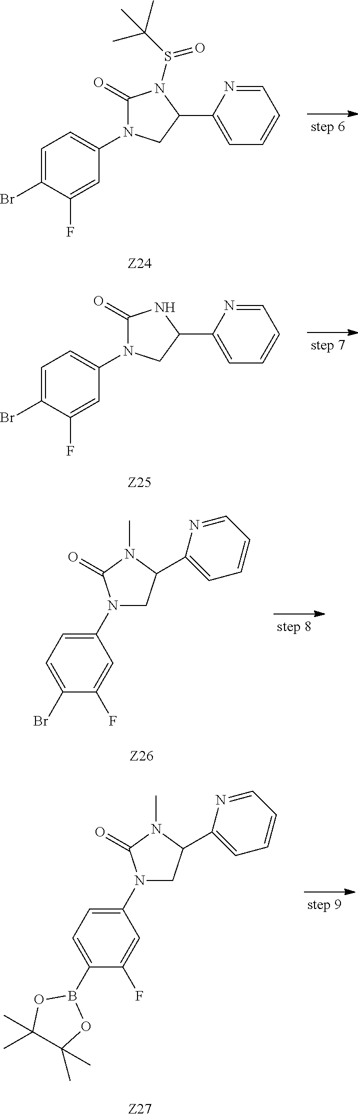 [SMILES: CC(C)(C)S(=O)N1C(=O)N(c2ccc(Br)c(F)c2)CC1c1ccccn1 ; O=C1NC(c2ccccn2)CN1c1ccc(Br)c(F)c1 ; CN1C(=O)N(c2ccc(Br)c(F)c2)CC1c1ccccn1 ; CN1C(=O)N(c2ccc(B3OC(C)(C)C(C)(C)O3)c(F)c2)CC1c1ccccn1]

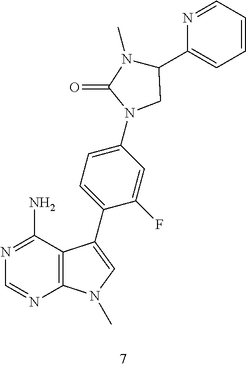 [SMILES: CN1C(=O)N(c2ccc(-c3cn(C)c4ncnc(N)c34)c(F)c2)CC1c1ccccn1]

### Step 1

To a stirred solution of picolinaldehyde (5.0 g, 46.728 mmol, 1 equiv), 2-methylpropane-2-sulfinamide (8.48 g, 70.0934 mmol, 1.5 equiv) in THF (100 mL) was added Ti(OEt)₄ (21.38 g, 93.4579 mmol, 1.5 equiv) at room temperature. The reaction mixture was heated to 75° C. and stirred for 16 h. After consumption of starting material the reaction mixture was cooled to room temperature, and quenched with water and stirred rapidly. The reaction mixture was filtered through celite pad, and filtrate was concentrated to give crude product which was purified by flash column chromatography using a silica gel column. The compound was eluted at 5% EtOAc in Hexane as mobile phase. Fractions containing product was concentrated to obtain (E)-2-methyl-N-(pyridin-2-ylmethylene)propane-2-sulfinamide (6.8 g, 69.1%) as colour less liquid. LC-MS (ES) m/z=211.1 [M+H]⁺. ¹H NMR (400 MHz, CDCl₃) δ ppm 1.28 (s, 9H), 7.37-7.40 (m, 1H), 7.78-7.82 (m, 1H), 8.01 (d, J=8.0 Hz, 1H), 8.70 (s, 1H), 8.74 (d, J=4.4 Hz, 1H).

### Step 2

To a stirred solution of 2M LDA (31.5 mL, 63.0 mmol, 2.1 equiv), in MTBE (30 mL) was added EtOAc (5.88 mL, 60.0 mmol, 2.0 equiv) in MTBE (10 mL) slowly at −78° C., and the mixture was stirred for 30 minutes. A solution of (E)-2-methyl-N-(pyridin-2-ylmethylene)propane-2-sulfinamide (6.3 g, 30.0 mmol, 1.0 equiv) in MTBE (30 mL) was slowly added to the reaction mixture at −78° C. and the reaction mixture was stirred for 2 h at −78° C. After consumption of the starting material, the reaction mixture was quenched with NH₄Cl solution and extracted with EtOAc. The combined organic layers were washed with water, brine and dried over Na₂SO₄, and concentrated to give ethyl 3-(1,1-dimethylethylsulfinamido)-3-(pyridin-2-yl)propanoate (8.0 g, crude) as pale brown liquid.

LC-MS (ES) m/z=299.1 [M+H]⁺. ¹H NMR (400 MHz, DMSO-d6) δ ppm 1.09 (s, 9H), 1.10-1.15 (m, 3H), 2.64-2.71 (m, 0.5H), 2.88-3.03 (m, 1.5H), 3.97-4.08 (m, 2H), 4.68-4.78 (m, 1H), 5.73 (d, J=8.0 Hz, 0.5H), 5.84 (d, J=8.0 Hz, 0.5H), 7.23-7.27 (m, 1H), 7.45 (d, J=8.0 Hz, 0.5H), 7.62 (d, J=8.0 Hz, 0.5H), 7.74-7.79 (m, 1H), 8.45-8.48 (m, 1H).

### Step 3

Run1: To a stirred solution of ethyl 3-(1,1-dimethylethylsulfinamido)-3-(pyridin-2-yl)propanoate (1.0 g, 3.351 mmol, 1 equiv), in MeOH (8 mL) and THF (8 mL) was added LiOH.H₂O (0.335 g, 6.71 mmol, 2.0 equiv) at at room temperature. Then the reaction mixture was stirred at RT for 4 h. After consumption of the starting material, the reaction mixture was concentrated and acidified with citric acid solution to pH-4-5, then extracted with EtOAc, and the organic layer was washed with brine and dried over Na₂SO₄ and concentrated to give 3-(1,1-dimethylethylsulfinamido)-3-(pyridin-2-yl)propanoic acid (1.2 g, crude) as brown solid.

LC-MS (ES) m/z=271.1 [M+H]⁺.

Run 2: To a stirred solution of ethyl 3-(1,1-dimethylethylsulfinamido)-3-(pyridin-2-yl)propanoate (3.5 g, 11.73 mmol, 1 equiv), in MeOH (25 mL) and THF (25 mL) was added LiOH.H₂O (1.17 g, 23.46 mmol, 2.0 equiv) at room temperature. The reaction mixture was stirred at room temperature for 4 h. After consumption of the starting material, the reaction mixture was concentrated and the residue obtained was acidified with citric acid solution to PH ˜4. The reaction mixture was extracted with EtOAc. The organic layer was separated and washed with brine, dried over Na₂SO₄ and concentrated to give 3-(1,1-dimethylethylsulfinamido)-3-(pyridin-2-yl)propanoic acid (2.2 g, crude) as brown solid. LC-MS (ES) m/z=271.1 [M+H]⁺.

### Step 4

To a stirred solution of 3-(1,1-dimethylethylsulfinamido)-3-(pyridin-2-yl)propanoic acid (1.2 g, 4.43 mmol, 1 equiv), in Toluene (20 mL) was added DPPA (1.15 mL, 5.314 mmol, 1.2 equiv), TEA (1.55 mL, 11.1 mmol, 2.5 equiv), at room temperature, and stirred for 30 minutes at room temperature. The reaction mixture was then heated to 75° C. and stirred for 16 h. After consumption of the starting material, the reaction mixture was cooled, and concentrated to give the 1-(tert-butylsulfinyl)-5-(pyridin-2-yl)imidazolidin-2-one as brown colour oil (1.0 g crude), the crude material was purified along with run 2.

LC-MS (ES) m/z=268.1 [M+H]⁺.

Run 2: To a stirred solution of 3-(1,1-dimethylethylsulfinamido)-3-(pyridin-2-yl)propanoic acid (2.2 g, 8.12 mmol, 1 equiv), in Toluene (30 mL) was added DPPA (2.1 mL, 9.742 mmol, 1.2 equiv), TEA (2.83 mL, 20.3 mmol, 2.5 equiv), at room temperature, and then stirred the reaction mixture at room temperature for 30 minutes. The reaction mixture was heated to 75° C. and stirred for 16 h. After consumption of the starting material, the reaction mixture was cooled, and concentrated and purified by flash silica gel column chromatography. The compound was eluted at 2% MeOH:DCM, to give the 1-(tert-butylsulfinyl)-5-(pyridin-2-yl)imidazolidin-2-one as brown colour solid (2.0 g). LC-MS (ES) m/z=164.1 [M+H]⁺—103.

### Step 5

To a stirred solution of 1-(tert-butylsulfinyl)-5-(pyridin-2-yl)imidazolidin-2-one (2.0 g, 7.5 mmol, 1.0 equiv), 1-bromo-2-fluoro-4-iodobenzene (2.70 g, 9.0 mmol, 1.2 equiv), and CsF (2.84 g, 18.75 mmol, 2.5 equiv) in EtOAc (30 mL) was added DMEDA (0.08 mL, 0.75 mmol, 0.1 equiv) followed by the addition of CuI (0.071 g, 0.375 mmol, 0.05 equiv) reaction mixture was stirred at room temperature for 20 h. After consumption of the starting material the reaction mixture was filtered through Celite. The filtrate was washed with water, followed by the brine. The organic phase was dried over Na₂SO₄, filtered and evaporated. Purification: The crude product was purified by flash column chromatography using silicagel column compound was eluted at 70.0% EtOAc:Hexane as mobile phase to afford the product 1-(4-bromo-3-fluorophenyl)-3-(tert-butylsulfinyl)-4-(pyridin-2-yl)imidazolidin-2-one (0.75 g, 22.7%) as pale brown solid. LC-MS (ES) m/z=440.0, 442.0 [M+H]⁺. ¹H NMR (400 MHz, DMSO-d6) δ ppm 0.81 (s, 9H), 3.92-3.95 (m, 1H), 4.41 (t, J=9.60 Hz, 1H), 5.21-5.25 (m, 1H), 7.33-7.38 (m, 2H), 7.47 (d, J=7.6 Hz, 1H), 7.63-7.70 (m, 2H), 7.78-7.83 (m, 1H), 8.61-8.62 (m, 1H).

### Step 6

To a stirred solution of 1-(4-bromo-3-fluorophenyl)-3-(tert-butylsulfinyl)-4-(pyridin-2-yl)imidazolidin-2-one (0.75 g, 1.7033 mmol, 1.0 equiv), in 1,4-dioxane (10 mL) was added 20% HCl in dioxane (10 mL) at room temperature and the reaction mixture was stirred at room temperature for 7 h. After consumption of the starting material the reaction mixture was concentrated and basified with aq NaHCO₃ solution. The reaction mixture was extracted with DCM, and organic layer was dried over Na₂SO₄, and concentrated, to give the 1-(4-bromo-3-fluorophenyl)-4-(pyridin-2-yl)imidazolidin-2-one as pale brown solid (0.475 g, 57.25% yield). LC-MS (ES) m/z=336.0, 338.0 [M+H]⁺. ¹H NMR (400 MHz, DMSO-d6) δ ppm 3.82-3.85 (m, 1H), 4.27 (t, J=9.6 Hz, 1H), 4.90-4.93 (m, 1H), 7.29-7.39 (m, 2H), 7.46-7.48 (m, 1H), 7.54-7.60 (m, 1H), 7.66-7.76 (m, 1H), 7.87-7.87 (m, 1H), 7.93 (s, 1H), 8.57-8.57 (m, 1H).

### Step 7

To a stirred suspension of 1-(4-bromo-3-fluorophenyl)-4-(pyridin-2-yl)imidazolidin-2-one (0.475 g, 1.413 mmol, 1 equiv) in DMF (10 mL) was added 60% NaH (0.068 g, 1.7 mmol, 1.2 equiv) in portions at 0° C. under N₂ atmosphere. The mixture was then stirred for 20 minutes. Then a solution of Methyl Iodide (0.11 mL, 1.7 mmol, 1.2 equiv) in DMF was added and the reaction mixture stirred for 2 h at room temperature. After consumption of the starting material the reaction mixture was quenched with ice water, and extracted with EtOAc (2×30 mL). The combine organics was washed with water, brine and dried over Na₂SO₄ and concentrated to give the 1-(4-bromo-3-fluorophenyl)-3-methyl-4-(pyridin-2-yl)imidazolidin-2-one as pale brown solid (0.42 g, 84.88%). LCMS (ES) m/z=350.0, 352.0 [M+H]⁺. ¹H NMR (400 MHz, DMSO-d6) δ ppm 2.60 (s, 3H), 3.71-3.75 (m, 1H), 4.20 (t, J=9.2 Hz, 1H), 4.82-4.86 (m, 1H), 7.32-7.39 (m, 2H), 7.45 (d, J=8.0 Hz, 1H), 7.58 (t, J=8.8 Hz, 1H), 7.71-7.74 (m, 1H), 7.83-7.87 (m, 1H), 8.59-8.60 (m, 1H).

### Step 8

To a stirred solution of 1-(4-bromo-3-fluorophenyl)-3-methyl-4-(pyridin-2-yl)imidazolidin-2-one (0.42 g, 1.2 mmol, 1 equiv) in 1,4-Dioxane (15 mL) was added bis(pinacolato)diboron (0.305 g, 1.2 mmol, 1 equiv), and potassium acetate (0.353 g, 3.60 mmol, 3 equiv), The reaction mixture was degassed with N₂ for 10 minutes. PdCl₂(dppf)-CH₂Cl₂ adduct (0.098 g, 0.12 mmol, 0.1 equiv) was added and degassed with N₂ for further 10 minutes. The reaction mixture was heated to 100° C. in a sealed vessel and stirred for 16 h. The reaction mixture was cooled to room temperature and filtered through celite, the filtrate was concentrated and the crude product was purified by silica gel flash column chromatography. The compound eluted out in 2.5% MeOH:DCM. The pure fractions were evaporated to obtain 1-(3-fluoro-4-(4,4,5,5-tetramethyl-1,3,2-dioxaborolan-2-yl)phenyl)-3-methyl-4-(pyridin-2-yl)imidazolidin-2-one (0.28 g, 58.76%) as pale brown solid. LCMS (ES) m/z=398.2 [M+H]⁺.

### Step 9

To a stirred solution of 1-(3-fluoro-4-(4,4,5,5-tetramethyl-1,3,2-dioxaborolan-2-yl)phenyl)-3-methyl-4-(pyridin-2-yl)imidazolidin-2-one (0.280 g, 0.705 mmol, 1 equiv), 5-bromo-7-methyl-7H-pyrrolo[2,3-d]pyrimidin-4-amine (0.160 g, 0.705 mmol, 1 equiv) and potassium phosphate (0.3 g, 1.41 mmol, 2 equiv) in 1,4-Dioxane:water (10 mL: 2 mL), Pd₂(dba)₃ (0.032 g, 0.0353 mmol, 0.05 equiv) was added and the reaction mixture was degassed with N₂ for 5 minutes. Tri-tert-butylphosphonium tetrafluoroborate (0.0205 g, 0.0705 mmol, 0.1 equiv) was added and the reaction mixture was further degassed for 5 minutes. The vial was sealed and the reaction mixture was heated to 100° C. and stirred for 5 h. The reaction mixture was cooled to room temperature and filtered through celite, the filtrate was concentrated to obtain crude product. The crude product was purified by flash column chromatography using silica gel column, compound was eluted at 4.0% MeOH:DCM, the pure fractions were evaporated to obtain, 1-(4-(4-amino-7-methyl-7H-pyrrolo[2,3-d]pyrimidin-5-yl)-3-fluorophenyl)-3-methyl-4-(pyridin-2-yl)imidazolidin-2-one (0.07 g, 23.7%) as pale brown solid. LCMS (ES) m/z=418.2 [M+H]⁺. ¹H NMR (400 MHz, DMSO-d6) δ ppm 2.62 (s, 3H), 3.72 (s, 3H), 3.77-3.81 (m, 1H), 4.26 (t, J=9.2 Hz, 1H), 4.84-4.88 (m, 1H), 5.93 (br. s., 2H), 7.24 (s, 1H), 7.31 (t, J=8.8 Hz, 1H), 7.37-7.43 (m, 2H), 7.46 (d, J=7.6 Hz, 1H), 7.71 (d, J=12.0 Hz, 1H), 7.86 (d, J=7.6 Hz, 1H), 8.12 (s, 1H), 8.60-8.62 (m, 1H).; HPLC: 99.87% purity @264 nm.

### Example 8 and 9

### Enantiomers of 1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one

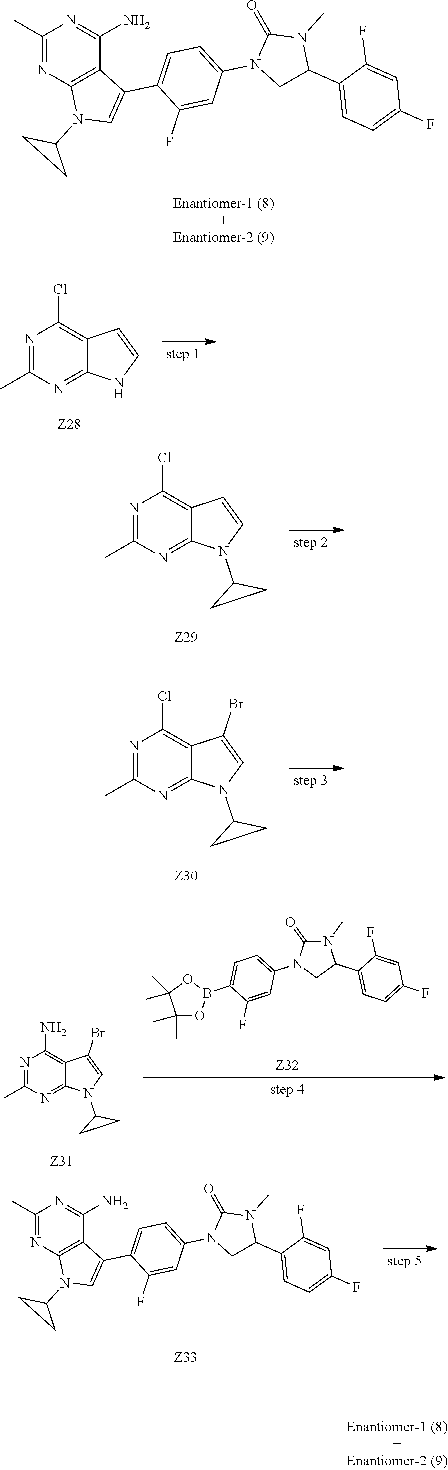

### Step 1

To a stirred solution of 4-chloro-2-methyl-7H-pyrrolo[2,3-d]pyrimidine (2.0 g, 11.9 mmol, 1.0 equiv) in pyridine (50 mL) was added cyclopropyl boronic acid (1.5 g, 17.9 mmol, 1.5 equiv) and copper(II)acetate (4.0 g, 17.9 mmol, 1.5 equiv) at room temperature. The resulted suspension was stirred at 90° C. for 16 h under oxygen atmosphere. The reaction mixture was quenched with water. The crude was extracted in ethyl acetate. The organic layer was dried over sodium sulphate and evaporated to obtain crude product, which was purified by silica gel flash column chromatography. The compound eluted out in 15% EtOAc:n-Hexane. Fractions containing product were concentrated to give 4-chloro-7-cyclopropyl-2-methyl-7H-pyrrolo[2,3-d]pyrimidine as yellow solid (1.0 g, 41%). LCMS (ES) m/z=208.1 [M+H]⁺. ¹H NMR (400 MHz, DMSO-d₆) δ ppm 1.03-1.05 (m, 4H), 2.63 (s, 3H), 3.58 (q, J=4.0 Hz, 1H), 6.48 (d, J=4.0 Hz, 1H), 7.54 (d, J=3.4 Hz, 1H).

### Step 2

To a stirred solution of 4-chloro-7-cyclopropyl-2-methyl-7H-pyrrolo[2,3-d]pyrimidine (1.0 g, 4.58 mmol, 1 equiv) in DCM (30 mL) was added NBS (0.9 g, 5.04 mmol, 1.1 equiv) at 0° C. The reaction mixture was warmed to room temperature and stirred for 2 h. The reaction mixture was quenched with water and extracted with ethyl acetate. The organic layer was dried over sodium sulphate and evaporated to obtain 5-bromo-4-chloro-7-cyclopropyl-2-methyl-7H-pyrrolo[2,3-d]pyrimidine (1.0 g, 72%) as pale yellow solid. LCMS (ES) m/z=286.5, 288.5 [M+H.]⁺. ¹H NMR (400 MHz, DMSO-d₆) δ ppm 0.99-1.08 (m, 4H), 2.63 (s, 3H), 3.51-3.61 (m, 1H), 7.82 (s, 1H).

### Step 3

To a stirred solution of 5-bromo-4-chloro-7-cyclopropyl-2-methyl-7H-pyrrolo[2,3-d]pyrimidine (1.0 g, 3.4 mmol, 1 equiv) in 1,4-dioxane (10 mL) was added NH₄OH (20 mL) at room temperature. The reaction mixture was heated to 100° C. in an autoclave for 16 h. The reaction mixture was cooled and the solids formed were filtered to obtain 5-bromo-7-cyclopropyl-2-methyl-7H-pyrrolo[2,3-d]pyrimidin-4-amine (0.6 g, 66%) as pale yellow solid. LCMS (ES) m/z=267.1, 269.1 [M+H.]⁺. ¹H NMR (400 MHz, DMSO-d₆) δ ppm 0.94-0.95 (d, J=7.9 Hz, 4H), 2.36 (s, 3H), 3.45-3.47 (m, 1H). 6.57 (br.s., 2H), 7.22 (s, 1H).

### Step 4

To a stirred solution of 4-(2,4-difluorophenyl)-1-(3-fluoro-4-(4,4,5,5-tetramethyl-1,3,2-dioxaborolan-2-yl)phenyl)-3-methylimidazolidin-2-one (0.47 g, 1.08 mmol, 1 equiv) (synthesized following procedure similar to example 1) in 1,4-dioxane (30 mL) was added 5-bromo-7-cyclopropyl-2-methyl-7H-pyrrolo[2,3-d]pyrimidin-4-amine (0.22 g, 0.81 mmol, 0.75 equiv), tri-potassium phosphate (0.46 g, 2.17 mmol, 2.0 equiv) and water (1 mL). The reaction mixture was degassed with N₂ for 15 minutes. Pd₂(dba)₃ (0.05 g, 0.054 mmol, 0.05 equiv) and tri-tert-butylphosphonium tetrafluoroborate (0.031 g, 0.108 mmol, 0.1 equiv) were added and degassed with N₂ for further 5 minutes. The reaction mixture was stirred for 5 h at 100° C. in a sealed vessel. The reaction mixture was cooled to room temperature and evaporated to obtain crude product, which was purified by silica gel flash column chromatography. The compound eluted out in 3% MeOH:DCM. Fractions obtained from column containing product were concentrated to give 1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one (0.2 g, 37%) as off white solid. LCMS (ES) m/z=493.1 [M+H]⁺. ¹H NMR (400 MHz, DMSO-d₆) δ ppm—0.99 (d, J=7.8 Hz, 4H), 2.30 (s, 3H), 2.63 (s, 3H), 3.50-3.62 (m, 1H), 3.68 (t, J=8.0 Hz, 1H), 4.28 (t, J=8.0 Hz, 1H), 4.99 (t, J=8.2 Hz, 1H), 5.82 (br.s, 2H), 7.06 (s, 1H), 7.15-7.17 (m, 1H), 7.31 (q, J=8.2 Hz, 2H), 7.39-7.40 (m, 1H), 7.46-7.47 (m, 1H), 7.66-7.69 (m, 1H).

### Step 5: Enantiomer Separation

0.2 g of racemic 1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one was separated using chiral HPLC purification to give enantiomers 1 and 2. Preparative HPLC conditions: Column: CHIRALPAK IA (250 mm×20 mm×5 μm); Mobile phase: n-Hexane:Ethanol 0.1% TFA (50:50); Flow rate: 16 mL/min. pure fractions at retention time 15.60 min were concentrated to obtain enantiomer 1 as white solid (0.07 g, 35% yield). LCMS (ES) m/z=493.1 [M+H]⁺. ¹H NMR (400 MHz, DMSO-d6) δ ppm 0.98-1.01 (m, 4H), 2.39 (s, 3H), 2.63 (s, 3H), 3.56 (t, J=4.0 Hz, 1H), 3.67 (q, J=8.0 Hz, 1H), 4.28 (t, J=8.0 Hz, 1H), 4.99 (q, J=8.0 Hz, 1H), 5.83 (br.s., 2H), 7.06 (s, 1H), 7.15 (t, J=8.2 Hz, 1H), 7.31 (q, J=8.4 Hz, 2H), 7.39 (d, J=8.4 Hz, 1H), 7.47 (q, J=8.2 Hz, 1H), 7.67 (d, J=12.0 Hz, 1H): HPLC Analytical conditions: Column: CHIRALPAK IA (250 mm×4.6 mm×5 μm); Mobile phase: n-Hexane:Ethanol 0.1% TFA (50:50); Flow rate: 1.0 mL/min: retention time 15.55 min, 98.06% purity @265 nm. Pure fractions at retention time 27.82 min were concentrated to obtain enantiomer 2 as off white solid (0.07 g, 35% yield). LCMS (ES) m/z=493.1 [M+H]⁺. ¹H NMR (400 MHz, DMSO-d6) δ ppm 0.98-1.01 (m, 4H), 2.30 (s, 3H), 2.63 (s, 3H), 3.53-3.56 (m, 1H), 3.67 (q, J=8.0 Hz, 1H), 4.28 (t, J=8.0 Hz, 1H), 4.99 (q, J=8.0 Hz, 1H), 5.83 (br.s, 2H), 7.06 (s, 1H), 7.15 (t, J=8.2 Hz, 1H), 7.31 (q, J=8.4 Hz, 2H), 7.39 (d, J=8.2 Hz, 1H), 7.47 (q, J=8.0 Hz, 1H), 7.67 (d, J=12.0 Hz, 1H): HPLC Analytical conditions: Column: CHIRALPAK IA (250 mm×4.6 mm×5 μm); Mobile phase: n-Hexane:Ethanol 0.1% TFA (50:50); Flow rate: 1.0 mL/min: retention time 25.83 min, 96.10% purity (3.8% enantiomer 1, retention time 14.85 min) @265 nm.

### Example 10 and 11

### Enantiomers of 1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one

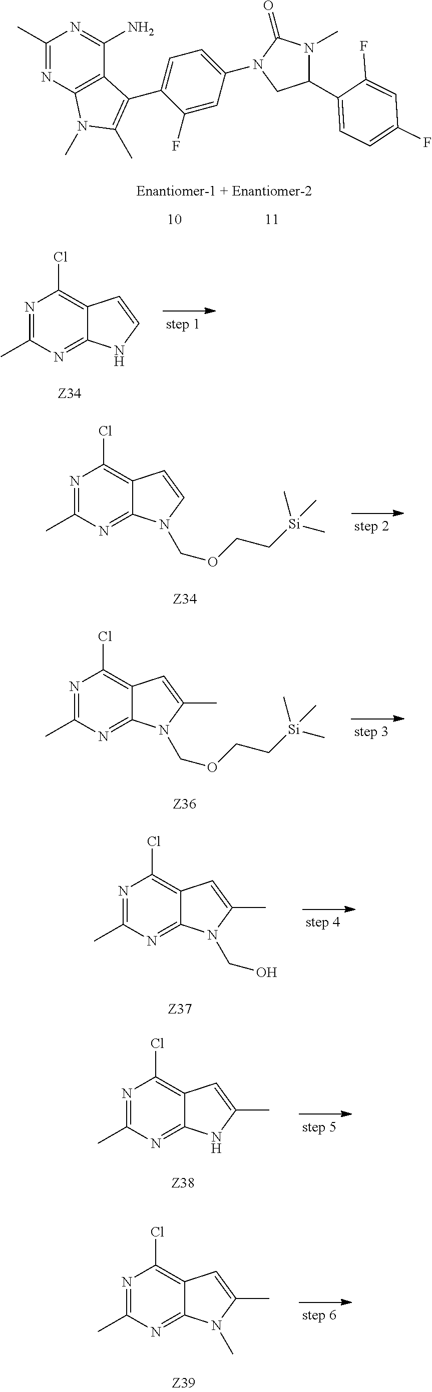

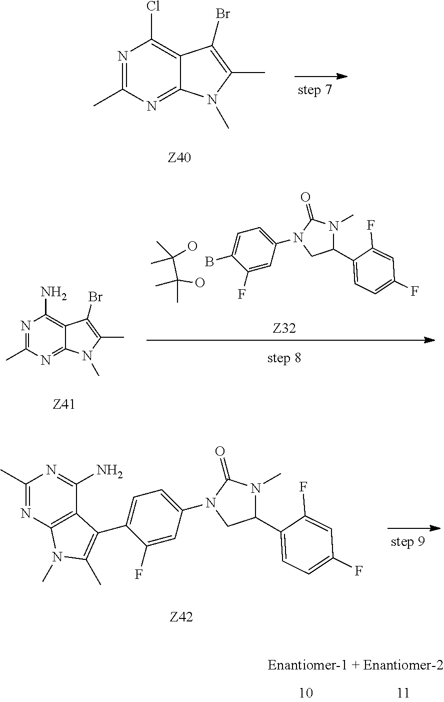

### Step 1

To a stirred solution of 4-chloro-2-methyl-7H-pyrrolo[2,3-d]pyrimidine (6.0 g, 35.9 mmol, 1 equiv) in DMF (70 mL) was added 60% sodium hydride (1.7 g, 43.3 mmol, 1.2 equiv) at 0° C. and stirred for 15 min. (2-(chloromethoxy)ethyl)trimethylsilane (6.4 mL, 35.9 mmol, 1.0 equiv) was added to the reaction mixture at 0° C. The reaction mixture was warmed to room temperature and stirred for 1 h. The reaction mixture was quenched with ice water. The crude product was extracted with ethyl acetate. The organic layer was dried over sodium sulphate and evaporated to obtain 4-chloro-2-methyl-7-((2-(trimethylsilyl)ethoxy)methyl)-7H-pyrrolo[2,3-d]pyrimidine as an brown liquid (7.0 g, Crude). LCMS (ES) m/z=298.1 [M+H]⁺.

### Step 2

To a stirred solution of 4-chloro-2-methyl-7-((2-(trimethylsilyl)ethoxy)methyl)-7H-pyrrolo[2,3-d]pyrimidine (7.0 g, 23.4 mmol, 1.0 equiv), in THF (70 mL) was added 2M LDA solution (17.5 mL, 1.5 equiv) at −78° C. under nitrogen atmosphere. The reaction mixture was stirred at same temperature for 15 minutes. Methyl iodide (8.7 mL, 140.9 mmol, 6.0 equiv) was added at −78° C. and the reaction mixture was stirred for 1 h. The reaction mixture was quenched with saturated ammonium chloride solution, extracted with ethyl acetate. The organic layer was dried over sodium sulphate and evaporated to obtain 4-chloro-2,6-dimethyl-7-((2-(trimethylsilyl)ethoxy)methyl)-7H-pyrrolo[2,3-d]pyrimidine as brown liquid (5.0 g, crude). LC-MS (ES) m/z=312.1 [M+H]⁺.

### Step 3

To a stirred mixture of 4-chloro-2,6-dimethyl-7-((2-(trimethylsilyl)ethoxy)methyl)-7H-pyrrolo[2,3-d]pyrimidine (5.0 g, 16.07 mmol, 1 equiv) in DCM (50 mL) was added TFA (4.8 mL, 64.3 mmol, 4.0 equiv) at 0° C. The reaction mixture was warmed to room temperature and stirred for 16 h. The reaction mixture was cooled to 0° C. and quenched with sat. NaHCO3 solution, extracted with ethyl acetate. The organic layer was dried over sodium sulphate and evaporated to obtain (4-chloro-2,6-dimethyl-7H-pyrrolo[2,3-d]pyrimidin-7-yl)methanol as pale brown solid (2.0 g, crude). LCMS (ES) m/z=212.1 [M+H]⁺.

### Step 4

To a stirred solution of (4-chloro-2,6-dimethyl-7H-pyrrolo[2,3-d]pyrimidin-7-yl)methanol (2.0 g, 9.47 mmol, 1.0 equiv) in THF (20 mL) was added borontrifluoride diethyl etherate (23.6 mL, 47.3 mmol, 5.0 equiv.) The reaction mixture was stirred for 24 h at room temperature. The reaction mixture was quenched with sat. NH4Cl solution and extracted with ethyl acetate. The organic layer was dried over Na₂SO₄ and evaporated to obtain 4-chloro-2,6,-trimethyl-7H-pyrrolo[2,3-d]pyrimidine (1.0 g, crude) as brown solid.

LCMS (ES) m/z=181.1 [M+H]⁺.

### Step 5

To a stirred solution of 4-chloro-2,6-trimethyl-7H-pyrrolo[2,3-d]pyrimidine (1.0 g, 5.5 mmol, 1.0 equiv), in DMF (10 mL) was added 60% sodium hydride (0.26 g, 6.6 mmol, 1.2 equiv) at 0° C. and stirred for 15 min. Methyl iodide (0.51 mL, 8.28 mmol, 1.5 equiv) was added to the reaction mixture at 0° C. and allowed to stir the reaction mixture at room temperature for 1 h. The reaction mixture was quenched with ice water and extracted with ethyl acetate. The organic layer was dried over sodium sulphate and evaporated to obtain 4-chloro-2,6,7-trimethyl-7H-pyrrolo[2,3-d]pyrimidine (0.8 g, crude) as brown solid. LCMS (ES) m/z=195.6 [M+H.]⁺.

### Step 6

To a stirred solution of 4-chloro-2,6,7-trimethyl-7H-pyrrolo[2,3-d]pyrimidine (0.7 g, 3.58 mmol, 1 equiv) in DCM (10 mL) was added NBS (0.64 g, 3.58 mmol, 1.0 equiv) at 000° C. The reaction mixture was warmed to room temperature and stirred for 2 h. The reaction mixture was quenched with water and extracted with ethyl acetate. The organic layer was dried over sodium sulphate and evaporated to obtain 5-bromo-4-chloro-2,6,7-trimethyl-7H-pyrrolo[2,3-d]pyrimidine (0.4 g, 42%) as pale yellow solid. LCMS (ES) m/z=274.5, 277.5 [M+H.]⁺. ¹H NMR (400 MHz, DMSO-d₆) δ ppm—2.42 (s, 3H), 2.62 (s, 3H), 3.72 (s, 3H).

### Step 7

To a stirred solution of 5-bromo-4-chloro-2,6,7-trimethyl-7H-pyrrolo[2,3-d]pyrimidine (0.4 g, 1.45 mmol, 1 equiv) in 1,4-dioxane (5 mL) was added NH₄OH (10 mL) at room temperature. The reaction mixture was heated at 100° C. in an autoclave for 16 h. The reaction mixture was cooled and the solids formed were filtered to obtain 5-bromo-2,6,7-trimethyl-7H-pyrrolo[2,3-d]pyrimidin-4-amine (0.2 g, 50%) as pale yellow solid. LCMS (ES) m/z=255.1, 257.1 [M+H.]⁺. ¹H NMR (400 MHz, DMSO-d₆) δ ppm—2.27 (s, 3H), 2.34 (s, 3H), 3.57 (s, 3H), 6.47 (br. s., 2H).

### Step 8

To a stirred solution of 4-(2,4-difluorophenyl)-1-(3-fluoro-4-(4,4,5,5-tetramethyl-1,3,2-dioxaborolan-2-yl)phenyl)-3-methylimidazolidin-2-one (0.3 g, 0.69 mmol, 1 equiv) (synthesized following procedure similar to example 1) in 1,4-dioxane (30 mL) was added 5-bromo-2,6,7-trimethyl-7H-pyrrolo[2,3-d]pyrimidin-4-amine (0.14 g, 0.52 mmol, 0.75 equiv), tri-potassium phosphate (0.29 g, 1.32 mmol, 2.0 equiv) and water (1 mL). The reaction mixture was degassed with N₂ for 15 minutes. Pd2(dba)3 (0.032 g, 0.034 mmol, 0.05 equiv) and (tBut)₃HPBF₄ (0.020 g, 0.069 mmol, 0.1 equiv) were added and the mixture was degassed with N₂ for further 5 minutes. The reaction mixture was stirred for 5 h at 100° C. in a sealed vessel. The reaction mixture was cooled to room temperature and evaporated to obtain crude product, which was purified over silica gel flash column chromatography. The compound eluted in 3% MeOH:DCM. Fractions obtained from column containing product were concentrated to give 1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one (0.2 g, 65%) as off white solid. LCMS (ES) m/z=481.1 [M+H]⁺. ¹H NMR (400 MHz, DMSO-d₆) δ ppm—2.16 (s, 3H), 2.37 (s, 3H), 2.63 (s, 3H), 3.62 (s, 3H), 3.69-3.78 (m, 1H), 4.29 (t, J=8.2 Hz, 1H), 4.98-5.02 (m, 1H), 5.57 (br. s., 2H), 7.16 (t, J=8.2 Hz, 1H), 7.25 (t, J=8.0 Hz, 1H), 7.30-7.35 (m, 1H), 7.41-7.51 (m, 2H), 7.67-7.71 (m, 1H).

### Step 9: Enantiomer Separation

0.2 g of racemic compound 1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one was separated using chiral HPLC purification to give enantiomers 1 and 2. Preparative HPLC conditions: Column: CHIRALPAK IA (250 mm×20 mm×5 μm); Mobile phase: n-Hexane:Ethanol 0.1% TFA (50:50); Flow rate: 12 mL/min. pure fractions at retention time 12.36 min were concentrated to obtain enantiomer 1 as white solid (0.05 g, 25% yield). LCMS (ES) m/z=481.1 [M+H]⁺. ¹H NMR (400 MHz, DMSO-d6) δ ppm 2.16 (s, 3H), 2.37 (s, 3H), 2.63 (s, 3H), 3.62 (s, 3H), 3.70-3.79 (m, 1H), 4.30 (t, J=8.0 Hz, 1H), 4.99 (t, J=8.2 Hz, 1H), 5.57 (br. s., 2H), 7.15 (t, J=8.0 Hz, 1H), 7.25 (t, J=8.0 Hz, 1H), 7.30-7.35 (m, 1H), 7.42-7.51 (m, 2H), 7.69-7.71 (m, 1H): HPLC Analytical conditions: Column: CHIRALPAK IA (250 mm×4.6 mm×5 μm); Mobile phase: n-Hexane:Ethanol 0.1% TFA (50:50); Flow rate: 1.0 mL/min: retention time 16.54 min, 99.95% purity @262 nm. Pure fractions at retention time 21.56 min were concentrated to obtain enantiomer 2 as off white solid (0.05 g, 25% yield). LCMS (ES) m/z=481.1 [M+H]⁺. ¹H NMR (400 MHz, DMSO-d6) δ ppm 2.16 (s, 3H), 2.37 (s, 3H), 2.65 (s, 3H), 3.62 (s, 3H), 3.70-3.79 (m, 1H), 4.30 (t, J=8.0 Hz, 1H), 4.98-5.02 (m, 1H), 5.57 (br. s., 2H), 7.12-7.18 (m, 1H), 7.25 (t, J=8.0 Hz, 1H), 7.29-7.35 (m, 1H), 7.42-7.49 (m, 2H), 7.69-7.71 (m, 1H): HPLC Analytical conditions: Column: CHIRALPAK IA (250 mm×4.6 mm×5 μm); Mobile phase: n-Hexane:Ethanol 0.1% TFA (50:50); Flow rate: 1.0 mL/min: retention time 30.01 min, 99.76% purity @262 nm.

### Example 12 and 13

### Enantiomers of 1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one

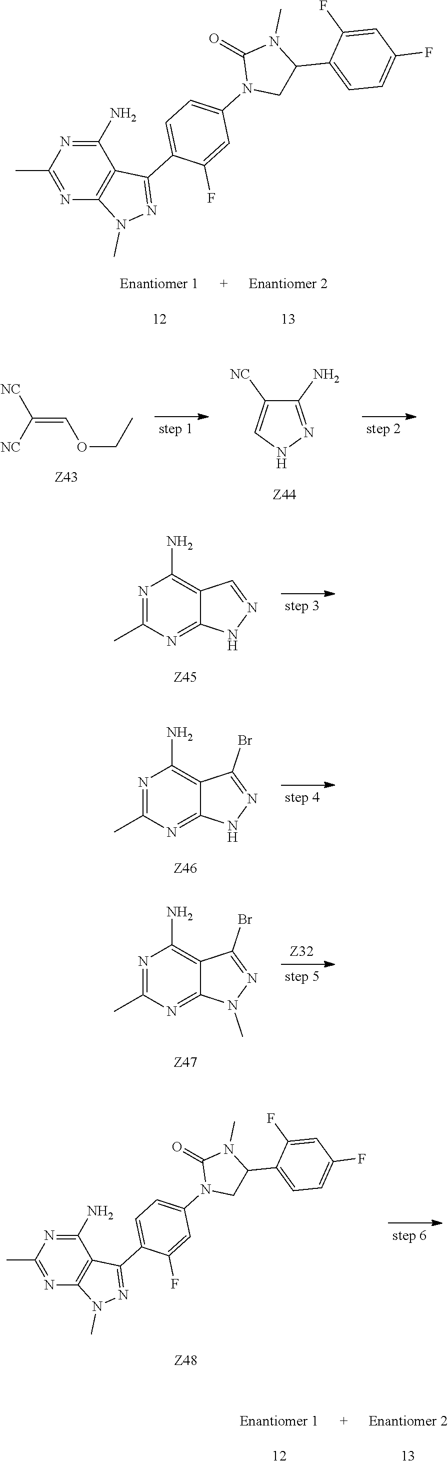

### Step 1

To a stirred solution of 2-(ethoxymethylene) malononitrile (5.0 g, 40.9 mmol, 1 equiv) in water (50 mL) was added hydrazine hydrate (5.0 mL, 102.4 mmol, 2.5 equiv) at room temperature. The resulting suspension was stirred at 110° C. for 16 h. The reaction mixture was cooled to room temperature and the solid formed was filtered under vacuum, and dried to obtain 3-amino-1H-pyrazole-4-carbonitrile as a brown solid (2.0 g, crude). LCMS (ES) m/z=109.1 [M+H]⁺. ¹H NMR (400 MHz, DMSO-d₆) δ ppm—6.09 (br.s., 2H), 7.63 (br.s., 1H), 12.03 (br.s., 1H).

### Step 2

To a stirred solution of 3-amino-1H-pyrazole-4-carbonitrile (2.0 g, 18.5 mmol, 1.0 equiv), in acetonitrile (15 mL) was added methanolic ammonia (40 mL) at room temperature. The reaction mixture was heated in an autoclave to 160° C. and stirred for 20 h. The reaction mixture was cooled to room temperature and the solid formed was filtered, and dried to obtain 6-methyl-1H-pyrazolo[3,4-d]pyrimidin-4-amine as brown solid (2.0 g, crude). LCMS (ES) m/z=150.1 [M+H]⁺. ¹H NMR (400 MHz, DMSO-d₆) δ ppm—3.14 (s, 3H), 7.43 (br.s., 2H), 7.96 (s, 1H), 12.0-13.0 (m, 1H).

### Step 3

To a stirred solution of 6-methyl-1H-pyrazolo[3,4-d]pyrimidin-4-amine (0.5 g, 3.35 mmol, 1 equiv) in DMF (10 mL) was added NBS (0.4 g, 2.01 mmol, 0.6 equiv) at 00° C. The reaction mixture was warmed to room temperature and stirred for 16 h. The reaction mixture was quenched with water and extracted with ethyl acetate. The organic layer was dried over sodium sulphate and evaporated to obtain 3-bromo-6-methyl-1H-pyrazolo[3,4-d]pyrimidin-4-amine (0.5 g, 65%) as pale yellow solid. LCMS (ES) m/z=228.1, 230.1 [M+H.]⁺. ¹H NMR (400 MHz, DMSO-d₆) 0 ppm—2.54 (s, 3H), 6.52-8.02 (br.s., 2H), 13.48 (s, 1H).

### Step 4

To a stirred solution of 3-bromo-6-methyl-1H-pyrazolo[3,4-d]pyrimidin-4-amine (0.5 g, 2.19 mmol, 1.0 equiv), in DMF (30 mL) was added 60% sodium hydride (0.08 g, 2.19 mmol, 1.0 equiv) at 0° C., and the mixture was stirred for 15 min. Methyl iodide (0.13 mL, 2.19 mmol, 1.0 equiv) was added at 0° C. The reaction mixture was warmed to room temperature and stirred for 1 h. The reaction mixture was quenched in ice water and extracted with ethyl acetate. The organic layer was dried over sodium sulphate and evaporated to obtain crude product which was purified by silica gel flash column chromatography. The compound eluted out in 3% MeOH:DCM. Fractions obtained from column containing product were concentrated to give 3-bromo-1,6-dimethyl-1H-pyrazolo[3,4-d]pyrimidin-4-amine (0.2 g, 45%) as yellow solid. LCMS (ES) m/z=244.1, 246.1 [M+H.]⁺.]⁺. ¹H NMR (400 MHz, DMSO-d₆) δ ppm—2.36 (s, 3H), 3.79 (s, 3H), 6.26-7.12 (br.s, 1H), 7.20-7.91 (br.s., 1H).

### Step 5

To a stirred solution of 4-(2,4-difluorophenyl)-1-(3-fluoro-4-(4,4,5,5-tetramethyl-1,3,2-dioxaborolan-2-yl)phenyl)-3-methylimidazolidin-2-one (0.3 g, 0.69 mmol, 1 equiv) (synthesized following procedure similar to example 1) (0.37 g, 0.85 mmol, 1 equiv) in 1,4-dioxane (30 mL) was added 3-bromo-1,6-dimethyl-1H-pyrazolo[3,4-d]pyrimidin-4-amine (0.15 g, 0.64 mmol, 0.75 equiv), tripotassium phosphate (0.36 g, 1.71 mmol, 2.0 equiv) and water (1 mL). The reaction mixture was degassed with N₂ for 15 minutes. Pd₂(dba)₃ (0.04 g, 0.042 mmol, 0.05 equiv) and (tBut)₃HPBF₄ (0.025 g, 0.085 mmol, 0.1 equiv) were added and degassed with N₂ for further 5 minutes. The reaction mixture was stirred for 5 h at 100° C. in a sealed vessel. The reaction was cooled to room temperature and evaporated to obtain crude product, which was purified over silica gel flash column chromatography. The compound eluted in 3% MeOH:DCM. Fractions obtained from column containg the product were concentrated to give 1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one (0.2 g, 65%) as off white solid (racemic compound). LCMS (ES) m/z=468.0 [M+H]⁺. ¹H NMR (400 MHz, DMSO-d₆) δ ppm—2.40 (s. 3H), 2.64 (s, 3H), 3.70 (t, J=8.0 Hz, 1H), 3.88 (s, 3H), 4.31 (t, J=8.2 Hz, 1H), 4.99-5.03 (m, 1H), 6.57 (br.s., 2H), 7.16 (t, J=8.0 Hz, 1H), 7.33 (t, J=8.5 Hz, 1H), 7.42-7.46 (m, 3H), 7.72 (d, J=12.2 Hz, 1H).

### Step 6: Enantiomer Separation

0.2 g of racemic compound 1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one was separated by using chiral HPLC to give enantiomers 1 and 2. Preparative HPLC conditions: Column: CHIRALPAK IA (250 mm×20 mm×5 mic); Mobile phase: MTBE:IPA with 0.1% DEA (90:10); Flow rate: 12 mL/min. Pure fractions at retention time 11.80 min were concentrated to obtain enantiomer 1 as white solid (0.05 g, 25%). LCMS (ES) m/z=468.1 [M+H]⁺. ¹H NMR (400 MHz, DMSO-d6) δ ppm 2.40 (s, 3H), 2.64 (s, 3H), 3.68-3.72 (m, 1H), 3.88 (s, 3H), 4.31 (t, J=8.2 Hz, 1H), 4.99-5.03 (m, 1H), 6.66 (br.s., 2H), 7.16 (t, J=8.0 Hz, 1H), 7.30-7.36 (m, 1H), 7.42-7.46 (m, 3H), 7.70-7.73 (m, 1H): HPLC Analytical conditions: Column: CHIRALPAK IA (250 mm×4.6 mm×5 μm); Mobile phase: MTBE:IPA with 0.1% DEA (90:10); Flow rate: 0.8 mL/min: 99.99% purity, retention time 11.57 min @264 nm. Pure fractions at retention time 21.56 min were concentrated to obtain enantiomer 2 as off white solid (0.05 g, 25%). LCMS (ES) m/z=468.1 [M+H]⁺. ¹H NMR (400 MHz, DMSO-d6) δ ppm 2.40 (s, 3H), 2.64 (s, 3H), 3.68-3.72 (m, 1H), 3.88 (s, 3H), 4.31 (t, J=8.0 Hz, 1H), 4.99-5.03 (m, 1H), 6.67 (br.s., 2H), 7.16 (t, J=8.0 Hz, 1H), 7.33 (t, J=8.2 Hz, 1H), 7.42-7.46 (m, 3H), 7.71 (d, J=12.2 Hz, 1H): HPLC Analytical conditions: Column: CHIRALPAK IA (250 mm×4.6 mm×5 μm); Mobile phase: MTBE:IPA with 0.1% DEA (90:10); Flow rate: 0.8 mL/min: 94.6% purity, retention time 14.59 min (5.3% enantiomer 1, retention time 11.59 min) @264 nm.

### Example 46

### 1-(4-(4-amino-7-methyl-7H-pyrrolo[2,3-d]pyrimidin-5-yl)-3-fluorophenyl)-4-(2,4-difluorophenyl)-3-methylimidazolidin-2-one (enantiomer 1)

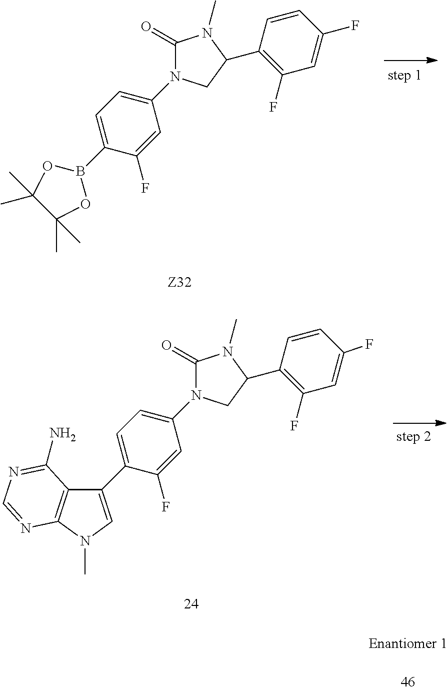 [SMILES: CN1C(=O)N(c2ccc(B3OC(C)(C)C(C)(C)O3)c(F)c2)CC1c1ccc(F)cc1F ; CN1C(=O)N(c2ccc(-c3cn(C)c4ncnc(N)c34)c(F)c2)CC1c1ccc(F)cc1F]

### Step 1

To a stirred solution of mixture of 4-(2,4-difluorophenyl)-1-(3-fluoro-4-(4,4,5,5-tetramethyl-1,3,2-dioxaborolan-2-yl)phenyl)-3-methylimidazolidin-2-one (0.2 g, 0.462 mmol, 1 equiv), in 1,4-dioxane:water (10 mL: 3 mL) was added 5-bromo-7-methyl-7H-pyrrolo[2,3-d]pyrimidin-4-amine (0.095 g, 0.416 mmol, 0.9 equiv), potassium phosphate (0.196 g, 0.924 mmol, 2 equiv), Pd₂(dba)₃ (0.021 g, 0.0231 mmol, 0.05 equiv) and tri-tert-butylphosphonium tetrafluoroborate (0.0133 g, 0.0462 mmol, 0.1 equiv) under argon atmosphere, then the mixture was heated to 100° C. for 6 h in a sealed tube. Reaction mixture was monitored by TLC and after consumption of the starting material, the reaction mixture was filtered through celite, dried over Na₂SO₄, and concentrated to obtain the crude product. Crude product was purified by flash column chromatography on silica gel, and the compound was eluted with 2% MeOH:DCM mobile phase. The pure fractions were evaporated to obtain 1-(4-(4-amino-7-methyl-7H-pyrrolo[2,3-d]pyrimidin-5-yl)-3-fluorophenyl)-4-(2,4-difluorophenyl)-3-methylimidazolidin-2-one as off white solid (0.07 g, 34.5%). LCMS (ES) m/z=453.1 [M+H]⁺. ¹H NMR (400 MHz, DMSO-d6) δ ppm 2.62 (s, 3H), 3.68-3.69 (m, 1H), 3.71 (s, 3H), 4.28 (t, J=9.6 Hz, 1H), 4.97-5.01 (m, 1H), 5.93 (br. S., 2H), 7.15 (t, J=6.4 Hz, 1H), 7.24 (s, 1H), 7.32 (t, J=8.8 Hz, 2H), 7.40-7.49 (m, 2H), 7.69 (d, J=13.2 Hz, 1H), 8.11 (s, 1H).

### Step 2: Enantiomer Separation

0.045 g of racemic compound 1-(4-(4-amino-7-methyl-7H-pyrrolo[2,3-d]pyrimidin-5-yl)-3-fluorophenyl)-4-(2,4-difluorophenyl)-3-methylimidazolidin-2-one was separated to get enantiomers 1 by chiral HPLC using preparative HPLC conditions: Column: CHIRALPAK IA (250 mm×20 mm×5 mic); Mobile phase: MTBE:EtOAc with 0.1% TFA (85:15); Flow rate: 18 mL/min. Pure fractions at retention time 17.10 min were concentrated. The residue obtained was diluted with DCM, washed with saturated NaHCO₃ and brine solution. The organic layer was dried over Na₂SO₄ and concentrated to afford the enantiomer 1 as off white solid (0.011 g, 25% yield). LCMS (ES) m/z=453.1 [M+H]⁺. ¹H NMR (400 MHz, DMSO-d6) δ ppm 2.63 (s, 3H), 3.67-3.70 (m, 1H), 3.73 (s, 3H), 4.28 (t, J=9.6 Hz, 1H), 4.98-5.02 (m, 1H), 6.22 (br. s., 2H), 7.13-7.17 (m, 1H), 7.30-7.34 (m, 3H), 7.40-7.49 (m, 2H), 7.68-7.71 (m, 1H), 8.17 (s, 1H). Analytical HPLC: Column: CHIRALPAK IA (250 mm×4.6 mm×5 μm), Mobile phase: 0.1% DEA in 100% MEOH Flow rate: 0.7 mL/min, Retention time: 17.101 min.

### Example 59

### 1-(4-(4-amino-7-methyl-7H-pyrrolo[2,3-d]pyrimidin-5-yl)-3-fluorophenyl)-4-(4-fluorophenyl)-3-methylimidazolidin-2-one (Enantiomer 1)

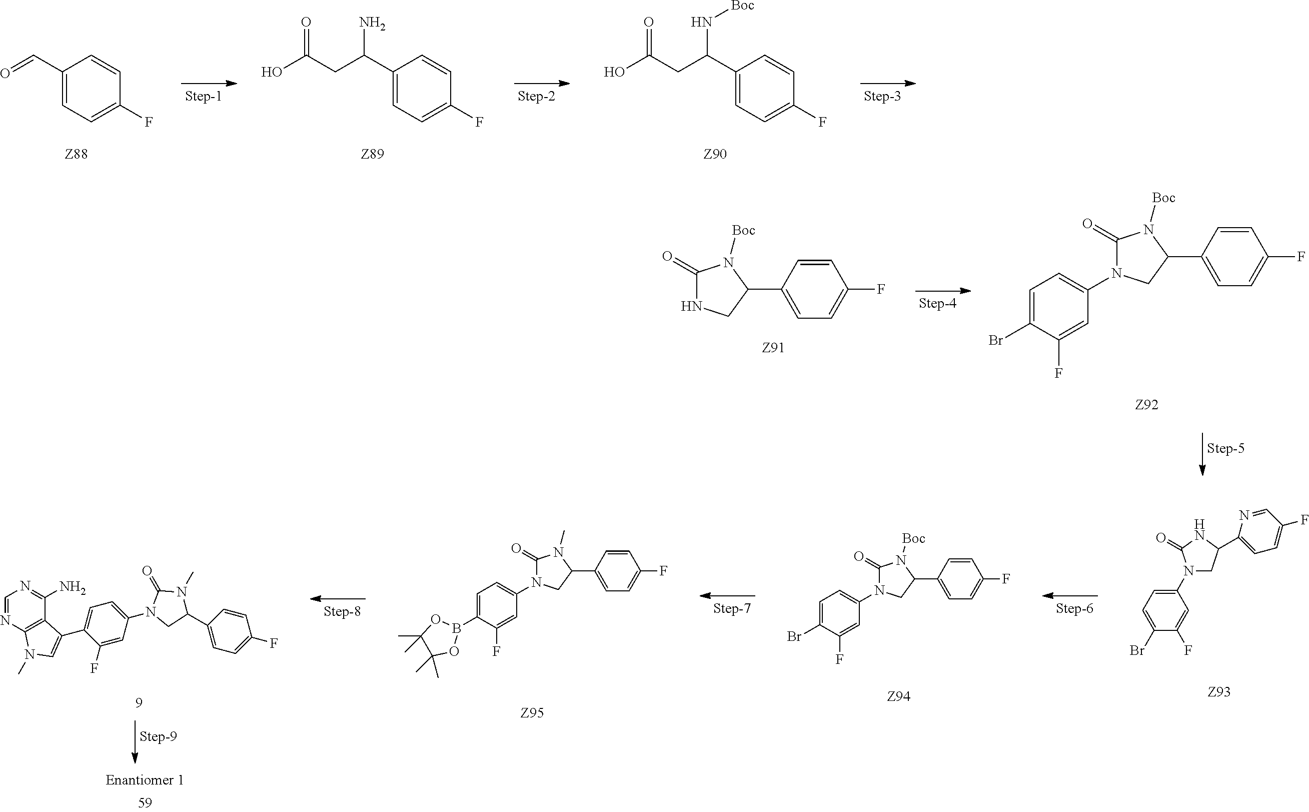

### Step 1

To a stirred solution of 4-fluorobenzaldehyde (5.0 g, 40.3 mmol, 1 equiv) in EtOH (60 mL) was added Malonic acid (5.03 g, 48.3 mmol, 1.2 equiv) and NH₄OAc (6.20 g, 80.6 mmol, 2.0 equiv), and the mixture was heated to 80° C. overnight. After the reaction mixture was cooled to room temperature, the formed solid was filtered and washed with EtOH and dried to obtain 3-amino-3-(4-fluorophenyl)propanoic acid crude compound as off white solid (6.1 g, crude). LC-MS (ES) m/z=184.1 [M+H]⁺.

### Step 2

To a stirred solution of 3-amino-3-(4-fluorophenyl)propanoic acid (5.1 g, 27.8 mmol, 1.0 equiv), in 1,4-Dioxane (50 mL) was added Boc₂O (9.3 mL, 41.7 mmol, 1.5 equiv) and sat.NaHCO₃ solution (50 mL) at 0° C., and the mixture was stirred overnight at room temperature. The reaction mixture was washed with Hexane and the aqueous layer was acidified with 10% citric acid solution and extracted with DCM (3×150 mL). The combined organic layers was washed with water and brine solution, dried over Na₂SO₄ filtered and concentrated to obtained 3-((tert-butoxycarbonyl)amino)-3-(4-fluorophenyl)propanoic acid as off white solid (5.2 g, 66.0%). LC-MS (ES) m/z=228.1 [M+H]⁺-56. ¹H NMR (400 MHz, DMSO-d6) δ ppm: 1.32 (s, 9H), 2.53-2.67 (m, 2H), 4.85 (d, J=6.8 Hz, 1H), 7.10 (t, J=8.8 Hz, 2H), 7.29-7.32 (m, 2H), 7.39 (d, J=7.6 Hz, 1H), 12.1 (s, 1H).

### Step 3

To a stirred solution of 3-((tert-butoxycarbonyl)amino)-3-(4-fluorophenyl)propanoic acid (5.0 g, 17.66 mmol, 1 equiv) in toluene (100 mL) was added TEA (6.2 mL, 44.15 mmol, 2.5 equiv) and DPPA (3.83 mL, 17.66 mmol, 1.0 equiv), and the mixture was stirred for 1 h at room temperature, and then was heated to 80° C. overnight. After consumption of the stating material, the reaction mixture was cooled to room temperature and diluted with EtOAc (200 mL) and the organic layer was washed with water, sat.NaHCO₃ and brine solution. The organic layer was dried over Na₂SO₄, filtered and concentrated to get crude product. The crude product was purified by flash column chromatography on silica gel and the compound was eluted with 30% EtOAc:Hexane. The pure fractions were evaporated to obtain tert-butyl 5-(4-fluorophenyl)-2-oxoimidazolidine-1-carboxylate as off white solid (5.0 g, crude). LC-MS (ES) m/z=225.1 [M+H]⁺-56.

### Step 4

To a stirred solution of tert-butyl 5-(4-fluorophenyl)-2-oxoimidazolidine-1-carboxylate (5.0 g, 17.8 mmol, 1.0 equiv) in EtOAc (100 mL), was added 1-bromo-2-fluoro-4-iodobenzene (8.06 g, 26.7 mmol, 1.5 equiv), DMEDA (0.38 mL, 3.56 mmol, 0.2 equiv), CsF (6.76 g, 44.5 mmol, 2.5 equiv), and CuI (0.67 g, 3.56 mmol, 0.2 equiv), the mixture was stirred at room temperature overnight. After consumption of the starting material the reaction mixture was diluted with EtOAc (100 mL) and washed with water, followed by the brine. The organic phase was dried over Na₂SO₄, filtered and concentrated to obtain the product tert-butyl 3-(4-bromo-3-fluorophenyl)-5-(4-fluorophenyl)-2-oxoimidazolidine-1-carboxylate (2.5 g, 31.2%) as off white solid. LC-MS (ES) m/z=397.0, 399.1 [M+H]⁺-56. ¹H NMR (400 MHz, DMSO-d6) δ ppm 1.24 (s, 9H), 3.58-3.61 (m, 1H), 4.23-4.28 (m, 1H), 5.24-5.28 (m, 1H), 7.20 (t, J=8.8 Hz, 2H), 7.38-7.40 (m, 3H), 7.63-7.71 (m, 2H).

### Step 5

To a stirred solution of tert-butyl 3-(4-bromo-3-fluorophenyl)-5-(4-fluorophenyl)-2-oxoimidazolidine-1-carboxylate (2.5 g, 5.53 mmol, 1.0 equiv), in MeOH (25 mL) was added 4M HCl in dioxane (15 mL) at at 0° C., and the reaction mixture was stirred at 0° C. to room temperature for 3 h. After consumption of the starting material the reaction mixture was concentrated to get crude product. Crude product was triturated with Et₂O to obtain 1-(4-bromo-3-fluorophenyl)-4-(4-fluorophenyl)imidazolidin-2-one as off white solid (2.0 g, crude). LC-MS (ES) m/z=353.0, 355.0 [M+H]⁺. ¹H NMR (400 MHz, DMSO-d6) δ ppm 3.57 (t, J=7.2 Hz, 1H), 4.24 (t, J=9.6 Hz, 1H), 4.88 (t, J=8.0 Hz, 1H), 7.21 (t, J=8.8 Hz, 2H), 7.29 (d, J=8.8 Hz, 1H), 7.42 (t, J=8.0 Hz, 2H), 7.56 (t, J=8.4 Hz, 1H), 7.70 (d, J=10.4 Hz, 1H), 7.83 (s, 1H).

### Step 6

To a stirred suspension of 1-(4-bromo-3-fluorophenyl)-4-(4-fluorophenyl)imidazolidin-2-one (1.0 g, 2.83 mmol, 1 equiv) in DMF (20 mL) was added 60% NaH (0.136 g, 3.39 mmol, 1.2 equiv) at 0° C. The mixture was stirred for 30 minutes, then added Methyl Iodide (0.22 mL, 3.4 mmol, 1.2 equiv) at 0° C. and stirred for 2 h at 0° C. After completion of starting material the reaction mixture was quenched with ice water, and filtered the obtained solid and dried to get the 1-(4-bromo-3-fluorophenyl)-4-(4-fluorophenyl)-3-methylimidazolidin-2-one as off white solid (0.97 g, 93.0%). LCMS (ES) m/z=367.0, 369.0 [M+H]⁺. ¹H NMR (400 MHz, DMSO-d6) δ ppm 2.56 (s, 3H), 3.55 (br.s, 1H), 4.19 (br.s, 1H), 4.72 (br.s, 1H), 7.24 (br.s, 2H), 7.33 (d, J=6.8 Hz, 1H), 7.42 (br.s, 2H), 7.58 (br.s, 1H), 7.71 (d, J=11.2 Hz, 1H).

### Step 7

To a stirred solution of 1-(4-bromo-3-fluorophenyl)-4-(4-fluorophenyl)-3-methylimidazolidin-2-one (0.97 g, 2.64 mmol, 1 equiv) in 1,4-Dioxane (20 mL) was added bis(pinacolato)diboron (0.805 g, 3.17 mmol, 1.2 equiv), and potassium acetate (0.65 g, 6.6 mmol, 3 equiv), under argon atmosphere, then PdCl₂(dppf)-CH₂Cl₂ adduct (0.215 g, 0.264 mmol, 0.1 equiv) was added under an argon atmosphere and the mixture was heated to 100° C. overnight in a sealed vessel. The reaction mixture was monitored by TLC and LCMS. After consumption of the stating material, the reaction mixture was filtered through celite, and the filtrate was concentrated to get crude product. The crude product was purified by flash column chromatography on silica gel and the compound was eluted with 30% EtOAc:Hexane. The pure fractions were evaporated to obtain 1-(3-fluoro-4-(4,4,5,5-tetramethyl-1,3,2-dioxaborolan-2-yl)phenyl)-4-(4-fluorophenyl)-3-methylimidazolidin-2-one (0.8 g, 73.0%) as off white solid. LCMS (ES) m/z=415.2 [M+H]⁺.

### Step 8

To a stirred solution of 1-(3-fluoro-4-(4,4,5,5-tetramethyl-1,3,2-dioxaborolan-2-yl)phenyl)-4-(4-fluorophenyl)-3-methylimidazolidin-2-one (0.4 g, 0.966 mmol, 1 equiv), in 1,4-Dioxane:water (8 mL: 2 mL), was added compound 5-bromo-7-methyl-7H-pyrrolo[2,3-d]pyrimidin-4-amine (0.219 g, 0.966 mmol, 1 equiv), potassium phosphate (0.41 g, 1.932 mmol, 2 equiv), Pd₂(dba)₃ (0.044 g, 0.0483 mmol, 0.05 equiv), and Tri-tert-butylphosphonium tetrafluoroborate (0.028 g, 0.0966 mmol, 0.1 equiv) under argon atmosphere. The mixture was then heated to 100° C. for 6 h in a sealed tube. After consumption of the starting material, the reaction mixture was filtered through celite, and the filtrate was dried over Na₂SO₄, and concentrated to get crude product, which was purified by flash column chromatography on a silica gel column. The compound was eluted with 2% MeOH:DCM. The pure fractions were evaporated to obtain 1-(4-(4-amino-7-methyl-7H-pyrrolo[2,3-d]pyrimidin-5-yl)-3-fluorophenyl)-4-(4-fluorophenyl)-3-methylimidazolidin-2-one (0.26 g, 62.0%) as off white solid. LCMS (ES) m/z=435.1 [M+H]⁺. ¹H NMR (400 MHz, DMSO-d6) δ ppm 2.58 (s, 3H), 3.60 (t, J=8.0 Hz, 1H), 3.72 (s, 3H), 4.25 (t, J=9.2 Hz, 1H), 4.74 (t, J=8.0 Hz, 1H), 5.93 (br.s., 2H), 7.23-7.27 (m, 3H), 7.32 (t, J=8.8 Hz, 1H), 7.41-7.46 (m, 3H), 7.70 (d, J=12.8 Hz, 1H), 8.12 (s, 1H).

### Step 9: Enantiomer Separation

0.2 g of racemic compound 1-(4-(4-amino-7-methyl-7H-pyrrolo[2,3-d]pyrimidin-5-yl)-3-fluorophenyl)-4-(4-fluorophenyl)-3-methylimidazolidin-2-one was separated to obtain pure enantiomers by chiral HPLC using preparative HPLC conditions: Column: CHIRALPAK IC (250 mm×20 mm×5 mic); Mobile phase: MTBE:IPA with 0.1% DEA (70:30); Flow rate: 18 mL/min. Pure fractions at retention time 15.069 min were concentrated, to afford the enantiomer 1 as off white solid (0.052 g, 52% yield). LCMS (ES) m/z=435.2 [M+H]⁺. ¹H NMR (400 MHz, DMSO-d6) δ ppm 2.58 (s, 3H), 3.59 (t, J=8.0 Hz, 1H), 3.73 (s, 3H), 4.25 (t, J=9.2 Hz, 1H), 4.75 (t, J=8.0 Hz, 1H), 6.1 (br.s., 2H), 7.24-7.35 (m, 4H), 7.41-7.46 (m, 3H), 7.70 (d, J=13.2 Hz, 1H), 8.16 (s, 1H). Analytical HPLC Column: Chiralpak IC (250 mm×4.6 mm×5 μm), Mobile phase: MTBE:IPA with 0.1% DEA (70:30), Flow rate: 1.0 mL/min, Retention time: 15.069 min.

### Example 100

### 1-(4-(4-amino-7-methyl-7H-pyrrolo[2,3-d]pyrimidin-5-yl)-3-fluorophenyl)-4-(3,5-difluorophenyl)-3-methylimidazolidin-2-one (enantiomer 1)

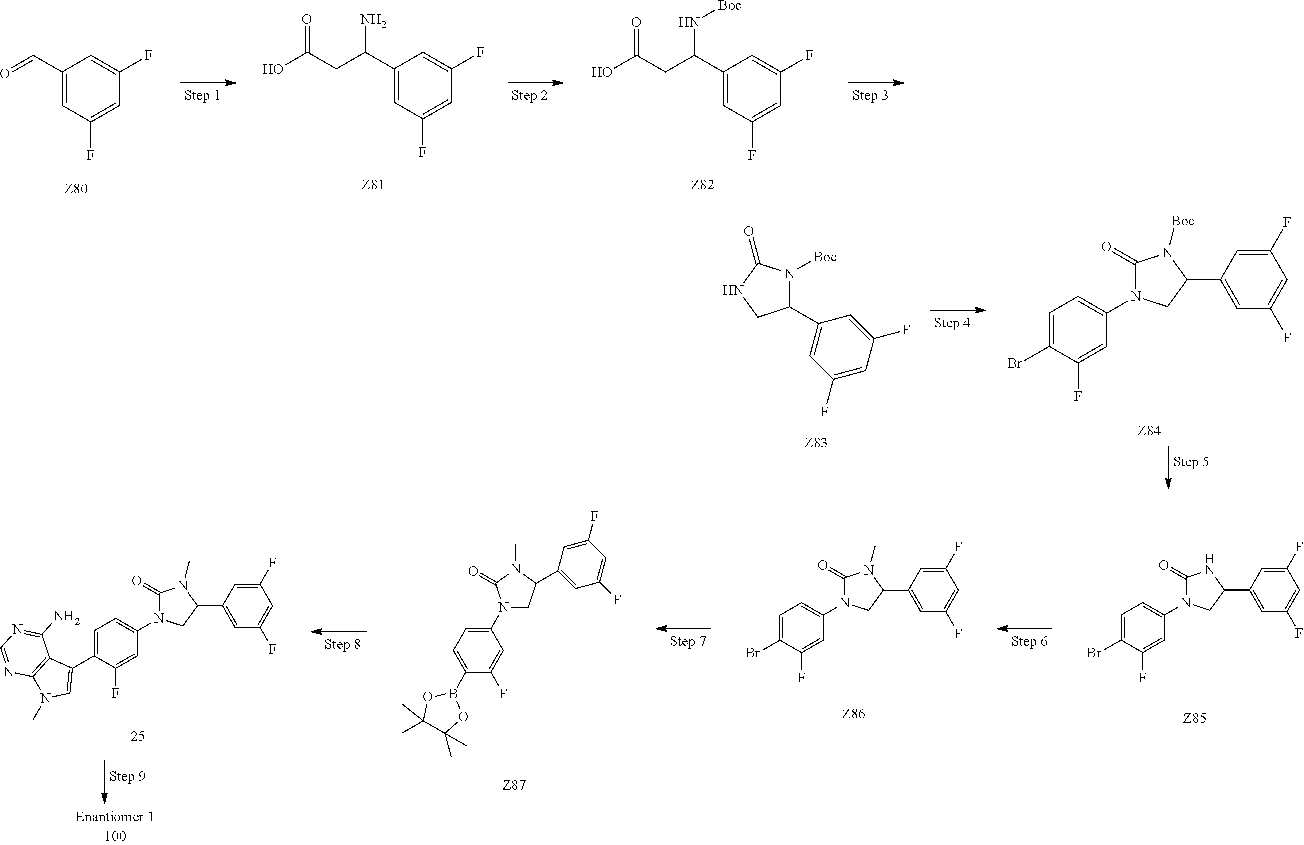

### Step 1

To a stirred solution of 3,5-Difluoro-benzaldehyde (7.0 g, 49.29 mmol, 1 equiv) in ethanol (100 mL) was added ammonium acetate (7.5 g, 98.59 mmol, 2.0 equiv) followed by malonic acid (5.12 g, 59.15 mmol, 1.2 equiv) at room temperature. The reaction mixture was stirred at 80° C. overnight. The reaction mixture was cooled to room temperature and the solids formed were filtered and washed with ethanol, dried under vacuum to afford 3-Amino-3-(3,5-difluoro-phenyl)-propionic acid as white solid (5.0 g, crude). LC-MS (ES) m/z=202.1 [M+H]⁺. ¹H NMR (400 MHz, DMSO-d6) δ ppm: 2.30-2.40 (m, 2H), 4.22-4.25 (m, 1H), 5.68-6.94 (m, 3H), 7.05-7.14 (m, 3H).

### Step 2

To a stirred solution of 3-Amino-3-(3,5-difluoro-phenyl)-propionic (5.0 g, 24.87 mmol, 1 equiv) in dioxane (50 mL) and sat. NaHCO₃ solution (50 mL) was added Boc₂O (8.5 mL, 37.31 mmol, 1.5 equiv) at room temperature. The reaction mixture was stirred at RT overnight. After consumption of the starting material, the reaction mixture was washed with EtOAc. The aqueous layer was acidified to pH 3 using citric acid solution and extracted in EtOAc. The combine organic layers was dried over Na₂SO₄ and concentrated under reduced vacuum to afford 3-tert-Butoxycarbonylamino-3-(3,5-difluoro-phenyl)-propionic acid as white solid (7.0 g, crude). LC-MS (ES) m/z=302.1 [M+H]⁺. ¹H NMR (400 MHz, DMSO-d6) δ ppm: 1.33 (s, 9H), 2.55-2.66 (m, 2H), 4.85-4.87 (m, 1H), 7.43-7.45 (m, 1H), 6.98-7.07 (m, 3H), 12.25 (s, 1H).

### Step 3

To a stirred solution of 3-tert-Butoxycarbonylamino-3-(3,5-difluoro-phenyl)-propionic acid (6.5 g, 21.5 mmol, 1 equiv) in toluene (80 mL) was added TEA (7.5 mL, 53.98 mmol, 2.5 equiv) at room temperature. DPPA (5.7 mL, 25.90 mmol, 1.2 equiv) was then added at room temperature. The reaction mixture was stirred at 75° C. overnight. The reaction mixture was cooled to room temperature, quenched with water and extracted in EtOAc. The combine organic layers was dried over Na₂SO₄ and concentrated under reduced vacuum to afford 5-(3,5-Difluoro-phenyl)-2-oxo-imidazolidine-1-carboxylic acid tert-butyl ester as pale brown solid (5.0 g, crude). LC-MS (ES) m/z=243.1 [M+H]⁺-56.

### Step 4

To a stirred solution of 5-(3,5-Difluoro-phenyl)-2-oxo-imidazolidine-1-carboxylic acid tert-butyl ester (4.2 g, 14.09 mmol, 1.0 equiv), 1-bromo-2-fluoro-4-iodobenzene (4.2 g, 14.09 mmol, 1.0 equiv), and CsF (5.3 g, 35.23 mmol, 2.5 equiv) in EtOAc (100 mL) was added CuI (0.26 g, 1.40 mmol, 0.1 equiv) followed by DMEDA (0.3 mL, 2.80 mmol, 0.2 equiv) at room temperature. The reaction mixture was stirred at room temperature for 24 h. The reaction mixture was quenched with water and extracted with EtOAc. The combine organic layers was dried over Na₂SO₄ and concentrated under reduced vacuum to afford 3-(4-Bromo-3-fluoro-phenyl)-5-(3,5-difluoro-phenyl)-2-oxo-imidazolidine-1-carboxylic acid tert-butyl ester as a pale yellow solid (1.4 g, Crude). LC-MS (ES) m/z=415.0, 417.0 [M+H]⁺-56.

### Step 5

To a stirred solution of 3-(4-Bromo-3-fluoro-phenyl)-5-(3,5-difluoro-phenyl)-2-oxo-imidazolidine-1-carboxylic acid tert-butyl ester (1.4 g, 2.97 mmol, 1.0 equiv), in 1,4-Dioxane (10 mL) was added 4M HCl in Dioxane (20 mL) at 0° C. and the reaction mixture was stirred at room temperature for 5 h. The reaction mixture was concentrated completely and diluted with DCM and basified with aq. NaHCO₃ solution. The reaction mixture was extracted in DCM. The combined organic layer dried over sodium sulphate and concentrated under reduced vacuum to afford 1-(4-Bromo-3-fluoro-phenyl)-4-(3,5-difluoro-phenyl)-imidazolidin-2-one as pale yellow solid (0.7 g, crude). LC-MS (ES) m/z=371.0, 373.0 [M+H]⁺.

### Step 6

To a stirred suspension of 1-(4-Bromo-3-fluoro-phenyl)-4-(3,5-difluoro-phenyl)-imidazolidin-2-one (0.7 g, 1.88 mmol, 1 equiv) in DMF (15 mL) was added 60% sodium hydride (0.09 g, 2.26 mmol, 1.2 equiv) at 0° C. under N₂ atmosphere. The reaction mixture was stirred for 20 min. Methyl Iodide (0.32 g, 2.26 mmol, 1.2 equiv) was added at 0° C. and the reaction mixture was stirred for 2 h at room temperature. After consumption of the starting material the reaction mixture was quenched with ice water and extracted in EtOAc. The combined organic layers was dried over Na₂SO₄ and concentrated under reduced vacuum to afford 1-(4-Bromo-3-fluoro-phenyl)-4-(3,5-difluoro-phenyl)-3-methyl-imidazolidin-2-one as pale yellow solid (0.6 g, crude). LCMS (ES) m/z=385.0, 387.0 [M+H]⁺. ¹H NMR (400 MHz, DMSO-d6) δ ppm: 2.60 (s, 3H), 3.56-3.60 (m, 1H), 4.17-4.22 (m, 1H), 4.75 (t, J=8.4 Hz, 1H), 7.14-7.16 (m, 2H), 7.23 (t, J=9.2 Hz, 1H), 7.30-7.32 (m, 1H), 7.59 (t, J=8.6 Hz, 1H), 7.69 (dd, J=2.0, 12.0 Hz, 1H)

### Step 7

To a stirred solution of 1-(4-Bromo-3-fluoro-phenyl)-4-(3,5-difluoro-phenyl)-3-methyl-imidazolidin-2-one (0.6 g, 1.55 mmol, 1 equiv) in 1,4-dioxane (50 mL) was added bis(pinacolato)diboron (0.59 g, 2.33 mmol, 1.5 equiv), and potassium acetate (0.45 g, 4.67 mmol, 3 equiv). The reaction mixture was degassed with argon for 15 minutes. PdCl₂(dppf).DCM adduct (0.19 g, 0.27 mmol, 0.15 equiv) was added and the mixture was degassed with argon for a further 15 minutes. The reaction mixture was stirred overnight at 100° C. in a sealed tube. The reaction mixture was filtered through celite and the filtrate was concentrated to obtain the crude product. The crude product was purified using silica gel flash column chromatography. The compound eluted out in 15-18% EtOAc:Hexanes. The pure fractions were evaporated to obtain 4-(3,5-Difluoro-phenyl)-1-[3-fluoro-4-(4,4,5,5-tetramethyl-[1,3,2]dioxaborolan-2-yl)-phenyl]-3-methyl-imidazolidin-2-one (0.35 g, crude) as a pale yellow solid. LCMS (ES) m/z=433.1 [M+H]⁺. ¹H NMR (400 MHz, DMSO-d6) δ ppm: 1.26 (s, 12H), 2.61 (s, 3H), 3.58 (t, J=8.4 Hz, 1H), 4.22 (t, J=9.4 Hz, 1H), 4.76 (t, J=8.0 Hz, 1H), 7.14-7.15 (m, 2H), 7.20-7.25 (m, 1H), 7.29-7.31 (m, 1H), 7.48-7.56 (m, 2H).

### Step 8

To a stirred solution of 4-(3,5-Difluoro-phenyl)-1-[3-fluoro-4-(4,4,5,5-tetramethyl-[1,3,2]dioxaborolan-2-yl)-phenyl]-3-methyl-imidazolidin-2-one (0.35 g, 0.81 mmol, 1 equiv) in 1,4-dioxane:water (25 mL: 1.0 mL), was added 5-bromo-7-methyl-7H-pyrrolo[2,3-d]pyrimidin-4-amine (0.138 g, 0.60 mmol, 0.75 equiv) and potassium phosphate (0.34 g, 1.62 mmol, 2 equiv) at RT. The reaction mixture was degassed with argon for 15 min.

Pd₂(dba)₃ (0.037 g, 0.04 mmol, 0.05 equiv) and tri-tert-butylphosphonium tetrafluoroborate (0.023 g, 0.081 mmol, 0.1 equiv) was added and the reaction mixture was further degassed for 15 minutes. The reaction mixture was heated at 100° C. for 5 h in a sealed tube. The reaction mixture was cooled & filtered through celite and the filtrate was concentrated to obtain crude compound. Crude compound was purified by flash column chromatography using a silica gel column, and the compound was eluted at 2.5% MeOH:DCM. The pure fractions were evaporated to obtain 1-[4-(4-Amino-7-methyl-7H-pyrrolo[2,3-d]pyrimidin-5-yl)-3-fluoro-phenyl]-4-(3,5-difluoro-phenyl)-3-methyl-imidazolidin-2-one (0.2 g, 20%) as off white solid. LCMS (ES) m/z=453.1 [M+H]⁺.]⁺. ¹H NMR (400 MHz, DMSO-d6) δ ppm: 2.48 (s, 3H), 3.61-3.65 (m, 1H), 3.72 (s, 3H), 4.23-4.28 (m, 1H), 4.78 (t, J=8.0 Hz, 1H), 5.92 (br.s, 2H), 7.15-7.17 (m, 2H), 7.22-7.26 (m, 2H), 7.30-7.35 (m, 1H), 7.39-7.42 (m, 1H), 7.67-7.70 (m, 1H), 8.12 (s, 1H).

### Step 9: Enantiomer Separation

0.2 g of racemic 1-[4-(4-Amino-7-methyl-7H-pyrrolo[2,3-d]pyrimidin-5-yl)-3-fluoro-phenyl]-4-(3,5-difluoro-phenyl)-3-methyl-imidazolidin-2-one was separated to obtain pure enantiomers by chiral HPLC using preparative HPLC conditions: Column: CHIRALPAK IA (250 mm×20 mm×5 mic); Mobile phase: n-Hexane: 0.1% DEA in Ethanol (560:50); Flow rate: 15 mL/min. Pure fractions at retention time 13.55 min were concentrated to afford the enantiomer 1 as off white solid (0.05 g, 25% yield). LCMS (ES) m/z=453.1[M+H]⁺. ¹H NMR (400 MHz, DMSO-d6) δ ppm: 2.63 (s, 3H), 3.63 (t, J=8.4 Hz, 1H), 3.72 (s, 3H), 4.25 (t, J=9.4 Hz, 1H), 4.76 (t, J=8.0 Hz, 1H), 5.92 (br.s., 2H), 7.16 (d, J=6.4 Hz, 2H), 7.22-7.25 (m, 2H), 7.33 (t, J=8.6 Hz, 1H), 7.39-7.41 (m, 1H), 7.68 (d, J=12.4 Hz, 1H), 8.12 (s, 1H). Analyical HPLC Column: CHIRALPAK IA (250 mm×4.6 mm×5 mic) Mobile phase: n-Hexane:0.1% DEA in Ethanol (50:50), Flow rate: 1.0 ml/min, Retention time: 13.324 min.

### Example 110

### 1-(4-(4-amino-7-methyl-7H-pyrrolo[2,3-d]pyrimidin-5-yl)-3-fluorophenyl)-3-methyl-4-(pyridin-4-yl)imidazolidin-2-one

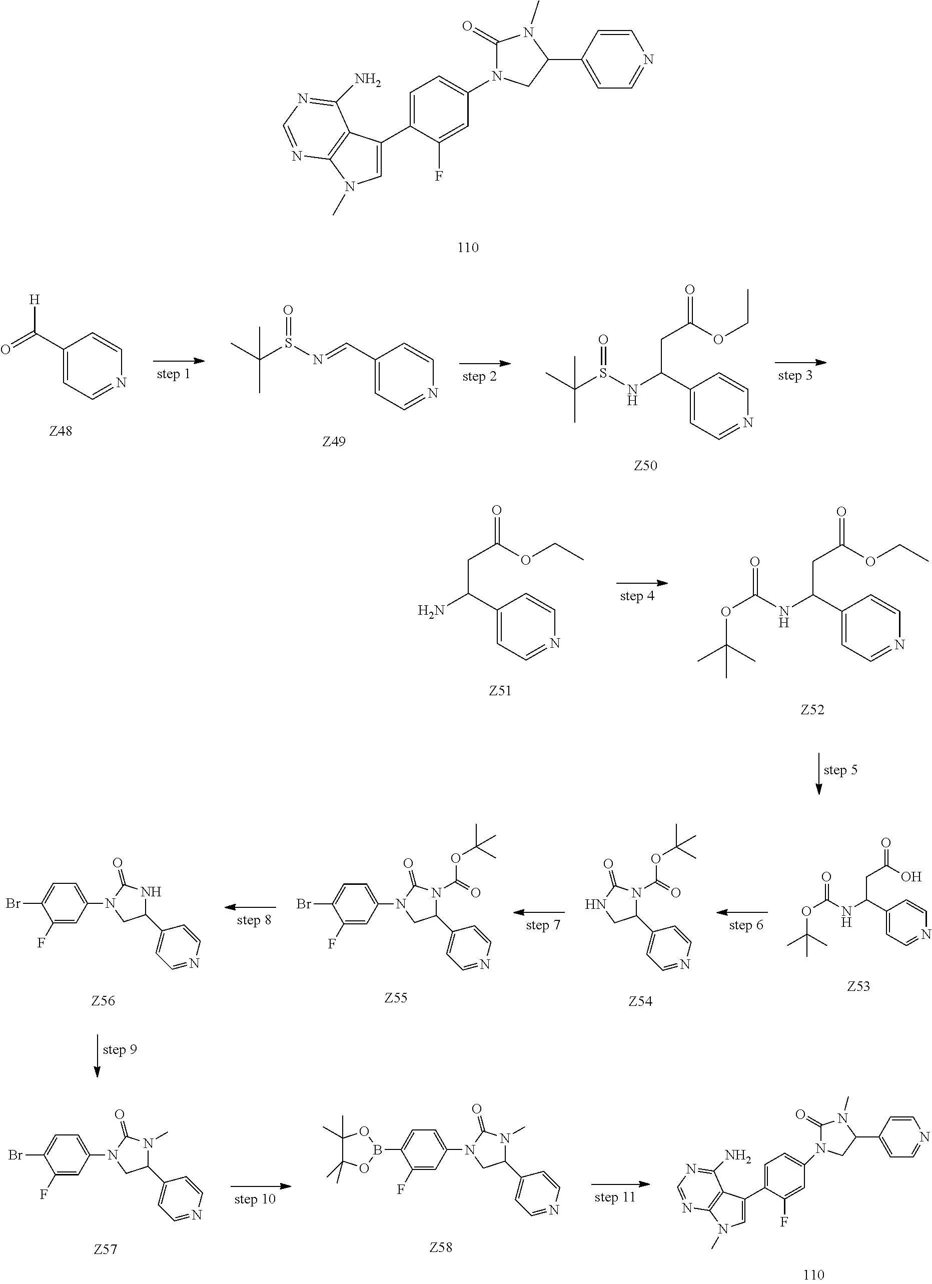

### Step 1

To a stirred solution of isonicotinaldehyde (5.0 g, 46.70 mmol, 1 equiv) in THF (50 mL) was added 2-methylpropane-2-sulfinamide (8.5 g, 70.00 mmol, 1.5 equiv) and titanium tetraethoxide (15.0 mL, 70.00 mmol, 1.5 equiv) at room temperature. The reaction mixture was stirred at 80° C. overnight. The reaction mixture was cooled to room temperature, and quenched with water and then EtOAc was added, and the mixture was stirred rapidly at room temperature. The reaction mixture was filtered through a celite bed and washed with EtOAc. The layers were separated and the aqueous layer was extracted with EtOAc. The combined organic layers were dried over Na₂SO₄ and concentrated under reduced vacuum to afford 2-methyl-N-(pyridin-4-ylmethylene)propane-2-sulfinamide as pale yellow liquid (9.0 g, crude). LC-MS (ES) m/z=211.1 [M+H]⁺. ¹H NMR (400 MHz, DMSO-d6) δ ppm: 1.18 (s, 9H), 7.84 (d, J=5.2 Hz, 2H), 8.59 (s, 1H), 8.76 (d, J=8.2 Hz, 2H)

### Step 2

To a stirred solution of LDA (2M solution in THF) (45 mL, 2.1 equiv) in MTBE (100 mL) was added ethyl acetate (8.5 mL, 88.70 mmol, 2.0 equiv) in MTBE (100 mL) dropwise over a period of 30 min at −78° C. under nitrogen atmosphere. The Reaction mixture was stirred for 1 h at −78° C. 2-methyl-N-(pyridin-4-ylmethylene)propane-2-sulfinamide (9.0 g, 42.80 mmol, 1 equiv) in MTBE (100 mL) was added to the reaction mixture dropwise over a period of 30 min at −78° C. The reaction mixture was stirred for 3 h at −78° C. The reaction mixture was quenched with NH₄Cl solution at −78° C., warmed to room temperature and extracted with EtOAc. The combined organic layers were dried over Na₂SO₄ and concentrated under reduced vacuum to afford ethyl 3-((tert-butylsulfinyl)amino)-3-(pyridin-4-yl)propanoate as pale red liquid (9.0 g, crude). LC-MS (ES) m/z=299.1 [M+H]⁺.

### Step 3

Run 1; To a stirred solution of ethyl 3-((tert-butylsulfinyl)amino)-3-(pyridin-4-yl)propanoate (2.0 g, 6.71 mmol, 1.0 equiv), in 1,4-Dioxane (10 mL) was added 20% HCl in Dioxane (15 mL) at 0° C. and the reaction mixture was stirred at room temperature for 8 h. After consumption of the starting material the reaction mixture was concentrated and basified with aq. NaHCO₃ solution to afford ethyl 3-amino-3-(pyridin-4-yl)propanoate. The reaction mixture was proceeded for next step without purification. LC-MS (ES) m/z=195.1 [M+H]⁺.

Run 2; To a stirred solution of ethyl 3-((tert-butylsulfinyl)amino)-3-(pyridin-4-yl)propanoate (4.0 g, 13.42 mmol, 1.0 equiv), in 1,4-Dioxane (15 mL) was added 20% HCl in Dioxane (20 mL) at 0° C. and the reaction mixture was stirred at room temperature for 8 h. After consumption of the starting material the reaction mixture was concentrated and basified with aq. NaHCO₃ solution to afford ethyl 3-amino-3-(pyridin-4-yl)propanoate. The reaction mixture was proceeded for next step without purification. LC-MS (ES) m/z=195.1 [M+H]⁺.

### Step 4

Run 1: To a stirred solution of ethyl 3-amino-3-(pyridin-4-yl)propanoate (1.0 g, 5.15 mmol, 1 equiv) in dioxane (10 mL) and sat.NaHCO₃ solution (10 mL) was added Di-tert-butyl dicarbonate (1.7 mL, 7.73 mmol, 1.5 equiv) at room temperature. The reaction mixture was stirred at room temperature for 16 h. After consumption of the starting material, the reaction mixture was diluted with water and extracted with EtOAc. The combined organic layers were dried over Na₂SO₄ and concentrated under reduced vacuum to afford ethyl 3-((tert-butoxycarbonyl)amino)-3-(pyridin-4-yl)propanoate as pale brown liquid (0.8 g, crude). LC-MS (ES) m/z=295.1 [M+H]⁺.

Run 2: To a stirred solution of ethyl 3-amino-3-(pyridin-4-yl)propanoate (3.0 g, 15.46 mmol, 1 equiv) in dioxane (30 mL) and sat.NaHCO₃ solution (30 mL) was added Di-tert-butyl dicarbonate (5.3 mL, 23.19 mmol, 1.5 equiv) at room temperature. The reaction mixture was stirred at room temperature for 16 h. After consumption of the starting material, the reaction mixture was diluted with water and extracted with EtOAc. The combined organic layers were dried over Na₂SO₄ and concentrated under reduced vacuum to afford ethyl 3-((tert-butoxycarbonyl)amino)-3-(pyridin-4-yl)propanoate as pale brown liquid (2.0 g, crude). LC-MS (ES) m/z=295.1 [M+H]⁺.

### Step 5

Run 1: To a stirred solution of ethyl 3-((tert-butoxycarbonyl)amino)-3-(pyridin-4-yl)propanoate (0.5 g, 1.70 mmol, 1.0 equiv) in MeOH:THF (1:1)(10 mL) was added lithium hydroxide (0.1 g, 2.22 mmol, 1.3 equiv) at room temperature. The resulted suspension was stirred overnight at room temperature. The reaction mixture was diluted with water, acidified to pH 7 using 2M citric acid solution and extracted with EtOAc. The combined organic layers were dried over Na₂SO₄ and concentrated under reduced vacuum to afford 3-((tert-butoxycarbonyl)amino)-3-(pyridin-4-yl)propanoic acid as pale yellow solid (0.3 g, crude). LC-MS (ES) m/z=267.1 [M+H]⁺.

Run 2: To a stirred solution of ethyl 3-((tert-butoxycarbonyl)amino)-3-(pyridin-4-yl)propanoate (2.0 g, 6.80 mmol, 1.0 equiv) in MeOH:THF (1:1)(30 mL) was added lithium hydroxide (0.4 g, 8.8 mmol, 1.3 equiv) at room temperature. The resulted suspension was stirred overnight at room temperature. The reaction mixture was diluted with water, acidified to pH 7 using 2M citric acid solution and extracted with EtOAc. The combined organic layers were dried over Na₂SO₄ and concentrated under reduced vacuum to afford 3-((tert-butoxycarbonyl)amino)-3-(pyridin-4-yl)propanoic acid as pale yellow solid (1.0 g, crude). LC-MS (ES) m/z=267.1 [M+H]⁺.

### Step 6

To a stirred solution of 3-((tert-butoxycarbonyl)amino)-3-(pyridin-4-yl)propanoic acid (1.0 g, 3.75 mmol, 1 equiv) in toluene (25 mL) was added TEA (1.3 mL, 9.39 mmol, 2.5 equiv) and DPPA (0.97 mL, 4.51 mmol, 1.2 equiv) at room temperature. The reaction mixture was stirred at 75° C. for 16 h. The reaction mixture was cooled to room temperature, quenched with water and extracted with EtOAc. The combine organic layers were dried over Na₂SO₄ and concentrated under reduced vacuum to afford tert-butyl 2-oxo-5-(pyridin-4-yl)imidazolidine-1-carboxylate as pale brown solid (0.8 g, crude). LC-MS (ES) m/z=264 [M+H]⁺.

### Step 7

To a stirred solution of tert-butyl 2-oxo-5-(pyridin-4-yl)imidazolidine-1-carboxylate (1.0 g, 3.87 mmol, 1.0 equiv), 1-bromo-2-fluoro-4-iodobenzene (1.14 g, 3.8 mmol, 1.0 equiv), and CsF (1.4 g, 9.5 mmol, 2.5 equiv) in EtOAc (30 mL) was added CuI (0.07 g, 0.38 mmol, 0.1 equiv) followed by DMEDA (0.08 mL, 0.76 mmol, 0.2 equiv). The reaction mixture was stirred at room temperature for 30 h. After consumption of the starting material the reaction mixture was filtered through Celite. The filtrate was washed with water, followed by brine. The organic phase was dried over Na₂SO₄, filtered and evaporated to obtain crude product. The crude product was purified by flash column chromatography using 25% EtOAc in Hexane as mobile phase to afford tert-butyl 3-(4-bromo-3-fluorophenyl)-2-oxo-5-(pyridin-4-yl)imidazolidine-1-carboxylate (1.1 g, 68%) as pale yellow solid. LC-MS (ES) m/z=336.8, 338.8 [M+H]⁺-56. ¹H NMR (400 MHz, DMSO-d6) δ ppm: 1.24 (s, 9H), 3.58-3.62 (m, 1H), 4.28 (t, J=8.2 Hz, 1H), 5.27-5.30 (m, 1H), 7.35-7.39 (m, 3H), 7.63-7.70 (m, 2H), 8.58 (s, 2H).

### Step 8

To a stirred solution of tert-butyl 3-(4-bromo-3-fluorophenyl)-2-oxo-5-(pyridin-4-yl)imidazolidine-1-carboxylate (1.1 g, 2.52 mmol, 1.0 equiv), in 1,4-Dioxane (10 mL) was added 20% HCl in Dioxane (15 mL) at 0° C. and the reaction mixture was stirred at room temperature for 8 h. The reaction mixture was concentrated and basified with aq. NaHCO₃ solution. The reaction mixture was extracted in EtOAc. The combined organic layer was dried over sodium sulphate and concentrated under reduced vacuum to afford 1-(4-bromo-3-fluorophenyl)-4-(pyridin-4-yl)imidazolidin-2-one as pale yellow solid (0.75 g, 89%). LC-MS (ES) m/z=336.1, 338.1 [M+H]⁺. ¹H NMR (400 MHz, DMSO-d6) δ ppm 3.59-3.63 (m, 1H), 4.30 (t, J=8.0 Hz, 1H), 4.90 (t, J=8.0 Hz, 1H), 7.28-7.30 (m, 1H), 7.39-7.40 (m, 2H), 7.57 (t, J=8.0 Hz, 1H), 7.64-7.70 (m, 1H), 7.93 (s, 1H), 8.57 (s, 2H)

### Step 9

To a stirred suspension of 1-(4-bromo-3-fluorophenyl)-4-(pyridin-4-yl)imidazolidin-2-one (0.75 g, 2.23 mmol, 1 equiv) in DMF (10 mL) was added 60% sodium hydride (0.1 g, 2.67 mmol, 1.2 equiv) at 0° C. under N₂ atmosphere. The reaction mixture was stirred for 20 min, methyl Iodide (0.16 mL, 2.67 mmol, 1.2 equiv) was added and the reaction mixture was stirred for 2 h at room temperature. After consumption of the starting material the reaction mixture was quenched with ice water and extracted with EtOAc. The combined organic layers were dried over Na₂SO₄ and concentrated under reduced vacuum to afford 1-(4-bromo-3-fluorophenyl)-3-methyl-4-(pyridin-4-yl)imidazolidin-2-one as pale brown solid (0.5 g, 68%). LCMS (ES) m/z=350.1, 352.1 [M+H]⁺. ¹H NMR (400 MHz, DMSO-d6) δ ppm: 2.48 (s, 3H), 3.53-3.57 (m, 1H), 4.23 (t, J=8.1 Hz, 1H), 4.76 (t, J=8.0 Hz, 1H), 7.31-7.33 (m, 1H), 7.38-7.40 (m, 2H), 7.59 (t, J=8.2 Hz, 1H), 7.68-7.71 (m, 1H), 8.60 (s, 2H).

### Step 10

Run 1; To a stirred solution of 1-(4-bromo-3-fluorophenyl)-3-methyl-4-(pyridin-4-yl)imidazolidin-2-one (0.25 g, 1.57 mmol, 1 equiv) in 1,4-dioxane (20 mL) was added bis(pinacolato)diboron (0.3 g, 2.35 mmol, 1.5 equiv), and potassium acetate (0.23 g, 4.71 mmol, 3 equiv). The reaction mixture was degassed with N₂ for 10 minutes. PdCl₂(dppf).DCM adduct (0.1 g, 0.23 mmol, 0.15 equiv) was added and degassed with N₂ for further 10 minutes. The reaction mixture was stirred for 16 hours at 100° C. The reaction mixture was filtered over celite and the filtrate was concentrated to obtain the crude product. The crude product was purified by silica gel flash column chromatography. The compound eluted out in 15-18% EtOAc:Hexanes. The pure fractions were evaporated to obtain 1-(3-fluoro-4-(4,4,5,5-tetramethyl-1,3,2-dioxaborolan-2-yl)phenyl)-3-methyl-4-(pyridin-4-yl)imidazolidin-2-one (0.1 g, crude) as a pale brown solid. LCMS (ES) m/z=398.2 [M+H]⁺. ¹H NMR (400 MHz, DMSO-d6) c ppm: 1.05 (s, 12H), 3.28 (s, 3H), 3.55 (t, J=8.2 Hz, 1H), 4.26 (t, J=8.2 Hz, 1H), 4.77 (t, J=8.0 Hz, 1H), 7.31 (d, J=8.0 Hz, 1H), 7.38-7.40 (m, 2H), 7.48-7.61 (m, 2H), 8.61 (s, 2H)

Run 2; To a stirred solution of 1-(4-bromo-3-fluorophenyl)-3-methyl-4-(pyridin-4-yl)imidazolidin-2-one (0.25 g, 1.57 mmol, 1 equiv) in 1,4-dioxane (20 mL) was added bis(pinacolato)diboron (0.3 g, 2.35 mmol, 1.5 equiv), and potassium acetate (0.23 g, 4.71 mmol, 3 equiv). The reaction mixture was degassed with N₂ for 10 minutes. PdCl₂(dppf).DCM adduct (0.1 g, 0.23 mmol, 0.15 equiv) was added and degassed with N₂ for further 10 minutes. The reaction mixture was stirred for 16 hours at 100° C. The reaction mixture was filtered over celite and the filtrate was concentrated to obtain the crude product. The crude product was purified by silica gel flash column chromatography.

The compound eluted out in 15-18% EtOAc:Hexanes. The pure fractions were evaporated to obtain 1-(3-fluoro-4-(4,4,5,5-tetramethyl-1,3,2-dioxaborolan-2-yl)phenyl)-3-methyl-4-(pyridin-4-yl)imidazolidin-2-one (0.1 g, crude) as a pale brown solid. LCMS (ES) m/z=398.2 [M+H]⁺. ¹H NMR (400 MHz, DMSO-d6) δ ppm: 1.05 (s, 12H), 3.15 (s, 3H), 3.55 (t, J=8.2 Hz, 1H), 4.24 (t, J=8.2 Hz, 1H), 4.77 (t, J=8.0 Hz, 1H), 7.31 (d, J=8.0 Hz, 1H), 7.38-7.40 (m, 2H), 7.48-7.56 (m, 2H), 8.60-8.61 (m, 2H)

### Step 11

To a stirred solution of 1-(3-fluoro-4-(4,4,5,5-tetramethyl-1,3,2-dioxaborolan-2-yl)phenyl)-3-methyl-4-(pyridin-4-yl)imidazolidin-2-one (0.1 g, 0.25 mmol, 1 equiv) in 1,4-dioxane:water (20 mL: 0.5 mL), was added 5-bromo-7-methyl-7H-pyrrolo[2,3-d]pyrimidin-4-amine (0.04 g, 0.18 mmol, 0.75 equiv) and potassium phosphate (0.1 g, 0.50 mmol, 2 equiv) at room temperature. The reaction mixture was degassed with N₂ for 10 minutes. Pd₂(dba)₃ (0.011 g, 0.012 mmol, 0.05 equiv) and tri-tert-butylphosphonium tetrafluoroborate (0.007 g, 0.025 mmol, 0.1 equiv) was added and the reaction mixture was further degassed for 10 minutes. The reaction mixture was heated at 100° C. for 2 h. The reaction mixture was cooled & filtered through celite and the filtrate was concentrated to obtain crude compound, which was purified by flash silica gel column chromatography. The compound was eluted at 4% MeOH in DCM. The pure fractions were evaporated to obtain 1-(4-(4-amino-7-methyl-7H-pyrrolo[2,3-d]pyrimidin-5-yl)-3-fluorophenyl)-3-methyl-4-(pyridin-4-yl)imidazolidin-2-one (4.0 mg, 3.8%) as off white solid. LCMS (ES) m/z=418.1 [M+H]⁺. ¹H NMR (400 MHz, DMSO-d6) δ ppm: 2.64 (s, 3H), 3.60 (t, J=8.0 Hz, 1H), 3.72 (s, 3H), 4.29 (t, J=8.0 Hz, 1H), 4.76-4.80 (m, 1H), 5.93 (br.s, 2H), 7.24 (s, 1H), 7.33 (t, J=8.2 Hz, 1H), 7.39-7.41 (m, 3H), 7.68 (d, J=12.0 Hz, 1H), 8.12 (s, 1H), 8.61 (d, J=4.0 Hz, 2H). HPLC: 99.67% purity @260 nm.

### Example 115

### 1-(4-(4-amino-7-methyl-7H-pyrrolo[2,3-d]pyrimidin-5-yl)-3-fluorophenyl)-4-(3,4-difluorophenyl)-3-methylimidazolidin-2-one (enantiomer 1)

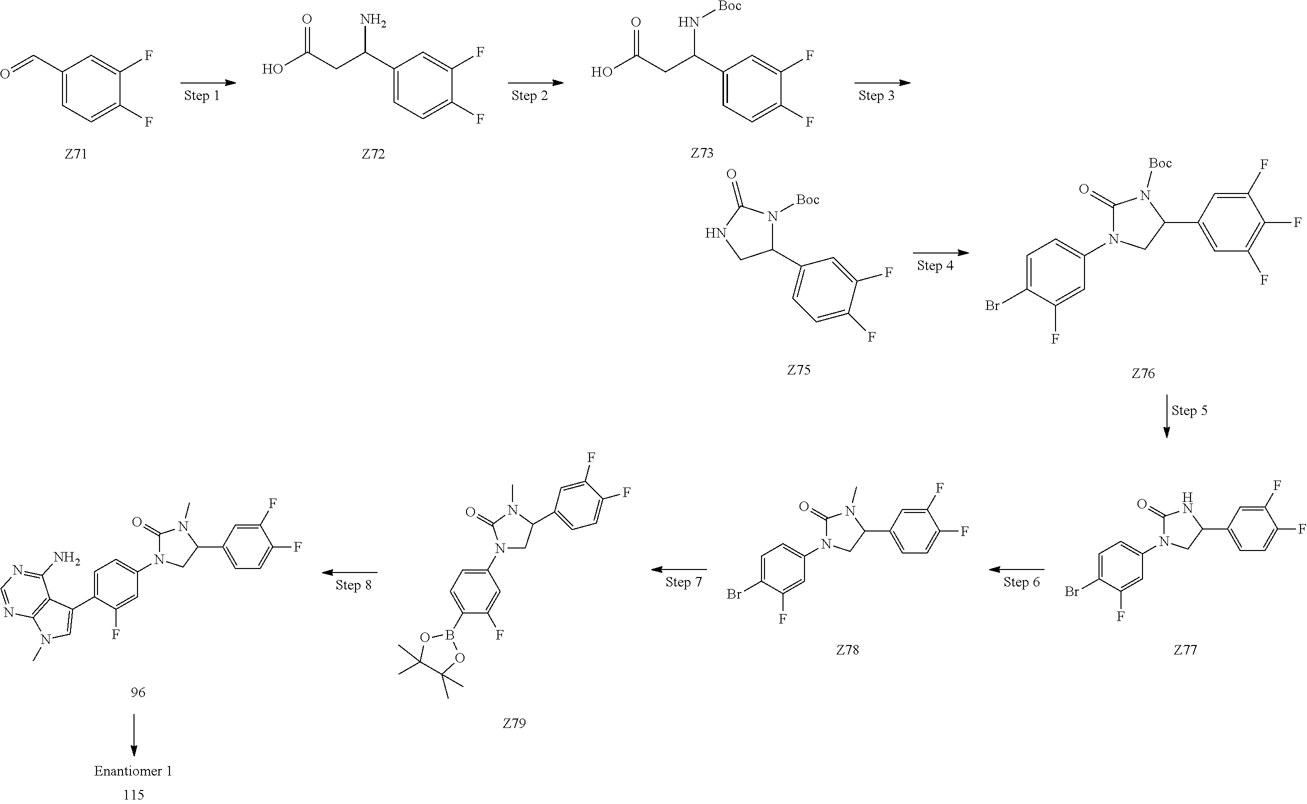

### Step 1

To a stirred solution of 3,4-Difluoro-benzaldehyde (25 g, 176.0 mmol, 1 equiv) in ethanol (250 mL) was added ammonium acetate (27.1 g, 352.0 mmol, 2.0 equiv) followed by malonic acid (21.9 g, 211.0 mmol, 1.2 equiv) at room temperature. The reaction mixture was stirred at 80° C. overnight. The reaction mixture was cooled to room temperature and the solids formed were filtered and washed with ethanol, dried under vacuum to afford 3-Amino-3-(3,4-difluoro-phenyl)-propionic acid as white solid (29.0 g, crude). LC-MS (ES) m/z=202.1 [M+H]⁺. ¹H NMR (400 MHz, DMSO-d6) δ ppm: 2.36 (d, J=8.0 Hz, 2H), 3.39-3.45 (m, 1H), 4.82-6.82 (br, 3H), 7.23 (br.s, 1H), 7.36 (q, J=8.0 Hz, 1H), 7.47 (t, J=8.2 Hz, 1H)

### Step 2

To a stirred solution of 3-Amino-3-(3,4-difluoro-phenyl)-propionic (29.0 g, 144.0 mmol, 1 equiv) in methanol (300 mL) was added triethylamine (40.0 mL, 288.0 mmol, 2.0 equiv) at room temperature. Boc₂O (50 mL, 216.0 mmol, 1.5 equiv) was added at room temperature. The reaction mixture was stirred at room temperature overnight. After consumption of the starting material, the reaction mixture was concentrated to remove the solvents. The resulted gummy mixture was triturated with n-Hexane to remove the excess Boc anhydride. The residue was dried under reduced vacuum to afford 3-tert-Butoxycarbonylamino-3-(3,4-difluoro-phenyl)-propionic acid as white solid (35.0 g, crude). LC-MS (ES) m/z=246.1 [M+H]⁺-56. ¹H NMR (400 MHz, DMSO-d6) δ ppm: 1.34 (s, 9H), 2.54-2.60 (m, 2H), 4.82-4.84 (m, 1H), 7.12 (br.s, 1H), 7.29-7.37 (m, 2H), 7.43-7.53 (m, 1H).

### Step 3

To a stirred solution of 3-tert-Butoxycarbonylamino-3-(3,4-difluoro-phenyl)-propionic acid (20.0 g, 66.4 mmol, 1 equiv) in toluene (250 mL) was added triethylamine (23.0 mL, 166.1 mmol, 2.5 equiv) at room temperature. DPPA (17.2 mL, 79.1 mmol, 1.2 equiv) was added at room temperature. The reaction mixture was stirred at 75° C. overnight. The reaction mixture was cooled to room temperature, quenched with water and extracted with EtOAc. The combined organic layers was dried over Na₂SO₄ and concentrated under reduced vacuum to afford 5-(3,4-Difluoro-phenyl)-2-oxo-imidazolidine-1-carboxylic acid tert-butyl ester as pale brown solid (15.0 g, 75%). LC-MS (ES) m/z=243.1 [M+H]⁺-56. ¹H NMR (400 MHz, DMSO-d6) δ ppm: 1.22 (s, 9H), 2.95-2.99 (m, 1H), 3.69 (t, J=8.2 Hz, 1H), 5.12-5.15 (m, 1H), 7.09-7.16 (m, 1H), 7.29-7.33 (m, 1H), 7.39-7.48 (m, 2H)

### Step 4

To a stirred solution of 5-(3,4-Difluoro-phenyl)-2-oxo-imidazolidine-1-carboxylic acid tert-butyl ester (15.0 g, 50.5 mmol, 1.0 equiv), 1-bromo-2-fluoro-4-iodobenzene (16.6 g, 55.3 mmol, 1.1 equiv), and CsF (19.0 g, 125.8 mmol, 2.5 equiv) in EtOAc (300 mL) was added CuI (1.0 g, 5.03 mmol, 0.1 equiv) followed by DMEDA (1.1 mL, 10.06 mmol, 0.2 equiv) at room temperature. The reaction mixture was stirred at room temperature overnight. The reaction mixture was quenched with water and extracted with EtOAc. The combined organic layers were dried over Na₂SO₄ and concentrated under reduced vacuum to afford 3-(4-Bromo-3-fluoro-phenyl)-5-(3,4-difluoro-phenyl)-2-oxo-imidazolidine-1-carboxylic acid tert-butyl ester as a pale yellow solid (15.0 g, 55%). LC-MS (ES) m/z=415.1, 417.1 [M+H]⁺-56. ¹H NMR (400 MHz, DMSO-d6) δ ppm: 1.25 (s, 9H), 3.59-3.63 (m, 1H), 4.25 (t, J=8.0 Hz, 1H), 5.25-5.28 (m, 1H), 7.19-7.21 (m, 1H), 7.34-7.37 (m, 1H), 7.41-7.52 (m, 2H), 7.63-7.71 (m, 2H)

### Step 5

To a stirred solution of 3-(4-Bromo-3-fluoro-phenyl)-5-(3,4-difluoro-phenyl)-2-oxo-imidazolidine-1-carboxylic acid tert-butyl ester (13.0 g, 27.6 mmol, 1.0 equiv), in 1,4-Dioxane (50 mL) was added 4M HCl in Dioxane (150 mL) at 0° C. and the reaction mixture was stirred at room temperature overnight. The reaction mixture was concentrated completely to remove the solvents. The solids formed were triturated with n-Pentane and filtered, dried under reduced vacuum to afford 1-(4-Bromo-3-fluoro-phenyl)-4-(3,4-difluoro-phenyl)-imidazolidin-2-one as pale yellow solid (10.0 g, 95%). LC-MS (ES) m/z=371.0, 373.0 [M+H]⁺. ¹H NMR (400 MHz, DMSO-d6) δ ppm: 3.58-3.62 (m, 1H), 4.24 (t, J=9.2 Hz, 1H), 4.89 (t, J=8.0 Hz, 1H), 7.27-7.29 (m, 2H), 7.41-7.49 (m, 2H), 7.57 (t, J=8.4 Hz, 1H), 7.66-7.70 (m, 1H), 7.86 (s, 1H).

### Step 6

To a stirred suspension of 1-(4-Bromo-3-fluoro-phenyl)-4-(3,4-difluoro-phenyl)-imidazolidin-2-one (11.0 g, 29.64 mmol, 1 equiv) in DMF (150 mL) was added 60% sodium hydride (1.4 g, 35.5 mmol, 1.2 equiv) at 0° C. under a N₂ atmosphere. The reaction mixture was stirred for 15 minutes. Methyl Iodide (2.2 mL, 35.5 mmol, 1.2 equiv) was added at 0° C. and the reaction mixture was stirred for 2 h at room temperature. After consumption of the starting material the reaction mixture was quenched with ice water and extracted with EtOAc. The combined organic layers were dried over Na₂SO₄ and concentrated under reduced vacuum to afford 1-(4-Bromo-3-fluoro-phenyl)-4-(3,4-difluoro-phenyl)-3-methyl-imidazolidin-2-one as pale yellow solid (11.0 g, 95%). LCMS (ES) m/z=385.1, 387.1 [M+H]⁺. ¹H NMR (400 MHz, DMSO-d6) δ ppm: 2.57 (s, 3H), 3.57 (t, J=8.4 Hz, 1H), 4.19 (t, J=9.6 Hz, 1H), 4.73 (t, J=8.4 Hz, 1H), 7.25 (br.s, 1H), 7.31-7.33 (m, 1H), 7.44-7.53 (m, 2H), 7.59 (t, J=8.4 Hz, 1H), 7.70 (dd, J=2.4, 12.0 Hz, 1H)

### Step 7

To a stirred solution of 1-(4-Bromo-3-fluoro-phenyl)-4-(3,4-difluoro-phenyl)-3-methyl-imidazolidin-2-one (5.0 g, 20.7 mmol, 1 equiv) in 1,4-dioxane (150 mL) was added bis(pinacolato)diboron (5.0 g, 31.16 mmol, 1.5 equiv), and potassium acetate (3.8 g, 62.33 mmol, 3 equiv). The reaction mixture was degassed with argon for 10 minutes. PdCl₂(dppf). DCM adduct (1.0 g, 2.07 mmol, 0.1 equiv) was added and the mixture was degassed with argon for a further 10 minutes. The reaction mixture was stirred for 24 h at 100° C. The reaction mixture was filtered through celite and washed with EtOAc. The filtrate was concentrated to obtain the crude product. The crude product was purified with silica gel flash column chromatography. The compound eluted out in 15% EtOAc:Hexanes. The pure fractions were evaporated to obtain 4-(3,4-Difluoro-phenyl)-1-[3-fluoro-4-(4,4,5,5-tetramethyl-[1,3,2]dioxaborolan-2-yl)-phenyl]-3-methyl-imidazolidin-2-one (3.4 g, 60%) as off white solid. LCMS (ES) m/z=433.1 [M+H]⁺. ¹H NMR (400 MHz, DMSO-d6) δ ppm: 1.14 (s, 12H), 2.58 (s, 3H), 3.57 (m, 1H), 4.21 (t, J=9.2 Hz, 1H), 4.73 (t, J=8.0 Hz, 1H), 7.29-7.33 (m, 1H), 7.44-7.56 (m, 4H), 7.88 (s, 1H)

### Step 8

To a stirred solution of 4-(3,4-Difluoro-phenyl)-1-[3-fluoro-4-(4,4,5,5-tetramethyl-[1,3,2]dioxaborolan-2-yl)-phenyl]-3-methyl-imidazolidin-2-one (1.7 g, 3.93 mmol, 1 equiv) in 1,4-dioxane:water (50 mL: 1.0 mL), was added 5-bromo-7-methyl-7H-pyrrolo[2,3-d]pyrimidin-4-amine (0.7 g, 3.14 mmol, 0.8 equiv) and tripotassium phosphate (1.7 g, 7.87 mmol, 2 equiv) at room temperature. The reaction mixture was degassed with argon for minutes. Pd₂(dba)₃ (0.18 g, 1.96 mmol, 0.05 equiv) and tri-tert-butylphosphoniumtetrafluoroborate (0.11 g, 0.39 mmol, 0.1 equiv) was added and the reaction mixture was further degassed for 10 minutes. The reaction mixture was heated at 100° C. for 4 h. The reaction mixture was cooled & filtered through celite and the filtrate was concentrated to obtain crude compound. Crude compound was purified by flash column chromatography using a silica gel column, and the compound was eluted with 3% MeOH:DCM, the pure fractions were evaporated to obtain 1-[4-(4-Amino-7-methyl-7H-pyrrolo[2,3-d]pyrimidin-5-yl)-3-fluoro-phenyl]-4-(3,4-difluoro-phenyl)-3-methyl-imidazolidin-2-one (0.7 g, 40%) as off white solid. LCMS (ES) m/z=453.4 [M+H]⁺.]⁺. ¹H NMR (400 MHz, DMSO-d6) δ ppm: 2.65 (s, 3H), 3.62 (m, 1H), 3.72 (s, 3H), 4.25 (t, J=9.4 Hz, 1H), 4.75 (t, J=8.2 Hz, 1H), 5.92 (br.s, 2H), 7.25-7.29 (m, 2H), 7.33 (t, J=8.6 Hz, 1H), 7.41 (d, J=8.4 Hz, 1H), 7.45-7.54 (m, 2H), 7.69 (d, J=13.2 Hz, 1H), 8.12 (s, 1H).

### Step 9: Enantiomer Separation

1.0 g of racemic 1-[4-(4-Amino-7-methyl-7H-pyrrolo[2,3-d]pyrimidin-5-yl)-3-fluoro-phenyl]-4-(3,4-difluoro-phenyl)-3-methyl-imidazolidin-2-one was separated to obtain pure enantiomers by chiral HPLC using preparative HPLC conditions: Column: CHIRALPAK IC (250 mm×20 mm×5 μm); Mobile phase: MTBE:Ethanol with 0.1% DEA (80:20); Flow rate: 15 mL/min. Pure fractions at retention time 13.50 min were concentrated to afford the enantiomer 1 as an off white solid (0.34 g, 34% yield). LCMS (ES) m/z=453.4 [M+H]⁺. ¹H NMR (400 MHz, DMSO-d6) δ ppm: 2.60 (s, 3H), 3.62 (t, J=8.4 Hz, 1H), 3.72 (s, 3H), 4.25 (t, J=9.2 Hz, 1H), 4.75 (t, J=8.2 Hz, 1H), 5.95 (br.s, 2H), 7.25-7.29 (m, 2H), 7.33 (t, J=8.6 Hz, 1H), 7.41 (d, J=8.0 Hz, 1H), 7.45-7.54 (m, 2H), 7.70 (d, J=13.2 Hz, 1H), 8.13 (s, 1H). Analytical HPLC Column: CHIRALPAK IC (250 mm×4.6 mm×5 mic), Mobile phase: MTBE:Ethanol with 0.1% DEA (80:20), Flow rate: 1.0 mL/min, Retention time: 9.086 min.

### Example 137

### 1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one (enantiomer 2)

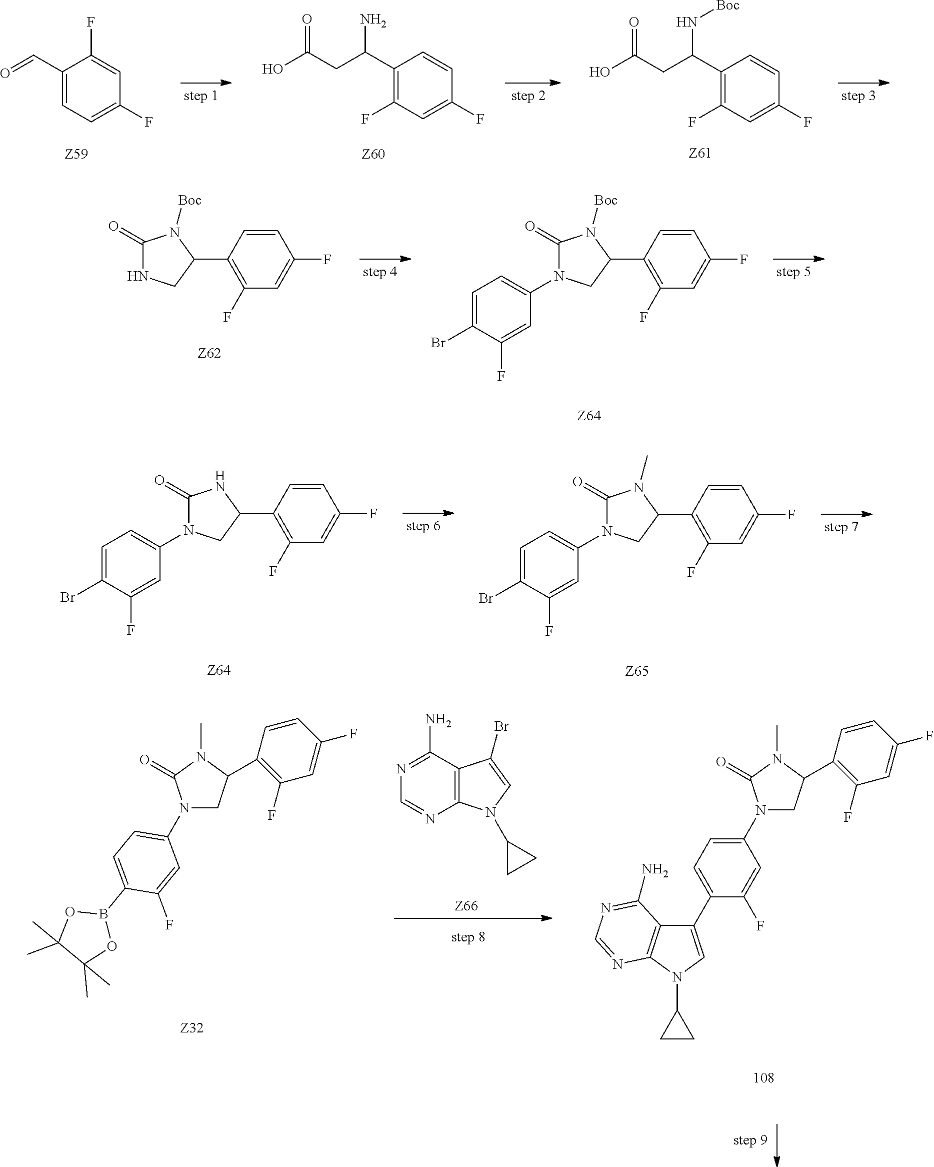

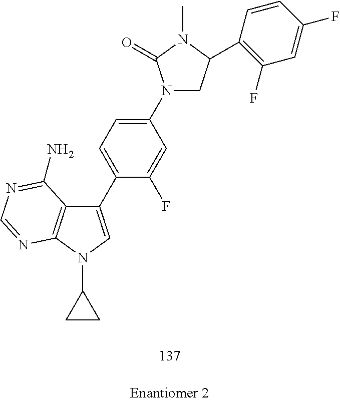 [SMILES: CN1C(=O)N(c2ccc(-c3cn(C4CC4)c4ncnc(N)c34)c(F)c2)CC1c1ccc(F)cc1F]

### Step 1

To a stirred solution of 2,4-difluorobenzaldehyde (10.0 g, 70.4 mmol, 1 equiv), malonic acid (8.78 g, 84.45 mmol, 1.2 equiv) and ammonium acetate (10.84 g, 140.8 mmol, 2 equiv) in EtOH (100 mL) was heated at 80° C. for 16 h. The solid was filtered off and washed with n-pentane to give the 3-amino-3-(2,4-difluorophenyl)propanoic acid as off white solid (11.5 g, crude). LC-MS (ES) m/z=202.1 [M+H]⁺

### Step 2

To a stirred solution of 3-amino-3-(2,4-difluorophenyl)propanoic acid (11.5 g, 57.2 mmol, 1 equiv) in dioxane (100 mL) was added sat.NaHCO₃ solution (100 mL) and then added Boc₂O (19.7 mL, 86 mmol, 1.5 equiv) at room temperature. The reaction mixture was stirred at room temperature for 16 h. The reaction mixture was washed with hexane and then the aqueous layer was acidified with citric acid solution, extracted with ethyl acetate, and then dried over Na₂SO₄ and concentrated to give the 3-((tert-butoxycarbonyl)amino)-3-(2,4-difluorophenyl)propanoic acid as white solid (11.5 g, crude). LC-MS (ES) m/z=246.1 [M+H]⁺—56.

### Step 3

To a stirred solution of 3-((tert-butoxycarbonyl)amino)-3-(2,4-difluorophenyl)propanoic acid (11.5 g, 38.17 mmol, 1 equiv) in toluene (130 mL) was added TEA (13.32 mL, 95.43 mmol, 2.5 equiv) and DPPA (9.92 mL, 45.8 mmol, 1.2 equiv) at room temperature, then the mixture was stirred for 30 min at room temperature, and then at 75° C. for 16 h. The reaction mixture was cooled, diluted with EtOAc and washed with water. The organic layer was dried over Na₂SO₄ and concentrated to obtain the crude product. Purification: Triturated the crude compound with ether and filtered and dried to give the tert-butyl 5-(2,4-difluorophenyl)-2-oxoimidazolidine-1-carboxylate as brown solid (7.3 g, 64.1%). LC-MS (ES) m/z=243.1 [M+H]⁺—56. ¹H NMR (400 MHz, DMSO-d6) δ ppm 1.28 (s, 9H), 2.98-3.02 (m, 1H), 3.74 (t, J=9.6 Hz, 1H), 5.30-5.34 (m, 1H), 7.08-7.13 (m, 1H), 7.23-7.33 (m, 2H), 7.44 (s, 1H).

### Step 4

To a stirred solution of tert-butyl 5-(2,4-difluorophenyl)-2-oxoimidazolidine-1-carboxylate (7.3 g, 24.5 mmol, 1.0 equiv), 1-bromo-2-fluoro-4-iodobenzene (8.84 g, 0.02937 mol, 1.2 equiv), DMEDA (0.26 mL, 2.45 mmol, 0.1 equiv), CsF (9.3 g, 61.25 mmol, 2.5 equiv), in EtOAc (100 mL) was added CuI (0.233 g, 1.225 mmol, 0.05 equiv), and the reaction mixture was stirred at room temperature for 20 h. After consumption of the starting material the reaction mixture was quenched with water and extracted with ethyl acetate. The organic layer was washed with brine and dried over Na₂SO₄ and concentrated. The crude product was purified by flash column chromatography using a silica gel column, and the compound was eluted at 16% EtOAc in Hexane as mobile phase to afford the desired product tert-butyl 3-(4-bromo-3-fluorophenyl)-5-(2,4-difluorophenyl)-2-oxoimidazolidine-1-carboxylate as a pale brown solid (5 g, crude). LC-MS (ES) m/z=371.0, 373.0 [M+H]⁺.

### Step 5

To a stirred solution of tert-butyl 3-(4-bromo-3-fluorophenyl)-5-(2,4-difluorophenyl)-2-oxoimidazolidine-1-carboxylate (5 g, 10.6157 mmol, 1.0 equiv), in 1,4-dioxane (10 mL) was added 4M HCl in dioxane (20 mL) at room temperature and the mixture was stirred overnight. Excess of dioxane was evaporated, and the crude compound was triturated with n-pentane and dried to give the 1-(4-bromo-3-fluorophenyl)-4-(2,4-difluorophenyl)imidazolidin-2-one as off white solid (3.3 g, 83%). LC-MS (ES) m/z=371.0, 373.0 [M+H]⁺. ¹H NMR (400 MHz, DMSO-d6) δ ppm 3.64-3.67 (m, 1H), 4.29 (t, J=9.6 Hz, 1H), 5.04-5.08 (m, 1H), 7.09-7.14 (m, 1H), 7.25-7.31 (m, 2H), 7.46-7.52 (m, 1H), 7.56 (t, J=8.4 Hz, 1H), 7.67-7.71 (m, 1H), 7.82 (s, 1H).

### Step 6

To a stirred solution of 1-(4-bromo-3-fluorophenyl)-4-(2,4-difluorophenyl)imidazolidin-2-one (1.4 g, 3.7735 mmol, 1 equiv) in DMF (10 mL) was added 60% NaH (0.22 g, 5.6603 mmol, 1.5 equiv) at 0° C., then the mixture was stirred for 15 minutes. Methyl Iodide (0.35 mL, 5.6603 mmol, 1.5 equiv) was added at 0° C. and the mixture was stirred at room temperature for 1.5 h. The reaction mixture was quenched with ice water, extracted with ethyl acetate, dried and concentrated to give the 1-(4-bromo-3-fluorophenyl)-4-(2,4-difluorophenyl)-3-methylimidazolidin-2-one as colourless liquid (1.5 g, crude). LCMS (ES) m/z=385.0, 387.0 [M+H]⁺. ¹H NMR (400 MHz, CDCl₃) δ ppm 2.78 (s, 3H), 3.53-3.57 (m, 1H), 4.17 (t, J=9.2 Hz, 1H), 4.91-4.95 (m, 1H), 6.86-6.97 (m, 2H), 7.13-7.16 (m, 1H), 7.27-7.33 (m, 1H), 7.44 (t, J=8.4 Hz, 1H), 7.54-7.57 (m, 1H).

### Step 7

A mixture of 1-(4-bromo-3-fluorophenyl)-4-(2,4-difluorophenyl)-3-methylimidazolidin-2-one (1.5 g, 3.8961 mmol), bis(pinacolato)diboron (1.48 g, 5.8441 mmol, 1.5 equiv), and potassium acetate (0.76 g, 7.7922 mmol, 2 equiv) in 1,4-dioxane (50 mL) in a sealed tube was bubbled with N₂ for 10 min, then PdCl₂(dppf)-CH₂Cl₂ adduct (0.158 g, 0.1948 mmol, 0.05 equiv). The reaction mixture was heated at 100° C. and stirred overnight. The reaction mixture was cooled to room temperature and concentrated under vacuum. The crude was purified by flash column chromatography with silica gel and 20-50% EtOAc:Hexanes mobile phase. The pure fractions were evaporated to obtain 4-(2,4-difluorophenyl)-1-(3-fluoro-4-(4,4,5,5-tetramethyl-1,3,2-dioxaborolan-2-yl)phenyl)-3-methylimidazolidin-2-one (1.5 g, 89%) as off-white solid. LCMS (ES) m/z=433.2 [M+H]⁺. ¹H NMR (400 MHz, DMSO-d6) δ ppm 1.26 (s, 12H), 2.61 (s, 3H), 3.62-3.66 (m, 1H), 4.25 (t, J=9.6 Hz, 1H), 4.96-5.00 (m, 1H), 7.14 (t, J=8.4 Hz, 1H), 7.29-7.33 (m, 2H), 7.42-7.49 (m, 2H), 7.52-7.55 (m, 1H).

### Step 8

A mixture of 4-(2,4-difluorophenyl)-1-(3-fluoro-4-(4,4,5,5-tetramethyl-1,3,2-dioxaborolan-2-yl)phenyl)-3-methylimidazolidin-2-one (0.277 g, 0.6422 mmol, 1.3 equiv), 5-bromo-7-cyclopropyl-7H-pyrrolo[2,3-d]pyrimidin-4-amine (0.125 g, 0.4940 mmol, 1 equiv), potassium phosphate (0.209 g, 0.9881 mmol, 2 equiv) and Pd₂(dba)₃ (0.022 g, 0.0247 mmol, 0.05 equiv) in 1,4-dioxane:water (8 mL: 2 mL) in a sealed tube was bubbled with N₂ for 10 minutes and then tri-tert-butylphosphonium tetrafluoroborate (0.0143 g, 0.0494 mmol, 0.1 equiv) was added, and the mixture was heated to 100° C. overnight. The mixture was filtered through celite, and the filtrate was concentrated in vacuo. The crude was purified by flash column chromatography on silica gel with 2% MeOH:DCM mobile phase, the pure fractions were evaporated to obtain 1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one as off white solid (0.14 g, 59%). LCMS (ES) m/z=479.2 [M+H]⁺. ¹H NMR (400 MHz, DMSO-d6) δ ppm 1.01 (s, 4H), 2.63 (s, 3H), 3.53-3.54 (m, 1H), 3.68 (t, J=9.2 Hz, 1H), 4.29 (t, J=9.6 Hz, 1H), 4.99 (t, J=9.6 Hz, 1H), 5.91 (br.s., 2H), 7.10-7.19 (m, 2H), 7.32 (t, J=8.8 Hz, 2H), 7.40-7.42 (m, 1H), 7.44-7.50 (m, 1H), 7.69 (d, J=13.6 Hz, 1H), 8.12 (s, 1H). HPLC: 99.68% purity @254 nm.

### Step 9: Enantiomer Separation

0.1 g of racemic compound 1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one was separated to isolate enantiomer 2 by chiral HPLC using preparative HPLC conditions: Column: CHIRALPAK IC (250 mm×20 mm×5 mic); Mobile phase: n-hexane:Ethanol with 0.1% TFA (50:50); Flow rate: 18 mL/min. Pure fractions at retention time 28.06 min were concentrated. The residue obtained was diluted with DCM, washed with saturated NaHCO₃. The organic layer was dried and concentrated to afford the enantiomer 2 as off white solid (0.028 g, 56% yield). LCMS (ES) m/z=479.1 [M+H]*. ¹H NMR (400 MHz, DMSO-d6) δ ppm 0.95-1.05 (m, 4H), 2.63 (s, 3H), 3.50-3.60 (m, 1H), 3.68 (t, J=9.2 Hz, 1H), 4.29 (t, J=9.6 Hz, 1H), 5.00 (t, J=6.4 Hz, 1H), 5.91 (br. s., 2H), 7.13-7.20 (m, 2H), 7.30-7.37 (m, 2H), 7.40-7.43 (m, 1H), 7.44-7.50 (m, 1H), 7.69 (d, J=12.8 Hz, 1H), 8.12 (s, 1H). 99.56% purity by chiral HPLC. Analytical Column: CHIRALPAK IC (250 mm×4.6 mm×5 mic), Mobile phase (A:B): n-Hexane:Ethanol with 0.1% TFA:(50:50), Flow rate: 1.0 ml/min, Retention time: 28.065 min.

### Synthesis of Intermediate Z66

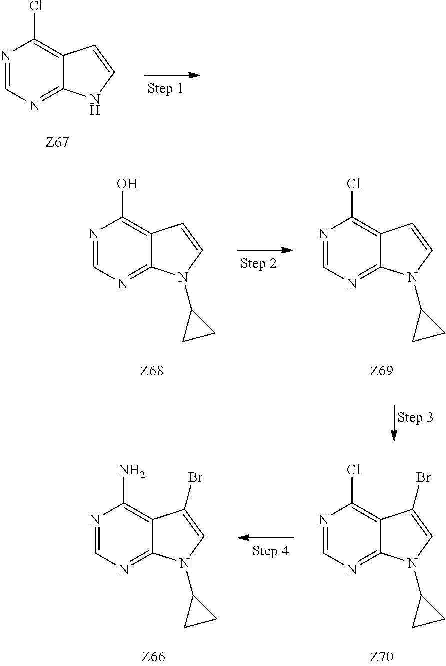 [SMILES: Clc1ncnc2[nH]ccc12 ; Oc1ncnc2c1ccn2C1CC1 ; Clc1ncnc2c1ccn2C1CC1 ; Nc1ncnc2c1c(Br)cn2C1CC1 ; Clc1ncnc2c1c(Br)cn2C1CC1]

### Step 1

To a stirred solution of 4-chloro-7H-pyrrolo[2,3-d]pyrimidine (5 g, 32.6797 mmol) in pyridine (50 mL) was added cyclopropylboronic acid (7 g, 84.6993 mmol, 2.5 equiv) and Cu(OAc)₂ (8.8 g, 49.0196 mmol, 1.5 equiv) at room temperature, then heated at 90° C. for overnight. The excess of pyridine was removed by concentration. The crude product was purified by flash column chromatography with silica gel and 50% EtOAc:Hexanes mobile phase. The fractions were evaporated to obtain 7-cyclopropyl-7H-pyrrolo[2,3-d]pyrimidin-4-ol as brown colour solid (4 g, crude). LCMS (ES) m/z=176.2 [M+H]⁺.

### Step 2

To a stirred solution of 7-cyclopropyl-7H-pyrrolo[2,3-d]pyrimidin-4-ol (4 g, 22.8571 mmol) in POCl₃ (40 mL) was refluxed for 6 h, then cooled to room temperature and concentrated excess of POCl₃. Ice was added and extracted with DCM (100 mL×3). The organics were combined and dried over Na₂SO₄. The organic layer was concentrated to get crude product. The crude product was purified by flash column chromatography with silica gel and 30% EtOAc:Hexanes mobile phase. The pure fractions were evaporated to obtain 4-chloro-7-cyclopropyl-7H-pyrrolo[2,3-d]pyrimidine as an off white solid (0.4 g, 9% yield). LCMS (ES) m/z=194.2 [M+H]⁺. ¹H NMR (400 MHz, CDCl₃) δ ppm 1.10-1.06 (m, 2H), 1.16-1.19 (m, 2H), 3.50-3.55 (m, 1H), 6.54 (d, J=3.2 Hz, 1H), 7.22 (d, J=4 Hz, 1H), 8.67 (S, 1H).

### Step 3

To a stirred solution of 4-chloro-7-cyclopropyl-7H-pyrrolo[2,3-d]pyrimidine (0.4 g, 2.0618 mmol) in dichloromethane (10 mL) was added N-bromosuccinimide (0.55 g, 3.0927 mmol) at 0° C., and the reaction was stirred for 2 h. The reaction mixture was diluted with dichloromethane, washed with water and brine solution. The organic layer was dried over Na₂SO₄ and concentrated to get crude product. The crude was purified by flash column chromatography with silica gel and 20% EtOAc:Hexanes mobile phase. The pure fractions were evaporated to obtain 5-bromo-4-chloro-7-cyclopropyl-7H-pyrrolo[2,3-d]pyrimidine as off white solid (0.47 g, 83% yield). LCMS (ES) m/z=272.0, 274.0 [M+H]⁺. ¹H NMR (400 MHz, CDCl₃) δ ppm 1.02-1.09 (m, 2H), 1.16-1.21 (m, 2H), 3.48-3.54 (m, 1H), 7.27 (s, 1H), 8.66 (S, 1H).

### Step 4

To a stirred solution of 5-bromo-4-chloro-7-cyclopropyl-7H-pyrrolo[2,3-d]pyrimidine (0.47 g, 1.7279 mmol) in aqueous NH₄OH (10 mL) and 1,4-dioxane (5 mL) was heated in a stainless steel autoclave at 100° C. for overnight. After overnight the reaction mixture was cooled to room temperature, filtered and dried to give 5-bromo-7-cyclopropyl-7H-pyrrolo[2,3-d]pyrimidin-4-amine as off white solid (0.25 g, 57% yield). LCMS (ES) m/z=253.0, 255.0 [M+H]⁺. ¹H NMR (400 MHz, DMSO-d6) δ ppm 0.97 (s, 4H), 3.45-3.50 (m, 1H), 6.65 (br.s, 2H), 7.32 (s, 1H), 8.08 (s, 1H).

### Example 175, 176 and 177

### 1-(4-(4-amino-7-cyclopropyl-7H-pvrrolo[2,3-d]pyrimidin-5-yl)-3-fluorophenyl)-4-(3,5-difluorophenyl)-3-methylimidazolidin-2-one and enantiomers

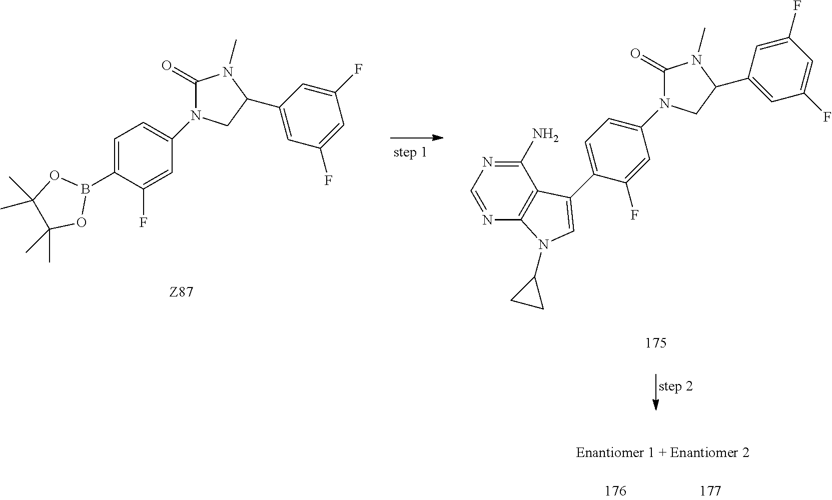 [SMILES: CN1C(=O)N(c2ccc(B3OC(C)(C)C(C)(C)O3)c(F)c2)CC1c1cc(F)cc(F)c1 ; CN1C(=O)N(c2ccc(-c3cn(C4CC4)c4ncnc(N)c34)c(F)c2)CC1c1cc(F)cc(F)c1]

### Step 1

A stirred solution of 4-(3,5-difluorophenyl)-1-(3-fluoro-4-(4,4,5,5-tetramethyl-1,3,2-dioxaborolan-2-yl)phenyl)-3-methylimidazolidin-2-one (1.8 g, 4.16 mmol, 1 equiv), 5-bromo-7-cyclopropyl-7H-pyrrolo[2,3-d]pyrimidin-4-amine (0.945 g, 3.74 mmol, 0.9 equiv) and potassium phosphate (1.76 g, 8.32 mmol, 2 equiv) in 1,4-dioxane:water (40 mL: 10 mL), was degassed with N₂ for 15 minutes. Pd₂(dba)₃ (0.19 g, 0.208 mmol, 0.05 equiv) and Tri-tert-butylphosphonium tetrafluoroborate (0.12 g, 0.416 mmol, 0.1 equiv) were added and the reaction mixture was heated to 100° C. and stirred for 3 h. The reaction mixture was cooled & filtered through celite and the layers were separated. The aqueous layer was extracted with EtOAc. The combined organics was dried over sodium sulphate, filtered and concentrated to obtain the crude compound, which was purified by flash column chromatography using a silica gel column, and the compound was eluted at 3.5% MeOH:DCM. The pure fractions were evaporated to obtain 1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one (1.0 g, 50%) as off white solid. LCMS (ES) m/z=479.0 [M+H]⁺. ¹H NMR (400 MHz, DMSO-d6) δ ppm 0.99-1.01 (m, 4H), 2.63 (s, 3H), 3.54 (s, 1H), 3.61-3.65 (m, 1H), 4.25 (t, J=9.6 Hz, 1H), 4.78 (t, J=7.8 Hz, 1H), 5.90 (br. s., 2 H), 7.15-7.17 (m, 3H), 7.24 (t, J=9.0 Hz, 1H), 7.34 (d, J=8.4 Hz, 1H), 7.39 (d, J=7.6 Hz, 1H), 7.68 (d, J=12.8 Hz, 1H), 8.12 (s, 1H); HPLC purity: 99.02%

### Step 2: Enantiomer Separation

0.9 g of racemic compound 1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one was separated to enantiomers 1 and 2 by chiral HPLC. Preparative HPLC conditions: Column: CHIRALPAK IC (250 mm×20 mm×5 μm); Mobile phase: MTBE:Ethanol with 0.1% DEA (80:20); Flow rate: 15 mL/min. Pure fractions at retention time 19.857 min were concentrated to obtain enantiomer 1 as Off white solid (0.29 g, 64% yield). LCMS (ES) m/z=479.2 [M+H]⁺. ¹H NMR (400 MHz, DMSO-d6) δ ppm 0.99-1.01 (m, 4H), 2.63 (s, 3H), 3.53-3.55 (m, 1H), 3.61-3.65 (m, 1H), 4.25 (t, J=9.6 Hz, 1H), 4.78 (t, J=7.8 Hz, 1H), 5.90 (br. s., 2H), 7.15-7.17 (m, 3H), 7.24 (t, J=9.0 Hz, 1H), 7.33 (t, J=8.6 Hz, 1H), 7.40 (d, J=6.4 Hz, 1H), 7.68 (d, J=13.2 Hz, 1H), 8.13 (s, 1H); Chiral HPLC purity: 99.89% Pure fractions at retention time 29.132 min were concentrated to obtain enantiomer 2 as off white solid (0.28 g, 62% yield). LCMS (ES) m/z=479.2 [M+H]⁺. ¹H NMR (400 MHz, DMSO-d6) δ ppm 0.99-1.01 (m, 4H), 2.63 (s, 3H), 3.53-3.55 (m, 1H), 3.63 (t, J=8.4 Hz, 1H), 4.25 (t, J=9.6 Hz, 1H), 4.78 (t, J=8.2 Hz, 1H), 5.90 (br. s., 2H), 7.15-7.17 (m, 3H), 7.24 (t, J=9.0 Hz, 1H), 7.33 (t, J=8.6 Hz, 1H), 7.39 (d, J=8.8 Hz, 1H), 7.68 (d, J=12.8 Hz, 1H), 8.13 (s, 1H); Chiral HPLC purity: 99.06%

The Compounds 14 to 45, 47 to 58, 60 to 99, 101 to 109, 111 to 114, 116 to 136, 138 to 174 were prepared generally according to the above Schemes and the procedures described for Examples 1 to 13, 46, 59, 100, 110, 115, 137 and 175 to 177.

TABLE 1

| Com- | | | LCMS | ¹H-NMR (400 MHz, |
|---|---|---|---|---|
| pound | | | m/z | DMSO-d₆), Chiral HPLC |
| # | Structure | Name | [M + H]⁺) | data |
| 14 | 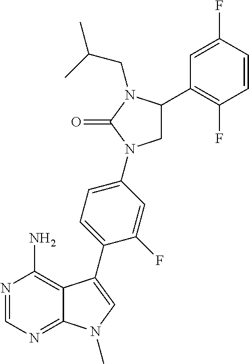 [SMILES: CC(C)CN1C(=O)N(c2ccc(-c3cn(C)c4ncnc(N)c34)c(F)c2)CC1c1cc(F)ccc1F] | 1-(4-(4-amino-7-methyl-7H- pyrrolo[2,3-d]pyrimidin-5-yl)-3- fluorophenyl)-4-(2,5-difluorophenyl)- 3-isobutylimidazolidin-2-one | 495.1 | 0.78-0.80 (m, 6H), 1.71-1.74 (m, 1H), 2.53-2.58 (m, 1H), 3.15-3.20 (m, 1H), 3.71 (s, 3H), 3.76-3.80 (m, 1H), 4.32 (t, J = 8.4 Hz, 1H), 5.05-5.09 (m, 1H), 5.93 (br. S., 2H), 7.24 (s, 1H), 7.27-7.36 (m, 4H) 7.41 (d, J = 8.4 Hz, 1H), 7.70 (d, J = 12.8 Hz, 1H), 8.11 (s, 1H). |
| 15 | 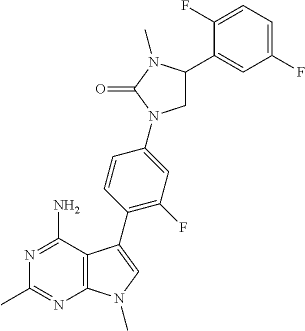 [SMILES: Cc1nc(N)c2c(-c3ccc(N4CC(c5cc(F)ccc5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(2,5-difluorophenyl)- 3-methylimidazolidin-2-one | 467.1 | 2.35 (s, 3H), 2.65 (s, 3H), 3.67 (s, 3H), 3.69-3.71 (m, 1H), 4.26-4.31 (m, 1H), 4.98-5.02 (m, 1H), 5.83 (br. S., 2H), 7.14 (s, 1H), 7.24-7.37 (m, 4H), 7.39-7.41 (m, 1H), 7.66-7.70 (m, 1H. |
| 16 | 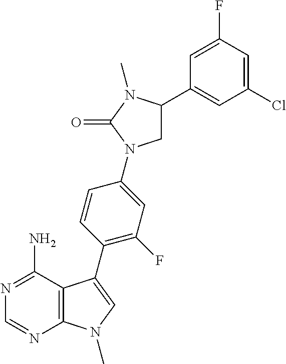 [SMILES: CN1C(=O)N(c2ccc(-c3cn(C)c4ncnc(N)c34)c(F)c2)CC1c1cc(F)cc(Cl)c1] | 1-(4-(4-amino-7-methyl-7H- pyrrolo[2,3-d]pyrimidin-5-yl)-3- fluorophenyl)-4-(3-chloro-5- fluorophenyl)-3-methylimidazolidin- 2-one | 469.1 | 2.62 (s, 3H), 3.64 (t, J = 8.4 Hz, 1H), 3.71 (s, 3H), 4.24 (t, J = 9.6 Hz, 1H), 4.78 (t, J = 8.0 Hz, 1H), 5.92 (br. S., 2H), 7.24 (s, 1H), 7.26-7.35 (m, 2H), 7.39-7.42 (m, 3H), 7.68 (d, J = 12.8 Hz, 1H), 8.19 (s, 1H). |
| 17 | 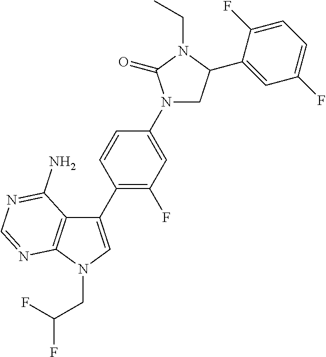 [SMILES: CCN1C(=O)N(c2ccc(-c3cn(CC(F)F)c4ncnc(N)c34)c(F)c2)CC1c1cc(F)ccc1F] | 1-(4-(4-amino-7-(2,2-difluoroethyl)- 7H-pyrrolo[2,3-d]pyrimidin-5-yl)-3- fluorophenyl)-4-(2,5-difluorophenyl)- 3-ethylimidazolidin-2-one | 517.1 | 0.97 (t, J = 7.2 Hz, 3H), 2.73-2.82 (m, 1H), 3.35-3.46 (m, 1H), 3.48-3.76 (m, 4H), 4.29 (t, J = 10.0 Hz, 1H), 4.59-4.66 (m, 2H), 5.10-5.14 (m, 1H), 6.04 (br. s., 2H), 6.40 (t, J = 55.2 Hz, 1H), 7.27-7.36 (m, 5H), 7.43 (d, J = 7.2 Hz, 1H), 7.71 (d, J = 12.8 Hz, 1H), 8.14 (s, 1H) |
| 18 | 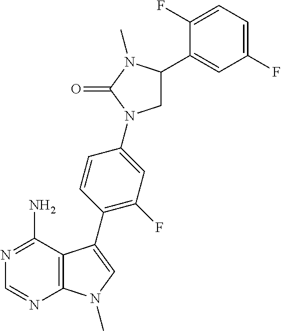 [SMILES: CN1C(=O)N(c2ccc(-c3cn(C)c4ncnc(N)c34)c(F)c2)CC1c1cc(F)ccc1F] | 1-(4-(4-amino-7-methyl-7H- pyrrolo[2,3-d]pyrimidin-5-yl)-3- fluorophenyl)-4-(2,5-difluorophenyl)- 3-methylimidazolidin-2-one | 453 | 2.65 (s, 3H), 3.68 (m, 1H), 3.71 (s, 3H), 4.29 (t, J = 10 Hz, 1H), 5.00 (t, J = 8.4 Hz, 1H), 5.92 (br. S., 2H), 7.11-7.15 (m, 1H), 7.20-7.30 (m, 4H), 7.32-7.34 (m, 2H), 7.40-7.42 (m, 1H), 7.69 (d, J = 12.8 Hz, 1H), 8.11 (s, 1H). |
| 19 | 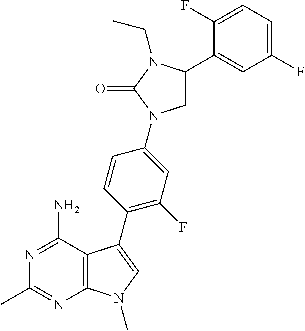 [SMILES: CCN1C(=O)N(c2ccc(-c3cn(C)c4nc(C)nc(N)c34)c(F)c2)CC1c1cc(F)ccc1F] | 1-(4-(4-amino-2,7-dimethyl-7H- pyrrolo[2,3-d]pyrimidin-5-yl)-3- fluorophenyl)-4-(2,5-difluorophenyl)- 3-ethylimidazolidin-2-one | 481.1 | 0.97 (t, J = 6.8 Hz, 3H), 2.38 (s, 3H), 2.73-2.82 (m, 1H), 3.38-3.47 (m, 1H), 3.67 (s, 3H), 3.71-3.75 (m, 1H), 4.28 (t, J = 9.6 Hz, 1H), 5.09-5.13 (m, 1H), 5.84 (br. S., 2H), 7.14 (s, 1H), 7.24-7.36 (m, 4H), 7.39-7.41 (m, 1H), 7.66-7.70 (m, 1H). |
| 20 | 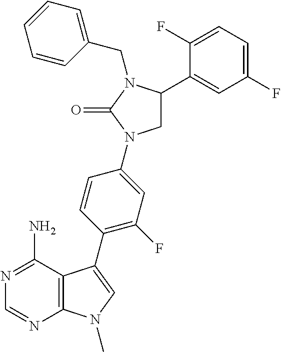 [SMILES: Cn1cc(-c2ccc(N3CC(c4cc(F)ccc4F)N(Cc4ccccc4)C3=O)cc2F)c2c(N)ncnc21] | 1-(4-(4-amino-7-methyl-7H- pyrrolo[2,3-d]pyrimidin-5-yl)-3- fluorophenyl)-3-benzyl-4-(2,5- difluorophenyl)imidazolidin-2-one | 529.1 | 3.72 (s, 3H), 3.74-3.78 (m, 1H), 3.89 (d, J = 15.6 Hz, 1H), 4.32 (t, J = 9.6 Hz, 1H), 4.66 (d, J = 15.6 Hz, 1H), 4.86-4.90 (m, 1H), 5.94 (br. S., 2H), 7.13-7.14 (m, 2H), 7.21-7.29 (m, 7H), 7.34 (t, J = 8.8 Hz, 1H), 7.43-7.46 (m, 1H), 7.73 (dd, J = 2, 12.8 Hz, 1H), 8.12 (s, 1H). |
| 21 | 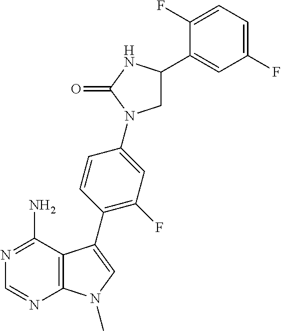 [SMILES: Cn1cc(-c2ccc(N3CC(c4cc(F)ccc4F)NC3=O)cc2F)c2c(N)ncnc21] | 1-(4-(4-amino-7-methyl-7H- pyrrolo[2,3-d]pyrimidin-5-yl)-3- fluorophenyl)-4-(2,5- difluorophenyl)imidazolidin-2-one | 439.0 | 3.71 (s, 4H), 4.36 (t, J = 9.6 Hz, 1H), 5.10 (t, J = 6.8 Hz, 1H), 5.92 (br. S., 2H), 7.23-7.32 (m, 5H), 7.37-7.39 (m, 1H), 7.67 (d, J = 12.8 Hz, 1H), 7.77 (s, 1H), 8.11 (s, 1H). |
| 22 | 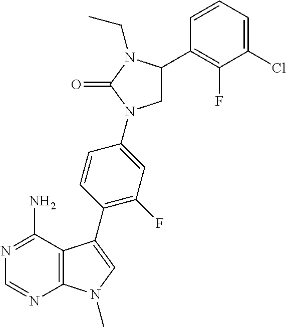 [SMILES: CCN1C(=O)N(c2ccc(-c3cn(C)c4ncnc(N)c34)c(F)c2)CC1c1cccc(Cl)c1F] | 1-(4-(4-amino-7-methyl-7H- pyrrolo[2,3-d]pyrimidin-5-yl)-3- fluorophenyl)-4-(3-chloro-2- fluorophenyl)-3-ethylimidazolidin-2- one | 483.1 | 0.97 (t, J = 7.2 Hz, 3H), 2.74-2.79 (m, 1H), 3.41-3.47 (m, 1H), 3.71-3.77 (m, 4H), 4.30 (t, J = 10.0 Hz, 1H), 5.17 (t, J = 10.0 Hz, 1H), 5.93 (br. S., 2H), 7.24-7.34 (m, 3H), 7.40-7.42 (m, 2H), 7.60 (t, J = 7.6 Hz, 1H), 7.70 (d, J = 12.4 Hz, 1H), 8.11 (s, 1H) |
| 23 | 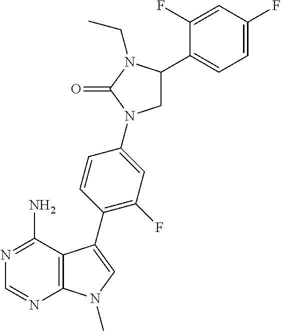 [SMILES: CCN1C(=O)N(c2ccc(-c3cn(C)c4ncnc(N)c34)c(F)c2)CC1c1ccc(F)cc1F] | 1-(4-(4-amino-7-methyl-7H- pyrrolo[2,3-d]pyrimidin-5-yl)-3- fluorophenyl)-4-(2,4-difluorophenyl)- 3-ethylimidazolidin-2-one | 467.1 | 0.96 (t, J = 6.8 Hz, 3H), 2.72-2.79 (m, 1H), 3.36-3.45 (m, 1H), 3.71 (s, 4H), 4.28 (t, J = 9.6 Hz, 1H), 5.09-5.13 (m, 1H), 5.92 (br. s., 2H), 7.12-7.17 (m, 1H), 7.24 (s, 1H), 7.31 (t, J = 8.4 Hz, 2H), 7.41 (d, J = 8.8 Hz, 1H), 7.48-7.54 (m, 1H), 7.70 (d, J = 12.8 Hz, 1H), 8.11 (s, 1H). |
| 24 | 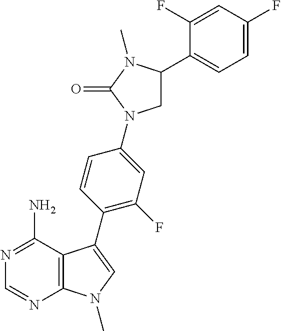 [SMILES: CN1C(=O)N(c2ccc(-c3cn(C)c4ncnc(N)c34)c(F)c2)CC1c1ccc(F)cc1F] | 1-(4-(4-amino-7-methyl-7H- pyrrolo[2,3-d]pyrimidin-5-yl)-3- fluorophenyl)-4-(2,4-difluorophenyl)- 3-methylimidazolidin-2-one | 453.1 | 2.62 (s, 3H), 3.68-3.69 (m, 1H), 3.71 (s, 3H), 4.28 (t, J = 9.6 Hz, 1H), 4.97-5.01 (m, 1H), 5.93 (br. S., 2H), 7.15 (t, J = 6.4 Hz, 1H), 7.24 (s, 1H), 7.32 (t, J = 8.8 Hz, 2H), 7.40-7.49 (m, 2H), 7.69 (d, J = 13.2 Hz, 1H), 8.11 (s, 1H) |
| 25 | 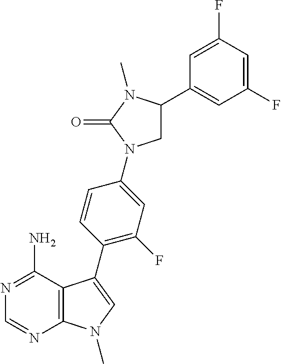 [SMILES: CN1C(=O)N(c2ccc(-c3cn(C)c4ncnc(N)c34)c(F)c2)CC1c1cc(F)cc(F)c1] | 1-(4-(4-amino-7-methyl-7H- pyrrolo[2,3-d]pyrimidin-5-yl)-3- fluorophenyl)-4-(3,5-difluorophenyl)- 3-methylimidazolidin-2-one | 453.1 | 2.62 (s, 3H), 3.63-3.65 (m, 1H), 3.71 (s, 3H), 4.25 (t, J = 9.6 Hz, 1H), 4.78 (t, J = 8.0 Hz, 1H), 5.92 (br. S., 2H), 7.15 (d, J = 6.8 Hz, 2H), 7.21-7.24 (m, 2H), 7.32 (t, J = 8.4 Hz, 1H), 7.39-7.41 (m, 1H), 7.69 (d, J = 12.0 Hz, 1H), 8.12 (s, 1H) |
| 26 | 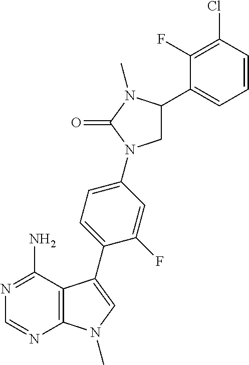 [SMILES: CN1C(=O)N(c2ccc(-c3cn(C)c4ncnc(N)c34)c(F)c2)CC1c1cccc(Cl)c1F] | 1-(4-(4-amino-7-methyl-7H- pyrrolo[2,3-d]pyrimidin-5-yl)-3- fluorophenyl)-4-(3-chloro-2- fluorophenyl)-3-methylimidazolidin- 2-one | 469.1 | 2.66 (s, 3H), 3.69-3.73 (m, 4H), 4.31 (t, J = 9.6 Hz, 1H), 5.04-5.08 (m, 1H), 5.92 (br. s, 2H), 7.24 (s, 1H), 7.27-7.34 (m, 2H), 7.35-7.38 (m, 1H), 7.40-7.42 (m, 1H), 7.60 (t, J = 7.0 Hz, 1H), 7.68 (d, J = 12.0 Hz, 1H), 8.18 (s, 1H) |
| 27 | 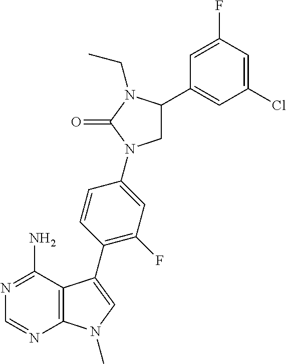 [SMILES: CCN1C(=O)N(c2ccc(-c3cn(C)c4ncnc(N)c34)c(F)c2)CC1c1cc(F)cc(Cl)c1] | 1-(4-(4-amino-7-methyl-7H- pyrrolo[2,3-d]pyrimidin-5-yl)-3- fluorophenyl)-4-(3-chloro-5- fluorophenyl)-3-ethylimidazolidin-2- one | 483.1 | 0.96 (t, J = 7.2 Hz, 3H), 2.72-2.76 (m, 1H), 3.39-3.43 (m, 1H), 3.66-3.68 (m, 1H), 3.72 (s, 3H), 4.29-4.30 (m, 1H), 4.93-4.95 (m, 1H), 5.96 (br. s., 2H), 7.25 (s, 1H), 7.30-7.34 (m, 2H), 7.39-7.44 (m, 3H), 7.68 (d, J = 12 Hz, 1H), 8.12 (s, 1H). |
| 28 | 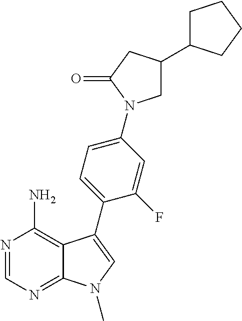 [SMILES: Cn1cc(-c2ccc(N3CC(C4CCCC4)CC3=O)cc2F)c2c(N)ncnc21] | 1-(4-(4-amino-7-methyl-7H- pyrrolo[2,3-d]pyrimidin-5-yl)-3- fluorophenyl)-4- cyclopentylpyrrolidin-2-one | 394.2 | 1.17-1.19 (m, 2H), 1.46-1.54 (m, 2H), 1.55-1.64 (m, 2H), 1.69-1.78 (m, 2H), 1.81-1.87 (m, 1H), 2.25 (d, J = 7.6 Hz, 1H), 2.34 (d, J = 8.8 Hz, 1H), 2.59 (t, J = 8.0 Hz, 1H), 3.55 (t, J = 7.6 Hz, 1H), 3.72 (s, 3H), 3.94 (t, J = 8.8 Hz, 1H), 5.94 (br. s., 2H), 7.27 (s, 1H), 7.37 (t, J = 8.8 Hz, 1H), 7.52 (d, J = 7.6 Hz, 1H), 7.76 (d, J = 12.8 Hz, 1H), 8.12 (s, 1H) |
| 29 | 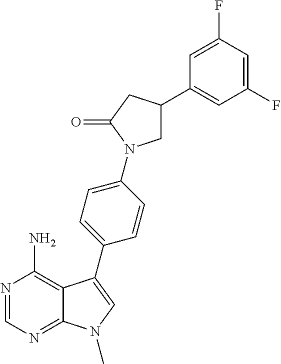 [SMILES: Cn1cc(-c2ccc(N3CC(c4cc(F)cc(F)c4)CC3=O)cc2)c2c(N)ncnc21] | 1-(4-(4-amino-7-methyl-7H- pyrrolo[2,3-d]pyrimidin-5- yl)phenyl)-4-(3,5- difluorophenyl)pyrrolidin-2-one | 420.2 | 2.74-2.81 (m, 1H), 2.88-2.94 (m, 1H), 3.73 (s, 3H), 3.77-3.90 (m, 1H), 3.92 (t, J = 8.8 Hz, 1H), 4.23 (t, J = 8.8 Hz, 1H), 6.03 (br. s., 2H), 7.13 (t, J = 9.6 Hz, 1H), 7.19 (d, J = 6.8 Hz, 2H), 7.29 (s, 1H), 7.45 (d, J = 8.4 Hz, 2H), 7.7 (d, J = 8.4 Hz, 2H), 8.14 (s, 1H). |
| 30 | 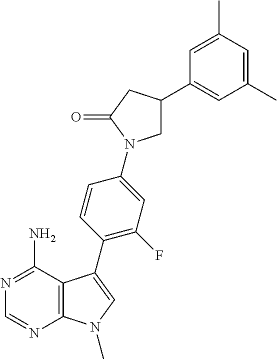 [SMILES: Cc1cc(C)cc(C2CC(=O)N(c3ccc(-c4cn(C)c5ncnc(N)c45)c(F)c3)C2)c1] | 1-(4-(4-amino-7-methyl-7H- pyrrolo[2,3-d]pyrimidin-5-yl)-3- fluorophenyl)-4-(3,5- dimethylphenyl)pyrrolidin-2-one | 430.2 | 2.26 (s, 6H), 2.65-2.75 (m, 1H), 2.87-2.94 (m, 1H), 3.61-3.69 (m, 1H), 3.73 (s, 3H), 3.86 (t, J = 9.2 Hz, 1H), 4.23 (t, J = 8.8 Hz, 1H) 5.97 (br. s., 2H), 6.89 (s, 1H), 6.99 (s, 2H), 7.30 (s, 1H), 7.39 (t, J = 8.4 Hz, 1H), 7.56-7.59 (m, 1H), 7.79-7.82 (m, 1H), 8.14 (s, 1H). |
| 31 | 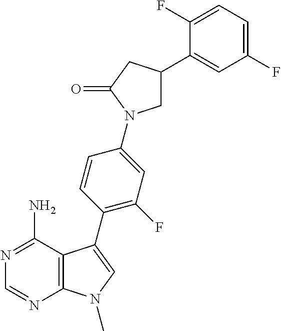 [SMILES: Cn1cc(-c2ccc(N3CC(c4cc(F)ccc4F)CC3=O)cc2F)c2c(N)ncnc21] | 1-(4-(4-amino-7-methyl-7H- pyrrolo[2,3-d]pyrimidin-5-yl)-3- fluorophenyl)-4-(2,5- difluorophenyl)pyrrolidin-2-one | 438.2 | 2.76-2.82 (m, 1H), 2.90-2.96 (m, 1H), 3.72 (s, 3H), 3.94 (s, 2H), 4.23 (t, J = 12.4 Hz, 1H), 5.96 (br. s., 2H), 7.16-7.18 (m, 1H), 7.24-7.29 (m, 2H), 7.39 (t, J = 8.0 Hz, 2H), 7.56 (d, J = 8.4 Hz, 1H), 7.78 (d, J = 12.8 Hz, 1H), 8.13 (s, 1H). |
| 32 | 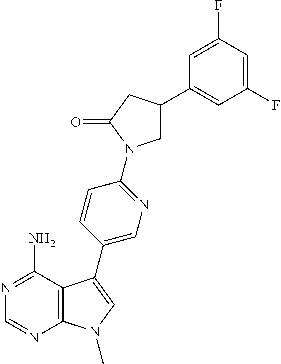 [SMILES: Cn1cc(-c2ccc(N3CC(c4cc(F)cc(F)c4)CC3=O)nc2)c2c(N)ncnc21] | 1-(5-(4-amino-7-methyl-7H- pyrrolo[2,3-d]pyrimidin-5-yl)pyridin- 2-yl)-4-(3,5- difluorophenyl)pyrrolidin-2-one | 421.1 | 2.85-2.89 (m, 1H), 2.94-3.01 (m, 1H), 3.72 (s, 3H), 3.78 (t, J = 8.8 Hz, 1H), 3.93 (t, J = 10.8 Hz, 1H), 4.48-4.52 (m, 1H), 6.10 (br. s., 2H), 7.08-7.13 (m, 1H), 7.16 (d, J = 7.2 Hz, 2H), 7.35 (s, 1H), 7.85-7.88 (m, 1H), 8.14 (s, 1H), 8.36 (d, J = 8.8 Hz, 1H), 8.42 (s, 1H). |
| 33 | 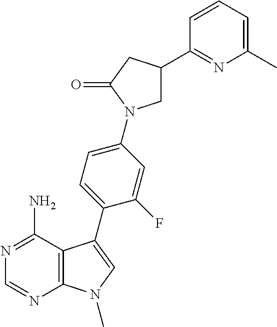 [SMILES: Cc1cccc(C2CC(=O)N(c3ccc(-c4cn(C)c5ncnc(N)c45)c(F)c3)C2)n1] | 1-(4-(4-amino-7-methyl-7H- pyrrolo[2,3-d]pyrimidin-5-yl)-3- fluorophenyl)-4-(6-methylpyridin-2- yl)pyrrolidin-2-one | 417.1 | 2.44 (s, 3H), 2.90 (t, J = 8.0 Hz, 2H), 3.72 (s, 3H), 3.82-3.86 (m, 1H), 3.99 (t, J = 7.6 Hz, 1H), 4.25 (t, J = 8.8 Hz, 1H), 5.96 (br. s., 2H), 7.14 (d, J = 7.6 Hz, 1H), 7.20 (d, J = 6.8 Hz, 1H), 7.28 (s, 1H), 7.37 (t J = 9.2 Hz, 1H), 7.54 (d, J = 8.0 Hz, 1H), 7.65 (t, J = 7.6 Hz, 1H), 7.80 (d, J = 12.8 Hz, 1H), 8.12 (s, 1H) |
| 34 | 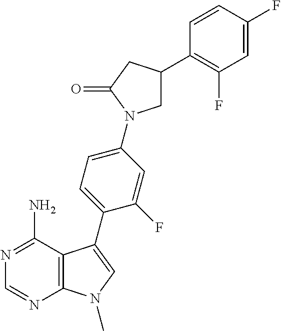 [SMILES: Cn1cc(-c2ccc(N3CC(c4ccc(F)cc4F)CC3=O)cc2F)c2c(N)ncnc21] | 1-(4-(4-amino-7-methyl-7H- pyrrolo[2,3-d]pyrimidin-5-yl)-3- fluorophenyl)-4-(2,4- difluorophenyl)pyrrolidin-2-one | 438.1 | 2.72-2.78 (m, 1H), 2.91-2.98 (m, 1H), 3.72 (s, 3H), 3.88-3.93 (m, 2H), 4.21-4.27 (m, 1H), 5.96 (br. s., 2H), 7.07-7.12 (m, 1H), 7.22-7.28 (m, 2H), 7.38 (t, J = 8.4 Hz, 1H), 7.50-7.56 (m, 2H), 7.77-7.80 (m, 1H), 8.12 (s, 1H) |
| 35 | 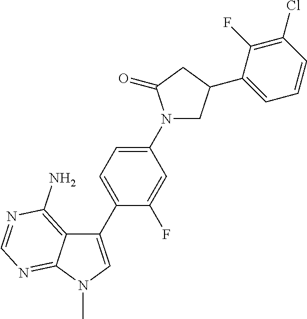 [SMILES: Cn1cc(-c2ccc(N3CC(c4cccc(Cl)c4F)CC3=O)cc2F)c2c(N)ncnc21] | 1-(4-(4-amino-7-methyl-7H- pyrrolo[2,3-d]pyrimidin-5-yl)-3- fluorophenyl)-4-(3-chloro-2- fluorophenyl)pyrrolidin-2-one | 454.2 | 2.73-2.80 (m, 1H), 2.94-3.0 (m, 1H), 3.71 (s, 3H), 3.92-4.0 (m, 2H), 4.24-4.26 (m, 1H), 5.95 (br, s., 2H), 7.21-7.28 (m, 2H), 7.38 (t, J = 8.4 Hz, 1H), 7.44-7.56 (m, 3H), 7.78 (d, J = 12.8 Hz, 1H), 8.12 (s, 1H). |
| 36 | 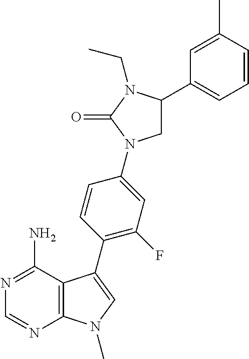 [SMILES: CCN1C(=O)N(c2ccc(-c3cn(C)c4ncnc(N)c34)c(F)c2)CC1c1cccc(C)c1] | 1-(4-(4-amino-7-methyl-7H- pyrrolo[2,3-d]pyrimidin-5-yl)-3- fluorophenyl)-3-ethyl-4-(m- tolyl)imidazolidin-2-one | 445.2 | 0.96-0.93 (m, 3H), 2.31 (s, 3H), 2.63-2.73 (m, 1H), 3.36-3.42 (m, 1H), 3.58-3.62 (m, 1H), 3.71 (s, 3H), 4.21-4.25 (m, 1H), 4.80-4.84 (m, 1H), 5.92 (br. s., 2H), 7.16-7.28 (m, 4H), 7.30-7.33 (m, 2H), 7.40-7.42 (m, 1H), 7.70 (d, J = 13.2 Hz, 1H), 8.11 (s, 1H). |
| 37 | 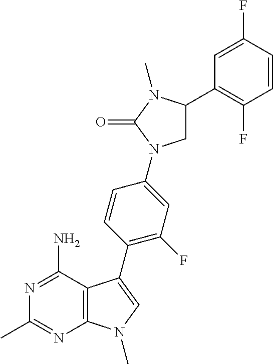 [SMILES: Cc1nc(N)c2c(-c3ccc(N4CC(c5cc(F)ccc5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(2,5-difluorophenyl)- 3-methylimidazolidin-2-one (enantiomer 1) | 467.2 | 2.38 (s, 3H), 2.65 (s, 3H), 3.67-3.71 (m, 4H), 4.26-4.31 (m, 1H), 4.98-5.02 (m, 1H), 5.84 (br. s., 2H), 7.14 (s, 1H), 7.26-7.34 (m, 4H), 7.39-7.41 (m, 1H), 7.66-7.69 (m, 1H). HPLC Column: CHIRALPAK IA (250 mm × 4.6 mm × 5 μm) Mobile phase: n-Hexane: 0.1% DEA in Ethanol (70:30) Flow rate: 1.0 mL/min Retention time: 23.618 min |
| 38 | 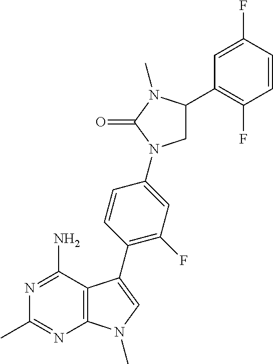 [SMILES: Cc1nc(N)c2c(-c3ccc(N4CC(c5cc(F)ccc5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(2,5-difluorophenyl)- 3-methylimidazolidin-2-one (enantiomer 2) | 467.2 | 2.38 (s, 3H), 2.65 (s, 3H), 3.67-3.71 (m, 4H), 4.26-4.31 (m, 1H), 4.98-5.02 (m, 1H), 5.83 (br. s., 2H), 7.14 (s, 1H), 7.26-7.34 (m, 4H), 7.39-7.41 (m, 1H), 7.66-7.69 (m, 1H). HPLC Column: CHIRALPAK IA (250 mm × 4.6 mm × 5 μm) Mobile phase: n-Hexane: 0.1% DEA in Ethanol (70:30) Flow rate: 1.0 mL/min Retention time: 30.384 min |
| 39 | 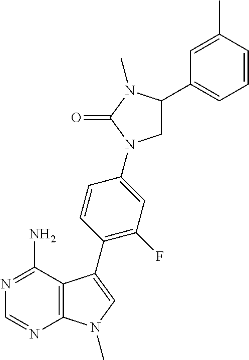 [SMILES: Cc1cccc(C2CN(c3ccc(-c4cn(C)c5ncnc(N)c45)c(F)c3)C(=O)N2C)c1] | 1-(4-(4-amino-7-methyl-7H- pyrrolo[2,3-d]pyrimidin-5-yl)-3- fluorophenyl)-3-methyl-4-(m- tolyl)imidazolidin-2-one | 431.2 | 2.30 (s, 3H), 2.59 (s, 3H), 3.56-3.60 (m, 1H), 3.71 (s, 3H), 4.22-4.26 (m, 1H), 4.65-4.69 (m, 1H), 5.92 (br. s., 2H), 7.15-7.19 (m, 3H), 7.24 (s, 1H), 7.28-7.40 (m, 2H), 7.40-7.42 (m, 1H), 7.68-7.72 (m, 1H), 8.12 (s, 1H). |
| 40 | 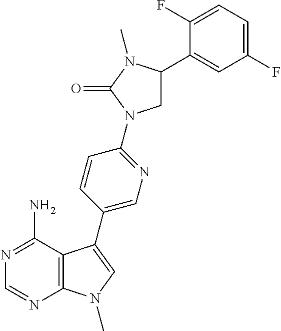 [SMILES: CN1C(=O)N(c2ccc(-c3cn(C)c4ncnc(N)c34)cn2)CC1c1cc(F)ccc1F] | 1-(5-(4-amino-7-methyl-7H- pyrrolo[2,3-d]pyrimidin-5-yl)pyridin- 2-yl)-4-(2,5-difluorophenyl)-3- methylimidazolidin-2-one | 436.1 | 2.68 (s, 3H), 3.71 (s, 3H), 3.79-3.83 (m, 1H), 4.40-4.45 (m, 1H), 4.98-5.01 (m, 1H), 6.06 (br. s., 2H), 7.24-7.33 (m, 4H), 7.79 (d, J = 8 Hz, 1H), 8.13 (s, 1H), 8.27-8.31 (m, 2H). |
| 41 | 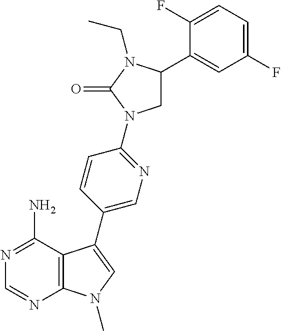 [SMILES: CCN1C(=O)N(c2ccc(-c3cn(C)c4ncnc(N)c34)cn2)CC1c1cc(F)ccc1F] | 1-(5-(4-amino-7-methyl-7H- pyrrolo[2,3-d]pyrimidin-5-yl)pyridin- 2-yl)-4-(2,5-difluorophenyl)-3- ethylimidazolidin-2-one | 450.2 | 0.97-1.0 (m, 3H), 2.77-2.82 (m, 1H), 3.44-3.49 (m, 1H), 3.71 (s, 3H), 3.83-3.88 (m, 1H), 4.40-4.45 (m, 1H), 5.09-5.12 (m, 1H), 6.06 (br. s., 2H), 7.25-7.35 (m, 4H), 7.78-7.80 (m, 1H), 8.13 (s, 1H), 8.28-8.31 (m, 2H). |
| 42 | 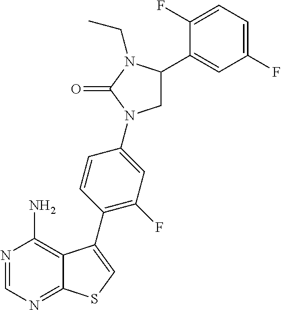 [SMILES: CCN1C(=O)N(c2ccc(-c3csc4ncnc(N)c34)c(F)c2)CC1c1cc(F)ccc1F] | 1-(4-(4-aminothieno[2,3-d]pyrimidin- 5-yl)-3-fluorophenyl)-4-(2,5- difluorophenyl)-3-ethylimidazolidin- 2-one | 470.1 | 0.97 (t, J = 7.6 Hz, 3H), 2.75-2.81 (m, 1H), 3.31-3.46 (m, 1H), 3.74-3.78 (m, 1H), 4.31 (t, J = 9.6 Hz, 1H), 5.11-5.15 (m, 1H), 5.80-6.60 (br. s., 2H), 7.25-7.30 (m, 1H), 7.31-7.37 (m, 2H), 7.39-7.42 (m, 1H), 7.47-7.49 (m, 2H), 7.73 (d, J = 11.6 Hz, 1H), 8.31 (s, 1H) |
| 43 | 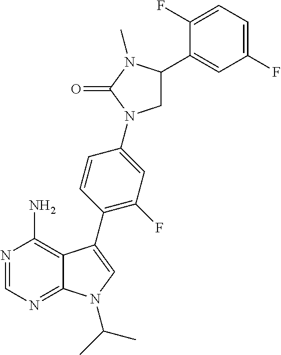 [SMILES: CC(C)n1cc(-c2ccc(N3CC(c4cc(F)ccc4F)N(C)C3=O)cc2F)c2c(N)ncnc21] | 1-(4-(4-amino-7-isopropyl-7H- pyrrolo[2,3-d]pyrimidin-5-yl)-3- fluorophenyl)-4-(2,5-difluorophenyl)- 3-methylimidazolidin-2-one | 481.2 | 1.43 (d, J = 6.8 Hz, 6H), 3.42 (s, 3H), 3.68-3.72 (m, 1H), 4.30 (t, J = 9.4 Hz, 1H), 4.90-4.95 (m, 1H), 5.00-5.02 (m, 1H), 5.89 (br. s., 2H), 7.25-7.29 (m, 2H), 7.31-7.40 (m, 3H), 7.42-7.43 (m, 1H), 7.69 (dd, J = 1.6, 13.2 Hz, 1H), 8.09 (s, 1H) |
| 44 | 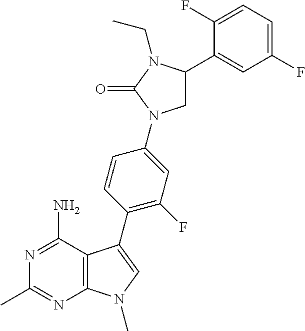 [SMILES: CCN1C(=O)N(c2ccc(-c3cn(C)c4nc(C)nc(N)c34)c(F)c2)CC1c1cc(F)ccc1F] | 1-(4-(4-amino-2,7-dimethyl-7H- pyrrolo[2,3-d]pyrimidin-5-yl)-3- fluorophenyl)-4-(2,5-difluorophenyl)- 3-ethylimidazolidin-2-one (enantiomer 1) | 481.2 | 0.97 (t, J = 7.2 Hz, 3H), 2.38 (s, 3H), 2.73-2.82 (m, 1H), 3.35-3.47 (m, 1H), 3.67 (s, 3H), 3.71-3.75 (m, 1H), 4.29 (t, J = 9.6 Hz, 1H), 5.06-5.13 (m, 1H), 5.84 (br. s., 2H), 7.14 (s, 1H), 7.24-7.36 (m, 4H), 7.39-7.41 (m, 1H), 7.67-7.70 (m, 1H). HPLC Column: CHIRALPAK IA (250 mm × 4.6 mm × 5 μm) Mobile phase: 0.1% DEA in 100% MEOH Flow rate: 0.5 mL/min Retention time: 19.456 min |
| 45 | 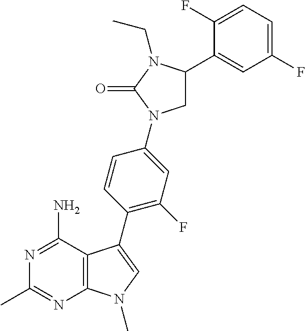 [SMILES: CCN1C(=O)N(c2ccc(-c3cn(C)c4nc(C)nc(N)c34)c(F)c2)CC1c1cc(F)ccc1F] | 1-(4-(4-amino-2,7-dimethyl-7H- pyrrolo[2,3-d]pyrimidin-5-yl)-3- fluorophenyl)-4-(2,5-difluorophenyl)- 3-ethylimidazolidin-2-one (enantiomer 2) | 481.2 | 0.97 (t, J = 7.2 Hz, 3H), 2.38 (s, 3H), 2.75-2.82 (m, 1H), 3.40-3.45 (m, 1H), 3.67 (s, 3H), 3.71-3.75 (m, 1H), 4.29 (t, J = 9.6 Hz, 1H), 5.11 (t, J = 9.2 Hz, 1H), 5.83 (br. s., 2H), 7.14 (s, 1H), 7.26-7.36 (m, 4H), 7.39-7.41 (m, 1H), 7.68 (d, J = 12.0 Hz, 1H). HPLC Column: CHIRALPAK IA (250 mm × 4.6 mm × 5 μm) Mobile phase: 0.1% DEA in 100% MEOH Flow rate: 0.5 mL/min Retention time: 22.169 min |
| 47 | 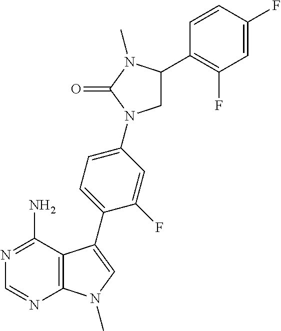 [SMILES: CN1C(=O)N(c2ccc(-c3cn(C)c4ncnc(N)c34)c(F)c2)CC1c1ccc(F)cc1F] | 1-(4-(4-amino-7-methyl-7H- pyrrolo[2,3-d]pyrimidin-5-yl)-3- fluorophenyl)-4-(2,4-difluorophenyl)- 3-methylimidazolidin-2-one (enantiomer 2) | 453.1 | 2.63 (s, 3H), 3.66-3.69 (m, 1H), 3.71 (s, 3H), 4.28 (t, J = 9.6 Hz, 1H), 4.97-5.12 (m, 1H),5.92 (br. s., 2H), 7.13-7.17 (m, 1H), 7.24 (s, 1H), 7.32 (t, J = 8.8 Hz, 2H), 7.40-7.49 (m, 2H), 7.66-7.70 (m, 1H), 8.11 (s, 1H) HPLC Column: CHIRALPAK IA (250 mm × 4.6 mm × 5 μm) Mobile phase: 0.1% DEA in 100% MEOH Flow rate: 0.7 mL/min Retention time: 18.986 min |
| 48 | 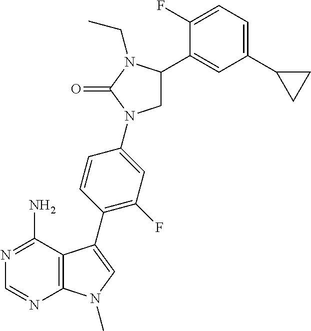 [SMILES: CCN1C(=O)N(c2ccc(-c3cn(C)c4ncnc(N)c34)c(F)c2)CC1c1cc(C2CC2)ccc1F] | 1-(4-(4-amino-7-methyl-7H- pyrrolo[2,3-d]pyrimidin-5-yl)-3- fluorophenyl)-4-(5-cyclopropyl-2- fluorophenyl)-3-ethylimidazolidin-2- one | 489.2 | 0.61-0.64 (m, 2H), 0.90-1.0 (m, 5H), 1.9-1.94 (m, 1H), 2.70-2.77 (m, 1H), 3.35-3.42 (m, 1H), 3.68 (s, 3H), 3.69-3.71 (m, 1H), 4.24-4.28 (m, 1H), 5.05-5.09 (m, 1H), 5.93 (br. s., 2H), 7.07-7.09 (m, 1H), 7.10 (s, 1H), 7.15-7.17 (m, 1H), 7.24 (s, 1H), 7.29-7.34 (m, 1H), 7.41-7.43 (m, 1H), 7.69-7.72 (m, 1H), 8.12 (s, 1H). |
| 49 | 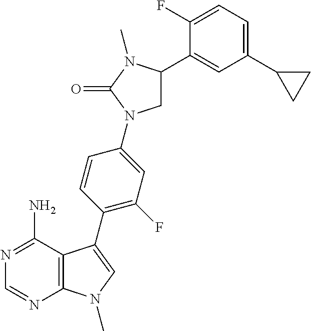 [SMILES: CN1C(=O)N(c2ccc(-c3cn(C)c4ncnc(N)c34)c(F)c2)CC1c1cc(C2CC2)ccc1F] | 1-(4-(4-amino-7-methyl-7H- pyrrolo[2,3-d]pyrimidin-5-yl)-3- fluorophenyl)-4-(5-cyclopropyl-2- fluorophenyl)-3-methylimidazolidin- 2-one | 475.2 | 0.59-0.67 (m, 2H), 0.90-0.92 (m, 2H), 1.9-1.95 (m, 1H), 2.64 (s, 3H), 3.65-3.69 (m, 1H), 3.71 (s, 3H), 4.24-4.29 (m, 1H), 4.92-4.96 (m, 1H), 5.93 (br. s., 2H), 7.05-7.15 (m, 3H), 7.24 (s, 1H), 7.29-7.34 (m, 1H), 7.40-7.43 (m, 1H), 7.68-7.72 (m, 1H), 8.12 (s, 1H). |
| 50 | 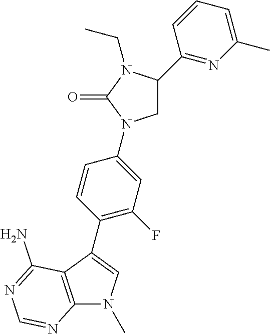 [SMILES: CCN1C(=O)N(c2ccc(-c3cn(C)c4ncnc(N)c34)c(F)c2)CC1c1cccc(C)n1] | 1-(4-(4-amino-7-methyl-7H- pyrrolo[2,3-d]pyrimidin-5-yl)-3- fluorophenyl)-3-ethyl-4-(6- methylpyridin-2-yl)imidazolidin-2- one | 446.2 | 0.93 (t, J = 6.8 Hz, 3H), 2.70-2.79 (m, 1H), 3.26-3.43 (m, 4H), 3.71 (s, 3H), 3.70-3.80 (m, 1H), 4.23 (t, J = 9.2 Hz, 1H), 4.90-4.94 (m, 1H), 5.92 (br. s, 2H), 7.22-7.28 (m, 3H), 7.31-7.33 (m, 1H), 7.40-7.42 (m, 1H), 7.70-7.75 (m, 2H), 8.11 (s, 1H). |
| 51 | 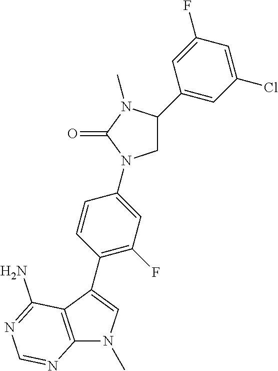 [SMILES: CN1C(=O)N(c2ccc(-c3cn(C)c4ncnc(N)c34)c(F)c2)CC1c1cc(F)cc(Cl)c1] | 1-(4-(4-amino-7-methyl-7H- pyrrolo[2,3-d]pyrimidin-5-yl)-3- fluorophenyl)-4-(3-chloro-5- fluorophenyl)-3-methylimidazolidin- 2-one (enantiomer 1) | 469.1 | 2.62 (s, 3H), 3.64 (t, J = 8.4 Hz, 1H), 3.71 (s, 3H), 4.24 (t, J = 9.6 Hz, 1H), 4.78 (t, J = 8.0 Hz, 1H), 5.92 (br. s., 2H), 7.24-7.26 (m, 1H), 7.28-7.30 (m, 1H), 7.32-7.39 (m, 3H), 7.41-7.45 (m, 1H), 7.68 (d, J = 13.6 Hz, 1H), 8.12 (s, 1H). HPLC Column: CHIRALPAK IA (250 mm × 4.6 mm × 5 im) Mobile phase (A): n-Hexane Mobile phase (B): Ethanol with 0.1% DEA Composition (A:B): (50:50) Retention time: 12.460 min |
| 52 | 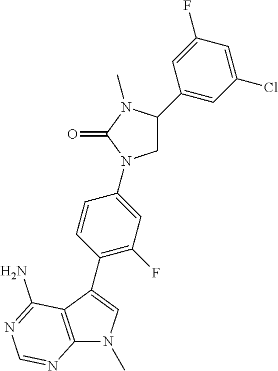 [SMILES: CN1C(=O)N(c2ccc(-c3cn(C)c4ncnc(N)c34)c(F)c2)CC1c1cc(F)cc(Cl)c1] | 1-(4-(4-amino-7-methyl-7H- pyrrolo[2,3-d]pyrimidin-5-yl)-3- fluorophenyl)-4-(3-chloro-5- fluorophenyl)-3-methylimidazolidin- 2-one (enantiomer 2) | 469.1 | 2.62 (s, 3H), 3.64 (t, J = 8.4 Hz, 1H), 3.72 (s, 3H), 4.24 (t, J = 9.6 Hz, 1H), 4.78 (t, J = 8.0 Hz, 1H), 5.92 (br. s, 2H), 7.24-7.29 (m, 1H), 7.30-7.36 (m, 1H), 7.39-7.45 (m, 4H), 7.68-7.69 (m, 1H), 8.12 (s, 1H). HPLC Column: CHIRALPAK IA (250 mm × 4.6 mm × 5 im) Mobile phase (A): n-Hexane Mobile phase (B): Ethanol with 0.1% DEA Composition (A:B): (50:50) Retention time: 18.587 min |
| 53 | 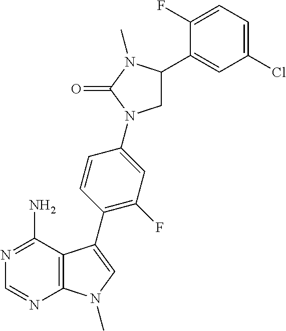 [SMILES: CN1C(=O)N(c2ccc(-c3cn(C)c4ncnc(N)c34)c(F)c2)CC1c1cc(Cl)ccc1F] | 1-(4-(4-amino-7-methyl-7H- pyrrolo[2,3-d]pyrimidin-5-yl)-3- fluorophenyl)-4-(5-chloro-2- fluorophenyl)-3-methylimidazolidin- 2-one | 469.2 | 2.65 (s, 3H), 3.71-3.74 (m, 4H), 4.29 (t, J = 9.6 Hz, 1H), 5.0 (t, J = 9.2 Hz, 1H), 5.92 (br. s., 2H), 7.24 (s, 1H), 7.30-7.36 (m, 2H), 7.40-7.42 (m, 1H), 7.48 (s, 2H), 7.68 (d, J = 12.8 Hz, 1H), 8.11 (s, 1H). |
| 54 | 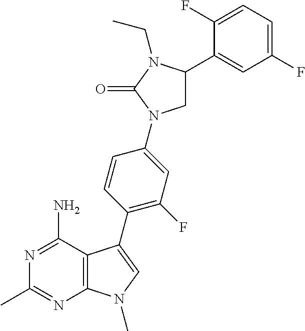 [SMILES: CCN1C(=O)N(c2ccc(-c3cn(C)c4nc(C)nc(N)c34)c(F)c2)CC1c1cc(F)ccc1F] | 1-(4-(4-amino-2,7-dimethyl-7H- pyrrolo[2,3-d]pyrimidin-5-yl)-3- fluorophenyl)-4-(2,5-difluorophenyl)- 3-ethylimidazolidin-2-one (enantiomer 1) | 481.2 | 0.97 (t, J = 7.2 Hz, 3H), 2.38 (s, 3H), 2.73-2.82 (m, 1H), 3.35-3.47 (m, 1H), 3.67 (s, 3H), 3.71-3.75 (m, 1H), 4.29 (t, J = 9.6 Hz, 1H), 5.06-5.13 (m, 1H), 5.84 (br. s., 2H), 7.14 (s, 1H), 7.24-7.36 (m, 4H), 7.39-7.41 (m, 1H), 7.67-7.70 (m, 1H). HPLC Column: CHIRALPAK IA (250 mm × 4.6 mm × 5 μm) Mobile phase: 0.1% DEA in 100% MEOH Flow rate: 0.5 mL/min Retention time: 19.456 min |
| 55 | 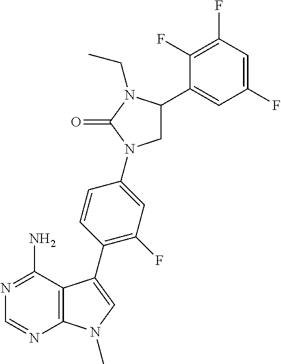 [SMILES: CCN1C(=O)N(c2ccc(-c3cn(C)c4ncnc(N)c34)c(F)c2)CC1c1cc(F)cc(F)c1F] | 1-(4-(4-amino-7-methyl-7H- pyrrolo[2,3-d]pyrimidin-5-yl)-3- fluorophenyl)-3-ethyl-4-(2,3,5- trifluorophenyl)imidazolidin-2-one | 485.1 | 0.98 (t, J = 7.2 Hz, 3H), 2.77-2.83 (m, 1H), 3.40-3.47 (m, 1H), 3.71 (s, 3H), 3.76-3.8 (m, 1H), 4.30 (t, J = 9.6 Hz, 1H), 5.18 (t, J = 9.2 Hz, 1H), 5.92 (br. s., 2H), 7.20 (s, 1H), 7.24 (s, 1H), 7.30-7.34 (m, 1H), 7.39-7.41 (m, 1H), 7.55-7.57 (m, 1H), 7.68 (d, J = 12.8 Hz, 1H), 8.12 (s, 1H). |
| 56 | 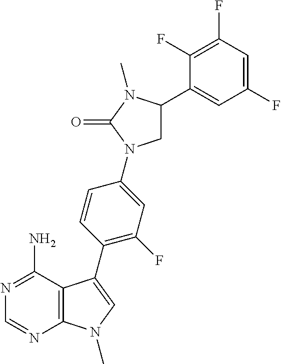 [SMILES: CN1C(=O)N(c2ccc(-c3cn(C)c4ncnc(N)c34)c(F)c2)CC1c1cc(F)cc(F)c1F] | 1-(4-(4-amino-7-methyl-7H- pyrrolo[2,3-d]pyrimidin-5-yl)-3- fluorophenyl)-3-methyl-4-(2,3,5- trifluorophenyl)imidazolidin-2-one | 471.1 | 2.67 (s, 3H), 3.72-3.76 (m, 4H), 4.31 (t, J = 9.6 Hz, 1H), 5.07 (t, J = 6.4 Hz, 1H), 5.92 (br. s., 2H), 7.15 (s, 1H), 7.25 (s, 1H), 7.33 (t, J = 8.8 Hz, 1H), 7.39-7.41 (m, 1H), 7.57 (s, 1H), 7.68 (d, J = 12.8 Hz, 1H), 8.12 (s, 1H). |
| 57 | 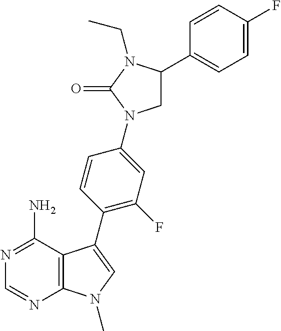 [SMILES: CCN1C(=O)N(c2ccc(-c3cn(C)c4ncnc(N)c34)c(F)c2)CC1c1ccc(F)cc1] | 1-(4-(4-amino-7-methyl-7H- pyrrolo[2,3-d]pyrimidin-5-yl)-3- fluorophenyl)-3- fluorophenyl)imidazolidin-2-one | 449.2 | 0.94 (t, J = 6.8 Hz, 3H), 2.67-2.74 (m 1H), 3.34-3.41 (m, 1H), 3.62 (t, J = 8.4 Hz, 1H), 3.72 (s, 3H), 4.24 (t, J = 8.8 Hz, 1H), 4.90 (t, J = 8.0 Hz, 1H), 5.92 (br. s., 2H), 7.25-7.27 (m, 3H), 7.32 (d, J = 8.4 Hz, 1H), 7.41- 7.49 (m, 3H), 7.70 (d, J = 13.2 Hz, 1H), 8.12 (s, 1H). |
| 58 | 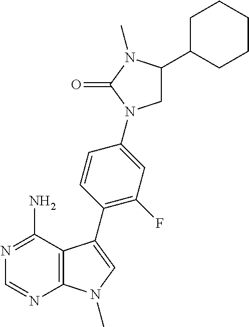 [SMILES: CN1C(=O)N(c2ccc(-c3cn(C)c4ncnc(N)c34)c(F)c2)CC1C1CCCCC1] | 1-(4-(4-amino-7-methyl-7H- pyrrolo[2,3-d]pyrimidin-5-yl)-3- fluorophenyl)-4-cyclohexyl-3- methylimidazolidin-2-one | 423.2 | 0.95-1.01 (m, 1H), 1.10-1.27 (m, 4H), 1.38-1.41 (m, 1H), 1.52-1.55 (m, 1H), 1.62-1.65 (m, 1H), 1.69-1.76 (m, 3H), 2.75 (s, 3H), 3.59-3.62 (m, 2H), 3.71-3.76 (m, 4H), 5.95 (br. s., 2H), 7.24 (s, 1H), 7.30 (d, J = 8.8 Hz, 1H), 7.42-7.44 (m, 1H), 7.68 (d, J = 13.2 Hz, 1H), 8.12 (s, 1H). |
| 60 | 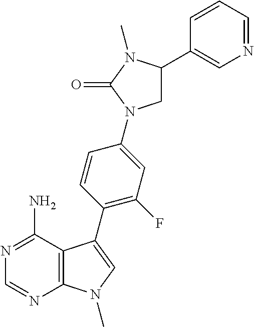 [SMILES: CN1C(=O)N(c2ccc(-c3cn(C)c4ncnc(N)c34)c(F)c2)CC1c1cccnc1] | 1-(4-(4-amino-7-methyl-7H- pyrrolo[2,3-d]pyrimidin-5-yl)-3- fluorophenyl)-3-methyl-4-(pyridin-3- yl)imidazolidin-2-one | 418.3 | 2.60 (s, 3H), 3.65-3.72 (m, 4H), 4.28 (t, J = 9.6 Hz, 1H), 4.80 (t, J = 8.0 Hz, 1H), 5.96 (br. s., 2H), 7.25 (s, 1H), 7.33 (t, J = 8.8 Hz, 1H), 7.41-7.48 (m, 2H), 7.70 (d, J = 12.8 Hz, 1H), 7.82-7.84 (m, 1H), 8.12 (s, 1H), 8.58-8.62 (m, 2H). |
| 61 | 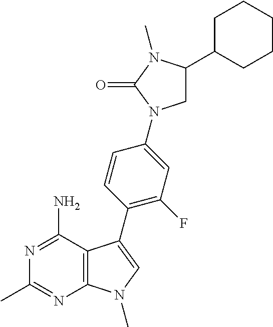 [SMILES: Cc1nc(N)c2c(-c3ccc(N4CC(C5CCCCC5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-cyclohexyl-3- methylimidazolidin-2-one (enantiomer 1) | 437.2 | 0.95-1.01 (m, 1H), 1.10-1.27 (m, 4H), 1.39-1.42 (m, 1H), 1.53-1.56 (m, 1H), 1.62-1.65 (m, 1H), 1.69-1.76 (m, 3H), 2.39 (s, 3H), 2.75 (s, 3H), 3.58-3.61 (m, 2H), 3.68 (s, 3H), 3.73-3.79 (m, 1H), 5.84 (br. s., 2H), 7.13 (s, 1H), 7.29 (d, J = 8.4 Hz, 1H), 7.41-7.42 (m, 1H), 7.67 (d, J = 12.0 Hz, 1H). HPLC Column: Chiralpak IC (250 mm × 4.6 mm × 5 μm) Mobile phase: MTBE:IPA with 0.1% DEA (70:30) Flow rate: 1.0 mL/min Retention time: 13.788 min |
| 62 | 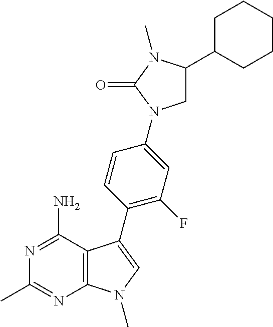 [SMILES: Cc1nc(N)c2c(-c3ccc(N4CC(C5CCCCC5)N(C)C4=O)cc3F)cn(C)c2n1] | 1-(4-(4-amino-7-methyl-7H- pyrrolo[2,3-d]pyrimidin-5-yl)-3- fluorophenyl)-4-cyclohexyl-3- methylimidazolidin-2-one (enantiomer 1) | 423.2 | 0.96-0.99 (m, 1H), 1.10-1.24 (m, 4H), 1.39-1.42 (m, 1H), 1.53-1.56 (m, 1H), 1.63-1.65 (m, 1H), 1.69-1.76 (m, 3H), 2.75 (s, 3H), 3.59-360 (m, 2H), 3.72-3.77 (m, 4H), 5.93 (br. s., 2H), 7.23 (s, 1H), 7.30 (t, J = 8.8 Hz, 1H), 7.42-7.44 (m, 1H), 7.68 (d, J = 12.4 Hz, 1H), 8.12 (s, 1H). HPLC Column: CHIRALPAK IA (250 mm × 4.6 mm × 5 μm) Mobile phase: 100% Ethanol with 0.1% TFA Flow rate: 0.5 mL/min Retention time: 8.815 min |
| 63 | 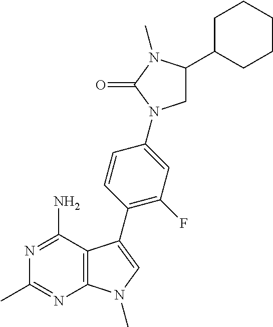 [SMILES: Cc1nc(N)c2c(-c3ccc(N4CC(C5CCCCC5)N(C)C4=O)cc3F)cn(C)c2n1] | 1-(4-(4-amino-7-methyl-7H- pyrrolo[2,3-d]pyrimidin-5-yl)-3- fluorophenyl)-4-cyclohexyl-3- methylimidazolidin-2-one (enantiomer 2) | 423.2 | 0.96-1.02 (m, 1H), 1.10-1.3 (m, 4H), 1.39-1.42 (m, 1H), 1.53-1.56 (m, 1H), 1.62-1.65 (m, 1H), 1.69-1.76 (m, 3H), 2.75 (s, 3H), 3.58 (s, 2H), 3.72-3.77 (m, 4H), 5.92 (br. S., 2H), 7.23 (s, 1H), 7.30 (t, J = 8.8 Hz, 1H), 7.42-7.44 (m, 1H), 7.68 (d, J = 12.8 Hz, 1H), 8.12 (s, 1H). HPLC Column: CHIRALPAK IA (250 mm × 4.6 mm × 5 μm) Mobile phase: 100% Ethanol with 0.1% TFA Flow rate: 0.5 mL/min Retention time: 13.333 min |
| 64 | 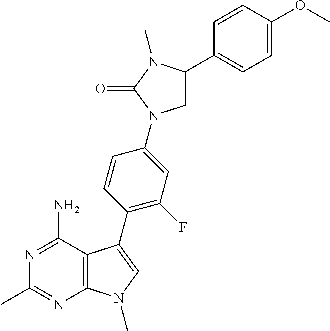 | 1-(4-(4-amino-2,7-dimethyl-7H- pyrrolo[2,3-d]pyrimidin-5-yl)-3- fluorophenyl)-4-(4-methoxyphenyl)- 3-methylimidazolidin-2-one | 461.2 | 2.39 (s, 3H), 2.56 (s, 3H), 3.58 (t, J = 8.0 Hz, 1H), 3.68 (s, 3H), 3.75 (s, 3H), 4.21 (t, J = 9.2 Hz, 1H), 4.65 (t, J = 8.4 Hz, 1H), 5.85 (br. s., 2H), 6.97 (d, J = 8.4 Hz, 2H), 7.14 (s, 1H), 7.29-7.32 (m, 3H), 7.39-7.41 (m, 1H), 7.7 (d, J = 12.8 Hz, 1H). |
| 65 | 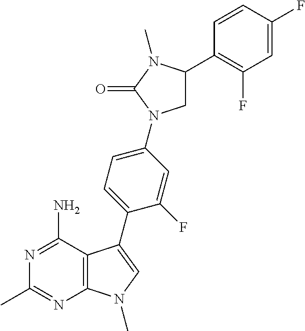 [SMILES: Cc1nc(N)c2c(-c3ccc(N4CC(c5ccc(F)cc5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(2,4-difluorophenyl)- 3-methylimidazolidin-2-one | 467.1 | 2.38 (s, 3H), 2.62 (s, 3H), 3.67 (s, 3H), 3.66-3.70 (m, 1H), 4.28 (t, J = 9.6 Hz, 1H), 4.97-5.01 (m, 1H), 5.84 (br. s., 2H), 7.13-7.17 (m, 2H), 7.28-7.35 (m, 2H), 7.39-7.41 (m, 1H), 7.43-7.49 (m, 1H), 7.66-7.70 (m, 1H). |
| 66 | 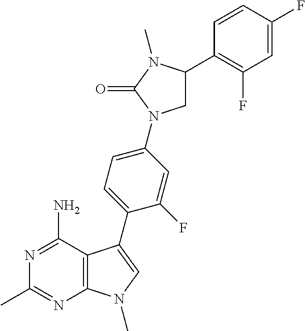 [SMILES: Cc1nc(N)c2c(-c3ccc(N4CC(c5ccc(F)cc5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(2,4-difluorophenyl)- 3-methylimidazolidin-2-one (enantiomer 1) | 467.1 | 2.38 (s, 3H), 2.62 (s, 3H), 3.67 (s, 3H), 3.67-3.70 (m, 1H), 4.28 (t, J = 9.6 Hz, 1H), 4.97-5.01 (m, 1H), 5.83 (br. s, 2H), 7.14-7.17 (m, 2H), 7.28-7.34 (m, 2H), 7.39-7.41 (m, 1H), 7.43-7.49 (m, 1H), 7.66-7.70 (m, 1H). HPLC Column: Chiralpak IA (250 mm × 4.6 mm × 5 μm) Mobile phase A: MTBE Mobile phase B: Ethanol 0.1% TFA Composition: (80:20) Flow rate: 1.0 mL/min Retention time: 7.839 min |
| 67 | 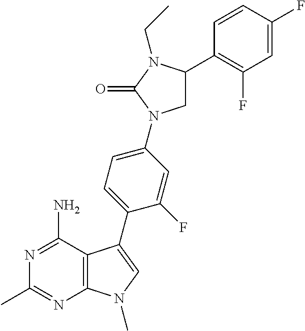 [SMILES: CCN1C(=O)N(c2ccc(-c3cn(C)c4nc(C)nc(N)c34)c(F)c2)CC1c1ccc(F)cc1F] | 1-(4-(4-amino-2,7-dimethyl-7H- pyrrolo[2,3-d]pyrimidin-5-yl)-3- fluorophenyl)-4-(2,4-difluorophenyl)- 3-ethylimidazolidin-2-one (enantiomer 2) | 481.2 | 0.96 (t, J = 6.8 Hz, 3H), 2.38 (s, 3H), 2.72-2.77 (m, 1H), 3.38-3.43 (m, 1H), 3.67 (s, 3H), 3.69-3.73 (m, 1H), 4.27 (t, J = 9.6 Hz, 1H), 5.09-5.13 (m, 1H), 5.84 (br. s., 2H), 7.12-7.17 (m, 2H), 7.28-7.34 (m, 2H), 7.38-7.41 (m, 1H), 7.48-7.54 (m, 1H), 7.67-7.71 (m, 1H). HPLC Column: CHIRALPAK IC (250 mm × 4.6 mm × 5 μm) Mobile phase: n-Hexane: 0.1% DEA in Ethanol (65:35) Flow rate: 1.0 mL/min Retention time: 34.211 min |
| 68 | 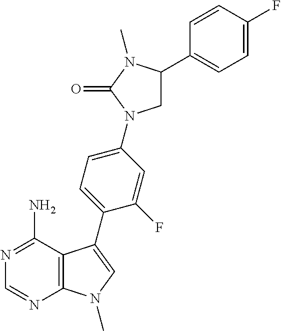 [SMILES: CN1C(=O)N(c2ccc(-c3cn(C)c4ncnc(N)c34)c(F)c2)CC1c1ccc(F)cc1] | 1-(4-(4-amino-7-methyl-7H- pyrrolo[2,3-d]pyrimidin-5-yl)-3- fluorophenyl)-4-(4-fluorophenyl)-3- methylimidazolidin-2-one | 435.1 | 2.58 (s, 3H), 3.60 (t, J = 8.0 Hz, 1H), 3.72 (s, 3H), 4.25 (t, J = 9.2 Hz, 1H), 4.74 (t, J = 8.0 Hz, 1H), 5.93 (br. s., 2H), 7.23-7.27 (m, 3H), 7.32 (t, J = 8.8 Hz, 1H), 7.41-7.46 (m, 3H), 7.70 (d, J = 12.8 Hz, 1H), 8.12 (s, 1H). |
| 69 | 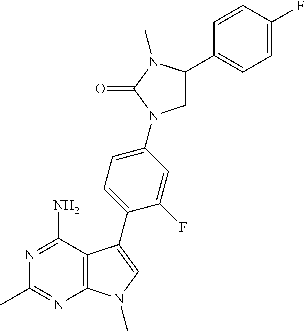 [SMILES: Cc1nc(N)c2c(-c3ccc(N4CC(c5ccc(F)cc5)N(C)C4=O)cc3F)cn(C)c2n1] | 1-(4-(4-amino-2,7-dimethyl-7H- pyrrolo[2,3-d]pynmidin-5-yl)-3- fluorophenyl)-4-(4-fluorophenyl)-3- methylimidazolidin-2-one | 449.1 | 2.39 (s, 3H), 2.58 (s, 3H), 3.60 (t, J = 8.0 Hz, 1H), 3.68 (s, 3H), 4.24 (t, J = 9.6 Hz, 1H), 4.74 (t, J = 8.0 Hz, 1H), 5.84 (br. s., 2H), 7.14 (s, 1H), 7.25 (t, J = 8.8 Hz, 2H), 7.31 (t, J = 8.8 Hz, 1H), 7.39-7.46 (m, 3H), 7.68-7.71 (m, 1H). |
| 70 | 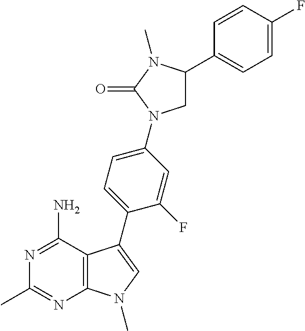 [SMILES: Cc1nc(N)c2c(-c3ccc(N4CC(c5ccc(F)cc5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(4-fluorophenyl)-3- methylimidazolidin-2-one (enantiomer 1) | 435.1 | 2.39 (s, 3H), 2.58 (s, 3H), 3.60 (t, J = 8.0 Hz, 1H), 3.68 (s, 3H), 4.25 (t, J = 9.6 Hz, 1H), 4.74 (t, J = 8.0 Hz, 1H), 5.85 (br. s., 2H), 7.15 (s, 1H), 7.25 (t, J = 8.8 Hz, 2H), 7.31 (t, J = 9.2 Hz, 1H), 7.40-7.46 (m, 3H), 7.68-7.71 (m, 1H). HPLC Column: Chiralpak IA (250 mm × 4.6 mm × 5 μm) Mobile phase: MtBe:IPA with 0.1% DEA (85:15) Flow rate: 1.0 mL/min Retention time: 13.490 min |
| 71 | 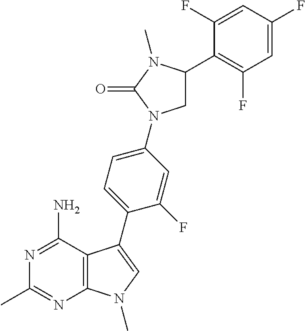 [SMILES: Cc1nc(N)c2c(-c3ccc(N4CC(c5c(F)cc(F)cc5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-3-methyl-4-(2,4,6- trifluorophenyl)imidazolidin-2-one (enantiomer 1) | 485.1 | 2.39 (s, 3H), 2.62 (s, 3H), 3.68 (s, 3H), 3.84-3.87 (m, 1H), 4.27-4.32 (m, 1H), 5.15-5.19 (m, 1H), 5.86 (br. s., 2H), 7.15 (s, 1H), 7.26-7.34 (m, 3H), 7.38-7.40 (m, 1H), 7.70 (d, J = 13.2 Hz, 1H). HPLC Column: CHIRALPAK IA (250 mm × 4.6 mm × 5 μm) Mobile phase A: MTBE Mobile phase B: 0.1% DEA in IPA Composition: 80:20 Flow rate: 1.0 mL/min Retention time: 10.282 min |
| 72 | 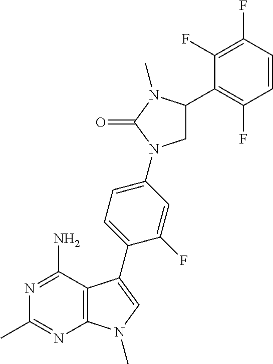 [SMILES: Cc1nc(N)c2c(-c3ccc(N4CC(c5c(F)ccc(F)c5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-3-methyl-4-(2,3,6- trifluorophenyl)imidazolidin-2-one | 485.1 | 2.39 (s, 3H), 2.64 (s, 3H), 3.68 (s, 3H), 3.88-3.92 (m, 1H), 4.32 (t, J = 10.0 Hz, 1H), 5.21-5.25 (m, 1H), 5.86 (br. s, 2H), 7.15 (s, 1H), 7.20-7.24 (m, 1H), 7.32 (t, J = 8.8 Hz, 1H), 7.37-7.40 (m, 1H), 7.52-7.60 (m, 1H), 7.67-7.70 (m, 1H). |
| 73 | 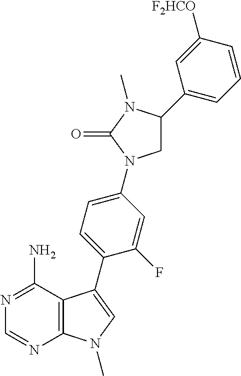 [SMILES: CN1C(=O)N(c2ccc(-c3cn(C)c4ncnc(N)c34)c(F)c2)CC1c1cccc(OC(F)F)c1] | 1-(4-(4-amino-7-methyl-7H- pyrrolo[2,3-d]pyrimidin-5-yl)-3- fluorophenyl)-4-(3- (difluoromethoxy)phenyl)-3- methylimidazolidin-2-one | 482.4 | 2.64 (s, 3H), 3.59-3.63 (m, 1H), 3.71 (s, 3H), 4.26 (t, J = 9.2 Hz, 1H), 4.74-4.78 (m, 1H), 5.92 (br. s., 2H), 7.21-7.16 (m, 2H), 7.24 (t, J = 66.0 Hz, 1H), 7.26 (t, J = 4.0 Hz, 2H), 7.32 (t, J = 8.8 Hz, 1H), 7.41 (dd, J = 2.0, 8.8 Hz, 1H), 7.48 (t, J = 8.0 Hz, 1H), 7.69 (dd, J = 2.4, 13.6 Hz, 8.12 (s, 1H). |
| 74 | 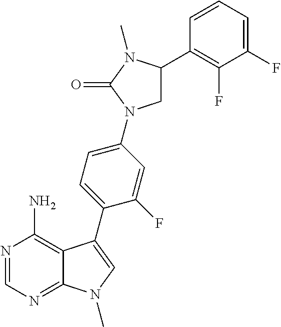 [SMILES: CN1C(=O)N(c2ccc(-c3cn(C)c4ncnc(N)c34)c(F)c2)CC1c1cccc(F)c1F] | 1-(4-(4-amino-7-methyl-7H- pyrrolo[2,3-d]pyrimidin-5-yl)-3- fluorophenyl)-4-(2,3-difluorophenyl)- 3-methylimidazolidin-2-one | 452.43 | 2.66 (s, 3H), 3.71 (s, 3H), 3.75-3.73 (m, 1H), 4.31 (t, J = 9.6 Hz, 1H), 5.05-5.09 (m, 1H), 5.93 (br. S., 2H), 7.19-7.34 (m, 4H), 7.40-7.47 (m, 2H), 7.69 (dd, J = 2.0, 13.2 Hz, 1H), 8.11 (s, 1H). |
| 75 | 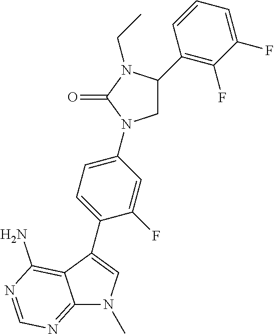 [SMILES: CCN1C(=O)N(c2ccc(-c3cn(C)c4ncnc(N)c34)c(F)c2)CC1c1cccc(F)c1F] | 1-(4-(4-amino-7-methyl-7H- pyrrolo[2,3-d]pyrimidin-5-yl)-3- fluorophenyl)-4-(2,3-difluorophenyl)- 3-ethylimidazolidin-2-one | 466.46 | 0.97 (t, J = 7.2 Hz, 3H), 2.75-2.80 (m, 1H), 3.41-3.46 (m, 1H), 3.71 (s, 3H), 3.76 (q, J = 6.4 Hz, 1H), 4.31 (t, J = 9.6 Hz, 1H), 5.18 (q, J = 6.0 Hz, 1H), 5.93 (br. s., 2H), 7.24-7.28 (m, 3H), 7.32 (t, J = 8.8 Hz, 1H), 7.40-7.48 (m, 2H), 7.69 (dd, J = 1.6, 13.2 Hz, 1H), 8.11 (s, 1H). |
| 76 | 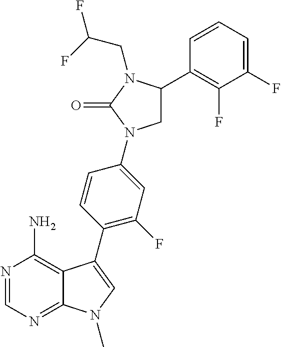 [SMILES: Cn1cc(-c2ccc(N3CC(c4cccc(F)c4F)N(CC(F)F)C3=O)cc2F)c2c(N)ncnc21] | 1-(4-(4-amino-7-methyl-7H- pyrrolo[2,3-d]pyrimidin-5-yl)-3- fluorophenyl)-3-(2,2-difluoroethyl)- 4-(2,4-difluorophenyl)imidazolidin-2- one | 502.44 | 3.08-3.18 (m, 1H), 3.72 (s, 3H), 3.77-3.84 (m, 2H), 4.36 (t, J = 10.4 Hz, 1H), 5.18 (q, J = 5.6 Hz, 1H), 5.94 (br. s., 2H), 6.09 (t, J = 55.6 Hz, 1H), 7.15 (t, J = 6.8 Hz, 1H), 7.25 (s, 1H), 7.33 (q, J = 8.8 Hz, 2H), 7.43-7.50 (m, 2H), 7.68 (d, J = 12.8 Hz, 1H), 8.12 (s, 1H). |
| 77 | 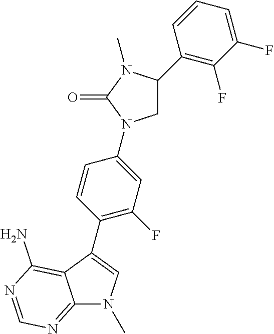 [SMILES: CN1C(=O)N(c2ccc(-c3cn(C)c4ncnc(N)c34)c(F)c2)CC1c1cccc(F)c1F] | 1-(4-(4-amino-7-methyl-7H- pyrrolo[2,3-d]pyrimidin-5-yl)-3- fluorophenyl)-4-(2,3-difluorophenyl)- 3-methylimidazolidin-2-one (enantiomer 1) | 452.43 | 2.66 (s, 3H), 3.75-3.73 (m, 1H), 3.71 (s, 3H), 4.32 (t, J = 9.6 Hz, 1H), 5.05-5.09 (m, 1H), 5.93 (br. s., 2H), 7.22-7.29 (m, 3H), 7.32 (t, J = 8.4 Hz, 1H), 7.41-7.47 (m, 2H), 7.69 (d, J = 13.2 Hz, 1H), 8.12 (s, 1H). HPLC Column: CHIRALPAK IA (250 mm × 4.6 mm × 5 μm) Mobile phase: 0.1% DEA in 100% MEOH Flow rate: 0.8 mL/min Retention time: 13.686 min |
| 78 | 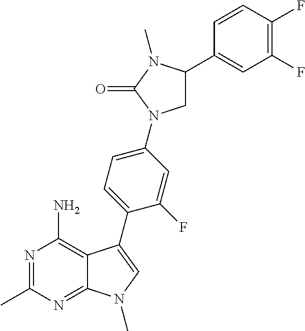 [SMILES: Cc1nc(N)c2c(-c3ccc(N4CC(c5ccc(F)c(F)c5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(3,4-difluorophenyl)- 3-methylimidazolidin-2-one | 466.46 | 2.39 (s, 3H), 2.60 (s, 3H), 3.59-3.64 (m, 1H), 3.68 (s, 3H), 4.24 (t, J = 9.2 Hz, 1H), 4.75 (t, J = 8.0 Hz, 1H), 5.84 (br. s., 2H), 7.15 (s, 1H), 7.27-7.33 (m, 2H), 7.38-7.41 (m, 1H), 7.45-7.54 (m, 2H), 7.66-7.70 (m, 1H), |
| 79 | 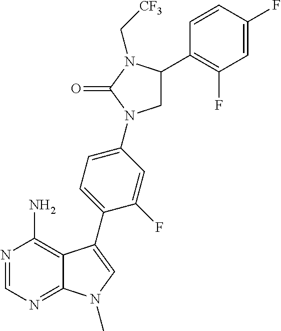 [SMILES: Cn1cc(-c2ccc(N3CC(c4ccc(F)cc4F)N(CC(F)(F)F)C3=O)cc2F)c2c(N)ncnc21] | 1-(4-(4-amino-7-methyl-7H- pyrrolo[2,3-d]pyrimidin-5-yl)-3- fluorophenyl)-4-(2,4-difluorophenyl)- 3-(2,2,2-trifluoroethyl)imidazolidin- 2-one | 520.43 | 3.57-3.63 (m, 1H), 3.72 (s, 3H), 3.86-3.89 (m, 1H), 4.11-4.18 (m, 1H), 4.38-4.43 (m, 1H), 5.15-5.19 (m, 1H), 5.96 (br. s., 2H), 7.14-7.18 (m, 1H), 7.27 (s, 1H), 7.31-7.39 (m, 2H), 7.45-7.53 (m, 2H), 7.67-7.71 (m, 1H), 8.13 (s, 1H). |
| 80 | 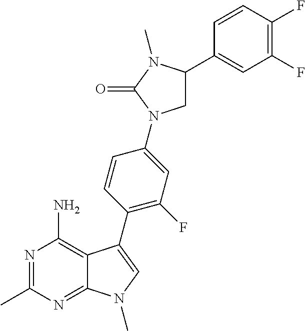 [SMILES: Cc1nc(N)c2c(-c3ccc(N4CC(c5ccc(F)c(F)c5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(3,4-difluorophenyl)- 3-methylimidazolidin-2-one (enantiomer 1) | 466.46 | 2.38 (s, 3H), 2.59 (s, 3H), 3.61 (t, J = 8.8 Hz, 1H), 3.68 (s, 3H), 4.23 (t, J = 9.2 Hz, 1H), 4.74 (t, J = 8.4 Hz, 1H), 5.87 (br. s., 2H), 7.15 (s, 1H), 7.24-7.29 (m, 1H), 7.32 (d, J = 9.2 Hz, 1H), 7.39 (t, J = 8.8 Hz, 1H), 7.45-7.54 (m, 2H), 7.68 (d, J = 13.2 Hz, 1H). HPLC Column: CHIRALPAK IC (250 mm × 4.6 mm × 5 μm) Mobile phase: MTBE:IPA with 0.1% DEA (80:20) Flow rate: 1.0 mL/min Retention time: 12.249 min |
| 81 | 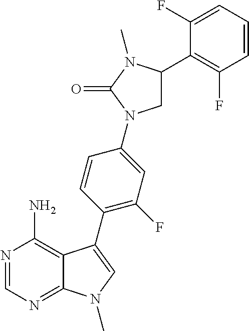 [SMILES: CN1C(=O)N(c2ccc(-c3cn(C)c4ncnc(N)c34)c(F)c2)CC1c1c(F)cccc1F] | 1-(4-(4-amino-7-methyl-7H- pyrrolo[2,3-d]pyrimidin-5-yl)-3- fluorophenyl)-4-(2,6-difluorophenyl)- 3-methylimidazolidin-2-one | 452.43 | 2.61 (s, 3H), 3.72 (s, 3H), 3.84-3.88 (m, 1H), 4.31 (t, J = 9.6 Hz, 1H), 5.20-5.24 (m, 1H), 5.97 (br. s., 2H), 7.17 (t, J = 9.2 Hz, 2H), 7.25 (s, 1H), 7.33 (t, J = 8.4 Hz, 1H), 7.41 (d, J = 8.4 Hz, 1H), 7.46-7.53 (m, 1H), 7.71 (d, J = 13.2 Hz, 1H), 8.12 (s, 1H). |
| 82 | 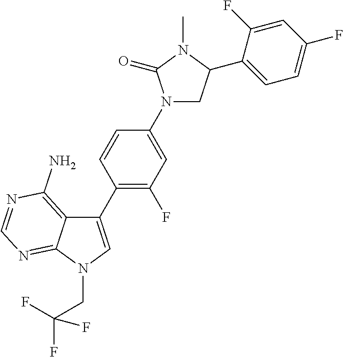 [SMILES: CN1C(=O)N(c2ccc(-c3cn(CC(F)(F)F)c4ncnc(N)c34)c(F)c2)CC1c1ccc(F)cc1F] | 1-(4-(4-amino-7-(2,2,2- trifluoroethyl)-7H-pyrrolo[2,3- d]pyrimidin-5-yl)-3-fluorophenyl)-4- (2,4-difluorophenyl)-3- methylimidazolidin-2-one | 520.43 | 2.63 (s, 3H), 3.67-3.71 (m, 1H), 4.29 (t, J = 9.6 Hz, 1H), 4.98-5.02 (m, 1H), 5.05-5.11 (m, 2H), 6.13 (br. s., 2H), 7.16 (t, J = 8.0 Hz, 1H), 7.32-7.37 (m, 3H), 7.43-7.50 (m, 2H), 7.71 (d, J = 12.8 Hz, 1H), 8.17 (s, 1H). |
| 83 | 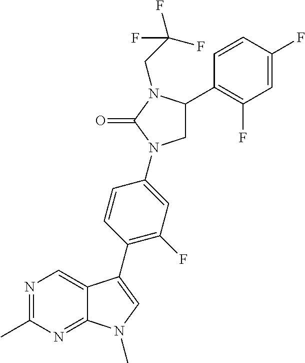 [SMILES: Cc1ncc2c(-c3ccc(N4CC(c5ccc(F)cc5F)N(CC(F)(F)F)C4=O)cc3F)cn(C)c2n1] | 1-(4-(4-amino-2,7-dimethyl-7H- pyrrolo[2,3-d]pyrimidin-5-yl)-3- fluorophenyl)-4-(2,4-difluorophenyl)- 3-trifluoroethyl)imidazolidin-2-one (enantiomer 1) | 534.46 | 3.68 (s, 3H), 2.39 (s, 3H), 3.56-3.63 (m, 1H), 3.85-3.88 (m, 1H), 4.10-4.17 (m, 1H), 4.40 (t, J = 10.0 Hz, 1H), 5.15-5.18 (m, 1H), 5.90 (br. s., 2H), 7.14-7.17 (m, 2H), 7.32-7.37 (m, 2H), 7.43-7.52 (m, 2H), 7.68 (d, J = 12.8 Hz, 1H). HPLC Column: CHIRALPAK IA (250 mm × 4.6 mm × 5 μM) Mobile phase: n- Hexane:Ethanol with 0.1% DEA (65:35) Flow: 1.0 ml/min Retention time: 13.020 min |
| 84 | 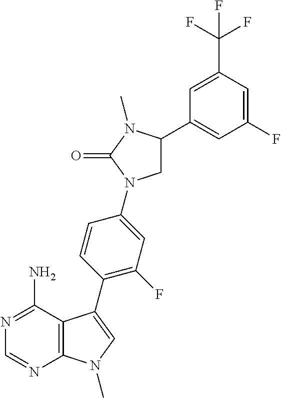 [SMILES: CN1C(=O)N(c2ccc(-c3cn(C)c4ncnc(N)c34)c(F)c2)CC1c1cc(F)cc(C(F)(F)F)c1] | 1-(4-(4-amino-7-methyl-7H- pyrrolo[2,3-d]pyrimidin-5-yl)-3- fluorophenyl)-4-(3-fluoro-5- (trifluoromethyl)phenyl)-3- methylimidazolidin-2-one | 503.1 | 2.64 (s, 3H), 3.65-3.69 (m, 1H), 3.71 (s, 3H), 4.27 (t, J = 9.4 Hz, 1H), 4.89 (t, J = 8.4 Hz, 1H), 5.95 (br. S., 2H), 7.24 (s, 1H), 7.32 (t, J = 8.8 Hz, 1H), 7.41-7.42 (m, 1H), 7.63 (d, J = 9.2 Hz, 1H), 7.67-7.70 (m, 3H), 8.12 (s, 1H). |
| 85 | 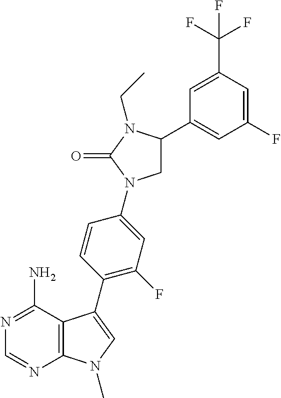 [SMILES: CCN1C(=O)N(c2ccc(-c3cn(C)c4ncnc(N)c34)c(F)c2)CC1c1cc(F)cc(C(F)(F)F)c1] | 1-(4-(4-amino-7-methyl-7H- pyrrolo[2,3-d]pyrimidin-5-yl)-3- fluorophenyl)-3-ethyl-4-(3-fluoro-5- (trifluoromethyl)phenyl)imidazolidin- 2-one | 517.1 | 0.95 (t, J = 6.8 Hz, 3H), 2.71-2.78 (m, 1H), 3.35-3.44 (m, 1H), 3.67-3.69 (m, 1H), 3.71 (s, 3H), 4.26 (t, J = 9.4 Hz, 1H), 5.05 (t, J = 8.2 Hz, 1H), 5.91 (br. S., 2H), 7.24 (s, 1H), 7.32 (t, J = 8.6 Hz, 1H), 7.43 (d, J = 8.4 Hz, 1H), 7.65-7.70 (m, 4H), 8.12 (s, 1H). |
| 86 | 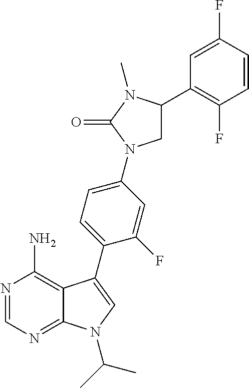 [SMILES: CC(C)n1cc(-c2ccc(N3CC(c4cc(F)ccc4F)N(C)C3=O)cc2F)c2c(N)ncnc21] | 1-(4-(4-amino-7-isopropyl-7H- pyrrolo[2,3-d]pyrimidin-5-yl)-3- fluorophenyl)-4-(2,5-difluorophenyl)- 3-methylimidazolidin-2-one (enantiomer 1) | 481.2 | 1.43 (d, J = 6.8 Hz, 6H), 2.65 (s, 3H), 3.68-3.72 (m, 1H), 4.30-4.32 (m, 1H), 4.92-5.02 (m, 2H), 5.90 (br. S., 2H), 7.27-7.31 (m, 2H), 7.34-7.37 (m, 3H), 7.39 (d, J = 13.2 Hz, 1H), 7.69 (d, J = 12.8 Hz, 1H), 8.10 (s, 1H). HPLC Column: Chiralpak IA (250 mm × 4.6 mm × 5 μm) Mobile phase: MTBE:IPA with 0.1% DEA (30:70) Flow rate: 1.0 mL/min Retention time: 16.405 min |
| 87 | 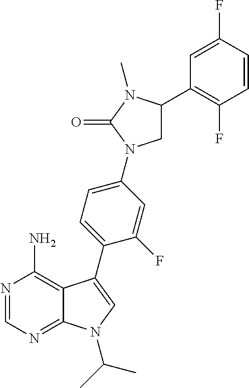 [SMILES: CC(C)n1cc(-c2ccc(N3CC(c4cc(F)ccc4F)N(C)C3=O)cc2F)c2c(N)ncnc21] | 1-(4-(4-amino-7-isopropyl-7H- pyrrolo[2,3-d]pyrimidin-5-yl)-3- fluorophenyl)-4-(2,5-difluorophenyl)- 3-methylimidazolidin-2-one (enantiomer 2) | 481.2 | 1.43 (d, J = 6.8 Hz, 6H), 2.65 (s, 3H), 3.68-3.72 (m, 1H), 4.30 (t, J = 9.6 Hz-1H), 4.92-4.95 (m, 1H), 4.98-5.02 (m, 1H), 5.89 (br. S., 2H), 7.25-7.29 (m, 2H), 7.35-7.40 (m, 3H), 7.42-7.43 (m, 1H), 7.67-7.71 (m, 1H), 8.10 (s, 1H). HPLC Column: Chiralpak IA (250 mm × 4.6 mm × 5 μm) Mobile phase: MTBE:IPA with 0.1% DEA (30:70) Flow rate: 1.0 mL/min Retention time: 20.205 min |
| 88 | 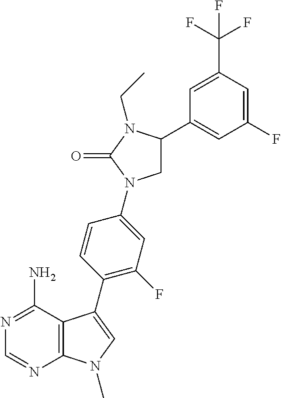 [SMILES: CCN1C(=O)N(c2ccc(-c3cn(C)c4ncnc(N)c34)c(F)c2)CC1c1cc(F)cc(C(F)(F)F)c1] | 1-(4-(4-amino-7-methyl-7H- pyrrolo[2,3-d]pyrimidin-5-yl)-3- fluorophenyl)-3-ethyl-4-(3-fluoro-5- (trifluoromethyl)phenyl)imidazolidin- 2-one (enantiomer 1) | 517.1 | 1.03 (t, J = 7.0 Hz, 3H), 2.73-2.79 (m, 1H), 3.37-3.44 (m, 1H), 3.67-3.69 (m, 1H), 3.72 (s, 3H), 4.27 (t, J = 9.6 Hz, 1H), 5.05 (t, J = 8.4 Hz, 1H), 5.92 (br. S., 2H), 7.25 (s, 1H), 7.32 (t, J = 8.6 Hz, 1H), 7.41 (d, J = 8.4 Hz, 1H), 7.65-7.71 (m, 4H), 8.12 (s, 1H). HPLC Column: CHIRALPAK IC (250 mm × 4.6 mm × 5 μM) Mobile phase (A:B): n- Hexane:Ethanol with 0.1% % TFA (60:40) Flow: 1.0 ml/min Retention time: 18.517 min |
| 89 | 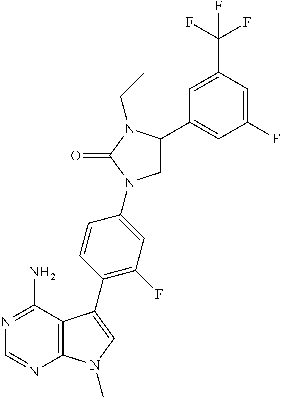 [SMILES: CCN1C(=O)N(c2ccc(-c3cn(C)c4ncnc(N)c34)c(F)c2)CC1c1cc(F)cc(C(F)(F)F)c1] | 1-(4-(4-amino-7-methyl-7H- pyrrolo[2,3-d]pyrimidin-5-yl)-3- fluorophenyl)-3-ethyl-4-(3-fluoro-5- (trifluoromethyl)phenyl)imidazolidin- 2-one (enantiomer 2) | 517.1 | 0.96 (t, J = 7.0 Hz, 3H), 2.75-2.77 (m, 1H), 3.39-3.42 (m, 1H), 3.67-3.69 (m, 1H), 3.72 (s, 3H), 4.27 (t, J = 9.4 Hz, 1H), 5.05 (t, J = 8.4 Hz, 1H), 5.92 (br. S., 2H), 7.25 (s, 1H), 7.32 (t, J = 8.6 Hz, 1H), 7.41 (d, J = 8.4 Hz, 1H), 7.65-7.71 (m, 4H), 8.12 (s, 1H). HPLC Column: CHIRALPAK IC (250 mm × 4.6 mm × 5 mic) Mobile phase (A:B): n- Hexane:Ethanol with 0.1% % TFA (60:40) Flow: 1.0 ml/min Retention time: 29.007 min |
| 90 | 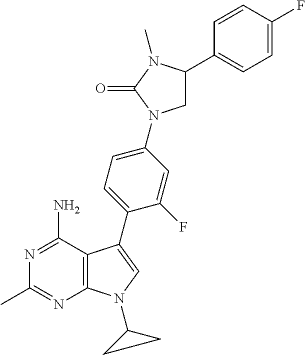 [SMILES: Cc1nc(N)c2c(-c3ccc(N4CC(c5ccc(F)cc5)N(C)C4=O)cc3F)cn(C3CC3)c2n1] | 1-(4-(4-amino-7-cyclopropyl-2- methyl-7H-pyrrolo[2,3-d]pyrimidin- 5-yl)-3-fluorophenyl)-4-(4- fluorophenyl)-3-methylimidazolidin- 2-one (enantiomer 1) | 475.2 | 0.95-1.05 (s, 4H), 2.31 (s, 3H), 2.58 (s, 3H), 3.54-3.62 (m, 2H), 4.25 (t, J = 8.2 Hz, 1H), 4.74 (t, J = 8.0 Hz, 1H), 5.83 (br. s., 2H), 7.07 (s, 1H), 7.23-7.33 (m, 3H), 7.39-7.44 (m, 3H), 7.69 (d, J = 12.8 Hz, 1H). HPLC Column: Chiralpak IA (250 mm × 4.6 mm × 5 mic) Mobile phase: MTBE:IPA with 0.1% DEA (70:30) Flow rate 1.0 mL/min Retention time: 10.110 min |
| 91 | 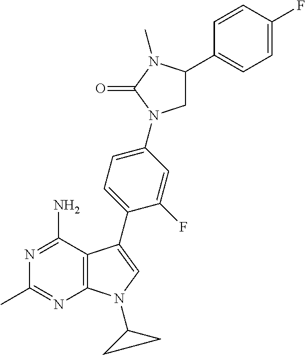 [SMILES: Cc1nc(N)c2c(-c3ccc(N4CC(c5ccc(F)cc5)N(C)C4=O)cc3F)cn(C3CC3)c2n1] | 1-(4-(4-amino-7-cyclopropyl-2- methyl-7H-pyrrolo[2,3-d]pyrimidin- 5-yl)-3-fluorophenyl)-4-(4- fluorophenyl)-3-methylimidazolidin- 2-one | 475.2 | 0.98-1.03 (s, 4H), 2.31 (s, 3H), 2.58 (s, 3H), 3.53-3.61 (m, 2H), 4.25 (t, J = 8.2 Hz, 1H), 4.74 (t, J = 8.0 Hz, 1H), 5.82 (br. s, 2H), 7.07 (s, 1H), 7.23-7.33 (m, 3H), 7.39-7.46 (m, 3H), 7.69 (d, J = 12.8 Hz, 1H) |
| 92 | 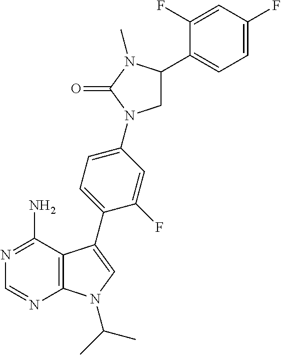 [SMILES: CC(C)n1cc(-c2ccc(N3CC(c4ccc(F)cc4F)N(C)C3=O)cc2F)c2c(N)ncnc21] | 1-(4-(4-amino-7-isopropyl-7H- pyrrolo[2,3-d]pyrimidin-5-yl)-3- fluorophenyl)-4-(2,4-difluorophenyl)- 3-methylimidazolidin-2-one | 481.2 | 1.43 (d, J = 6.4 Hz, 6H), 2.63 (s, 3H), 3.67-3.71 (m, 1H), 3.69 (t, J = 9.8 Hz, 1H), 4.92-5.02 (m, 2H), 5.90 (br. s., 2H), 7.15 (t, J = 8.0 Hz, 1H), 7.30-7.37 (m, 3H), 7.40-7.42 (m, 1H), 7.44-7.50 (m, 1H), 7.70 (d, J = 12.0 Hz, 1H), 8.10 (s, 1H) |
| 93 | 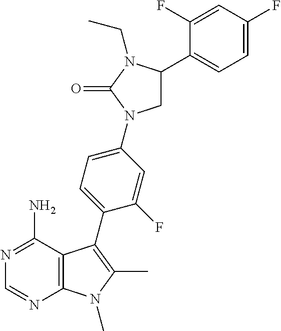 [SMILES: CCN1C(=O)N(c2ccc(-c3c(C)n(C)c4ncnc(N)c34)c(F)c2)CC1c1ccc(F)cc1F] | 1-(4-(4-amino-6,7-dimethyl-7H- pyrrolo[2,3-d]pyrimidin-5-yl)-3- fluorophenyl)-4-(2,4-difluorophenyl)- 3-ethylimidazolidin-2-one | 481.2 | 0.96 (t, J = 7.0 Hz, 3H), 2.19 (s, 3H), 2.72-2.78 (m, 1H), 3.37-3.44 (m, 1H), 3.66 (s, 3H), 3.67-3.74 (m, 1H), 4.29 (t, J = 9.6 Hz, 1H), 5.10-5.14 (m, 1H), 5.65 (br. s, 2H), 7.15 (t, J = 8.0 Hz, 1H), 7.24-7.34 (m, 2H), 7.44 (d, J = 8.4 Hz, 1H), 7.53 (q, J = 8.0 Hz, 1H), 7.70 (d, J = 12.8 Hz, 1H), 8.06 (s, 1H) |
| 94 | 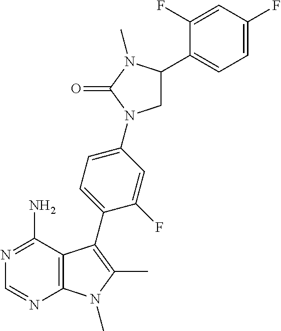 [SMILES: Cc1c(-c2ccc(N3CC(c4ccc(F)cc4F)N(C)C3=O)cc2F)c2c(N)ncnc2n1C] | 1-(4-(4-amino-6,7-dimethyl-7H- pyrrolo[2,3-d]pyrimidin-5-yl)-3- fluorophenyl)-4-(2,4-difluorophenyl)- 3-methylimidazolidin-2-one | 467.3 | 2.19 (s, 3H), 2.63 (s, 3H), 3.66-3.70 (m, 4H), 4.28-4.32 (m, 1H), 4.98-5.02 (m, 1H), 5.65 (br. s., 2H), 7.15 (t, J = 7.8 Hz, 1H), 7.25-7.35 (m, 2H), 7.42-7.51 (m, 2H), 7.67-7.72 (m, 1H), 8.06 (s, 1H) |
| 95 | 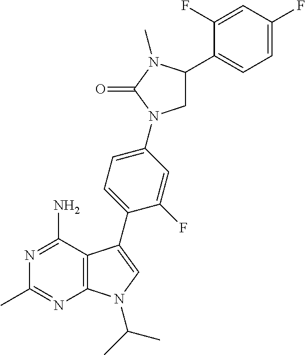 [SMILES: Cc1nc(N)c2c(-c3ccc(N4CC(c5ccc(F)cc5F)N(C)C4=O)cc3F)cn(C(C)C)c2n1] | 1-(4-(4-amino-7-isopropyl-2-methyl- 7H-pyrrolo[2,3-d]pyrimidin-5-yl)-3- fluorophenyl)-4-(2,4-difluorophenyl)- 3-methylimidazolidin-2-one (enantiomer 1) | 495.2 | 1.41 (d, J = 8.2 Hz, 6H), 2.38 (s, 3H), 2.63 (s, 3H), 3.27-3.70 (m, 1H), 4.28 (t, J = 8.0 Hz, 1H), 4.93-4.99 (m, 2H), 5.81 (br. s., 2H), 7.15 (t, J = 8.0 Hz, 1H), 7.27-7.34 (m, 3H), 7.40 (d, J = 8.0 Hz, 1H), 7.44-7.50 (m, 1H), 7.69 (d, J = 13.2 Hz, 1H). HPLC Column: CHIRALPAK IA (250 mm × 4.6 mm × 5 mic) Mobile phase: 0.1% DEA in Ethanol (100%) Flow rate: 0.5 mL/min Retention time: 17.621 min |
| 96 | 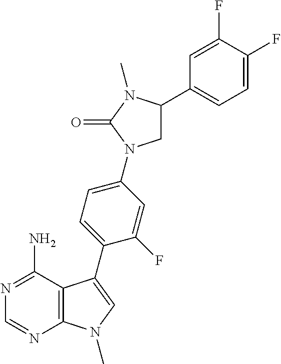 [SMILES: CN1C(=O)N(c2ccc(-c3cn(C)c4ncnc(N)c34)c(F)c2)CC1c1ccc(F)c(F)c1] | 1-(4-(4-amino-7-methyl-7H- pyrrolo[2,3-d]pyrimidin-5-yl)-3- fluorophenyl)-4-(3,4-difluorophenyl)- 3-methylimidazolidin-2-one | 453.2 | 2.60 (s, 3H), 3.62 (t, J = 8.0 Hz, 1H), 3.72 (s, 3H), 4.24 (t, J = 9.4 Hz, 1H), 4.75 (t, J = 8.0 Hz, 1H), 5.92 (br. s., 2H), 7.24-7.29 (m, 2H), 7.30-7.35 (m, 1H), 7.40 (d, J = 8.5 Hz, 1H), 7.45-7.54 (m, 2H), 7.69 (d, J = 13.2 Hz, 1H), 8.12 (s, 1H) |
| 97 | 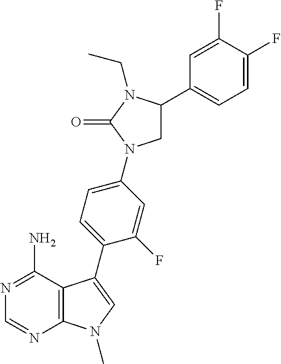 [SMILES: CCN1C(=O)N(c2ccc(-c3cn(C)c4ncnc(N)c34)c(F)c2)CC1c1ccc(F)c(F)c1] | 1-(4-(4-amino-7-methyl-7H- pyrrolo[2,3-d]pyrimidin-5-yl)-3- fluorophenyl)-4-(3,4-difluorophenyl)- 3-ethylimidazolidin-2-one | 467.1 | 0.95 (t, J = 8.0 Hz, 3H), 2.70-2.76 (m, 1H), 3.37-3.42 (m, 1H), 3.64 (t, J = 8.0 Hz, 1H), 3.72 (s, 3H), 4.23 (t, J = 9.5 Hz, 1H), 4.90 (t, J = 8.0 Hz, 1H), 5.92 (br. s., 2H), 7.24 (s, 1H), 7.30-7.34 (m, 2H), 7.41 (d, J = 8.0 Hz, 1H), 7.44-7.49 (m, 1H), 7.55 (t, J = 9.6 Hz, 1H), 7.69 (d, J = 12.0 Hz, 1H), 8.12 (s, 1H) |
| 98 | 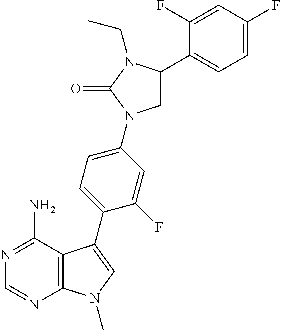 [SMILES: CCN1C(=O)N(c2ccc(-c3cn(C)c4ncnc(N)c34)c(F)c2)CC1c1ccc(F)cc1F] | 1-(4-(4-amino-1-methyl-1H- pyrazolo[3,4-d]pyrimidin-3-yl)-3- fluorophenyl)-4-(2,4-difluorophenyl)- 3-ethylimidazolidin-2-one | 468.2 | 0.97 (t, J = 8.0 Hz, 3H), 2.74-2.79 (m, 1H), 3.40-3.45 (m, 1H), 3.72-3.76 (m, 1H), 3.92 (s, 3H), 4.31 (t, J = 8.1 Hz, 1H), 5.12-5.15 (m, 1H), 6.00-7.14 (br. s., 2H), 7.15 (t, J = 8.2 Hz, 1H), 7.30-7.35 (m, 1H), 7.43-7.54 (m, 3H), 7.73 (d, J = 13.2 Hz, 1H), 8.21 (s, 1H) |
| 99 | 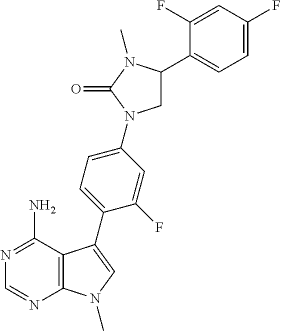 [SMILES: CN1C(=O)N(c2ccc(-c3cn(C)c4ncnc(N)c34)c(F)c2)CC1c1ccc(F)cc1F] | 1-(4-(4-amino-1-methyl-1H- pyrazolo[3,4-d]pyrimidin-3-yl)-3- fluorophenyl)-4-(2,4-difluorophenyl)- 3-methylimidazolidin-2-one | 454.3 | 2.64 (s, 3H, ) 3.69-3.73 (m, 1H), 3.92 (s, 3H), 4.31 (t, J = 8.1 Hz, 1H), 5.00-5.04 (m, 1H), 6.32-7.12 (br. s., 2H), 7.14-7.18 (m, 1H), 7.30-7.36 (m, 1H), 7.44-7.50 (m, 3H), 7.73 (d, J = 13.2 Hz, 1H), 8.21 (s, 1H) |
| 101 | 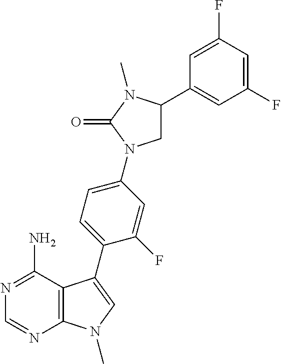 [SMILES: CN1C(=O)N(c2ccc(-c3cn(C)c4ncnc(N)c34)c(F)c2)CC1c1cc(F)cc(F)c1] | 1-(4-(4-amino-7-methyl-7H- pyrrolo[2,3-d]pyrimidin-5-yl)-3- fluorophenyl)-4-(3,5-difluorophenyl)- 3-methylimidazolidin-2-one (enantiomer 2) | 453.1 | 2.63 (s, 3H), 3.63 (t, J = 8.2 Hz,1H ), 3.72 (s, 3H), 4.25 (t, J = 8.4 Hz, 1H), 4.76 (t, J = 8.2 Hz, 1H), 5.92 (br. s., 2H), 7.16 (d, J = 8.0 Hz, 2H), 7.22-7.25 (m, 2H), 7.33 (t, J = 8.2 Hz, 1H), 7.39-7.41 (m, 1H), 7.68 (d, J = 12.4 Hz, 1H), 8.12 (s, 1H). HPLC Column: CHIRALPAK IA (250 mm × 4.6 mm × 5 mic) Mobile phase: n- Hexane:0.1% DEA in Ethanol (50:50) Flow rate:1.0 ml/min Retention time: 17.243 min |
| 102 | 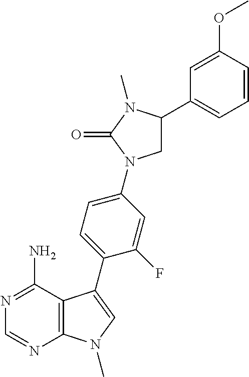 [SMILES: COc1cccc(C2CN(c3ccc(-c4cn(C)c5ncnc(N)c45)c(F)c3)C(=O)N2C)c1] | 1-(4-(4-amino-7-methyl-7H- pyrrolo[2,3-d]pyrimidin-5-yl)-3- fluorophenyl)-4-(3-methoxyphenyl)- 3-methylimidazolidin-2-one | 447.2 | 2.61 (s, 3H), 3.60 (t, J = 8.0 Hz, 1H), 3.72 (s, 3H), 3.76 (s, 3H), 4.22-4.27 (m, 1H), 4.69 (t, J = 8.2 Hz, 1H), 5.93 (br. s., 2H), 6.92-6.94 (m, 3H), 7.24 (s, 1H), 7.33 (q, J = 8.0 Hz, 2H), 7.40-7.43 (m, 1H), 7.71 (d, J = 8.5 Hz, 1H), 8.12 (s, 1H) |
| 103 | 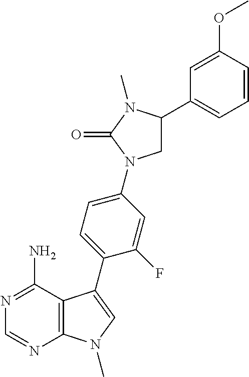 [SMILES: COc1cccc(C2CN(c3ccc(-c4cn(C)c5ncnc(N)c45)c(F)c3)C(=O)N2C)c1] | 1-(4-(4-amino-7-methyl-7H- pyrrolo[2,3-d]pyrimidin-5-yl)-3- fluorophenyl)-4-(3-methoxyphenyl)- 3-methylimidazolidin-2-one (enantiomer 1) | 461.2 | 2.41 (s, 3H), 2.61 (s, 3H), 3.58-3.62 (m, 1H), 3.69 (s, 3H), 3.76 (s, 3H), 4.24 (t, J = 8.0 Hz, 1H), 4.69 (t, J = 8.2 Hz, 1H), 6.01 (br. s, 2H), 6.92-6.94 (m, 3H), 7.18 (s, 1H), 7.29-7.36 (m, 2H), 7.41 (dd, J = 6.8, 1.2 Hz, 1H), 7.69 (dd, J = 11.2, 2.0 Hz, 1H). HPLC Column: chiralpak IA (250 mm × 4.6 mm × 5 mic) Mobile phase: MtBe:IPA with 0.1% DEA (70:30) Flow rate: 1.0 mL/min Retention time: 9.353 min |
| 104 | 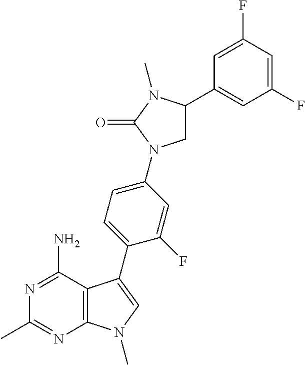 [SMILES: Cc1nc(N)c2c(-c3ccc(N4CC(c5cc(F)cc(F)c5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(3,5-difluorophenyl)- 3-methylimidazolidin-2-one (enantiomer 1) | 467.0 | 2.38 (s, 3H), 2.65 (s, 3H), 3.62 (t, J = 8.2 Hz, 1H), 3.68 (s, 3H), 4.22-4.27 (m, 1H), 4.78 (t, J = 8.0 Hz, 1H), 5.86 (br. s., 2H), 7.15-7.17 (m, 3H), 7.23-7.33 (m, 2H), 7.38-7.40 (m, 1H), 7.66-7.69 (m, 1H). HPLC Column: CHIRALPAK IA (250 mm × 4.6 mm × 5 μm) Mobile phase: n-Hexane 0.1% DEA in Ethanol (70:30) Flow rate: 1.0 mL/min Retention time: 16.144 min |
| 105 | 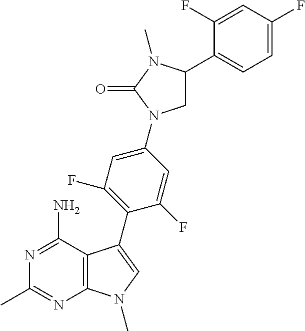 [SMILES: Cc1nc(N)c2c(-c3c(F)cc(N4CC(c5ccc(F)cc5F)N(C)C4=O)cc3F)cn(C)c2n1] | 1-(4-(4-amino-2,7-dimethyl-7H- pyrrolo[2,3-d]pyrimidin-5-yl)-3,5- difluorophenyl)-4-(2,4- difluorophenyl)-3- methylimidazolidin-2-one | 485.1 | 2.38 (s, 3H), 2.63 (s, 3H), 3.71 (s, 4H), 4.25-4.30 (m, 1H), 5.01 (t, J = 8.0 Hz, 1H), 5.85 (br. s., 2H), 7.19-7.20 (m, 2H), 7.26-7.39 (m, 1H), 7.45-7.47 (m, 3H) |
| 106 | 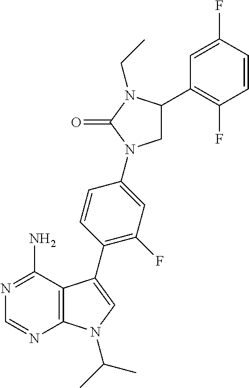 [SMILES: CCN1C(=O)N(c2ccc(-c3cn(C(C)C)c4ncnc(N)c34)c(F)c2)CC1c1cc(F)ccc1F] | 1-(4-(4-amino-7-isopropyl-7H- pyrrolo[2,3-d]pyrimidin-5-yl)-3- fluorophenyl)-4-(2,5-difluorophenyl)- 3-ethylimidazolidin-2-one | 495.2 | 0.97 (t, J = 7.6 Hz, 3H), 1.44 (d, J = 6.4 Hz, 6H), 2.73-2.82 (m, 1H), 3.38-3.46 (m, 1H), 3.72-3.76 (m, 1H), 4.29 (t, J = 9.6 Hz, 1H), 4.90-4.97 (m, 1H), 5.10-5.14 (m, 1H), 5.90 (br. s., 2H), 7.25-7.37 (m, 5H), 7.40-7.42 (m, 1H), 7.68-7.72 (m, 1H), 8.10 (s, 1H). |
| 107 | 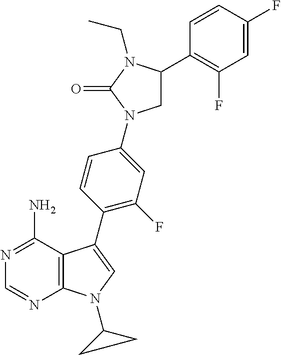 [SMILES: CCN1C(=O)N(c2ccc(-c3cn(C4CC4)c4ncnc(N)c34)c(F)c2)CC1c1ccc(F)cc1F] | 1-(4-(4-amino-7-cyclopropyl-7H- pyrrolo[2,3-d]pyrimidin-5-yl)-3- fluorophenyl)-4-(2,4-difluorophenyl)- 3-ethylimidazolidin-2-one | 493.2 | 0.96 (t, J = 7.2 Hz, 3H), 1.01 (s, 4H), 2.73-2.78 (m, 1H), 3.37-3.44 (m, 1H), 3.50-3.60 (m, 1H), 3.72 (t, J = 6.4 Hz, 1H), 4.28 (t, J = 9.6 Hz, 1H), 5.12 (t, J = 6.4 Hz, 1H), 5.91 (br. s., 2H), 7.13-7.17 (m, 2H), 7.32 (t, J = 8.8 Hz, 2H), 7.40-7.42 (m, 1H), 7.48-7.54 (m, 1H), 7.69 (d, J = 12.8 Hz, 1H), 8.12 (s, 1H). |
| 108 | 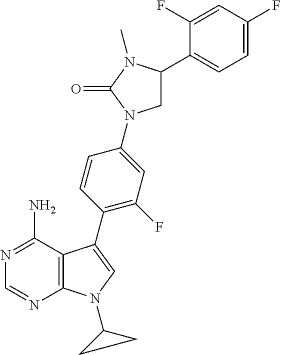 [SMILES: CN1C(=O)N(c2ccc(-c3cn(C4CC4)c4ncnc(N)c34)c(F)c2)CC1c1ccc(F)cc1F] | 1-(4-(4-amino-7-cyclopropyl-7H- pyrrolo[2,3-d]pyrimidin-5-yl)-3- fluorophenyl)-4-(2,4-difluorophenyl)- 3-methylimidazolidin-2-one | 479.2 | 1.01 (s, 4H), 2.63 (s, 3H), 3.53-3.54 (m, 1H), 3.68 (t, J = 9.2 Hz, 1H), 4.29 (t, J = 9.6 Hz, 1H), 4.99 (t, J = 9.6 Hz, 1H), 5.91 (br. s., 2H), 7.10-7.19 (m, 2H), 7.32 (t, J = 8.8 Hz, 2H), 7.40-7.42 (m, 1H), 7.44-7.50 (m, 1H), 7.69 (d, J = 13.6 Hz, 1H), 8.12 (s, 1H). |
| 109 | 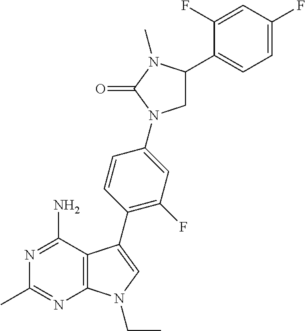 [SMILES: CCn1cc(-c2ccc(N3CC(c4ccc(F)cc4F)N(C)C3=O)cc2F)c2c(N)nc(C)nc21] | 1-(4-(4-amino-7-ethyl-2-methyl-7H- pyrrolo[2,3-d]pyrimidin-5-yl)-3- fluorophenyl)-4-(2,4-difluorophenyl)- 3-methylimidazolidin-2-one | 481.2 | 1.34 (t, J = 7.2 Hz, 3H), 2.38 (s, 3H), 2.63 (s, 3H), 3.69 (t, J = 7.2 Hz, 1H), 4.11-4.17 (m, 2H), 4.28 (t, J = 9.6 Hz, 1H), 5.0 (t, J = 6.8 Hz, 1H), 5.82 (br. s., 2H), 7.15 (t, J = 8 Hz, 1H), 7.21 (s, 1H), 7.29-7.34 (m, 2H), 7.39-7.41 (m, 1H), 7.44-7.50 (m, 1H), 7.69 (d, J = 12.8 Hz, 1H). |
| 111 | 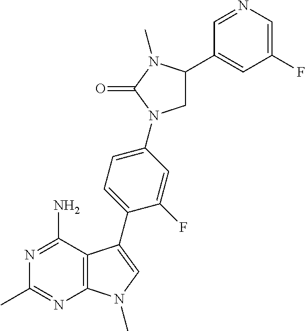 [SMILES: Cc1nc(N)c2c(-c3ccc(N4CC(c5cncc(F)c5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(5-fluoropyridin-3- yl)-3-methylimidazolidin-2-one (enantiomer 1) | 450.2 | 2.39 (s, 3H), 2.64 (s, 3H), 3.68-3.73 (m, 4H), 4.28 (t, J = 8.0 Hz, 1H), 4.86 (t, J = 8.0 Hz, 1H), 5.84 (br. s, 2H), 7.15 (s, 1H), 7.32 (t, J = 8.0 Hz, 1H), 7.39-7.41 (m, 1H), 7.68 (d, J = 12.0 Hz, 1H), 7.82 (d, J = 12.0 Hz, 1H), 8.51 (s, 1H), 8.59- 8.60 (m, 1H). HPLC Column: Chiralpak IA (250 mm × 4.6 mm × 5 mic) Mobile phase (A:B): MTBE:Ethanol with 0.1% DEA (80:30) Flow rate:1.0 mL/min Retention time: 8.264 min |
| 112 | 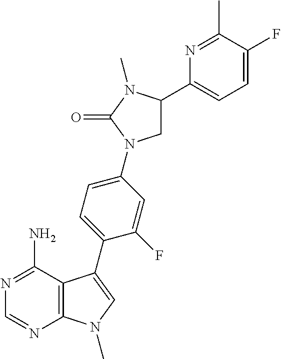 [SMILES: Cc1nc(C2CN(c3ccc(-c4cn(C)c5ncnc(N)c45)c(F)c3)C(=O)N2C)ccc1F] | 1-(4-(4-amino-7-methyl-7H- pyrrolo[2,3-d]pyrimidin-5-yl)-3- fluorophenyl)-4-(5-fluoro-6- methylpyridin-2-yl)-3- methylimidazolidin-2-one | 450.4 | 2.44 (s, 3H), 2.64 (s, 3H), 3.72 (s, 3H), 3.74-3.78 (m, 1H), 4.24 (t, J = 8.2 Hz, 1H), 4.81-4.85 (m, 1H), 5.93 (br. s, 2H), 7.24 (s, 1H), 7.30-7.42 (m, 3H), 7.66-7.73 (m, 2H), 8.12 (s, 1H) |
| 113 | 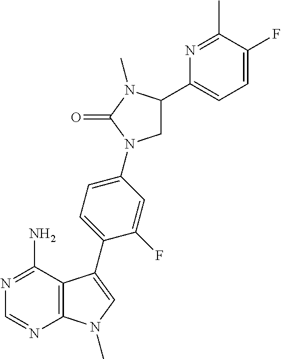 [SMILES: Cc1nc(C2CN(c3ccc(-c4cn(C)c5ncnc(N)c45)c(F)c3)C(=O)N2C)ccc1F] | 1-(4-(4-amino-7-methyl-7H- pyrrolo[2,3-d]pyrimidin-5-yl)-3- fluorophenyl)-4-(5-fluoro-6- methylpyridin-2-yl)-3- methylimidazolidin-2-one (enantiomer 1) | 450.4 | 2.40 (s, 3H), 2.62 (s, 3H), 3.72-3.78 (m, 4H), 4.24 (t, J = 8.0 Hz, 1H), 4.83 (t, J = 8.0 Hz, 1H), 5.93 (br. s, 2H), 7.24 (s, 1H), 7.30-7.42 (m, 3H), 7.67-7.73 (m, 2H), 8.12 (s, 1H). HPLC Column: CHIRALPAK IA (250 mm × 4.6 mm × 5 μm) Mobile phase: n-Hexane: Ethanol with 0.1% TFA (50:50) Flow rate: 1.0 mL/min Retention time: 14.078 min |
| 114 | 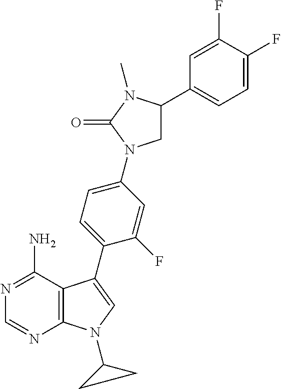 [SMILES: CN1C(=O)N(c2ccc(-c3cn(C4CC4)c4ncnc(N)c34)c(F)c2)CC1c1ccc(F)c(F)c1] | 1-(4-(4-amino-7-cyclopropyl-7H- pyrrolo[2,3-d]pyrimidin-5-yl)-3- fluorophenyl)-4-(3,4-difluorophenyl)- 3-methylimidazolidin-2-one | 479.1 | 0.99-1.01 (m, 4H), 2.60 (s, 3H), 3.27-3.55 (m, 1H), 3.57-3.64 (m, 1H), 4.24 (t, J = 8.0 Hz, 1H), 4.75 (t, J = 8.0 Hz, 1H), 5.91 (br. s, 2H), 7.17 (s, 1H), 7.27 (br, 1H), 7.33 (t, J = 8.2 Hz, 1H), 7.39-7.41 (m, 1H), 7.45-7.54 (m, 2H), 7.67-7.70 (m, 1H), 8.13 (s, 1H) |
| 116 | 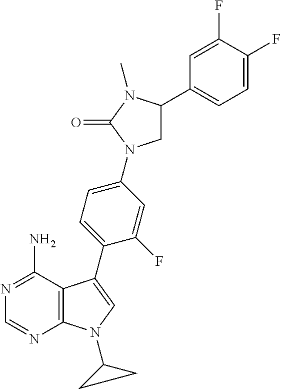 [SMILES: CN1C(=O)N(c2ccc(-c3cn(C4CC4)c4ncnc(N)c34)c(F)c2)CC1c1ccc(F)c(F)c1] | 1-(4-(4-amino-7-cyclopropyl-7H- pyrrolo[2,3-d]pyrimidin-5-yl)-3- fluorophenyl)-4-(3,4-difluorophenyl)- 3-methylimidazolidin-2-one (enantiomer 1) | 479.1 | 0.97-1.01 (m, 4H), 2.60 (s, 3H), 3.52-3.57 (m, 1H), 3.62 (t, J = 8.0 Hz, 1H), 4.24 (t, J = 8.0 Hz, 1H), 4.75 (t, J = 8.0 Hz, 1H), 5.91 (br. s, 2H), 7.17 (s, 1H), 7.27-7.29 (m, 1H), 7.33 (t, J = 8.2 Hz, 1H), 7.40 (dd, J = 2.0, 8.4 Hz, 1H), 7.45-7.54 (m, 2H), 7.68 (dd, J = 1.6, 13.2 Hz, 1H), 8.13 (s, 1H). HPLC Column: CHIRALPAK IC (250 mm × 4.6 mm × 5 mic) Mobile phase: MTBE:Ethanol with 0.1% DEA (80:20) Flow rate: 1.0 mL/min Retention time: 10.702 min |
| 117 | 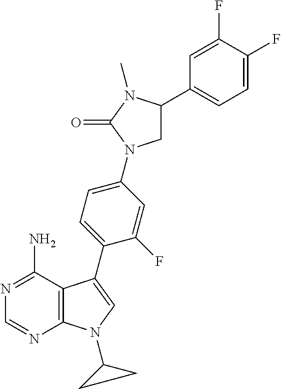 [SMILES: CN1C(=O)N(c2ccc(-c3cn(C4CC4)c4ncnc(N)c34)c(F)c2)CC1c1ccc(F)c(F)c1] | 1-(4-(4-amino-7-cyclopropyl-7H- pyrrolo[2,3-d]pyrimidin-5-yl)-3- fluorophenyl)-4-(3,4-difluorophenyl)- 3-methylimidazolidin-2-one (enantiomer 2) | 479.1 | 0.99-1.01 (m, 4H), 2.60 (s, 3H), 3.53-3.55 (m, 1H), 3.62 (t, J = 8.0 Hz, 1H), 4.24 (t, J = 8.0 Hz, 1H), 4.75 (t, J = 8.0 Hz, 1H), 5.90 (br. s, 2H), 7.17 (s, 1H), 7.27-7.29 (m, 1H), 7.33 (t, J = 8.0 Hz, 1H), 7.40 (d, J = 8.5 Hz, 1H), 7.45-7.54 (m, 2H), 7.67-7.70 (m, 1H), 8.13 (s, 1H). HPLC Column: CHIRALPAK IC (250 mm × 4.6 mm × 5 mic) Mobile phase: MTBE: Ethanol with 0.1% DEA (80:20) Flow rate: 1.0 mL/min Retention time: 17.265 min |
| 118 | 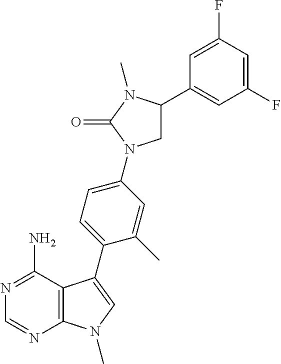 [SMILES: Cc1cc(N2CC(c3cc(F)cc(F)c3)N(C)C2=O)ccc1-c1cn(C)c2ncnc(N)c12] | 1-(4-(4-amino-7-methyl-7H- pyrrolo[2,3-d]pyrimidin-5-yl)-3- methylphenyl)-4-(3,5- difluorophenyl)-3- methylimidazolidin-2-one | 449.2 | 2.16 (s, 3H), 2.62 (s, 3H), 3.62 (t, J = 8.0 Hz, 1H), 3.71 (s, 3H), 4.24 (t, J = 8.2 Hz, 1H), 4.75 (t, J = 8.0 Hz, 1H), 5.62 (br. s, 2H), 7.10 (s, 1H), 7.15-7.18 (m, 3H), 7.23 (t, J = 8.2 Hz, 1H), 7.51-7.55 (m, 2H), 8.10 (s, 1H) |
| 119 | 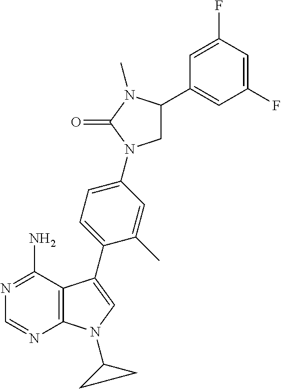 [SMILES: Cc1cc(N2CC(c3cc(F)cc(F)c3)N(C)C2=O)ccc1-c1cn(C2CC2)c2ncnc(N)c12] | 1-(4-(4-amino-7-cyclopropyl-7H- pyrrolo[2,3-d]pyrimidin-5-yl)-3- methylphenyl)-4-(3,5- difluorophenyl)-3- methylimidazolidin-2-one | 475.2 | 0.99-1.02 (m, 4H), 2.15 (s, 3H), 2.62 (s, 3H), 3.52-3.56 (m, 1H), 3.61 (t, J = 8.0 Hz, 1H), 4.23 (t, J = 8.0 Hz, 1H), 4.75 (t, J = 8.0 Hz, 1H), 5.64 (br. s, 2H), 7.04 (s, 1H), 7.15-7.17 (m, 3H), 7.23 (t, J = 8.0 Hz, 1H), 7.50-7.54 (m, 2H), 8.11 (s, 1H) |
| 120 | 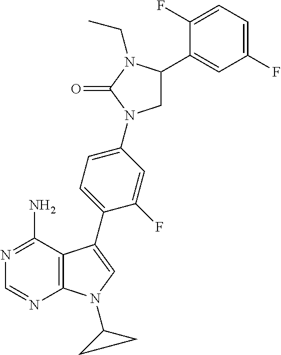 [SMILES: CCN1C(=O)N(c2ccc(-c3cn(C4CC4)c4ncnc(N)c34)c(F)c2)CC1c1cc(F)ccc1F] | 1-(4-(4-amino-7-cyclopropyl-7H- pyrrolo[2,3-d]pyrimidin-5-yl)-3- fluorophenyl)-4-(2,5-difluorophenyl)- 3-ethylimidazolidin-2-one | 493.2 | 0.95-1.01 (m, 7H), 2.74-2.82 (m, 1H), 3.41-3.48 (m, 1H), 3.52-3.57 (m, 1H), 3.72-3.76 (m, 1H), 4.29 (t, J = 8.0 Hz, 1H), 5.10-5.14 (m, 1H), 5.91 (br. s, 2H), 7.17 (s, 1H), 7.27-7.34 (m, 4H), 7.37-7.42 (m, 1H), 7.70 (d, J = 12.0 Hz, 1H), 8.12 (s, 1H) |
| 121 | 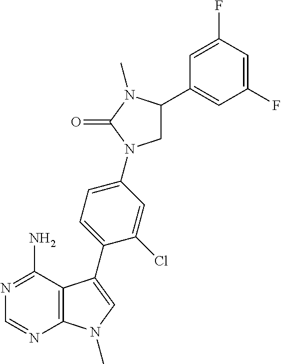 [SMILES: CN1C(=O)N(c2ccc(-c3cn(C)c4ncnc(N)c34)c(Cl)c2)CC1c1cc(F)cc(F)c1] | 1-(4-(4-amino-7-methyl-7H- pyrrolo[2,3-d]pyrimidin-5-yl)-3- chlorophenyl)-4-(3,5- difluorophenyl)-3- methylimidazolidin-2-one | 469.9 | 2.63 (s, 3H), 3.65 (t, J = 8.0 Hz, 1H), 3.72 (s, 3H), 4.26 (t, J = 8.0 Hz, 1H), 4.78 (t, J = 8.0 Hz, 1H), 5.78 (br. s, 2H), 7.16-7.26 (m, 4H), 7.34 (d, J = 8.0 Hz, 1H), 7.53-7.55 (m, 1H), 7.90-7.92 (m, 1H), 8.11 (s, 1H) |
| 122 | 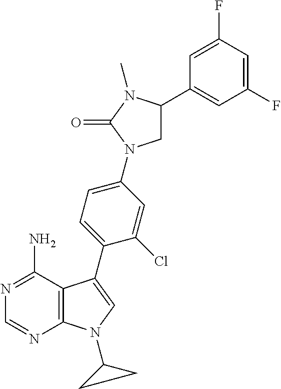 [SMILES: CN1C(=O)N(c2ccc(-c3cn(C4CC4)c4ncnc(N)c34)c(Cl)c2)CC1c1cc(F)cc(F)c1] | 1-(4-(4-amino-7-cyclopropyl-7H- pyrrolo[2,3-d]pyrimidin-5-yl)-3- chlorophenyl)-4-(3,5- difluorophenyl)-3- methylimidazolidin-2-one | 495.9 | 0.99-1.01 (m, 4H), 2.63 (s, 3H), 3.54-3.56 (m, 1H), 3.65 (t, J = 8.0 Hz, 1H), 4.26 (t, J = 8.0 Hz, 1H), 4.78 (t, J = 8.0 Hz, 1H), 5.78 (br. s, 2H), 7.14-7.17 (m, 3H), 7.22-7.26 (m, 1H), 7.34 (d, J = 8.0 Hz, 1H), 7.53-7.54 (m, 1H), 7.90 (s, 1H), 8.12 (s, 1H) |
| 123 | 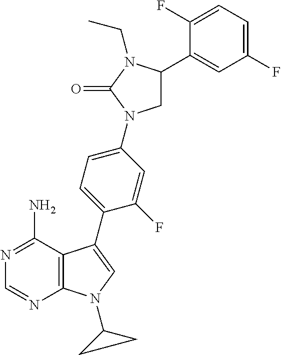 [SMILES: CCN1C(=O)N(c2ccc(-c3cn(C4CC4)c4ncnc(N)c34)c(F)c2)CC1c1cc(F)ccc1F] | 1-(4-(4-amino-7-cyclopropyl-7H- pyrrolo[2,3-d]pyrimidin-5-yl)-3- fluorophenyl)-4-(2,5-difluorophenyl)- 3-ethylimidazolidin-2-one (enantiomer 1) | 493.2 | 0.96-1.03 (m, 7H), 2.74-2.83 (m, 1H), 3.39-3.48 (m, 1H), 3.53-3.59 (m, 1H), 3.72-3.76 (m, 1H), 4.29 (t, J = 8.0 Hz, 1H), 5.10-5.14 (m, 1H), 6.07 (br. s, 2H), 7.20 (s, 1H), 7.25-7.37 (m, 4H), 7.41 (d, J = 8.0 Hz, 1H), 7.70 (dd, J = 1.6, 12.8 Hz, 1H), 8.15 (s, 1H). HPLC Column: CHIRALPAK IC (250 mm × 4.6 mm × 5 mic) Mobile phase: nHexane:Ethanol with 0.1% TFA (50:50) Flow rate: 1.0 mL/min Retention time: 13.757 min |
| 124 | 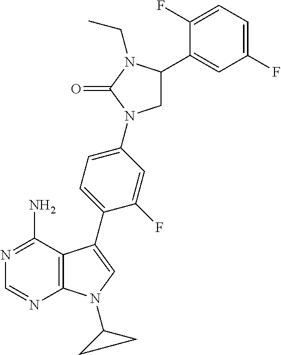 [SMILES: CCN1C(=O)N(c2ccc(-c3cn(C4CC4)c4ncnc(N)c34)c(F)c2)CC1c1cc(F)ccc1F] | 1-(4-(4-amino-7-cyclopropyl-7H- pyrrolo[2,3-d]pyrimidin-5-yl)-3- fluorophenyl)-4-(2,5-difluorophenyl)- 3-ethylimidazolidin-2-one (enantiomer 2) | 493.2 | 0.95-1.01 (m, 7H), 2.75-2.81 (m, 1H), 3.41-3.46 (m, 1H), 3.52-3.57 (m, 1H), 3.72-3.76 (m, 1H), 4.29 (t, J = 8.0 Hz, 1H), 5.10-5.14 (m, 1H), 5.91 (br. s, 2H), 7.17 (s, 1H), 7.25-7.37 (m, 4H), 7.41 (d, J = 8.0 Hz, 1H), 7.70 (d, J = 12.0 Hz, 1H), 8.13 (s, 1H). HPLC Column: CHIRALPAK IC (250 mm × 4.6 mm × 5 mic) Mobile phase: nHexane:Ethanol with 0.1% TFA (50:50) Flow rate: 1.0 mL/min Retention time: 21.750 min |
| 125 | 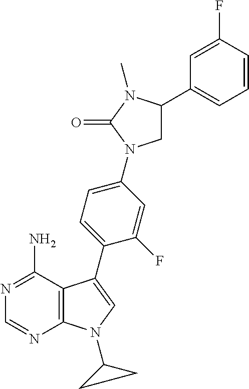 [SMILES: CN1C(=O)N(c2ccc(-c3cn(C4CC4)c4ncnc(N)c34)c(F)c2)CC1c1cccc(F)c1] | 1-(4-(4-amino-7-cyclopropyl-7H- pyrrolo[2,3-d]pyrimidin-5-yl)-3- fluorophenyl)-4-(3-fluorophenyl)-3- methylimidazolidin-2-one (enantiomer 1) | 461.1 | 0.99-1.01 (m, 4H), 2.61 (s, 3H), 3.54-3.56 (m, 1H), 3.62 (t, J = 8.0 Hz, 1H), 4.26 (t, J = 8.0 Hz, 1H), 4.76 (t, J = 8.0 Hz, 1H), 5.93 (br. s, 2H), 7.18 (s, 1H), 7.20-7.24 (m, 3H), 7.32 (t, J = 8.0 Hz, 1H), 7.41 (d, J = 8.0 Hz, 1H), 7.48 (q, J = 8.0 Hz, 1H), 7.69 (d, J = 12.8 Hz, 1H), 8.13 (s, 1H). HPLC Column: CHIRALPAK IC (250 mm × 4.6 mm × 5 mic) Mobile phase: MTBE:Methanol with 0.1% DEA (80:20) Flow rate: 1.0 mL/min Retention time: 8.276 min |
| 126 | 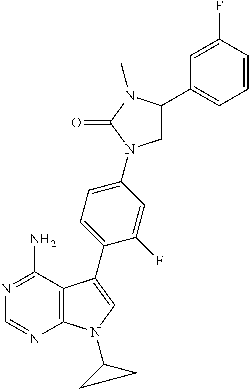 [SMILES: CN1C(=O)N(c2ccc(-c3cn(C4CC4)c4ncnc(N)c34)c(F)c2)CC1c1cccc(F)c1] | 1-(4-(4-amino-7-cyclopropyl-7H- pyrrolo[2,3-d]pyrimidin-5-yl)-3- fluorophenyl)-4-(3-fluorophenyl)-3- methylimidazolidin-2-one (enantiomer 2) | 461.1 | 1.00-1.01 (m, 4H), 2.61 (s, 3H), 3.54-3.58 (m, 1H), 3.62 (t, J = 8.0 Hz, 1H), 4.26 (t, J = 8.0 Hz, 1H), 4.76 (t, J = 8.0 Hz, 1H), 5.96 (br. s, 2H), 7.18 (s, 1H), 7.20-7.24 (m, 3H), 7.32 (t, J = 8.0 Hz, 1H), 7.41 (d, J = 8.0 Hz, 1H), 7.48 (q, J = 8.0 Hz, 1H), 7.69 (d, J = 12.8 Hz, 1H), 8.13 (s, 1H). HPLC Column: CHIRALPAK IC (250 mm × 4.6 mm × 5 mic) Mobile phase: MTBE:Methanol with 0.1% DEA (80:20 Flow rate: 1.0 mL/min Retention time: 10.981 min |
| 127 | 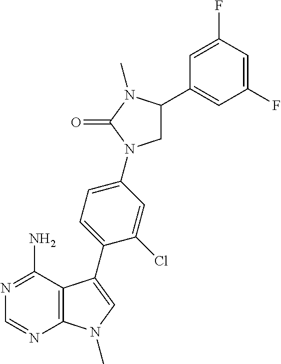 [SMILES: CN1C(=O)N(c2ccc(-c3cn(C)c4ncnc(N)c34)c(Cl)c2)CC1c1cc(F)cc(F)c1] | 1-(4-(4-amino-7-methyl-7H- pyrrolo[2,3-d]pyrimidin-5-yl)-3- chlorophenyl)-4-(3,5- difluorophenyl)-3- methylimidazolidin-2-one (enantiomer 1) | 469.8 | 2.63 (s, 3H), 3.65 (t, J = 8.0 Hz, 1H), 3.72 (s, 3H), 4.26 (t, J = 8.0 Hz, 1H), 4.78 (t, J = 8.0 Hz, 1H), 5.78 (br. s, 2H), 7.15-7.17 (m, 2H), 7.20-7.26 (m, 2H), 7.34 (d, J = 8.0 Hz, 1H), 7.54 (d, J = 8.0 Hz, 1H), 7.91 (s, 1H), 8.11 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 5.712 min |
| 128 | 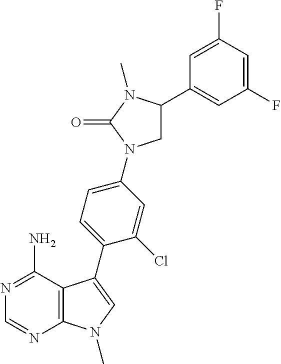 [SMILES: CN1C(=O)N(c2ccc(-c3cn(C)c4ncnc(N)c34)c(Cl)c2)CC1c1cc(F)cc(F)c1] | 1-(4-(4-amino-7-methyl-7H- pyrrolo[2,3-d]pyrimidin-5-yl)-3- chlorophenyl)-4-(3,5- difluorophenyl)-3- methylimidazolidin-2-one (enantiomer 2) | 469.8 | 2.63 (s, 3H), 3.63-3.67 (m, 1H), 3.72 (s, 3H), 4.26 (t, J = 8.0 Hz, 1H), 4.78 (t, J = 8.0 Hz, 1H), 5.77 (br. s, 2H), 7.16-7.17 (m, 2H), 7.21-7.26 (m, 2H), 7.34 (d, J = 8.0 Hz, 1H), 7.53-7.55 (m, 1H), 7.91 (s, 1H), 8.11 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 8.378 min |
| 129 | 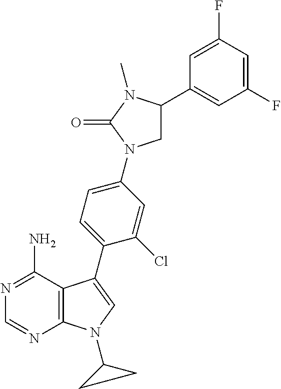 [SMILES: CN1C(=O)N(c2ccc(-c3cn(C4CC4)c4ncnc(N)c34)c(Cl)c2)CC1c1cc(F)cc(F)c1] | 1-(4-(4-amino-7-cyclopropyl-7H- pyrrolo[2,3-d]pyrimidin-5-yl)-3- chlorophenyl)-4-(3,5- difluorophenyl)-3- methylimidazolidin-2-one (enantiomer 1) | 495.9 | 0.99-1.01 (m, 4H), 2.63 (s, 3H), 3.54-3.56 (m, 1H), 3.64 (t, J = 8.0 Hz, 1H), 4.26 (t, J = 8.0 Hz, 1H), 4.78 (t, J = 8.0 Hz, 1H), 5.76 (br. s, 2H), 7.14-7.17 (m, 3H), 7.24 (t, J = 8.0 Hz, 1H), 7.34 (d, J = 8.2 Hz, 1H), 7.53 (d, J = 8.2 Hz, 1H), 7.90 (s, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 6.079 min |
| 130 | 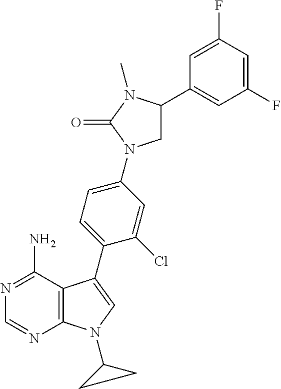 [SMILES: CN1C(=O)N(c2ccc(-c3cn(C4CC4)c4ncnc(N)c34)c(Cl)c2)CC1c1cc(F)cc(F)c1] | 1-(4-(4-amino-7-cyclopropyl-7H- pyrrolo[2,3-d]pyrimidin-5-yl)-3- chlorophenyl)-4-(3,5- difluorophenyl)-3- methylimidazolidin-2-one (enantiomer 2) | 495.9 | 0.99-1.01 (m, 4H), 2.63 (s, 3H), 3.54-3.58 (m, 1H), 3.64 (t, J = 8.0 Hz, 1H), 4.26 (t, J = 8.0 Hz, 1H), 4.78 (t, J = 8.0 Hz, 1H), 5.77 (br. s, 2H), 7.14-7.17 (m, 3H), 7.24 (t, J = 8.0 Hz, 1H), 7.34 (d, J = 8.2 Hz, 1H), 7.53-7.54 (m, 1H), 7.90 (s, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 8.722 min |
| 131 | 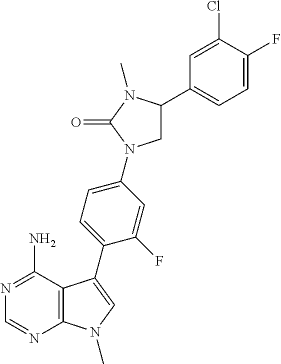 [SMILES: CN1C(=O)N(c2ccc(-c3cn(C)c4ncnc(N)c34)c(F)c2)CC1c1ccc(F)c(Cl)c1] | 1-(4-(4-amino-7-methyl-7H- pyrrolo[2,3-d]pyrimidin-5-yl)-3- fluorophenyl)-4-(3-chloro-4- fluorophenyl)-3-methylimidazolidin- 2-one | 469.8 | 2.60 (s, 3H), 3.64 (t, J = 8.0 Hz, 1H), 3.72 (s, 3H), 4.24 (t, J = 8.0 Hz, 1H), 4.76 (t, J = 8.0 Hz, 1H), 5.93 (br. s, 2H), 7.25 (s, 1H), 7.33 (t, J = 8.0 Hz, 1H), 7.40-7.49 (m, 3H), 7.65-7.71 (m, 2H), 8.12 (s, 1H) |
| 132 | 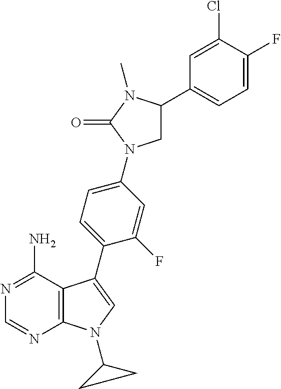 [SMILES: CN1C(=O)N(c2ccc(-c3cn(C4CC4)c4ncnc(N)c34)c(F)c2)CC1c1ccc(F)c(Cl)c1] | 1-(4-(4-amino-7-cyclopropyl-7H- pyrrolo[2,3-d]pyrimidin-5-yl)-3- fluorophenyl)-4-(3-chloro-4- fluorophenyl)-3-methylimidazolidin- 2-one | 495.2 | 0.99-1.01 (m, 4H), 2.60 (s, 3H), 3.53-3.55 (m, 1H), 3.63 (t, J = 8.0 Hz, 1H), 4.24 (t, J = 8.0 Hz, 1H), 4.76 (t, J = 8.0 Hz, 1H), 5.91 (br. s, 2H), 7.17 (s, 1H), 7.32 (t, J = 8.2 Hz, 1H), 7.39-7.49 (m, 3H), 7.65-7.70 (m, 2H), 8.13 (s, 1H) |
| 133 | 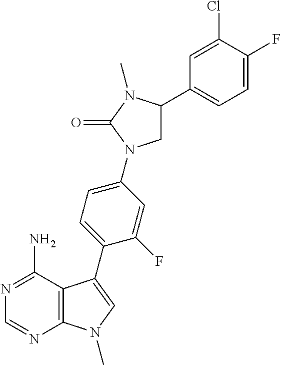 [SMILES: CN1C(=O)N(c2ccc(-c3cn(C)c4ncnc(N)c34)c(F)c2)CC1c1ccc(F)c(Cl)c1] | 1-(4-(4-amino-7-methyl-7H- pyrrolo[2,3-d]pyrimidin-5-yl)-3- fluorophenyl)-4-(3-chloro-4- fluorophenyl)-3-methylimidazolidin- 2-one (enantiomer 1) | 469.8 | 2.60 (s, 3H), 3.63-3.66 (m, 1H), 3.72 (s, 3H), 4.24 (t, J = 8.0 Hz, 1H), 4.76 (t, J = 8.0 Hz, 1H), 5.93 (br. s, 2H), 7.25 (s, 1H), 7.33 (t, J = 8.0 Hz, 1H), 7.42-7.49 (m, 3H), 7.65-7.71 (m, 2H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 7.401 min |
| 134 | 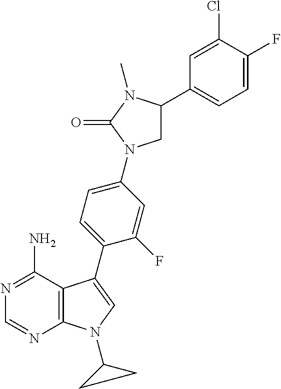 [SMILES: CN1C(=O)N(c2ccc(-c3cn(C4CC4)c4ncnc(N)c34)c(F)c2)CC1c1ccc(F)c(Cl)c1] | 1-(4-(4-amino-7-cyclopropyl-7H- pyrrolo[2,3-d]pyrimidin-5-yl)-3- fluorophenyl)-4-(3-chloro-4- fluorophenyl)-3-methylimidazolidin- 2-one (enantiomer 1) | 495.2 | 0.97-1.01 (m, 4H), 2.65 (s, 3H), 3.53-3.57 (m, 1H), 3.61-3.65 (m, 1H), 4.24 (t, J = 8.0 Hz, 1H), 4.76 (t, J = 8.0 Hz, 1H), 5.91 (br. s, 2H), 7.17 (s, 1H), 7.32 (t, J = 8.2 Hz, 1H), 7.38-7.49 (m, 3H), 7.65-7.70 (m, 2H), 8.13 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 8.065 min |
| 135 | 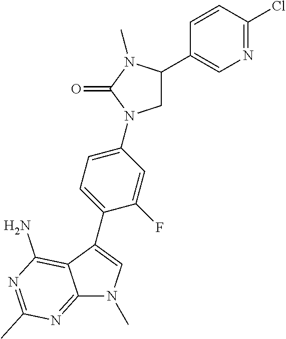 [SMILES: Cc1nc(N)c2c(-c3ccc(N4CC(c5ccc(Cl)nc5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(6-chloropyridin-3- yl)-3-methylimidazolidin-2-one | 466.1 | 2.39 (s, 3H), 2.60 (s, 3H), 3.65-3.68 (m, 1H), 3.68 (s, 3H), 4.26 (t, J = 9.6 Hz, 1H), 4.83 (t, J = 8 Hz, 1H), 5.84 (br. s, 2H), 7.15 (s, 1H), 7.32 (t, J = 8.8 Hz, 1H), 7.39 (d, J = 8.4 Hz, 1H), 7.58 (d, J = 8.4 Hz, 1H), 7.68 (d, J = 12.4 Hz, 1H), 7.90 (d, J = 8 Hz, 1H), 8.47 (s, 1H). |
| 136 | 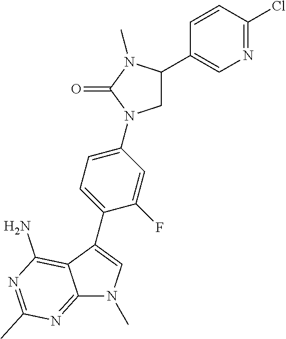 [SMILES: Cc1nc(N)c2c(-c3ccc(N4CC(c5ccc(Cl)nc5)N(C)C4=O)cc3F)cn(C)c2n1] | 1-(4-(4-amino-2,7-dimethyl-7H- pyrrolo[2,3-d]pyrimidin-5-yl)-3- fluorophenyl)-4-(6-chloropyridin-3- yl)-3-methylimidazolidin-2-one (enantiomer 2) | 466.1 | 2.41 (s, 3H), 2.60 (s, 3H), 3.65-3.69 (m, 1H), 3.69 (s, 3H), 4.27 (t, J = 9.2 Hz, 1H), 4.83 (t, J = 8.4 Hz, 1H), 5.99 (br. s, 2H), 7.19 (s, 1H), 7.32 (t, J = 8.8 Hz, 1H), 7.39-7.41 (m, 1H), 7.58 (d, J = 8.4 Hz, 1H), 7.68 (d, J = 12.8 Hz, 1H), 7.90 (d, J = 7.2 Hz, 1H), 8.47 (s, 1H). HPLC Column: CHIRALPAK IA (250 mm × 20 mm × 5 mic) Mobile phase: Ethanol with 0.1% TFA (100%) Flow rate: 0.5 mL/min Retention time: 14.822 min |
| 138 | 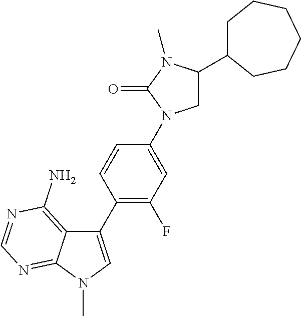 [SMILES: CN1C(=O)N(c2ccc(-c3cn(C)c4ncnc(N)c34)c(F)c2)CC1C1CCCCCC1] | 1-(4-(4-amino-7-methyl-7H- pyrrolo[2,3-d]pyrimidin-5-yl)-3- fluorophenyl)-4-cycloheptyl-3- methylimidazolidin-2-one | 437.2 | 1.15-1.18 (m, 1H), 1.32-1.70 (m, 11H), 1.90 (s, 1H), 2.73 (s, 3H), 3.49-3.53 (m, 1H), 3.62-3.64 (m, 1H), 3.72 (s, 3H), 3.80 (t, J = 9.6 Hz, 1H), 5.93 (br, s. 2H), 7.23 (s, 1H), 7.30 (t, J = 8.8 Hz, 1H), 7.41-7.43 (m, 1H), 7.68 (d, J = 13.2 Hz, 1H), 8.12 (s, 1H). |
| 139 | 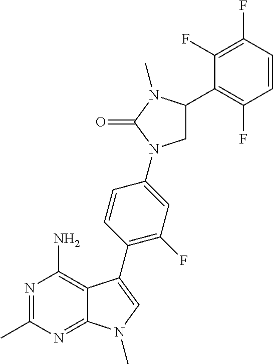 [SMILES: Cc1nc(N)c2c(-c3ccc(N4CC(c5c(F)ccc(F)c5F)N(C)C4=O)cc3F)cn(C)c2n1] | 1-(4-(4-amino-2,7-dimethyl-7H- pyrrolo[2,3-d]pyrimidin-5-yl)-3- fluorophenyl)-3-methyl-4-(2,3,6- trifluorophenyl)imidazolidin-2-one | 485.1 | 2.39 (s, 3H), 2.64 (s, 3H), 3.68 (s, 3H), 3.88-3.92 (m, 1H), 4.32 (t, J = 10.0 Hz, 1H), 5.21-5.25 (m, 1H), 5.88 (br, s. 2H), 7.15 (s, 1H), 7.22 (t, J = 9.2 Hz, 1H), 7.30-7.34 (m, 1H), 7.37-7.40 (m, 1H), 7.52-7.60 (m, 1H), 7.67-7.77 (m, 1H). |
| 140 | 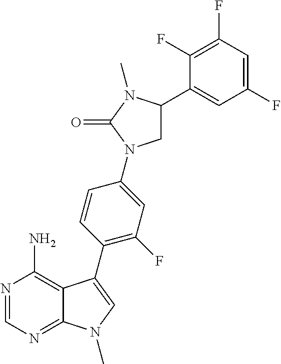 [SMILES: CN1C(=O)N(c2ccc(-c3cn(C)c4ncnc(N)c34)c(F)c2)CC1c1cc(F)cc(F)c1F] | 1-(4-(4-amino-7-methyl-7H- pyrrolo[2,3-d]pyrimidin-5-yl)-3- fluorophenyl)-3-methyl-4-(2,3,5- trifluorophenyl)imidazolidin-2-one (enantiomer 1) | 471.1 | 2.65-2.67 (m, 3H), 3.72-3.77 (m, 4H), 4.31 (t, J = 9.2 Hz, 1H), 5.07 (t, J = 9.2 Hz, 1H), 5.93 (br, s. 2H), 7.16 (s, 1H), 7.25 (s, 1H), 7.317.42 (m, 2H), 7.50-7.62 (m, 1H), 7.68 (d, J = 12.8 Hz, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 16.725 min |
| 141 | 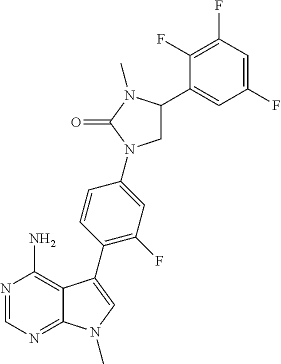 [SMILES: CN1C(=O)N(c2ccc(-c3cn(C)c4ncnc(N)c34)c(F)c2)CC1c1cc(F)cc(F)c1F] | 1-(4-(4-amino-7-methyl-7H- pyrrolo[2,3-d]pyrimidin-5-yl)-3- fluorophenyl)-3-methyl-4-(2,3,5- trifluorophenyl)imidazolidin-2-one (enantiomer 2) | 471.1 | 2.67 (s, 3H), 3.72-3.77 (m, 4H), 4.31 (t, J = 9.6 Hz, 1H), 5.07 (t, J = 6.8 Hz, 1H), 5.93 (br, 2H), 7.15 (s, 1H), 7.25 (s, 1H), 7.31-7.35 (m, 1H), 7.39-7.42 (m, 1H), 7.50-7.62 (m, 1H), 7.68 (d, J = 12.8 Hz, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 21.763 min |
| 142 | 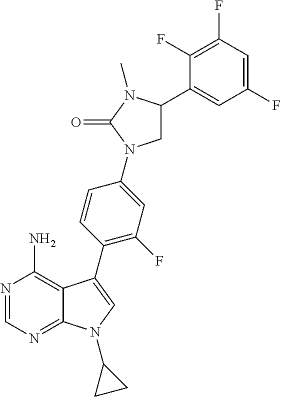 [SMILES: CN1C(=O)N(c2ccc(-c3cn(C4CC4)c4ncnc(N)c34)c(F)c2)CC1c1cc(F)cc(F)c1F] | 1-(4-(4-amino-7-cyclopropyl-7H- pyrrolo[2,3-d]pyrimidin-5-yl)-3- fluorophenyl)-3-methyl-4-(2,3,5- trifluorophenyl)imidazolidin-2-one (enantiomer 1) | 497.1 | 0.99-1.01 (m, 4H), 2.67 (s, 3H), 3.52-3.57 (m, 1H), 3.72-3.76 (m, 1H), 4.31 (t, J = 9.2 Hz, 1H), 5.05-5.09 (m, 1H), 5.91 (br, s. 2H), 7.17 (s, 2H), 7.31-7.41 (m, 2H), 7.55-7.57 (m, 1H), 7.67 (d, J = 12.8 Hz, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 10.134 min |
| 143 | 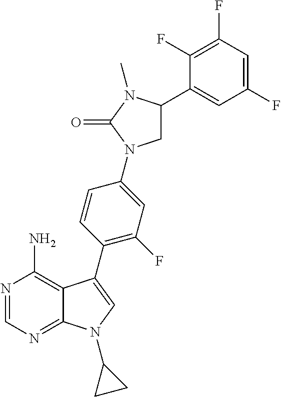 [SMILES: CN1C(=O)N(c2ccc(-c3cn(C4CC4)c4ncnc(N)c34)c(F)c2)CC1c1cc(F)cc(F)c1F] | 1-(4-(4-amino-7-cyclopropyl-7H- pyrrolo[2,3-d]pyrimidin-5-yl)-3- fluorophenyl)-3-methyl-4-(2,3,5- trifluorophenyl)imidazolidin-2-one (enantiomer 2) | 497.1 | 0.99-1.01 (m, 4H), 2.67 (s, 3H), 3.54-3.56 (m, 1H), 3.72-3.76 (m, 1H), 4.31 (t, J = 9.6 Hz, 1H), 5.07 (t, J = 7.2 Hz, 1H), 5.94 (br, s. 2H), 7.18 (s, 2H), 7.31-7.41 (m, 2H), 7.55-7.57 (m, 1H), 7.68 (d, J = 12.8 Hz, 1H), 8.13 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 12.874 min |
| 144 | 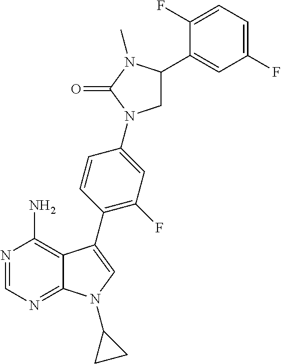 [SMILES: CN1C(=O)N(c2ccc(-c3cn(C4CC4)c4ncnc(N)c34)c(F)c2)CC1c1cc(F)ccc1F] | 1-(4-(4-amino-7-cyclopropyl-7H- pyrrolo[2,3-d]pyrimidin-5-yl)-3- fluorophenyl)-4-(2,5-difluorophenyl)- 3-methylimidazolidin-2-one | 479.2 | 0.99-1.01 (m, 4H), 2.65 (s, 3H), 3.53-3.55 (m, 1H), 3.68-3.72 (m, 1H), 4.30 (t, J = 10.0 Hz, 1H), 5.0 (t, J = 7.6 Hz, 1H), 5.91 (br, s. 2H), 7.17 (s, 1H), 7.27-7.42 (m, 5H), 7.69 (d, J = 13.2 Hz, 1H), 8.12 (s, 1H). |
| 145 | 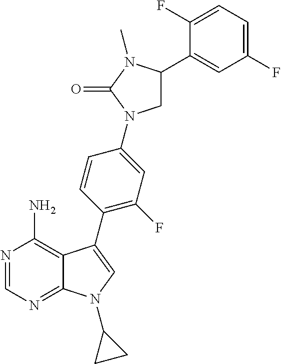 [SMILES: CN1C(=O)N(c2ccc(-c3cn(C4CC4)c4ncnc(N)c34)c(F)c2)CC1c1cc(F)ccc1F] | 1-(4-(4-amino-7-cyclopropyl-7H- pyrrolo[2,3-d]pyrimidin-5-yl)-3- fluorophenyl)-4-(2,5-difluorophenyl)- 3-methylimidazolidin-2-one (enantiomer 1) | 479.2 | 1.0-1.02 (m, 4H), 2.66 (s, 3H), 3.56 (s, 1H), 3.70 (t, J = 9.20 Hz, 1H), 4.30 (t, J = 9.6 Hz, 1H), 5.01 (t, J = 8.8 Hz, 1H), 6.11 (br, s. 2H), 7.21 (s, 1H), 7.26-7.42 (m, 5H), 7.69 (d, J = 12.0 Hz, 1H), 8.16 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 11.721 min |
| 146 | 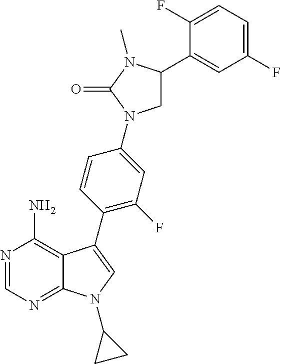 [SMILES: CN1C(=O)N(c2ccc(-c3cn(C4CC4)c4ncnc(N)c34)c(F)c2)CC1c1cc(F)ccc1F] | 1-(4-(4-amino-7-cyclopropyl-7H- pyrrolo[2,3-d]pyrimidin-5-yl)-3- fluorophenyl)-4-(2,5-difluorophenyl)- 3-methylimidazolidin-2-one (enantiomer 2) | 479.2 | 0.99-1.01 (m, 4H), 2.65 (s, 3H), 3.54 (s, 1H), 3.70 (t, J = 8.8 Hz, 1H), 4.30 (t, J = 9.2 Hz, 1H), 5.0 (t, J = 8.4 Hz, 1H), 5.91 (br, s. 2H), 7.17 (s, 1H), 7.27-7.42 (m, 5H), 7.69 (d, J = 12.8 Hz, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 14.363 min |
| 147 | 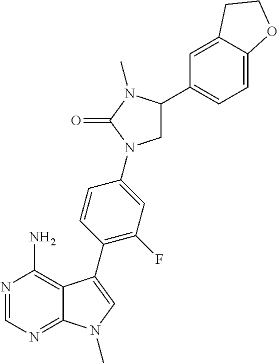 [SMILES: CN1C(=O)N(c2ccc(-c3cn(C)c4ncnc(N)c34)c(F)c2)CC1c1ccc2c(c1)CCO2] | 1-(4-(4-amino-7-methyl-7H- pyrrolo[2,3-d]pyrimidin-5-yl)-3- fluorophenyl)-4-(2,3- dihydrobenzofuran-5-yl)-3- methylimidazolidin-2-one | 459.1 | 2.57 (s, 3H), 3.17 (t, J = 8.4 Hz, 2H), 3.56-3.64 (m, 1H), 3.72 (s, 3H), 4.21 (t, J = 9.2 Hz, 1H), 4.52 (t, J = 8.4 Hz, 2H), 4.63 (t, J = 8.0 Hz, 1H), 5.93 (br, s. 2H), 6.77 (d, J = 8.4 Hz, 1H), 7.09 (d, J = 7.6 Hz, 1H),7.24 (s, 2H), 7.32 (t, J = 8.4 Hz, 1H), 7.40-7.42 (m, 1H), 7.71 (d, J = 13.2 Hz, 1H), 8.12 (s, 1H). |
| 148 | 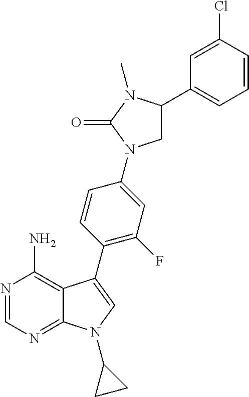 [SMILES: CN1C(=O)N(c2ccc(-c3cn(C4CC4)c4ncnc(N)c34)c(F)c2)CC1c1cccc(Cl)c1] | 1-(4-(4-amino-7-cyclopropyl-7H- pyrrolo[2,3-d]pyrimidin-5-yl)-3- fluorophenyl)-4-(3-chlorophenyl)-3- methylimidazolidin-2-one | 477.1 | 0.99-1.01 (m, 4H), 2.61 (s, 3H), 3.52-3.57 (m, 1H), 3.60-3.65 (m, 1H), 4.25 (t, J = 10.0 Hz, 1H), 4.75 (t, J = 8.0 Hz, 1H), 5.91 (br, s. 2H), 7.17 (s, 1H), 7.30-7.48 (m, 6H), 7.67-7.70 (m, 1H), 8.12 (s, 1H). |
| 149 | 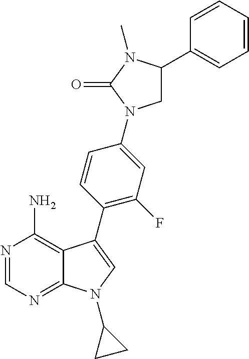 [SMILES: CN1C(=O)N(c2ccc(-c3cn(C4CC4)c4ncnc(N)c34)c(F)c2)CC1c1ccccc1] | 1-(4-(4-amino-7-cyclopropyl-7H- pyrrolo[2,3-d]pyrimidin-5-yl)-3- fluorophenyl)-3-methyl-4- phenylimidazolidin-2-one | 443.2 | 0.99-1.02 (m, 4H), 2.59 (s, 3H), 3.52-3.62 (m, 2H), 4.26 (t, J = 9.6 Hz, 1H), 4.72 (t, J = 8.8 Hz, 1H), 5.90 (br, s. 2H), 7.17 (s, 1H), 7.32 (t, J = 8.8 Hz, 1H), 7.37-7.45 (m, 6H), 7.68-7.72 (m, 1H), 8.12 (s, 1H). |
| 150 | 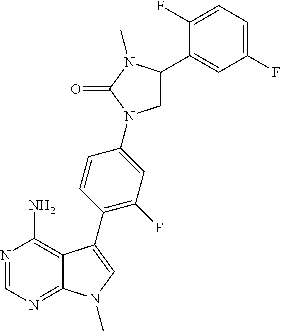 [SMILES: CN1C(=O)N(c2ccc(-c3cn(C)c4ncnc(N)c34)c(F)c2)CC1c1cc(F)ccc1F] | 1-(4-(4-amino-7-methyl-7H- pyrrolo[2,3-d]pyrimidin-5-yl)-3- fluorophenyl)-4-(2,5-difluorophenyl)- 3-methylimidazolidin-2-one (enantiomer 1) | 453.1 | 2.66 (s, 3H), 3.69-3.72 (m, 4H), 4.30 (t, J = 9.6 Hz, 1H), 5.00 (t, J = 7.2 Hz, 1H), 5.92 (br, s. 2H), 7.25-7.43 (m, 6H), 7.69 (d, J = 13.2 Hz, 1H), 8.12 (s, 1H). HPLC Column: CHIRALPAK IA (250 mm × 4.6 mm × 5 μm) Mobile phase: n- Hexane:0.1% DEA in Ethanol (60:40) Flow rate: 1.0 mL/min Retention time: 26.797 min |
| 151 | 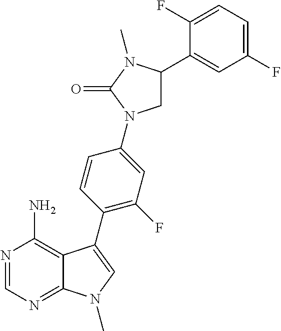 [SMILES: CN1C(=O)N(c2ccc(-c3cn(C)c4ncnc(N)c34)c(F)c2)CC1c1cc(F)ccc1F] | 1-(4-(4-amino-7-methyl-7H- pyrrolo[2,3-d]pyrimidin-5-yl)-3- fluorophenyl)-4-(2,5-difluorophenyl)- 3-methylimidazolidin-2-one (enantiomer 2) | 453.1 | 2.66 (s, 3H), 3.69-3.72 (m, 4H), 4.30 (t, J = 9.6 Hz, 1H), 5.00 (t, J = 6.8 Hz, 1H), 5.93 (br, s. 2H), 7.25-7.38 (m, 5H), 7.40-7.43 (m, 1H), 7.68-7.71 (m, 1H), 8.12 (s, 1H). HPLC Column: CHIRALPAK IA (250 mm × 4.6 mm × 5 μm) Mobile phase: n- Hexane:0.1% DEA in Ethanol (60:40) Flow rate: 1.0 mL/min Retention time: 32.737 min |
| 152 | 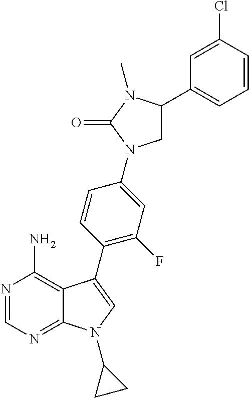 [SMILES: CN1C(=O)N(c2ccc(-c3cn(C4CC4)c4ncnc(N)c34)c(F)c2)CC1c1cccc(Cl)c1] | 1-(4-(4-amino-7-cyclopropyl-7H- pyrrolo[2,3-d]pyrimidin-5-yl)-3- fluorophenyl)-4-(3-chlorophenyl)-3- methylimidazolidin-2-one (enantiomer 1) | 477.1 | 0.99-1.01 (m, 4H), 2.61 (s, 3H), 3.53-3.65 (m, 2H), 4.25 (t, J = 9.2 Hz, 1H), 4.76 (t, J = 8.0 Hz, 1H), 5.91 (br, s. 2H), 7.17 (s, 1H), 7.30-7.47 (m, 6H), 7.68 (d, J = 12.8 Hz, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 13.505 min |
| 153 | 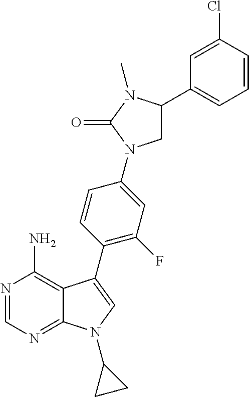 [SMILES: CN1C(=O)N(c2ccc(-c3cn(C4CC4)c4ncnc(N)c34)c(F)c2)CC1c1cccc(Cl)c1] | 1-(4-(4-amino-7-cyclopropyl-7H- pyrrolo[2,3-d]pyrimidin-5-yl)-3- fluorophenyl)-4-(3-chlorophenyl)-3- methylimidazolidin-2-one (enantiomer 2) | 477.1 | 0.99-1.01 (m, 4H),2.61 (s, 3H), 3.53-3.55 (m, 1H), 3.62 (t, J = 7.2 Hz, 1H), 4.25 (t, J = 9.2 Hz, 1H), 4.75 (t, J = 7.6 Hz, 1H), 5.91 (br, s. 2H), 7.17 (s, 1H), 7.30-7.47 (m, 6H), 7.68 (d, J = 12.8 Hz, 1H), 8.12 (s, 1H). HPLC Column: CHIRALPAK IC (250 mm × 20 mm × 5 mic) Mobile phase: MTBE:Methanol with 0.1% DEA (80:20) Flow rate: 1.0 mL/min Retention time: 20.550 min |
| 154 | 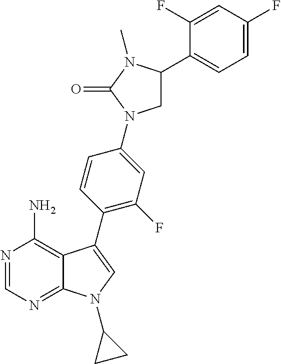 [SMILES: CN1C(=O)N(c2ccc(-c3cn(C4CC4)c4ncnc(N)c34)c(F)c2)CC1c1ccc(F)cc1F] | 1-(4-(4-amino-7-cyclopropyl-7H- pyrrolo[2,3-d]pyrimidin-5-yl)-3- fluorophenyl)-4-(2,4-difluorophenyl)- 3-methylimidazolidin-2-one (enantiomer 1) | 479.1 | 0.97-1.01 (m, 4H), 2.63-2.65 (m, 3H), 3.52-357 (m, 1H), 3.67-3.71 (m, 1H), 4.29 (t, J = 9.6 Hz, 1H), 4.98-5.02 (m, 1H), 5.92 (br, s. 2H), 7.13-7.17 (m, 2H), 7.30-7.35 (m, 2H), 7.39-7.50 (m, 2H), 7.67-7.71 (m, 1H), 8.12 (s, 1H). HPLC Column: CHIRALPAK IC (250 mm × 20 mm × 5 mic) Mobile phase: nHexane:Ethanol with 0.1% TFA (50:50) Flow rate: 1.0 mL/min Retention time: 16.970 min |
| 155 | 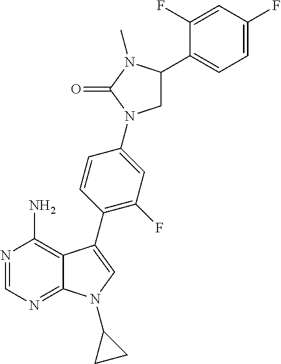 [SMILES: CN1C(=O)N(c2ccc(-c3cn(C4CC4)c4ncnc(N)c34)c(F)c2)CC1c1ccc(F)cc1F] | 1-(4-(4-amino-7-cyclopropyl-7H- pyrrolo[2,3-d]pyrimidin-5-yl)-3- fluorophenyl)-4-(2,4-difluorophenyl)- 3-methylimidazolidin-2-one (enantiomer 2) | 479.2 | 0.97-1.01 (m, 4H), 2.63-2.65 (m, 3H), 3.52-357 (m, 1H), 3.67-3.71 (m, 1H), 4.29 (t, J = 9.6 Hz, 1H), 4.98-5.02 (m, 1H), 5.91 (br, s. 2H), 7.13-7.17 (m, 2H), 7.30-7.35 (m, 2H), 7.39-7.50 (m, 2H), 7.67-7.71 (m, 1H), 8.12 (s, 1H). HPLC Column: CHIRALPAK IC (250 mm × 20 mm × 5 mic) Mobile phase: nHexane:Ethanol with 0.1% TFA (50:50) Flow rate: 1.0 mi./min Retention time: 25.708 min |
| 156 | 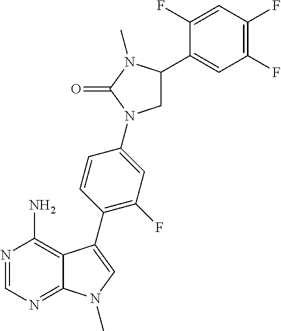 [SMILES: CN1C(=O)N(c2ccc(-c3cn(C)c4ncnc(N)c34)c(F)c2)CC1c1cc(F)c(F)cc1F] | 1-(4-(4-amino-7-methyl-7H- pyrrolo[2,3-d]pyrimidin-5-yl)-3- fluorophenyl)-3-methyl-4-(2,4,5- trifluorophenyl)imidazolidin-2-one | 471.1 | 2.64 (s, 3H), 3.69-3.72 (m, 4H), 4.28 (t, J = 9.6 Hz, 1H), 4.99 (t, J = 7.2 Hz, 1H), 5.93 (br, s. 2H), 7.25 (s, 1H), 7.33 (t, J = 8.8 Hz, 1H), 7.40-7.42 (m, 1H), 7.54-7.71 (m, 3H), 8.12 (s, 1H). |
| 157 | 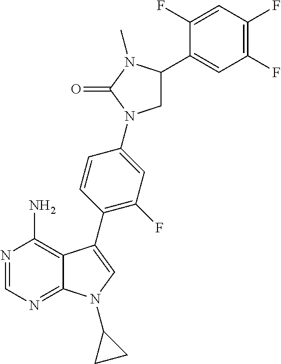 [SMILES: CN1C(=O)N(c2ccc(-c3cn(C4CC4)c4ncnc(N)c34)c(F)c2)CC1c1cc(F)c(F)cc1F] | 1-(4-(4-amino-7-cyclopropyl-7H- pyrrolo[2,3-d]pyrimidin-5-yl)-3- fluorophenyl)-3-methyl-4-(2,4,5- trifluorophenyl)imidazolidin-2-one | 497.1 | 0.99-1.01 (m, 4H), 2.64 (s, 3H), 3.53-355 (m, 1H), 3.70 (t, J = 9.2 Hz, 1H), 4.28 (t, J = 9.2 Hz, 1H), 4.99 (t, J = 9.2 Hz, 1H), 5.90 (br, s. 2H), 7.17 (s, 1H), 7.33 (t, J = 8.4 Hz, 1H), 7.39-7.41 (m, 1H), 7.54-7.70 (m, 3H), 8.13 (s, 1H). |
| 158 | 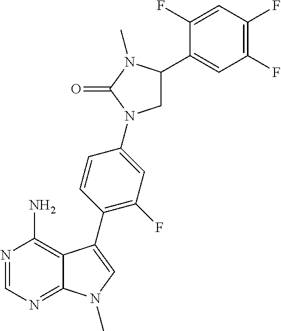 [SMILES: CN1C(=O)N(c2ccc(-c3cn(C)c4ncnc(N)c34)c(F)c2)CC1c1cc(F)c(F)cc1F] | 1-(4-(4-amino-7-methyl-7H- pyrrolo[2,3-d]pyrimidin-5-yl)-3- fluorophenyl)-3-methyl-4-(2,4,5- trifluorophenyl)imidazolidin-2-one (enantiomer 1) | 471.0 | 2.64 (s, 3H), 3.69-3.72 (m, 4H), 4.28 (t, J = 10.0 Hz, 1H), 4.99 (t, J = 8.8 Hz, 1H), 5.93 (br, s. 2H), 7.25 (s, 1H), 7.33 (t, J = 8.4 Hz, 1H), 7.40-7.42 (m, 1H), 7.54-7.71 (m, 3H), 8.12 (s, 1H). HPLC Column: CHIRALPAK IC (250 mm × 4.6 mm × 5 mic) Mobile phase: MTBE:Ethanol with 0.1% DEA (85:15) Flow rate: 1.0 mL/min Retention time: 11.343 min |
| 159 | 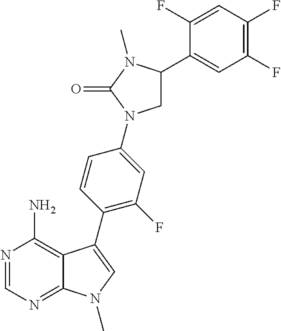 [SMILES: CN1C(=O)N(c2ccc(-c3cn(C)c4ncnc(N)c34)c(F)c2)CC1c1cc(F)c(F)cc1F] | 1-(4-(4-amino-7-methyl-7H- pyrrolo[2,3-d]pyrimidin-5-yl)-3- fluorophenyl)-3-methyl-4-(2,4,5- trifluorophenyl)imidazolidin-2-one (enantiomer 2) | 471.0 | 2.64 (s, 3H), 3.69-3.72 (m, 4H), 4.28 (t, J = 9.6 Hz, 1H), 4.99 (t, J = 7.6 Hz, 1H), 5.92 (br, s. 2H), 7.25 (s, 1H), 7.33 (t, J = 8.8 Hz, 1H), 7.40-7.42 (m, 1H), 7.54-7.71 (m, 3H), 8.12 (s, 1H). HPLC Column: CHIRALPAK IC (250 mm × 4.6 mm × 5 mic) Mobile phase: MTBE:Ethanol with 0.1% DEA (85:15) Flow rate: 1.0 mL/min Retention time: 15.831 min |
| 160 | 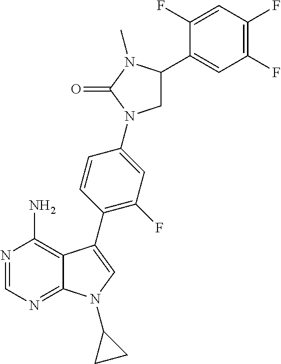 [SMILES: CN1C(=O)N(c2ccc(-c3cn(C4CC4)c4ncnc(N)c34)c(F)c2)CC1c1cc(F)c(F)cc1F] | 1-(4-(4-amino-7-cyclopropyl-7H- pyrrolo[2,3-d]pyrimidin-5-yl)-3- fluorophenyl)-3-methyl-4-(2,4,5- trifluorophenyl)imidazolidin-2-one (enantiomer 1) | 497.1 | 0.99-1.01 (m, 4H), 2.64 (s, 3H), 3.53-356 (m, 1H), 3.68-3.72 (m, 1H), 4.28 (t, J = 9.6 Hz, 1H), 4.98 (t, J = 7.2 Hz, 1H), 5.91 (br, s. 2H), 7.17 (s, 1H), 7.33 (t, J = 8.8 Hz, 1H), 7.39-7.41 (m, 1H), 7.56-7.70 (m, 3H), 8.13 (s, 1H). HPLC Column: CHIRALPAK IC (250 mm × 4.6 mm × 5 mic) Mobile phase: MTBE:Ethanol with 0.1% DEA (85:15) Flow rate: 1.0 mL/min Retention time: 14.179 min |
| 161 | 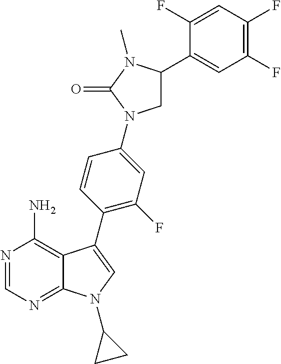 [SMILES: CN1C(=O)N(c2ccc(-c3cn(C4CC4)c4ncnc(N)c34)c(F)c2)CC1c1cc(F)c(F)cc1F] | 1-(4-(4-amino-7-cyclopropyl-7H- pyrrolo[2,3-d]pyrimidin-5-yl)-3- fluorophenyl)-3-methyl-4-(2,4,5- trifluorophenyl)imidazolidin-2-one (enantiomer 2) | 497.1 | 0.99-1.01 (m, 4H), 2.64 (s, 3H), 3.53-354 (m, 1H), 3.68-3.72 (m, 1H), 4.28 (t, J = 9.6 Hz, 1H), 4.98 (t, J = 6.8 Hz, 1H), 5.90 (br, s. 2H), 7.17 (s, 1H), 7.33 (t, J = 8.8 Hz, 1H), 7.39-7.41 (m, 1H), 7.54-7.70 (m, 3H), 8.13 (s, 1H). HPLC Column: CHIRALPAK IC (250 mm × 4.6 mm × 5 mic) Mobile phase: MTBE:Ethanol with 0.1% DEA (85:15) Flow rate: 1.0 mL/min Retention time: 19.518 min |
| 162 | 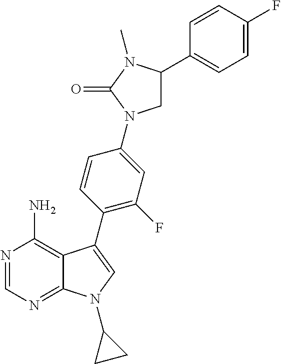 [SMILES: CN1C(=O)N(c2ccc(-c3cn(C4CC4)c4ncnc(N)c34)c(F)c2)CC1c1ccc(F)cc1] | 1-(4-(4-amino-7-cyclopropyl-7H- pyrrolo[2,3-d]pyrimidin-5-yl)-3- fluorophenyl)-4-(4-fluorophenyl)-3- methylimidazolidin-2-one | 461.2 | 0.97-1.01 (m, 4H), 2.58 (s, 3H), 3.52-362 (m, 2H), 4.25 (t, J = 9.6 Hz, 1H), 4.74 (t, J = 7.6 Hz, 1H), 5.91 (br, s. 2H), 7.17 (s, 1H), 7.25 (t, J = 8.8 Hz, 2H), 7.32 (t, J = 8.8 Hz, 1H), 7.39-7.46 (m, 3H), 7.68-7.71 (m, 1H), 8.12 (s, 1H). |
| 163 | 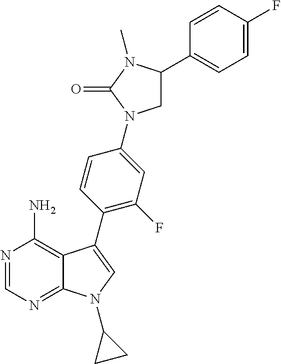 [SMILES: CN1C(=O)N(c2ccc(-c3cn(C4CC4)c4ncnc(N)c34)c(F)c2)CC1c1ccc(F)cc1] | 1-(4-(4-amino-7-cyclopropyl-7H- pyrrolo[2,3-d]pyrimidin-5-yl)-3- fluorophenyl)-4-(4-fluorophenyl)-3- methylimidazolidin-2-one (enantiomer 1) | 461.2 | 0.99-1.01 (m, 4H), 2.58 (s, 3H), 3.53-362 (m, 2H), 4.25 (t, J = 9.2 Hz, 1H), 4.74 (t, J = 8.4 Hz, 1H), 5.91 (br, s. 2H), 7.17 (s, 1H), 7.25 (t, J = 8.8 Hz, 2H), 7.32 (t, J = 8.4 Hz, 1H), 7.39-7.46 (m, 3H), 7.69 (d, J = 12.0 Hz, 1H), 8.12 (s, 1H). HPLC Column: CHIRALPAK IC (250 mm × 4.6 mm × 5 mic) Mobile phase: MTBE:IPA with 0.1% DEA (65:35) Flow rate: 1.0 mL/min Retention time: 15.497 min |
| 164 | 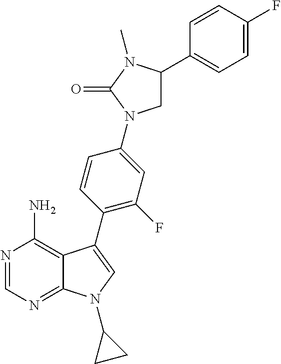 [SMILES: CN1C(=O)N(c2ccc(-c3cn(C4CC4)c4ncnc(N)c34)c(F)c2)CC1c1ccc(F)cc1] | 1-(4-(4-amino-7-cyclopropyl-7H- pyrrolo[2,3-d]pyrimidin-5-yl)-3- fluorophenyl)-4-(4-fluorophenyl)-3- methylimidazolidin-2-one (enantiomer 2) | 461.2 | 0.99-1.01 (m, 4H), 2.58 (s, 3H), 3.53-362 (m, 2H), 4.25 (t, J = 9.2 Hz, 1H), 4.74 (t, J = 8.4 Hz, 1H), 5.91 (br, s. 2H), 7.17 (s, 1H), 7.25 (t, J = 8.4 Hz, 2H), 7.32 (t, J = 8.8 Hz, 1H), 7.40-7.46 (m, 3H), 7.69 (d, J = 12.0 Hz, 1H), 8.12 (s, 1H). HPLC Column: CHIRALPAK IC (250 mm × 4.6 mm × 5 mic) Mobile phase: MTBE:IPA with 0.1% DEA (65:35) Flow rate: 1.0 mL/min Retention time: 25.310 min |
| 165 | 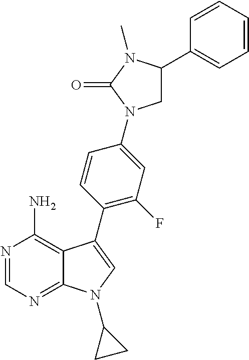 [SMILES: CN1C(=O)N(c2ccc(-c3cn(C4CC4)c4ncnc(N)c34)c(F)c2)CC1c1ccccc1] | 1-(4-(4-amino-7-cyclopropyl-7H- pyrrolo[2,3-d]pyrimidin-5-yl)-3- fluorophenyl)-3-methyl-4- phenylimidazolidin-2-one (enantiomer 1) | 443.2 | 0.99-1.01 (m, 4H), 2.59 (s, 3H), 3.54-360 (m, 2H), 4.26 (t, J = 9.6 Hz, 1H), 4.72 (t, J = 8.4 Hz, 1H), 5.91 (br, s. 2H), 7.17 (s, 1H), 7.30-7.43 (m, 7H), 7.69 (d, J = 13.6 Hz, 1H), 8.12 (s, 1H). HPLC Column: CHIRALPAK IC (250 mm × 4.6 mm × 5 mic) Mobile phase: MTBE:IPA with 0.1% DEA (50:50) Flow rate: 1.0 mL/min Retention time: 14.798 min |
| 166 | 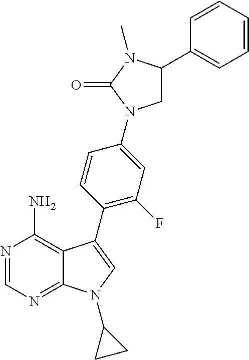 [SMILES: CN1C(=O)N(c2ccc(-c3cn(C4CC4)c4ncnc(N)c34)c(F)c2)CC1c1ccccc1] | 1-(4-(4-amino-7-cyclopropyl-7H- pyrrolo[2,3-d]pyrimidin-5-yl)-3- fluorophenyl)-3-methyl-4- phenylimidazolidin-2-one (enantiomer 2) | 443.2 | 0.99-1.01 (m, 4H), 2.59 (s, 3H), 3.53-362 (m, 2H), 4.26 (t, J = 10.0 Hz, 1H), 4.72 (t, J = 9.2 Hz, 1H), 5.91 (br, s. 2H), 7.17 (s, 1H), 7.30-7.45 (m, 7H), 7.68-7.71 (m, 1H), 8.12 (s, 1H). HPLC Column: CHIRALPAK IC (250 mm × 4.6 mm × 5 mic) Mobile phase: MTBE:IPA with 0.1% DEA (50:50) Flow rate: 1.0 mL/min Retention time: 20.426 min |
| 167 | 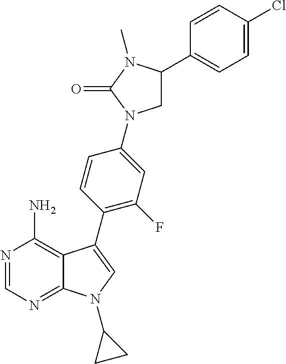 [SMILES: CN1C(=O)N(c2ccc(-c3cn(C4CC4)c4ncnc(N)c34)c(F)c2)CC1c1ccc(Cl)cc1] | 1-(4-(4-amino-7-cyclopropyl-7H- pyrrolo[2,3-d]pyrimidin-5-yl)-3- fluorophenyl)-4-(4-chlorophenyl)-3- methylimidazolidin-2-one | 477.1 | 0.97-1.01 (m, 4H), 2.59 (s, 3H), 3.52-361 (m, 2H), 4.25 (t, J = 9.2 Hz, 1H), 4.75 (t, J = 8.0 Hz, 1H), 5.91 (br, s. 2H), 7.17 (s, 1H), 7.32 (t, J = 8.4 Hz, 1H), 7.39-7.42 (m, 3H), 7.49 (d, J = 8.4 Hz, 2H), 7.67-7.70 (m, 1H), 8.12 (s, 1H). |
| 168 | 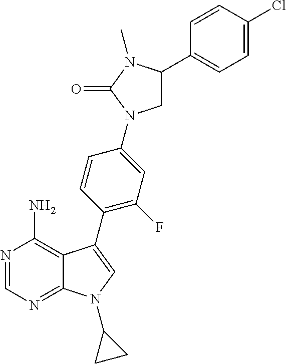 [SMILES: CN1C(=O)N(c2ccc(-c3cn(C4CC4)c4ncnc(N)c34)c(F)c2)CC1c1ccc(Cl)cc1] | 1-(4-(4-amino-7-cyclopropyl-7H- pyrrolo[2,3-d]pyrimidin-5-yl)-3- fluorophenyl)-4-(4-chlorophenyl)-3- methylimidazolidin-2-one (enantiomer 1) | 477.1 | 0.99-1.01 (m, 4H), 2.59 (s, 3H), 3.53-361 (m, 2H), 4.25 (t, J = 9.2 Hz, 1H), 4.75 (t, J = 8.4 Hz, 1H), 5.91 (br, s. 2H), 7.17 (s, 1H), 7.32 (t, J = 8.4 Hz, 1H), 7.39-7.42 (m, 3H), 7.49 (d, J = 8.4 Hz, 2H), 7.69 (d, J = 13.2 Hz, 1H), 8.12 (s, 1H). HPLC Column: CHIRALPAK IC (250 mm × 4.6 mm × 5 mic) Mobile phase (A:B): MTBE:Ethanol with 0.1% DEA: (80:20) Flow: 1.0 ml/min Retention time: 14.097 min |
| 169 | 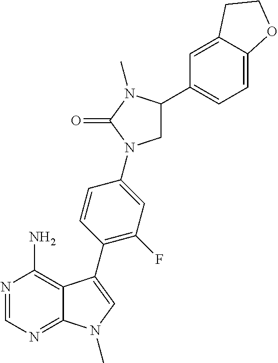 [SMILES: CN1C(=O)N(c2ccc(-c3cn(C)c4ncnc(N)c34)c(F)c2)CC1c1ccc2c(c1)CCO2] | 1-(4-(4-amino-7-methyl-7H- pyrrolo[2,3-d]pyrimidin-5-yl)-3- fluorophenyl)-4-(2,3- dihydrobenzofuran-5-yl)-3- methylimidazolidin-2-one (enantiomer 1) | 459.1 | 2.57 (s, 3H), 3.17 (t, J = 8.8 Hz, 1H), 3.58 (t, J = 8.0 Hz, 1H), 3.72 (s, 3H), 4.21 (t, J = 9.2 Hz, 1H), 4.52 (t, J = 8.4 Hz, 2H), 4.63 (t, J = 8.0 Hz, 1H), 5.92 (br, s. 2H), 6.77 (d, J = 8.0 Hz, 1H), 7.43 (d, J = 8.4 Hz, 1H), 7.24 (s, 2H), 7.32 (t, J = 8.8 Hz, 1H), 7.41 (d, J = 8.4 Hz, 1H), 7.71 (d, J = 13.2 Hz, 1H), 8.12 (s, 1H). HPLC Column: CHIRALPAK IC (250 mm × 4.6 mm × 5 mic) Mobile phase: MTBE:Methanol with 0.1% DEA (85:15) Flow rate: 1.0 mL/min Retention time: 14.508 min |
| 170 | 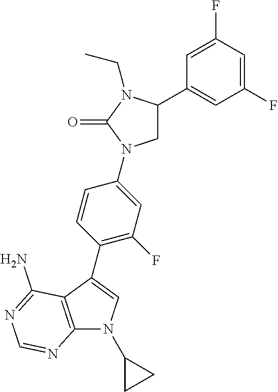 [SMILES: CCN1C(=O)N(c2ccc(-c3cn(C4CC4)c4ncnc(N)c34)c(F)c2)CC1c1cc(F)cc(F)c1] | 1-(4-(4-amino-7-cyclopropyl-7H- pyrrolo[2,3-d]pyrimidin-5-yl)-3- fluorophenyl)-4-(3,5-difluorophenyl)- 3-ethylimidazolidin-2-one | 493.2 | 0.95-1.01 (m, 7H), 2.76 (q, J = 7.6 Hz, 1H), 3.42 (q, J = 7.0 Hz, 1H), 3.54 (s, 1H), 3.65 (t, J = 8.2 Hz, 1H), 4.24 (t, J = 9.4 Hz, 1H), 4.93 (t, J = 7.6 Hz, 1H), 5.90 (br. s, 2H), 7.16-7.26 (m, 4H), 7.32 (t, J = 84 Hz, 1H), 7.40 (d, J = 8.0 Hz, 1H), 7.69 (d, J = 12.8 Hz, 1H), 8.13 (s, 1H). |
| 171 | 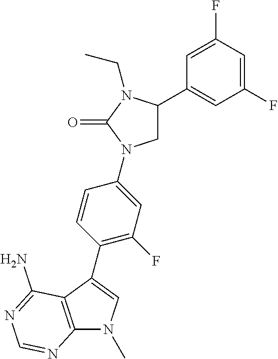 [SMILES: CCN1C(=O)N(c2ccc(-c3cn(C)c4ncnc(N)c34)c(F)c2)CC1c1cc(F)cc(F)c1] | 1-(4-(4-amino-7-methyl-7H- pyrrolo[2,3-d]pyrimidin-5-yl)-3- fluorophenyl)-4-(3,5-difluorophenyl)- 3-ethylimidazolidin-2-one (enantiomer 1) | 467.2 | 0.97 (t, J = 6.8 Hz, 3H), 2.73-2.79 (m, 1H), 3.41-3.45 (m, 1H), 3.63-3.67 (m, 1H), 3.72 (s, 3H), 4.25 (t, J = 9.4 Hz, 1H), 4.94 (t, J = 7.6 Hz, 1H), 5.92 (br. s, 2H), 7.20 (d, J = 6.4 Hz, 2H), 7.25 (s, 2H), 7.32 (t, J = 8.8 Hz, 1H), 7.41 (d, J = 8.4 Hz, 1H), 7.69 (d, J = 13.2 Hz, 1H), 8.12 (s, 1H). HPLC Column: CHIRALPAK IA (250 mm × 4.6 mm × 5 μm) Mobile phase: n- Hexane:Methanol with 0.1% DEA (90:10) Flow rate: 1.0 mL/min Retention time: 11.207 min |
| 172 | 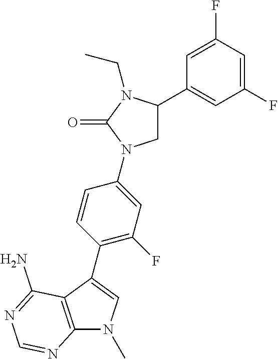 [SMILES: CCN1C(=O)N(c2ccc(-c3cn(C)c4ncnc(N)c34)c(F)c2)CC1c1cc(F)cc(F)c1] | 1-(4-(4-amino-7-methyl-7H- pyrrolo[2,3-d]pyrimidin-5-yl)-3- fluorophenyl)-4-(3,5-difluorophenyl)- 3-ethylimidazolidin-2-one (enantiomer 2) | 467.2 | 0.97 (t, J = 7.0 Hz, 3H), 2.75-2.79 (m, 1H), 3.41-3.45 (m, 1H), 3.63-3.67 (m, 1H), 3.72 (s, 3H), 4.25 (t, J = 9.4 Hz, 1H), 4.93 (t, J = 8.0 Hz, 1H), 5.92 (br. s, 2H), 7.20 (d, J = 6.4 Hz,, 2H), 7.24 (s, 2H), 7.32 (t, J = 8.8 Hz, 1H), 7.41 (d, J = 8.4 Hz, 1H), 7.69 (d, J = 13.2 Hz, 1H), 8.12 (s, 1H). HPLC Column: CHIRALPAK IA (250 mm × 4.6 mm × 5 μm) Mobile phase: n- Hexane:Methanol with 0.1% DEA (90:10) Flow rate: 1.0 mL/min Retention time: 11.591 min |
| 173 | 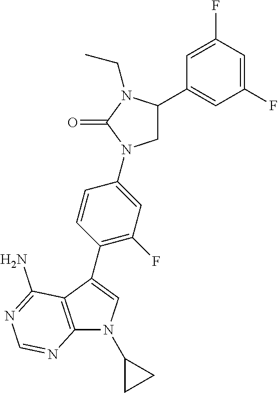 [SMILES: CCN1C(=O)N(c2ccc(-c3cn(C4CC4)c4ncnc(N)c34)c(F)c2)CC1c1cc(F)cc(F)c1] | 1-(4-(4-amino-7-cyclopropyl-7H- pyrrolo[2,3-d]pyrimidin-5-yl)-3- fluorophenyl)-4-(3,5-difluorophenyl)- 3-ethylimidazolidin-2-one (enantiomer 1) | 493.1 | 0.95-0.97 (m, 3H), 0.98-1.01 (m, 4H), 2.72-2.80 (m, 1H), 3.41-3.47 (m, 1H), 3.52-3.57 (m, 1H), 3.63-3.67 (m, 1H), 4.24 (t, J = 9.8 Hz, 1H), 4.91-4.95 (m, 1H), 5.91 (br. s, 2H), 7.17-7.26 (m, 4H), 7.32 (t, J = 8.6 Hz, 1H), 7.40 (dd, J = 2.0, 8.8 Hz, 1H), 7.68 (dd, J = 2.0, 13.2 Hz, 1H), 8.12 (s, 1H). HPLC Column: CHIRALPAK IA (250 mm × 4.6 mm × 5 μm) Mobile phase: n- Hexane:Methanol with 0.1% DEA (90:10) Flow rate: 1.0 mL/min Retention time: 10.477 min |
| 174 | 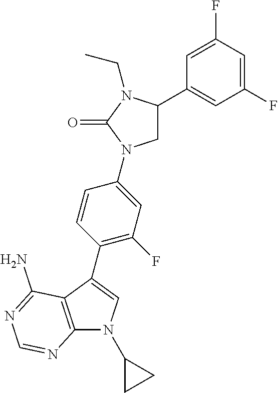 [SMILES: CCN1C(=O)N(c2ccc(-c3cn(C4CC4)c4ncnc(N)c34)c(F)c2)CC1c1cc(F)cc(F)c1] | 1-(4-(4-amino-7-cyclopropyl-7H- pyrrolo[2,3-d]pyrimidin-5-yl)-3- fluorophenyl)-4-(3,5-difluorophenyl)- 3-ethylimidazolidin-2-one (enantiomer 2) | 493.2 | 0.95-1.07 (m, 7H), 2.73-3.27 (m, 1H), 3.37-3.45 (m, 1H), 3.52-3.57 (m, 1H), 3.63-3.67 (m, 1H), 4.24 (t, J = 9.8 Hz, 1H), 4.91-4.95 (m, 1H), 5.91 (br. s, 2H), 7.17-7.26 (m, 4H), 7.32 (t, J = 8.8 Hz, 1H), 7.40 (dd, J = 2.0, 8.4 Hz, 1H), 7.69 (dd, J = 2.0, 12.8 Hz, 1H), 8.12 (s, 1H). HPLC Column: CHIRALPAK IA (250 mm × 4.6 mm × 5 μm) Mobile phase: n- Hexane:Methanol with 0.1% DEA (90:10) Flow rate: 1.0 mL/min Retention time: 12.051 min |

Compounds 178 to 195 are prepared generally according to the above Schemes and the procedures described for Examples 1 to 13, 46, 59, 100, 110, 115, 137 and 175 to 177.

TABLE 2

| | | | LCMS | |
|---|---|---|---|---|
| | | | m/z | ¹H-NMR (400 MHz, |
| Compound # | Structure | Name | [M + H]⁺) | DMSO-d₆) |
| 178 | 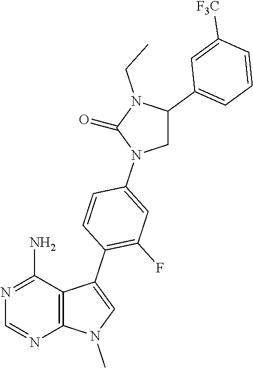 [SMILES: CCN1C(=O)N(c2ccc(-c3cn(C)c4ncnc(N)c34)c(F)c2)CC1c1cccc(C(F)(F)F)c1] | 1-(4-(4-amino-7- methyl-7H- pyrrolo[2,3- d]pyrimidin-5-yl)-3- fluorophenyl)-3- ethyl-4-(3- (trifluoromethyl) phenyl)imidazolidin- 2-one | | |
| 179 | 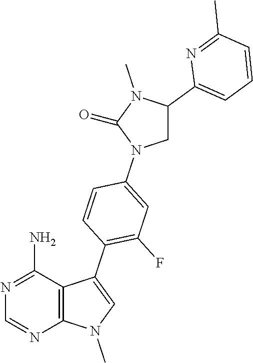 [SMILES: Cc1cccc(C2CN(c3ccc(-c4cn(C)c5ncnc(N)c45)c(F)c3)C(=O)N2C)n1] | 1-(4-(4-amino-7- methyl-7H- pyrrolo[2,3- d]pyrimidin-5-yl)-3- fluorophenyl)-3- methyl-4-(6- methylpyridin-2- yl)imidazolidin-2- one | | |
| 180 | 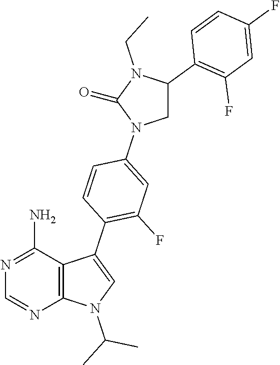 [SMILES: CCN1C(=O)N(c2ccc(-c3cn(C(C)C)c4ncnc(N)c34)c(F)c2)CC1c1ccc(F)cc1F] | 1-(4-(4-amino-7- isopropyl-7H- pyrrolo[2,3- d]pyrimidin-5-yl)-3- fluorophenyl)-4- (2,4- difluorophenyl)-3- ethylimidazolidin- 2-one | | |
| 181 | 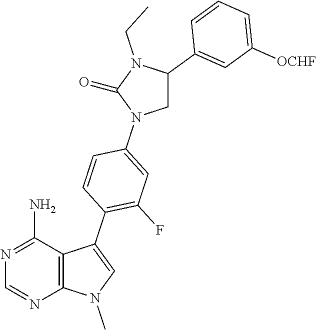 | 1-(4-(4-amino-7- methyl-7H- pyrrolo[2,3- d]pyrimidin-5-yl)-3- fluorophenyl)-4-(3- (difluoromethoxy) phenyl)-3- ethylimidazolidin- 2-one | | |
| 182 | 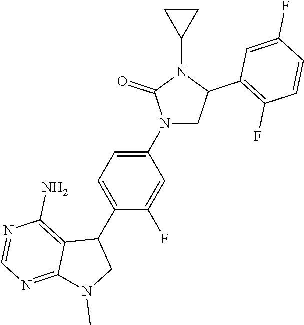 [SMILES: CN1CC(c2ccc(N3CC(c4cc(F)ccc4F)N(C4CC4)C3=O)cc2F)c2c(N)ncnc21] | 1-(4-(4-amino-7- methyl-7H- pyrrolo[2,3- d]pyrimidin-5-yl)-3- fluorophenyl)-3- cyclopropyl-4-(2,5- difluorophenyl) imidazolidin-2-one | | |
| 183 | 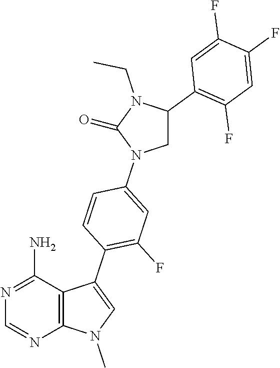 [SMILES: CCN1C(=O)N(c2ccc(-c3cn(C)c4ncnc(N)c34)c(F)c2)CC1c1cc(F)c(F)cc1F] | 1-(4-(4-amino-7- methyl-7H- pyrrolo[2,3- d]pyrimidin-5-yl)-3- fluorophenyl)-3- ethyl-4-(2,4,5- trifluorophenyl) imidazolidin-2-one | | |
| 184 | 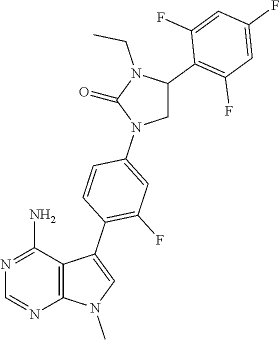 [SMILES: CCN1C(=O)N(c2ccc(-c3cn(C)c4ncnc(N)c34)c(F)c2)CC1c1c(F)cc(F)cc1F] | 1-(4-(4-amino-7- methyl-7H- pyrrolo[2,3- d]pyrimidin-5-yl)-3- fluorophenyl)-3- ethyl-4-(2,4,6- trifluorophenyl) imidazolidin-2-one | | |
| 185 | 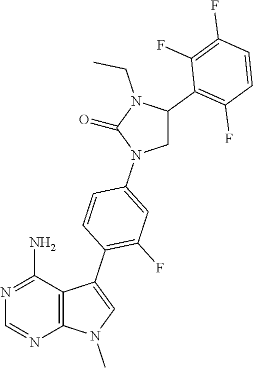 [SMILES: CCN1C(=O)N(c2ccc(-c3cn(C)c4ncnc(N)c34)c(F)c2)CC1c1c(F)ccc(F)c1F] | 1-(4-(4-amino-7- methyl-7H- pyrrolo[2,3- d]pyrimidin-5-yl)-3- fluorophenyl)-3- ethyl-4-(2,3,6- trifluorophenyl) imidazolidin-2-one | | |
| 186 | 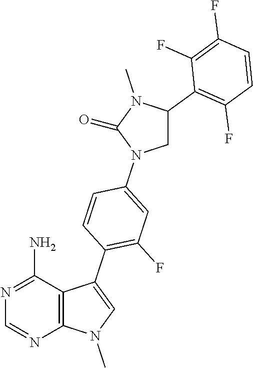 [SMILES: CN1C(=O)N(c2ccc(-c3cn(C)c4ncnc(N)c34)c(F)c2)CC1c1c(F)ccc(F)c1F] | 1-(4-(4-amino-7- methyl-7H- pyrrolo[2,3- d]pyrimidin-5-yl)-3- fluorophenyl)-3- methyl-4-(2,3,6- trifluorophenyl) imidazolidin-2-one | | |
| 187 | 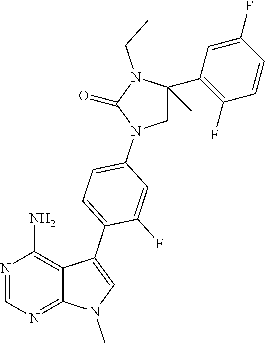 [SMILES: CCN1C(=O)N(c2ccc(-c3cn(C)c4ncnc(N)c34)c(F)c2)CC1(C)c1cc(F)ccc1F] | 1-(4-(4-amino-7- methyl-7H- pyrrolo[2,3- d]pyrimidin-5-yl)-3- fluorophenyl)-4- (2,5- difluorophenyl)-3- ethyl-4- methylimidazolidin- 2-one | | |
| 188 | 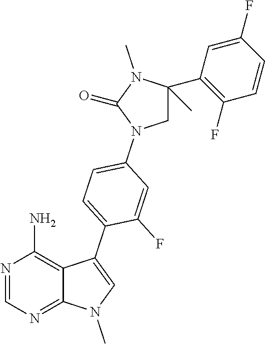 [SMILES: CN1C(=O)N(c2ccc(-c3cn(C)c4ncnc(N)c34)c(F)c2)CC1(C)c1cc(F)ccc1F] | 1-(4-(4-amino-7- methyl-7H- pyrrolo[2,3- d]pyrimidin-5-yl)-3- fluorophenyl)-4- (2,5- difluorophenyl)- 3,4- dimethylimidazolidin- 2-one | | |
| 189 | 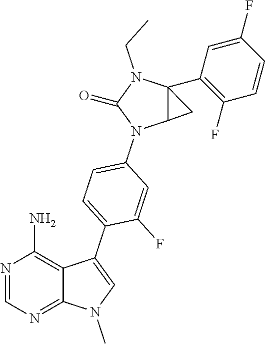 [SMILES: CCN1C(=O)N(c2ccc(-c3cn(C)c4ncnc(N)c34)c(F)c2)C2CC21c1cc(F)ccc1F] | 4-(4-(4-amino-7- methyl-7H- pyrrolo[2,3- d]pyrimidin-5-yl)-3- fluorophenyl)-1- (2,5- difluorophenyl)-2- ethyl-2,4- diazabicyclo[3.1.0] hexan-3-one | | |
| 190 | 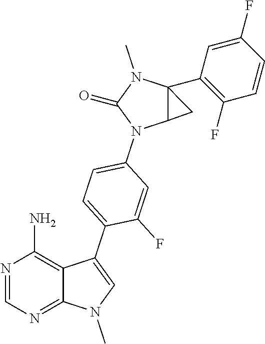 [SMILES: CN1C(=O)N(c2ccc(-c3cn(C)c4ncnc(N)c34)c(F)c2)C2CC21c1cc(F)ccc1F] | 4-(4-(4-amino-7- methyl-7H- pyrrolo[2,3- d]pyrimidin-5-yl)-3- fluorophenyl)-1- (2,5- difluorophenyl)-2- methyl-2,4- diazabicyclo[3.1.0] hexan-3-one | | |
| 191 | 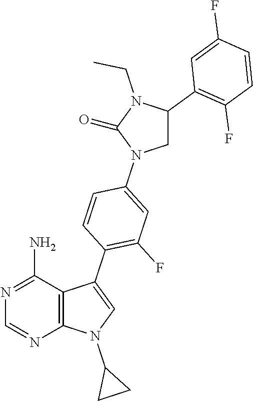 [SMILES: CCN1C(=O)N(c2ccc(-c3cn(C4CC4)c4ncnc(N)c34)c(F)c2)CC1c1cc(F)ccc1F] | 1-(4-(4-amino-7- cyclopropyl-7H- pyrrolo[2,3- d]pyrimidin-5-yl)-3- fluorophenyl)-4- (2,5- difluorophenyl)-3- ethylimidazolidin- 2-one | | |
| 192 | 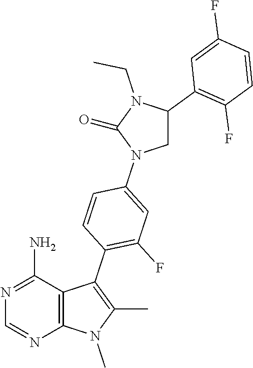 [SMILES: CCN1C(=O)N(c2ccc(-c3c(C)n(C)c4ncnc(N)c34)c(F)c2)CC1c1cc(F)ccc1F] | 1-(4-(4-amino-6,7- dimethyl-7H- pyrrolo[2,3- d]pyrimidin-5-yl)-3- fluorophenyl)-4- (2,5- difluorophenyl)-3- ethylimidazolidin- 2-one | | |
| 193 | 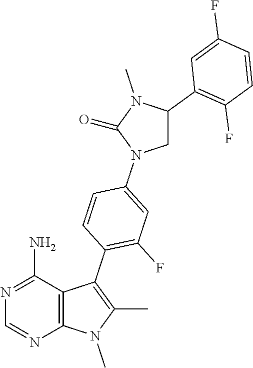 [SMILES: Cc1c(-c2ccc(N3CC(c4cc(F)ccc4F)N(C)C3=O)cc2F)c2c(N)ncnc2n1C] | 1-(4-(4-amino-6,7- dimethyl-7H- pyrrolo[2,3- d]pyrimidin-5-yl)-3- fluorophenyl)-4- (2,5- difluorophenyl)-3- methylimidazolidin- 2-one | | |
| 194 | 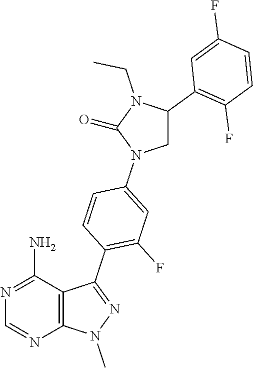 [SMILES: CCN1C(=O)N(c2ccc(-c3nn(C)c4ncnc(N)c34)c(F)c2)CC1c1cc(F)ccc1F] | 1-(4-(4-amino-1- methyl-1H- pyrazolo[3,4- d]pyrimidin-3-yl)-3- fluorophenyl)-4- (2,5- difluorophenyl)-3- ethylimidazolidin- 2-one | | |
| 195 | 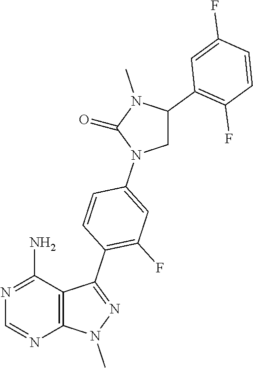 [SMILES: CN1C(=O)N(c2ccc(-c3nn(C)c4ncnc(N)c34)c(F)c2)CC1c1cc(F)ccc1F] | 1-(4-(4-amino-1- methyl-1H- pyrazolo[3,4- d]pyrimidin-3-yl)-3- fluorophenyl)-4- (2,5- difluorophenyl)-3- methylimidazolidin- 2-one | | |

### Example 196: PERK Enzyme Assay

Compounds of the invention were assayed for PERK enzyme inhibitory activity with modifications to previously reported conditions (Axten et al. J. Med. Chem., 2012, 55, 7193-7207). Briefly, various concentrations of compounds (maximum 1% DMSO) were dispensed into 384-well plates containing GST-PERK enzyme. After 30-60 minutes of compound pre-incubation, ATP and biotin-eIF2α were added and after 60 minutes the reaction was quenched. After 2 hrs, a fluorescence plate reader was used to measure inhibition and IC50s were calculated.

### Enzyme Assay Protocol for PKR-Like Endoplasmic Reticulum Kinase (PERK)—HTRF —% Inhibition

Assay Buffer contains HEPES (pH7.5) 10 mM, CHAPS 2 mM, MgCl2 5 mM and DTT 1 mM in water

Detection Buffer contains HEPES (pH7.5) 10 mM and CHAPS 2 mM in water Assay Plate Preparation:

1. Enzyme Preparation:
4× Enzyme solution was prepared immediately prior to adding to compound plates.
3 nM of GST-PERK in Assay buffer. Final [PERK] in 10 μl assay volume=0.75 nM

2. Substrate Preparation:
4× Substrate solution was prepared immediately prior to adding to compound plates.
   4× Substrate solution in assay buffer 2000 μM ATP and 160 nM Biotin-eIF2α
Final [ATP] in 10 μl assay volume=500 μM
Final [biotin-eIF2a] in 10 μl assay volume=40 nM.

3. Quench/Detection Solution:
16 nM eIF2 Phospho-Antibody
16 nM Eu anti-Rabbit IgG
160 nM Streptavidin-APC
60 mM EDTA
Final concentration in 10 μL assay volume: 4 nM eIF2 Phospho-Antibody, 4 nM Eu anti-Rabbit IgG 40 nM Streptavidin-APC

The activity of compounds in the PERK enzyme assay was determined at PERK Enzyme (500 μM ATP) IC50 (nM).

### Example 197—Capsule Composition

An oral dosage form for administering the present invention is produced by filing a standard two piece hard gelatin capsule with the ingredients in the proportions shown in Table 3, below.

TABLE 3

| INGREDIENTS | AMOUNTS |
|---|---|
| 1-(4-(4-amino-7-methyl-7H-pyrrolo[2,3-d]pyrimidin-5-yl)- | 7 mg |
| 3-fluorophenyl)-4-(2,5-difluorophenyl)-3- | |
| isobutylimidazolidin-2-one (Compound of Example 14) | |
| Lactose | 53 mg |
| Talc | 16 mg |
| Magnesium Stearate | 4 mg |

### Example 198—Injectable Parenteral Composition

An injectable form for administering the present invention is produced by stirring 1.7% by weight of 1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one (Compound of Example 15) in 10% by volume propylene glycol in water.

### Example 199 Tablet Composition

The sucrose, calcium sulfate dihydrate and a PERK inhibitor as shown in Table 4 below, are mixed and granulated in the proportions shown with a 10% gelatin solution. The wet granules are screened, dried, mixed with the starch, talc and stearic acid, screened and compressed into a tablet.

TABLE 4

| INGREDIENTS | AMOUNTS |
|---|---|
| 1-(4-(4-amino-7-methyl-7H-pyrrolo[2,3-d]pyrimidin-5-yl)- | 12 mg |
| 3-fluorophenyl)-4-(3-chloro-5-fluorophenyl)-3- | |
| methylimidazolidin-2-one (Compound of Example 16) | |
| calcium sulfate dihydrate | 30 mg |
| sucrose | 4 mg |
| starch | 2 mg |
| talc | 1 mg |
| stearic acid | 0.5 mg |

### Biological Activity

Compounds of the invention are tested for activity against PERK in the above assay.

The compounds of Examples 1 to 177 were tested generally according to the above PERK enzyme assay and in at least one experimental run exhibited the PERK Enzyme (500 μM ATP) IC50 (nM) value indicated in Table 5.

TABLE 5
PERK activity for examples of the invention^{a}

| | PERK |
|---|---|
| Example # | Activity |
| 1 | ++ |
| 2 | +++ |
| 3 | ++ |
| 4 | ++ |
| 5 | ++ |
| 6 | + |
| 7 | +++ |
| 8 | +++ |
| 9 | + |
| 10 | + |
| 11 | +++ |
| 12 | +++ |
| 13 | + |
| 14 | ++ |
| 15 | +++ |
| 16 | ++ |
| 17 | ++ |
| 18 | ++ |
| 19 | + |
| 20 | + |
| 21 | ++ |
| 22 | ++ |
| 23 | ++ |
| 24 | ++ |
| 25 | +++ |
| 26 | ++ |
| 27 | ++ |
| 28 | ++ |
| 29 | ++ |
| 30 | +++ |
| 31 | ++ |
| 32 | ++ |
| 33 | ++ |
| 34 | ++ |
| 35 | ++ |
| 36 | ++ |
| 37 | ++ |
| 38 | + |
| 39 | ++ |
| 40 | ++ |
| 41 | ++ |
| 42 | ++ |
| 43 | +++ |
| 44 | +++ |
| 45 | + |
| 46 | +++ |
| 47 | + |
| 48 | ++ |
| 49 | ++ |
| 50 | ++ |
| 51 | +++ |
| 52 | ++ |
| 53 | ++ |
| 54 | +++ |
| 55 | ++ |
| 56 | ++ |
| 57 | +++ |
| 58 | +++ |
| 59 | +++ |
| 60 | +++ |
| 61 | ++ |
| 62 | +++ |
| 63 | +++ |
| 64 | ++ |
| 65 | ++ |
| 66 | +++ |
| 67 | ++ |
| 68 | +++ |
| 69 | +++ |
| 70 | +++ |
| 71 | ++ |
| 72 | ++ |
| 73 | ++ |
| 74 | ++ |
| 75 | ++ |
| 76 | ++ |
| 77 | +++ |
| 78 | +++ |
| 79 | +++ |
| 80 | +++ |
| 81 | +++ |
| 82 | ++ |
| 83 | ++ |
| 84 | ++ |
| 85 | ++ |
| 86 | ++ |
| 87 | ++ |
| 88 | ++ |
| 89 | ++ |
| 90 | +++ |
| 91 | +++ |
| 92 | ++ |
| 93 | ++ |
| 94 | ++ |
| 95 | ++ |
| 96 | +++ |
| 97 | +++ |
| 98 | ++ |
| 99 | +++ |
| 100 | +++ |
| 101 | ++ |
| 102 | ++ |
| 103 | +++ |
| 104 | +++ |
| 105 | ++ |
| 106 | ++ |
| 107 | +++ |
| 108 | +++ |
| 109 | +++ |
| 110 | + |
| 111 | ++ |
| 112 | ++ |
| 113 | +++ |
| 114 | +++ |
| 115 | +++ |
| 116 | +++ |
| 117 | ++ |
| 118 | +++ |
| 119 | +++ |
| 120 | +++ |
| 121 | +++ |
| 122 | +++ |
| 123 | ++ |
| 124 | +++ |
| 125 | +++ |
| 126 | +++ |
| 127 | +++ |
| 128 | +++ |
| 129 | +++ |
| 130 | +++ |
| 131 | +++ |
| 132 | +++ |
| 133 | +++ |
| 134 | +++ |
| 135 | +++ |
| 136 | ++ |
| 137 | +++ |
| 138 | +++ |
| 139 | ++ |
| 140 | +++ |
| 141 | ++ |
| 142 | +++ |
| 143 | ++ |
| 144 | +++ |
| 145 | +++ |
| 146 | +++ |
| 147 | +++ |
| 148 | +++ |
| 149 | +++ |
| 150 | +++ |
| 151 | ++ |
| 152 | +++ |
| 153 | ++ |
| 154 | ++ |
| 155 | +++ |
| 156 | +++ |
| 157 | +++ |
| 158 | +++ |
| 159 | ++ |
| 160 | +++ |
| 161 | +++ |
| 162 | +++ |
| 163 | +++ |
| 164 | ++ |
| 165 | +++ |
| 166 | +++ |
| 167 | +++ |
| 168 | +++ |
| 169 | +++ |
| 170 | +++ |
| 171 | +++ |
| 172 | +++ |
| 173 | +++ |
| 174 | +++ |
| 175 | +++ |
| 176 | +++ |
| 177 | +++ |

^{a}PERK IC50 <100 nM = “+++”; PERK IC50 100-1000 nM = “++”; PERK IC50 >1000 nM = “+”

The compound of Example 2 was tested generally according to the above PERK enzyme assay and in at least one experimental run exhibited a PERK Enzyme (500 μM ATP) IC50 (nM) value of 77.4 against PERK

The compound of Example 14 was tested generally according to the above PERK enzyme assay and in at least one experimental run exhibited a PERK Enzyme (500 μM ATP) IC50 (nM) value of 903.4 against PERK.

The compound of Example 20 was tested generally according to the above PERK enzyme assay and in at least one experimental run exhibited a PERK Enzyme (500 μM ATP) IC50 (nM) value of 3644.9 against PERK.

The compound of Example 25 was tested generally according to the above PERK enzyme assay and in at least one experimental run exhibited a PERK Enzyme (500 μM ATP) IC50 (nM) value of 74.2 against PERK.

The compound of Example 36 was tested generally according to the above PERK enzyme assay and in at least one experimental run exhibited a PERK Enzyme (500 μM ATP) IC50 (nM) value of 462 against PERK.

The compound of Example 46 was tested generally according to the above PERK enzyme assay and in at least one experimental run exhibited a PERK Enzyme (500 μM ATP) IC50 (nM) value of 13.8 against PERK.

The compound of Example 59 was tested generally according to the above PERK enzyme assay and in at least one experimental run exhibited a PERK Enzyme (500 μM ATP) IC50 (nM) value of 3.9 against PERK.

The compound of Example 66 was tested generally according to the above PERK enzyme assay and in at least one experimental run exhibited a PERK Enzyme (500 μM ATP) IC50 (nM) value of 86.9 against PERK.

The compound of Example 70 was tested generally according to the above PERK enzyme assay and in at least one experimental run exhibited a PERK Enzyme (500 μM ATP) IC50 (nM) value of 18.6 against PERK.

The compound of Example 88 was tested generally according to the above PERK enzyme assay and in at least one experimental run exhibited a PERK Enzyme (500 μM ATP) IC50 (nM) value of 192 against PERK.

The compound of Example 92 was tested generally according to the above PERK enzyme assay and in at least one experimental run exhibited a PERK Enzyme (500 μM ATP) IC50 (nM) value of 350.9 against PERK.

The compound of Example 100 was tested generally according to the above PERK enzyme assay and in at least one experimental run exhibited a PERK Enzyme (500 μM ATP) IC50 (nM) value of 3 against PERK.

The compound of Example 110 was tested generally according to the above PERK enzyme assay and in at least one experimental run exhibited a PERK Enzyme (500 μM ATP) IC50 (nM) value of 1981.3 against PERK.

The compound of Example 115 was tested generally according to the above PERK enzyme assay and in at least one experimental run exhibited a PERK Enzyme (500 μM ATP) IC50 (nM) value of 2.4 against PERK.

The compound of Example 127 was tested generally according to the above PERK enzyme assay and in at least one experimental run exhibited a PERK Enzyme (500 μM ATP) IC50 (nM) value of 6.1 against PERK.

The compound of Example 137 was tested generally according to the above PERK enzyme assay and in at least one experimental run exhibited a PERK Enzyme (500 μM ATP) IC50 (nM) value of 17.8 against PERK.

The compound of Example 154 was tested generally according to the above PERK enzyme assay and in at least one experimental run exhibited a PERK Enzyme (500 μM ATP) IC50 (nM) value of 988.4 against PERK.

The compound of Example 165 was tested generally according to the above PERK enzyme assay and in at least one experimental run exhibited a PERK Enzyme (500 μM ATP) IC50 (nM) value of 4 against PERK.

The compound of Example 172 was tested generally according to the above PERK enzyme assay and in at least one experimental run exhibited a PERK Enzyme (500 μM ATP) IC50 (nM) value of 30.1 against PERK.

While the preferred embodiments of the invention are illustrated by the above, it is to be understood that the invention is not limited to the precise instructions herein disclosed and that the right to all modifications coming within the scope of the following claims is reserved.

1. A compound according to Formula I: 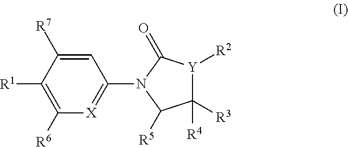
wherein:
R¹ is selected from:
bicycloheteroaryl,
substituted bicycloheteroaryl,
heteroaryl, and
substituted heteroaryl,
where said substituted bicycloheteroaryl and said substituted heteroaryl are substituted with from one to five substituents independently selected from:
fluoro,
chloro,
bromo,
iodo,
C₁₋₆alkyl,
C₁₋₆alkyl substituted with from 1 to 5 substituents independently selected from: fluoro, chloro, bromo, iodo, C₁₋₄alkyloxy, —OH, C₁₋₄alkyl, cycloalkyl, —COOH, —CF₃, —NO₂, —NH₂ and —CN,
—OH,
hydroxyC₁₋₆alkyl,
—COOH,
tetrazole,
cycloalkyl,
oxo,
—OC₁₋₆alkyl,
—CF₃,
—CF₂H,
—CFH₂,
—C₁₋₆alkylOC₁₋₄alkyl,
—CONH₂,
—CON(H)C₁₋₃alkyl,
—CH₂CH₂N(H)C(O)OCH₂aryl,
diC₁₋₄alkylaminoC₁₋₄alkyl,
aminoC₁₋₆alkyl,
—CN,
heterocycloalkyl,
heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, oxo, —NO₂, —NH₂ and —CN,
—NO₂,
—NH₂,
—N(H)C₁₋₃alkyl, and
—N(C₁₋₃alkyl)₂;
R² is selected from:
hydrogen,
—NH₂,
—N(H)C₁₋₃alkyl,
—N(C₁₋₃alkyl)₂,
—OH,
cycloalkyl,
benzyl,
aryl,
heterocycloalkyl,
heteroaryl,
C₁₋₆alkyl, and
C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R³ is selected from:
aryl,
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
heteroaryl,
heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
bicycloheteroaryl,
bicycloheteroaryl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂, cycloalkyl, —OC(H)F₂, —C(H)F₂, —OCH₂F, —CH₂F, —OCF₃, and —CN, and
cycloalkyl;
R⁴ and R⁵ are each independently selected from hydrogen and C₁₋₆alkyl,
or R⁴ and R⁵ taken together with the carbon atoms to which they are attached represent a 3 or 4 member cycloalkyl; and
R⁶ is selected from: hydrogen, C₁₋₄alkyl, —CF₃, —C(H)F₂, —CH₂F, fluoro, chloro, bromo and iodo;
R⁷ is selected from: hydrogen, C₁₋₄alkyl, —CF₃, —C(H)F₂, —CH₂F, fluoro, chloro, bromo and iodo;
Y is CR⁹⁰ or N,
where R⁹⁰ is selected from: hydrogen, C₁₋₄alkyl, cycloalkyl, —OH, —NH₂, —CN, and —CF₃; and
X is CR¹⁰⁰ or N,
where R¹⁰⁰ is selected from: hydrogen, —CH₃, —CF₃, fluoro, chloro, bromo and iodo;
or a salt thereof including a pharmaceutically acceptable salt thereof.

2. The compound of claim 1 represented by the following Formula (IIa): 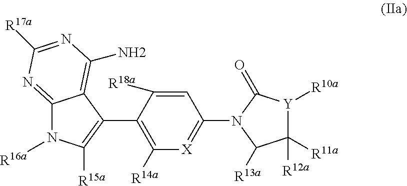
wherein:
R^{10a} is selected from:
hydrogen,
—NH₂,
—N(H)C₁₋₃alkyl,
—N(C₁₋₃alkyl)₂,
—OH,
cycloalkyl,
phenyl,
benzyl,
heterocycloalkyl,
heteroaryl,
C₁₋₆alkyl, and
C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R^{11a} is selected from:
aryl,
aryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —NH₂, —OC(H)F₂, —C(H)F₂, —OCH₂F, —CH₂F, —OCF₃, and —CN,
cycloalkyl,
heteroaryl, and
heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —C₁₋₄alkylOC₁₋₄alkyl, —NO₂, —OC(H)F₂, —C(H)F₂, —OCH₂F, —CH₂F, —OCF₃, —NH₂ and —CN;
R^{12a} and R^{13a} are each independently selected from hydrogen and C₁₋₆alkyl,
or R^{12a} and R^{13a} taken together with the carbon atoms to which they are attached represent a 3 or 4 member cycloalkyl;
R^{14a} is selected from: hydrogen, C₁₋₄alkyl, —CF₃, —C(H)F₂, —CH₂F, fluoro and chloro;
R^{15a} is selected from hydrogen and C₁₋₆alkyl;
R^{16a} is selected from:
hydrogen,
cycloalkyl,
heterocycloalkyl,
heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, —NO₂, —NH₂ and —CN,
C₁₋₆alkyl, and
C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R^{17a} is selected from: hydrogen and —CH₃;
R^{18a} is selected from: hydrogen, C₁₋₄alkyl, —CF₃, —C(H)F₂, —CH₂F, fluoro and chloro;
Y is CR^{91a} or N,
where R^{91a} is selected from: hydrogen, C₁₋₄alkyl, cycloalkyl, —OH, —NH₂, —CN, and —CF₃; and
X is CR^{101a} or N,
where R^{10a} is selected from: hydrogen, fluoro and chloro;
or a salt thereof including a pharmaceutically acceptable salt thereof.

3. The compound of claim 1 represented by the following Formula (IIIa): 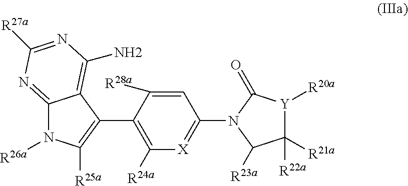
wherein:
R^{20a} is selected from:
hydrogen,
cycloalkyl,
benzyl,
C₁₋₆alkyl, and
C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R^{21a} is selected from:
aryl,
aryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN,
cycloalkyl,
heteroaryl, and
heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN;
R^{22a} and R^{23a} are each independently selected from hydrogen and C₁₋₆alkyl,
or R^{22a} and R^{23a} taken together with the carbon atoms to which they are attached represent a 3 or 4 member cycloalkyl;
R^{24a} is selected from: hydrogen, methyl, —CF₃, fluoro and chloro;
R^{25a} is selected from hydrogen and C₁₋₆alkyl;
R^{26a} is selected from:
hydrogen,
cycloalkyl,
heterocycloalkyl,
heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, —NO₂, —NH₂ and —CN,
C₁₋₆alkyl, and
C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R^{27a} is selected from: hydrogen and —CH₃;
R^{28a} is selected from: hydrogen, methyl, —CF₃, fluoro and chloro;
Y is CH or N; and
X is CR^{102a} or N,
where R^{102a} is selected from: hydrogen, fluoro and chloro;
or a salt thereof including a pharmaceutically acceptable salt thereof.

4. The compound of claim 1 represented by the following Formula (IVa): 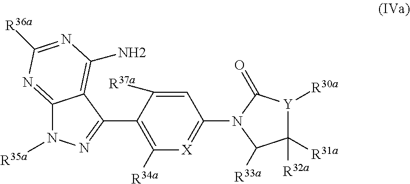
wherein:
R^{30a} is selected from:
hydrogen,
cycloalkyl,
phenyl,
benzyl,
heterocycloalkyl,
heteroaryl,
C₁₋₆alkyl, and
C₁₋₆alkyl substituted with from one to five substituents independently selected from: fluoro, chloro, bromo, iodo, C₁₋₄alkyl, C₁₋₄alkyloxy, —OH, —COOH, —CF₃, —C₁₋₄alkylOC₁₋₄alkyl, —NO₂, —NH₂ and —CN;
R^{31a} is selected from:
aryl,
aryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN,
cycloalkyl,
heteroaryl, and
heteroaryl substituted with from one to five substituents independently selected from: fluoro, chloro, bromo, iodo, C₁₋₄alkyl, cycloalkyl, C₁₋₄alkyloxy, —OH, —COOH, —CF₃, —OC(H)F₂, —C(H)F₂, —OCH₂F, —CH₂F, —OCF₃, —C₁₋₄alkylOC₁₋₄alkyl, —NO₂, —NH₂ and —CN;
R^{32a} and R^{33a} are each independently selected from hydrogen and C₁₋₆alkyl,
or R^{32a} and R^{33a} taken together with the carbon atoms to which they are attached represent a 3 or 4 member cycloalkyl;
R^{34a} is selected from: hydrogen, methyl, —CF₃, fluoro and chloro;
R^{35a} is selected from:
hydrogen,
cycloalkyl,
heterocycloalkyl,
heterocycloalkyl substituted with from 1 to 4 substituents independently selected from: C₁₋₄alkyl, C₁₋₄alkyloxy, —OH, —COOH, —CF₃, —C₁₋₄alkylOC₁₋₄alkyl, —NO₂, —NH₂ and —CN,
C₁₋₆alkyl, and
C₁₋₆alkyl substituted with from 1 to 4 substituents independently selected from: fluoro, chloro, bromo, iodo, C₁₋₄alkyloxy, —OH, —CF₃, —COOH, —NO₂, —NH₂ and —CN;
R^{36a} is selected from: hydrogen and —CH₃;
R^{37a} is selected from: hydrogen, methyl, —CF₃, fluoro and chloro;
Y is CH or N; and
X is CR^{103a} or N,
where R^{103a} is selected from: hydrogen, fluoro and chloro;
or a salt thereof including a pharmaceutically acceptable salt thereof.

5. The compound of claim 1 selected from:
1-(4-(4-amino-7-methyl-7H-pyrrolo[2,3-d]pyrimidin-5-yl)-3-fluorophenyl)-4-(2,5-difluorophenyl)-3-isobutylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one;
1-(4-(4-amino-7-(2,2-difluoroethyl)-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-3-benzyl-4-(2,5-difluorophenyl)imidazolidin-2-one;
1-(4-(4-amino-7-methyl-7H-pyrrolo[2,3-d]pyrimidin-5-yl)-3-fluorophenyl)-4-(2,5-difluorophenyl)imidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-ethylimidazolidin-2-one;
1-(4-(4-amino-7-methyl-7H-pyrrolo[2,3-d]pyrimidin-5-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-2-fluorophenyl)-3-methylimidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-ethylimidazolidin-2-one;
1-(4-(4-amino-7-methyl-7H-pyrrolo[2,3-d]pyrimidin-5-yl)-3-fluorophenyl)-4-cyclopentylpyrrolidin-2-one;
1-(4-(4-amino-7-methyl-7H-pyrrolo[2,3-d]pyrimidin-5-yl)phenyl)-4-(3,5-difluorophenyl)pyrrolidin-2-one;
1-(4-(4-amino-7-methyl-7H-pyrrolo[2,3-d]pyrimidin-5-yl)-3-fluorophenyl)-4-(3,5-dimethylphenyl)pyrrolidin-2-one;
1-(4-(4-amino-7-methyl-7H-pyrrolo[2,3-d]pyrimidin-5-yl)-3-fluorophenyl)-4-(2,5-difluorophenyl)pyrrolidin-2-one;
1-(5-(4-amino-7-methyl-7H-pyrrolo[2,3-d]pyrimidin-5-yl)pyridin-2-yl)-4-(2,5-difluorophenyl)pyrrolidin-2-one;
1-(4-(4-amino-7-methyl-7H-pyrrolo[2,3-d]pyrimidin-5-yl)-3-fluorophenyl)-4-(6-methylpyridin-2-yl)pyrrolidin-2-one;
1-(4-(4-amino-7-methyl-7H-pyrrolo[2,3-d]pyrimidin-5-yl)-3-fluorophenyl)-(2,4-difluorophenyl)pyrrolidin-2-one;
1-(4-(4-amino-7-methyl-7H-pyrrolo[2,3-d]pyrimidin-5-yl)-3-fluorophenyl)-4-(2,4-fluorophenyl)pyrrolidin-2-one;
1-(4-(4-amino-7-methyl-7H-pyrrolo[2,3-d]pyrimidin-5-yl)-3-fluorophenyl)-4-(2,5-tolyl)imidazolidin-2-one;
1-(4-(4-amino-2,7-dimethyl-7H-pyrrolo[2,3-d]pyrimidin-5-yl)-3-fluorophenyl-4-(2,5-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl-4-(2,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-methyl-4-(m-tolyl)imidazolidin-2-one;
1-(5-(4-amino-7-methyl-7H-pyrrolo[2,3-d]pyrimidin-5-yl)pyridin-2-yl)-4-(2,5-difluorophenyl)-3-methylimidazolidin-2-one;
1-(5-(4-amino-7-methyl-7H-pyrrolo[2,3-d]pyrimidin-5-yl)pyridin-2-yl)-4-(2,5-difluorophenyl)-3-ethylimidazolidin-2-one;
1-(4-(4-aminothieno[2,3-d]pyrimidin-5-yl)-3-fluorophenyl)-4-(2,5-difluorophenyl)-3-ethylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one;
1-(4-(4-amino-2,7-dimethyl-7H-pyrrolo[2,3-d]pyrimidin-5-yl)-3-fluorophenyl-4-(2,5-difluorophenyl)-3-ethylimidazolidin-2-one; (enantiomer 1)
1-(4-(4-amino-2,7-dimethyl-7H-pyrrolo[2,3-d]pyrimidin-5-yl)-3-fluorophenyl-4-(2,5-difluorophenyl)-3-ethylimidazolidin-2-one; (enantiomer 2)
1-(4-(4-amino-7-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(5-cyclopropyl-2-fluorophenyl)-3-ethylimidazolidin-2-one;
1-(4-(4-amino-7-methyl-7H-pyrrolo[2,3-d]pyrimidin-5-yl)-3-fluorophenyl)-4-(5-cyclopropyl-2-fluorophenyl)-3-methylimidazolidin-2-one;
1-(4-(4-amino-7-methyl-7H-pyrrolo[2,3-d]pyrimidin-5-yl)-3-fluorophenyl)-3-ethyl-4-(6-methylpyridin-2-yl)imidazolidin-2-one;
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3-chloro-5-fluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(5-chloro-2-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,5-difluorophenyl)-3-ethylimidazolidin-2-one; (enantiomer1)
1-(4-(4-amino-7-methyl-7H-pyrrolo[2,3-d]pyrimidin-5-yl)-3-fluorophenyl)-3-ethyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one;
1-(4-(4-amino-7-methyl-7H-pyrrolo[2,3-d]pyrimidin-5-yl)-3-fluorophenyl)-3-ethyl-4-(4-fluorophenyl)imidazolidin-2-one;
1-(4-(4-amino-7-methyl-7H-pyrrolo[2,3-d]pyrimidin-5-yl)-3-fluorophenyl)-4-cyclohexyl-3-methylimidazolidin-2-one;
1-(4-(4-amino-7-methyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-3-methyl-4-(pyridin-3-yl)imidazolidin-2-one;
1-(4-(4-amino-2,7-dimethyl-7H-pyrrolo[2,3-d]pyrimidin-5-yl)-3-fluorophenyl)-4-cyclohexyl-3-methylimidazolidin-2-one (enantiomer 1)
1-(4-(4-amino-7-methyl-7H-pyrrolo[2,3-d]pyrimidin-5-yl)-3-fluorophenyl)-4-cyclohexyl-3-methylimidazolidin-2-one; (enantiomer 1)
1-(4-(4-amino-7-methyl-7H-pyrrolo[2,3-d]pyrimidin-5-yl)-3-fluorophenyl)-4-cyclohexyl-3-methylimidazolidin-2-one; (enantiomer 2)
1-(4-(4-amino-2,7-dimethyl-7H-pyrrolo[2,3-d]pyrimidin-5-yl)-3-fluorophenyl)-4-(4-methoxyphenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(2,4-difluorophenyl)-3-ethylimidazolidin-2-one; (enantiomer 2)
1-(4-(4-amino-7-methyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-3-methyl-4-(2,4,6-trifluorophenyl)imidazolidin-2-one; (enantiomer 1)
1-(4-(4-amino-2,7-dimethyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-4-(3-(difluoromethoxy)phenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one;
1-(4-(4-amino-7-methyl-7H-pyrrolo[2,3-d]pyrimidin-5-yl)-3-fluorophenyl)-4-(2,3-difluorophenyl)-3-ethylimidazolidin-2-one;
1-(4-(4-amino-7-methyl-7H-pyrrolo[2,3-d]pyrimidin-5-yl)-3-fluorophenyl)-3-(2,2-difluoroethyl)-4-(2,4-difluorophenyl)imidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(2,4-difluorophenyl)-3-(2,2,2-trifluoroethyl)imidazolidin-2-one;
1-(4-(4-amino-2,7-dimethyl-7H-pyrrolo[2,3-d]pyrimidin-5-yl)-3-fluorophenyl)-4-(3,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,6-difluorophenyl)-3-methylimidazolidin-2-one;
1-(4-(4-amino-7-(2,2,2-trifluoroethyl)-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(2,4-difluorophenyl)-3-(2,2,2-trifluoroethyl)imidazolidin-2-one; (enantiomer 1)
1-(4-(4-amino-7-methyl-7H-pyrrolo[2,3-d]pyrimidin-5-yl)-3-fluorophenyl)-4-(3-fluoro-5-(trifluoromethyl)phenyl)-3-methylimidazolidin-2-one;
1-(4-(4-amino-7-methyl-7H-pyrrolo[2,3-d]pyrimidin-5-yl)-3-fluorophenyl)-3-ethyl-4-fluoro-5-(trifluoromethyl)phenyl)imidazolidin-2-one;
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one; (enantiomer 1)
1-(4-(4-amino-7-isopropyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-ethyl-4-fluoro-5-(trifluoromethyl)phenyl)imidazolidin-2-one; (enantiomer 1)
1-(4-(4-amino-7-methyl-7H-pyrrolo[2,3-d]pyrimidin-5-yl)-3-fluorophenyl)-3-ethyl-4-fluoro-5-(trifluoromethyl)phenyl)imidazolidin-2-one; (enantiomer 1)
1-(4-(4-amino-7-cyclopropyl-2-methyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-7-cyclopropyl-2-methyl-7H-pyrrolo[2,3-d]pyrimidin-5-yl)-3-fluorophenyl)-4-(4-fluorophenyl)-3-methylimidazolidin-2-one;
1-(4-(4-amino-7-isopropyl-7H-pyrrolo[2,3-d]pyrimidin-5-yl)-3-fluorophenyl)-4-(2,4-difluorophenyl)-3-methylimidazolidin-2-one;
1-(4-(4-amino-6,7-dimethyl-7H-pyrrolo[2,3-d]pyrimidin-5-yl)-3-fluorophenyl)-4-(2,4-difluorophenyl)-3-ethylimidazolidin-2-one;
1-(4-(4-amino-6,7-dimethyl-7H-pyrrolo[2,3-d]pyrimidin-5-yl)-3-fluorophenyl)-4-(2,4-difluorophenyl)-3-methylimidazolidin-2-one;
1-(4-(4-amino-7-isopropyl-2-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,4-difluorophenyl)-3-ethylimidazolidin-2-one;
1-(4-(4-amino-1-methyl-1H-pyrazolo[3,4-d]pyrimidin-3-yl)-3-fluorophenyl)-4-(2,4-difluorophenyl)-3-ethylimidazolidin-2-one;
1-(4-(4-amino-1-methyl-1H-pyrazolo[3,4-d]pyrimidin-3-yl)-3-fluorophenyl)-4-(2,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,5-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(3-methoxyphenyl)-3-methylimidazolidin-2-one;
1-(4-(4-amino-7-methyl-7H-pyrrolo[2,3-d]pyrimidin-5-yl)-3-fluorophenyl)-4-(3-methoxyphenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(3,5-difluorophenyl)-3-methylimidazolidin-2-one; (enantiomer 1)
1-(4-(4-amino-2,7-dimethyl-7H-pyrrolo[2,3-d]pyrimidin-5-yl)-3,5-difluorophenyl)-4-(2,4-difluorophenyl)-3-methylimidazolidin-2-one;
1-(4-(4-amino-7-isopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,4-difluorophenyl)-3-ethylimidazolidin-2-one;
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one;
1-(4-(4-amino-7-ethyl-2-methyl-7H-pyrrolo[2,3-d]pyrimidin-5-yl)-3-fluorophenyl)-4-(2,4-difluorophenyl)-3-methylimidazolidin-2-one;
1-(4-(4-amino-2,7-dimethyl-7H-pyrrolo[2,3-d]pyrimidin-5-yl)-3-fluorophenyl)-4-(5-fluoropyridin-3-yl)-3-methylimidazolidin-2-one; (enantiomer 1)
1-(4-(4-amino-7-methyl-7H-pyrrolo[2,3-d]pyrimidin-5-yl)-3-fluorophenyl)-4-(5-fluoro-6-methylpyridin-2-yl)-3-methylimidazolidin-2-one;
1-(4-(4-amino-7-methyl-7H-pyrrolo[2,3-d]pyrimidin-5-yl)-3-fluorophenyl)-4-(5-fluoro-6-methylpyridin-2-yl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,4-difluorophenyl)-3-methylimidazolidin-2-one;
1-(4-(4-amino-7-methyl-7H-pyrrolo[2,3-d]pyrimidin-5-yl)-3-fluorophenyl)-4-(3,4-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,4-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,4-difluorophenyl)-3-methylimidazolidin-2-one; (enantiomer 2)
1-(4-(4-amino-7-methyl-7H-pyrrolo[2,3-d]pyrimidin-5-yl)-3-methylphenyl)-4-(3,5-difluorophenyl)-3-methylimidazolidin-2-one;
1-(4-(4-amino-7-cyclopropyl-7H-pyrrolo[2,3-d]pyrimidin-5-yl)-3-methylphenyl)-4-(3,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-chlorophenyl)-4-(3,5-difluorophenyl)-3-methylimidazolidin-2-one;
1-(4-(4-amino-7-cyclopropyl-7H-pyrrolo[2,3-d]pyrimidin-5-yl)-3-chlorophenyl)-4-(3,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one; (enantiomer 1)
1-(4-(4-amino-7-cyclopropyl-7H-pyrrolo[2,3-d]pyrimidin-5-yl)-3-fluorophenyl)-4-(2,5-difluorophenyl)-3-ethylimidazolidin-2-one; (enantiomer 2)
1-(4-(4-amino-7-cyclopropyl-7H-pyrrolo[2,3-d]pyrimidin-5-yl)-3-fluorophenyl)-4-(3-fluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3-fluorophenyl)-3-methylimidazolidin-2-one; (enantiomer 2)
1-(4-(4-amino-7-methyl-7H-pyrrolo[2,3-d]pyrimidin-5-yl)-3-chlorophenyl)-4-(3,5-difluorophenyl)-3-methylimidazolidin-2-one; (enantiomer 1)
1-(4-(4-amino-7-methyl-7H-pyrrolo[2,3-d]pyrimidin-5-yl)-3-chlorophenyl)-4-(3,5-difluorophenyl)-3-methylimidazolidin-2-one; (enantiomer 2)
1-(4-(4-amino-7-cyclopropyl-7H-pyrrolo[2,3-d]pyrimidin-5-yl)-3-chlorophenyl)-4-(3,5-difluorophenyl)-3-methylimidazolidin-2-one; (enantiomer 1)
1-(4-(4-amino-7-cyclopropyl-7H-pyrrolo[2,3-d]pyrimidin-5-yl)-3-chlorophenyl)-4-(3,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(3-chlorophenyl)-4-fluorophenyl)-3-methylimidazolidin-2-one;
1-(4-(4-amino-7-cyclopropyl-7H-pyrrolo[2,3-d]pyrimidin-5-yl)-3-fluorophenyl)-4-(3-chloro-4-fluorophenyl)-3-methylimidazolidin-2-one;
1-(4-(4-amino-7-methyl-7H-pyrrolo[2,3-d]pyrimidin-5-yl)-3-fluorophenyl)-4-(3-chlorophenyl)-4-fluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3-chloro-4-fluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(6-chloropyridin-3-yl)-3-methylimidazolidin-2-one;
1-(4-(4-amino-2,7-dimethyl-7H-pyrrolo[2,3-d]pyrimidin-5-yl)-3-fluorophenyl)-4-(6-chloropyridin-3-yl)-3-methylimidazolidin-2-one; (enantiomer 2)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-cycloheptyl-3-methylimidazolidin-2-one;
1-(4-(4-amino-2,7-dimethyl-7H-pyrrolo[2,3-d]pyrimidin-5-yl)-3-fluorophenyl)-3-methyl-4-(2,3,6-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one; (enantiomer 1)
1-(4-(4-amino-7-methyl-7H-pyrrolo[2,3-d]pyrimidin-5-yl)-3-fluorophenyl)-3-methyl-4-(2,3,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-3-methyl-4-(2,3,5-trifluorophenyl)imidazolidin-2-one; (enantiomer 1)
1-(4-(4-amino-7-cyclopropyl-7H-pyrrolo[2,3-d]pyrimidin-5-yl)-3-fluorophenyl)-3-methyl-4-(2,3,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(2,5-dihydrobenzofuran-5-yl)-3-methylimidazolidin-2-one;
1-(4-(4-amino-7-cyclopropyl-7H-pyrrolo[2,3-d]pyrimidin-5-yl)-3-fluorophenyl)-4-(2,5-chlorophenyl)-3-methylimidazolidin-2-one;
1-(4-(4-amino-7-cyclopropyl-7H-pyrrolo[2,3-d]pyrimidin-5-yl)-3-fluorophenyl)-4-(2,3-methyl-4-phenylimidazolidin-2-one;
1-(4-(4-amino-7-methyl-7H-pyrrolo[2,3-d]pyrimidin-5-yl)-3-fluorophenyl)-4-(2,3-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,5-difluorophenyl)-3-methylimidazolidin-2-one; (enantiomer 2)
1-(4-(4-amino-7-cyclopropyl-7H-pyrrolo[2,3-d]pyrimidin-5-yl)-3-fluorophenyl)-4-(3-chlorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3-chlorophenyl)-3-methylimidazolidin-2-one; (enantiomer 2)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one;
1-(4-(4-amino-7-cyclopropyl-7H-pyrrolo[2,3-d]pyrimidin-5-yl)-3-fluorophenyl)-3-methyl-4-(2,4,5-trifluorophenyl)imidazolidin-2-one;
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one; (enantiomer 1)
1-(4-(4-amino-7-methyl-7H-pyrrolo[2,3-d]pyrimidin-5-yl)-3-fluorophenyl)-3-methyl-4-(2,4,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-3-methyl-4-(2,4,5-trifluorophenyl)imidazolidin-2-one; (enantiomer 1)
1-(4-(4-amino-7-cyclopropyl-7H-pyrrolo[2,3-d]pyrimidin-5-yl)-3-fluorophenyl)-3-methyl-4-(2,4,5-trifluorophenyl)imidazolidin-2-one; (enantiomer 2)
1-(4-(4-amino-7-cyclopropyl-7H-pyrrolo[2,3-d]pyrimidin-5-yl)-3-fluorophenyl)-4-(4-fluorophenyl)-3-methylimidazolidin-2-one;
1-(4-(4-amino-7-cyclopropyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(4-fluorophenyl)-3-methylimidazolidin-2-one; (enantiomer 2)
1-(4-(4-amino-7-cyclopropyl-7H-pyrrolo[2,3-d]pyrimidin-5-yl)-3-fluorophenyl)-3-methyl-4-phenylimidazolidin-2-one; (enantiomer 1)
1-(4-(4-amino-7-cyclopropyl-7H-pyrrolo[2,3-d]pyrimidin-5-yl)-3-fluorophenyl)-3-methyl-4-phenylimidazolidin-2-one; (enantiomer 2)
1-(4-(4-amino-7-cyclopropyl-7H-pyrrolo[2,3-d]pyrimidin-5-yl)-3-fluorophenyl)-4-(4-chlorophenyl)-3-methylimidazolidin-2-one;
1-(4-(4-amino-7-cyclopropyl-7H-pyrrolo[2,3-d]pyrimidin-5-yl)-3-fluorophenyl)-4-(4-chlorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(2,3-dihydrobenzofuran-5-yl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(3,5-difluorophenyl)-3-ethylimidazolidin-2-one;
1-(4-(4-amino-7-methyl-7H-pyrrolo[2,3-d]pyrimidin-5-yl)-3-fluorophenyl)-4-(3,5-difluorophenyl)-3-ethylimidazolidin-2-one; (enantiomer 1)
1-(4-(4-amino-7-methyl-7H-pyrrolo[2,3-d]pyrimidin-5-yl)-3-fluorophenyl)-4-(3,5-difluorophenyl)-3-ethylimidazolidin-2-one; (enantiomer 2)
1-(4-(4-amino-7-cyclopropyl-7H-pyrrolo[2,3-d]pyrimidin-5-yl)-3-fluorophenyl)-4-(3,5-difluorophenyl)-3-ethylimidazolidin-2-one; (enantiomer 1) and
1-(4-(4-amino-7-cyclopropyl-7H-pyrrolo[2,3-d]pyrimidin-5-yl)-3-fluorophenyl)-4-(3,5-difluorophenyl)-3-ethylimidazolidin-2-one; (enantiomer 2)
or a salt thereof including a pharmaceutically acceptable salt thereof.

6. The compound of claim 1 selected from:
1-(4-(4-amino-7-methyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-ethylimidazolidin-2-one; (enantiomer 1)
1-(4-(4-amino-7-methyl-7H-pyrrolo[2,3-d]pyrimidin-5-yl)-3-fluorophenyl)-4-(2,5-difluorophenyl)-3-ethylimidazolidin-2-one; (enantiomer 2)
1-(4-(4-Amino-7-methyl-7H-pyrrolo[2,3-d]pyrimidin-5-yl)-3-fluorophenyl)-4-(3,5-difluorophenyl)pyrrolidin-2-one;
1-(4-(4-Amino-7-methyl-7H-pyrrolo[2,3-d]pyrimidin-5-yl)-3-methylphenyl)-4-(3,5-difluorophenyl)pyrrolidin-2-one;
1-(4-(4-Amino-7-methyl-7H-pyrrolo[2,3-d]pyrimidin-5-yl)-3-fluorophenyl)-4-cyclohexylpyrrolidin-2-one;
1-(4-(4-amino-7-methyl-7H-pyrrolo[2,3-d]pyrimidin-5-yl)-3-fluorophenyl)-3-methyl-4-(pyridin-2-yl)imidazolidin-2-one;
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-2,6,7-trimethyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one; (enantiomer 1)
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-3-methyl-4-(pyridin-4-yl)imidazolidin-2-one;
1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one;
1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one; (enantiomer 1) and
1-(4-(4-amino-7-cyclopropyl-7H-pyrrolo[2,3-d]pyrimidin-5-yl)-3-fluorophenyl)-4-(3,5-difluorophenyl)-3-methylimidazolidin-2-one; (enantiomer 2)
or a salt thereof including a pharmaceutically acceptable salt thereof.

7. The compound of claim 1 selected from:
1-(4-(4-amino-7-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-7-methyl-7H-pyrrolo[2,3-d]pyrimidin-5-yl)-3-fluorophenyl)-4-(3,5-difluorophenyl)-3-methylimidazolidin-2-one; (enantiomer 1)
1-(4-(4-amino-7-cyclopropyl-7H-pyrrolo[2,3-d]pyrimidin-5-yl)-3-fluorophenyl)-4-(2,4-difluorophenyl)-3-methylimidazolidin-2-one; (enantiomer 2)
1-(4-(4-amino-7-methyl-7H-pyrrolo[2,3-d]pyrimidin-5-yl)-3-fluorophenyl)-4-(3,4-difluorophenyl)-3-methylimidazolidin-2-one; (enantiomer 1) and
1-(4-(4-amino-7-methyl-7H-pyrrolo[2,3-d]pyrimidin-5-yl)-3-fluorophenyl)-4-(4-fluorophenyl)-3-methylimidazolidin-2-one; (enantiomer 1) or a salt thereof including a pharmaceutically acceptable salt thereof.

8. The compound of claim 1 selected from:
1-(4-(4-amino-7-methyl-7H-pyrrolo[2,3-d]pyrimidin-5-yl)-3-fluorophenyl)-4-(2,5-difluorophenyl)-3-ethylimidazolidin-2-one;
1-(4-(4-amino-7-methyl-7H-pyrrolo[2,3-d]pyrimidin-5-yl)-3-fluorophenyl)-4-(2,5-difluorophenyl)-3-ethylimidazolidin-2-one; (enantiomer 1)
1-(4-(4-amino-7-methyl-7H-pyrrolo[2,3-d]pyrimidin-5-yl)-3-fluorophenyl)-4-(2,5-difluorophenyl)-3-ethylimidazolidin-2-one; (enantiomer 2)
1-(4-(4-amino-7-cyclopropyl-2-methyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-1,6-dimethyl-1H-pyrazolo[3,4-d]pyrimidin-3-yl)-3-fluorophenyl)-4-(2,4-difluorophenyl)-3-methylimidazolidin-2-one, (enantiomer 1);
1-(4-(4-amino-7-methyl-7H-pyrrolo[2,3-d]pyrimidin-5-yl)-3-fluorophenyl)-4-cyclohexyl-3-methylimidazolidin-2-one;
1-(4-(4-amino-7-methyl-7H-pyrrolo[2,3-d]pyrimidin-5-yl)-3-fluorophenyl)-4-(4-fluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(2,4-difluorophenyl)-3-methylimidazolidin-2-one; (enantiomer 1)
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one;
1-(4-(4-amino-2,7-dimethyl-7H-pyrrolo[2,3-d]pyrimidin-5-yl)-3-fluorophenyl)-4-(4-fluorophenyl)-3-methylimidazolidin-2-one; (enantiomer 1) and
1-(4-(4-amino-7-methyl-7H-pyrrolo[2,3-d]pyrimidin-5-yl)-3-fluorophenyl)-4-(3,5-difluorophenyl)-3-methylimidazolidin-2-one; (enantiomer 1)
or a salt thereof including a pharmaceutically acceptable salt thereof.

9. A pharmaceutical composition comprising a compound of Formula (I) according to claim 1 or a pharmaceutically acceptable salt thereof and a pharmaceutically acceptable excipient.

10. A method of treating a disease selected from: cancer, pre-cancerous syndromes, spinal cord injury, traumatic brain injury, ischemic stroke, stroke, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, cognitive impairment, atherosclerosis, ocular diseases, arrhythmias, in organ transplantation and in the transportation of organs for transplantation, in a mammal in need thereof, which comprises administering to such mammal a therapeutically effective amount of a compound of Formula I, as described in claim 1 or a pharmaceutically acceptable salt thereof.

11. The method of claim 10 wherein the mammal is a human.

12. A method of treating a disease selected from: cancer, pre-cancerous syndromes, spinal cord injury, traumatic brain injury, ischemic stroke, stroke, diabetes, metabolic syndrome, metabolic disorders, Huntington's disease, Creutzfeldt-Jakob Disease, fatal familial insomnia, Gerstmann-Sträussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, cognitive impairment, atherosclerosis, ocular diseases, arrhythmias, in organ transplantation and in the transportation of organs for transplantation, in a mammal in need thereof, which comprises administering to such mammal a therapeutically effective amount of a compound of claim 5 or a pharmaceutically acceptable salt thereof.

13. The method of claim 12 wherein the mammal is a human.

14. (canceled)

15. (canceled)

16. (canceled)

17. The method of inhibiting PERK activity in a human in need thereof, which comprises administering to such human a therapeutically effective amount of a compound of Formula I, as described in claim 1 or a pharmaceutically acceptable salt thereof.

18. (canceled)

19. A method of treating cancer in a mammal in need thereof, which comprises: administering to such mammal a therapeutically effective amount of
a) a compound of Formula (I), as described in claim 1 or a pharmaceutically acceptable salt thereof; and
b) at least one anti-neoplastic agent.

20. (canceled)

21. (canceled)

22. (canceled)

23. The method according to claim 191 wherein said cancer is selected from: breast cancer, inflammatory breast cancer, ductal carcinoma, lobular carcinoma, colon cancer, pancreatic cancer, insulinomas, adenocarcinoma, ductal adenocarcinoma, adenosquamous carcinoma, acinar cell carcinoma, glucagonoma, skin cancer, melanoma, metastatic melanoma, lung cancer, small cell lung cancer, non-small cell lung cancer, squamous cell carcinoma, adenocarcinoma, large cell carcinoma, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid,
lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia,
malignant lymphoma, hodgkins lymphoma, non-hodgkins lymphoma, lymphoblastic T cell lymphoma, Burkitt's lymphoma, follicular lymphoma,
neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

24. (canceled)

25. A process for preparing a pharmaceutical composition containing a pharmaceutically acceptable excipient and an effective amount of a compound of Formula (I) as described in claim 1 or a pharmaceutically acceptable salt thereof, which process comprises bringing the compound of Formula (I) or a pharmaceutically acceptable salt thereof into association with a pharmaceutically acceptable excipient.

26. (canceled)

27. (canceled)

28. A method of treating ocular diseases in a human in need thereof, which comprises administering to such human a therapeutically effective amount of a compound of Formula I, as described in claim 1 or a pharmaceutically acceptable salt thereof.

29. (canceled)

30. (canceled)

31. A method of treating neurodegeneration in a human in need thereof, which comprises administering to such human a therapeutically effective amount of a compound of Formula I, as described in claim 1 or a pharmaceutically acceptable salt thereof.

32. A method of preventing organ damage during the transportation of organs for transplantation, which comprises adding a compound of Formula (I) as described in claim 1 to the solution housing the organ during transportation.

33. (canceled)

34. (canceled)

35. (canceled)
